(19) United States
(12) Patent Application Publication
(10) Pub. No.: US 2006/0003931 A1
(43) Pub. Date: Jan. 5, 2006

(54) Crystal structure of the hepatocyte growth factor and methods of use

(75) Inventors: Charles W. Eigenbrot JR., Burlingame, CA (US); Daniel K. Kirchhofer, Los Altos, CA (US); Robert A. Lazarus, Millbrae, CA (US)

Correspondence Address: MERCHANT & GOULD PC, P.O. BOX 2903, MINNEAPOLIS, MN, 55402-0903, US

(73) Assignee: Genentech, Inc., South San Francisco, CA (US)

(21) Appl. No.: 11/124,635

(22) Filed: May 6, 2005

Related U.S. Application Data
(60) Provisional application No. 60/569,301, filed on May 6, 2004.

(51) Int. Cl. A61K 38/18; G06F 19/00; C07K 14/475

(52) U.S. Cl. 514/12; 530/399; 702/19

(57) ABSTRACT

The disclosure provides a crystal and crystal structure of the Hepatocyte Growth Factor Beta (HGF β) Chain, as well as use of the crystal structure in the design, identification, and selection of modulators of HGF or Met activity.

### CROSS REFERENCE TO RELATED APPLICATIONS

This application claims the benefit of 35 USC 119(e) to U.S. Ser. No. 60/569,301, filed May 6, 2004, which application is incorporated herein by reference.

### BACKGROUND

Hepatocyte growth factor (HGF) also known as scatter factor (SF), is the ligand for Met (Bottaro et al., 1991), a receptor tyrosine kinase encoded by the c-met protooncogene (Cooper et al., 1984). HGF binding to Met induces phosphorylation of the intracellular kinase domain resulting in activation of a complex set of intracellular pathways that lead to cell growth, differentiation and migration in a variety of cell types; several recently published reviews provide a comprehensive overview (Birchmeier et al., 2003; Trusolino and Comoglio, 2002; Maulik et al., 2002). In addition to its fundamental importance in embryonic development and tissue regeneration, the HGF/Met signaling pathway has also been implicated in invasive tumor growth and metastasis and as such represents an interesting therapeutic target (Birchmeier et al., 2003; Trusolino and Comoglio, 2002; Danilkovitch-Miagkova and Zbar, 2002; Ma et al., 2003).

HGF belongs to the plasminogen-related growth factor family and comprises a 69 kDa α-chain, containing the N-terminal finger domain (N) and four Kringle (K1-K4) domains, and a 34 kDa β-chain, which has strong similarity to protease domains of chymotrypsin-like serine proteases from Clan PA(S)/FamilyS1 (Nakamura et al., 1989; Donate et al., 1994; Rawlings et al., 2002). Like plasminogen and other serine protease zymogens, HGF is secreted as a single chain precursor form (scHGF). scHGF binds to heparin sulfate proteoglycans, such as syndecan-1 (Derksen et al., 2002) on cell surfaces or in the extracellular matrix. Heparin sulfate proteoglycans bind to the N domain (Hartmann et al., 1998), which also contributes to the high affinity Met binding together with amino acids located in K1 (Lokker et al., 1994). Although scHGF is able to bind Met with high affinity, it cannot activate the receptor (Lokker et al., 1992; Hartmann et al., 1992). Acquisition of HGF signaling activity is contingent upon proteolytic cleavage (activation) of scHGF at Arg494-Val495 resulting in the formation of mature HGF, a disulfide-linked α/β heterodimer (Lokker et al., 1992; Hartmann et al., 1992; Naldini et al., 1992). The protease-like domain of HGF (HGF β-chain) lacks the Asp [c102]-His [c57]-Ser [c195] (standard chymotrypsinogen numbering in brackets used throughout) catalytic triad found in all serine proteases (Perona and Craik, 1995; Hedstrom, 2002), having a Gln534 [c57] and Tyr673 [c195], and thus is devoid of any enzymatic activity.

Currently, there is no detailed structural information about HGF β-chain or HGF β-chain binding and activation of Met. A completely solved crystal structure of the HGF β-chain can be used, for example, in assays for Met-ligand (e.g., HGF β-chain) interaction and function, modeling the structure-function relationship of Met and other molecules, diagnostic assays for mutation-induced pathologies, and rational design of agents useful in modulating Met or HGF activity.

### SUMMARY

In some embodiments, the present disclosure provides a crystal of hepatocyte growth factor beta chain (HGF β) and the structural coordinates of the crystal. Coordinates of the crystal structure are listed in Table 5. In some embodiments, HGF β has an amino acid sequence of SEQ ID NO:1, or conservative substitutions thereof.

In some embodiments, the disclosure provides a crystal structure of hepatocyte growth factor beta chain (HGF β), as well as use of the crystal structure to model HGF activity. This use of the structure includes modeling the interaction of ligands with the HGF β; activation and inhibition of HGF β; and the rational design of modulators of HGF and/or HGF β activity. For example, these modulators include ligands that interact with HGF β and modulate HGF β activities, such as cell migration, HGF β binding to Met, and Met phosphorylation, as well as molecules that mimic HGF β that can bind to a ligand but have altered ability to modulate the activity of a ligand.

In other embodiments, amino acid residues that form the binding site for the Met receptor on HGF β are identified and are useful, for example, in methods to model the structure of HGF binding site and to identify agents that can associate with, bind or fit into the binding site. Other structural features of HGF β have also been identified, including the active site, activation domain, a tunnel, and a HGF β dimerization region. Amino acid residues that form these structural features can also be used in methods to model the structure and to identify agents that can interact with these structural features.

### BRIEF DESCRIPTION OF FIGURES

FIG. 1A shows binding of HGF β to the extracellular domain of Met (Met ECD) by surface plasmon resonance. Arrows indicate the onset of the association and dissociation phases for a series of concentrations of HGF β. Data were analyzed by Global Fit using a 1:1 binding model from which kₒₙ, k_{off} and K_{d} values were determined.

FIG. 1B shows HGF β/Met-IgG competition ELISA. Competition binding of immobilized Met-IgG with 250 nM maleimide-coupled biotinylated wildtype HGF β and unlabeled HGF β (●) and proHGF β (▪) was carried out according to experimental procedures. Data from at least 3 independent determinations each were normalized, averaged and fitted by a four parameter fit using Kaleidagraph, from which IC₅₀ values were determined; error bars represent standard deviations.

FIG. 1C shows HGF dependent phosphorylation of Met in A549 cells was carried out according to the described methods using HGF (●) and HGF β (▪).

FIG. 1D shows inhibition of HGF dependent phosphorylation of Met was carried out in duplicate according to the described methods using HGF at 0.5 nM (♦), 0.25 nM (▴) and 0.125 nM (▪) to stimulate A549 cells in the presence of increasing concentrations of HGF β.

FIG. 1E shows full length HGF/Met-IgG competition ELISA. This was carried out similar to (FIG. 1B) using 1 nM NHS-coupled biotinylated HGF and unlabeled HGF (◯), and HGF β (●). Data from 3 independent determinations each were normalized, averaged and fitted as above.

FIG. 2A shows representative purity of HGF mutants. The purity of all HGF mutants analyzed by SDS-PAGE under reducing conditions is illustrated for cation exchange purified HGF 1623A. Incomplete conversion of the secreted single-chain form by CHO expression in 1% FBS (v/v) is shown in lane 1. Additional exposure to 5% FBS completed the activation process yielding pure two-chain HGF 1623A (lane 2). Molecular weight markers are shown as Mᵣ×10³.

FIG. 2B shows migration of MDA-MB435 cells in a transwell assay in the presence of 1 nM HGF mutants. Activities are expressed as percent migration of control cells exposed to 1 nM wildtype HGF; full length HGF sequence numbering is shown. Values represent the averages of 4-8 independent experiments±SD.

FIG. 2C shows photographs of MDA-MB435 cell migration in the absence of wildtype HGF (a), with 1 nM wildtype HGF, (b), 1 nM HGF R695A (c), and 1 nM HGF G696A (d).

FIG. 3 shows HGF dependent phosphorylation of Met by HGF mutants, in embodiments. Phosphorylation of Met of A549 cells was carried out according to the described methods using various concentrations of HGF (●), proHGF (♦), HGF Q534A (◯), HGF D578A (▴), HGF Y673A (Δ), HGF V692A (⋄), HGF R695A (□), and HGF G696A (▾).

FIG. 4 shows Met competition binding of HGF β mutants, in embodiments. The HGF β/Met-IgG competition ELISA described in FIG. 1B was used to assess Met binding of wildtype HGF β (Δ), HGF β (●), I699A (♦), HGF β Q534A (◯), HGF β D578A (▴), HGF β Y619A (⋄), HGF β G696A (▾), and HGF β R695A (□). Data were fitted by a four four parameter fit using Kaleidagraph; representative individual competition assays are shown for multiple independent determinations where n≧3.

FIG. 5A shows structure/electron density of HGF β ‘active-site’ region.

FIG. 5B shows a stereo view of ‘active-site’ regions of HGF β (dark grey) and plasmin (light grey). The pseudo-substrate inhibitor Glu-Gly-Arg-chloromethylketone from the plasmin structure (ball-and-stick) fills the ‘S1 pocket’ and interacts with its Asp [c189] side chain. The main chain amides that stabilize the oxyanion hole (spheres on dark grey tube) are structurally conserved in HGF β. The ‘P1’ residue of a substrate for a true enzyme binds in a pocket of the enzyme called the S1 subsite. The HGF β tunnel starts near where this ‘P1’ residue would insert.

FIG. 5C shows location of Met binding site on HGF β. Worm depiction of HGF β showing mutated residues with <20% (circled), 20%-60% (boxed) and 60%-80% (underlined) and >80% (plain number) of wildtype HGF pro-migratory activity (FIG. 4B).

FIG. 5D shows solvent-accessible surface of HGF β showing residues coded as in FIG. 5C. The dotted line delimits the region contacted by Met receptor as described in U.S. Ser. No. 60/568,865, filed May 6, 2004.

FIG. 6A shows intermolecular contacts in the HGF β X-ray structure. The reference molecule has three contacts. The molecule labeled ‘S’ arises from a 2-fold axis relating the N-terminal regions Val496-Arg502 [c17-c23] and adjacent residues. The molecule labeled ‘T’ arises from a 2-fold axis relating ‘active-site’ regions. Residue Cys604Ser (sphere) in the molecule labeled ‘U’ contacts the reference molecule in the [c70]-loop.

FIG. 6B shows partial sequences for HGF and homologous proteins at the border between α and β chains. HGF and chymotrypsinogen numbering are shown above and below the sequences, respectively. The boxed Cys in the α chain forms a disulfide bond with a Cys the β chain. Asterisks show residues that use the same residues found at corresponding positions in plasmin and dots represent conservative substitutions (SEQ ID NOs:9-13).

FIG. 6C shows superposition of HGF β (dark grey, thick) with plasmin (dark grey, thin). The C-terminal portion of the plasmin α-chain and the corresponding section from plasminogen (Peisach et al., 1999) are shown. The backbone path from HGF β Cys604 to Val495 (sphere labeled ‘N’) would differ from that used by plasmin/plasminogen. The small dark grey and light grey spheres are the site of a rare deletion in HGF (and MSP).

### DETAILED DESCRIPTION

A. Abbreviations

(Å) Ångström
(AA or aa) Amino acids
KIRA is kinase receptor activation assay
HEPES is 4-(2-hydroxyethyl)-1-piperazineethanesulfonic acid buffer
Met is a receptor tyrosine kinase for HGF
Met ECD is a Met extracellular domain
Met-IgG fusion protein is a fusion protein of the Met extracellular domain to an Ig constant region
MSP is macrophage stimulating protein
NK1 is a region of the α-chain of a HGF variant, see for example U.S. Pat. No. 5,849,689.
NK4 is a region of the α-chain of HGF
Ni-NTA metal chelate refers to nickel nitrilotriacetic resin
proHGF β is a single chain zymogen-like form of HGF β that is resistant to processing by HGF activating proteases
proHGF is a single chain precursor form of hepatocyte growth factor
scHGF is a single chain hepatocyte growth factor
SDS-PAGE is sodium dodecyl sulfonate-polyacrylamide gel electrophoresis
RON or Ron is a receptor tyrosine kinase for MSP
Ron:MSP is a Ron and MSP complex
TNM-FH media is a standard insect media available from Pharminogen
   B. Definitions

The term “hepatocyte growth factor” or “HGF”, as used herein, refers, unless specifically or contextually indicated otherwise, to any native or variant (whether native or synthetic) HGF polypeptide that is capable of binding to Met and/or activating the HGF/Met signaling pathway under conditions that permit such process to occur, for example, conditions that allow for the formation of the two chain form. The term “wild type HGF sequence” generally refers to an amino acid sequence found in a naturally occurring HGF and includes naturally occurring truncated or secreted forms, variant forms (e.g. alternatively spliced forms) and naturally occurring allelic variants.

“HGF β” or “HGF β-chain”, “HGF-beta” or variations thereof, refers to any HGF β chain having the conformation that is adopted by wild type HGF β chain upon conversion of wild type HGF protein from a single chain form to a 2 chain form (i.e., α and β chain). In some embodiments, the conversion results at least in part from cleavage between residue 494 and residue 495 of the wild type HGF protein. In some embodiments, the conformation refers specifically to the conformation of the activation domain of the protease-like domain in the β chain. In some embodiments, the conformation refers even more specifically to the conformation of the active site of the protease-like domain in the HGF β chain. Generally, adoption of the conformation reveals a Met binding site, as described herein. HGF β includes variants of wild type HGF β, for example, those shown in Table 1 and in SEQ ID NO:1. The HGF β chain may be isolated from a variety of sources such as human tissue or prepared by recombinant or synthetic methods. One embodiment of HGF β chain comprises an amino acid sequence of SEQ ID NO:1 in Table 7. Another embodiment of HGF β chain comprises an amino acid sequence of SEQ ID NO:5 in Table 9.

“HGF β variant” as used herein refers to polypeptide that has a different sequence than a reference polypeptide. In some embodiments, the reference polypeptide is a HGF β polypeptide comprising SEQ ID NO: 1 in Table 7. In some embodiments, a variant has at least 80% amino acid sequence identity with the amino acid sequence of Table 7 (SEQ ID NO: 1). The variants include those polypeptides that have substitutions, additions or deletions. The variants also include those polypeptides that have at least one conservative amino acid substitution, and preferably all substitutions are conservative. In some embodiments, the HGF β variant has about 1-25 conservative amino amino acid substitutions, more preferably about 1-20 conservative amino acids substitutions, more preferably about 1-10 conservative amino acid substitions, more preferably about 1-5 conservative amino acid substitutions, and more preferably about 1-2 conservative amino acid substitutions. In some embodiments, the variants have the biological activity of binding to the Met receptor and/or activating it. In other embodiments, the variant can bind to the Met receptor but not activate it.

Ordinarily, a HGF β variant polypeptide will have at least 80% sequence identity, more preferably at least 81% sequence identity, more preferably at least 82% sequence identity, more preferably at least 83% sequence identity, more preferably at least 84% sequence identity; more preferably at least 85% sequence identity, more preferably at least 86% sequence identity, more preferably at least 87% sequence identity, more preferably at least 88% sequence identity, more preferably at least 89% sequence identity, more preferably at least 90% sequence identity, more preferably at least 91% sequence identity, more preferably at least 92% sequence identity, more preferably at least 93% sequence identity, more preferably at least 94% sequence identity, more preferably at least 95% sequence identity, more preferably at least 96% sequence identity, more preferably at least 97% sequence identity, more preferably at least 98% sequence identity, more preferably at least 99% sequence identity or greater with a HGF β polypeptide having an amino acid sequence comprising SEQ ID NO: 1 or SEQ ID NO:5.

“Binding site” as used herein, refers to a region of a molecule or molecular complex that, as a result of its shape, distribution of electrostatic charge and/or distribution of non-polar regions, favorably associates with a ligand. Thus, a binding site may include or consist of features such as cavities, surfaces, or interfaces between domains. Ligands that may associate with a binding site include, but are not limited to, cofactors, substrates, receptors, agonists, and antagonists. Binding site refers to a functional binding site and/or a structural binding site. A structural binding site includes “in contact” amino acid residues as determined from examination of a three-dimensional structure. “Contact” can be determined using van der Waals radii of atoms, or by proximity sufficient to exclude solvent, typically water, from the space between a ligand and the molecule or molecular complex. “In contact” amino acid residues may not cause changes, for example, in a biochemical assay, a cell-based assay, or an in vivo assay used to define a functional binding site, but may contribute to the formation of the three-dimensional structure. Typically, at least one or more of “in contact” amino acid residues do not cause any change in these assays. A functional binding site includes amino acid residues that are identified as binding site residues based upon loss or gain of function, for example, loss of binding to ligand upon mutation of the residue. In some embodiments, the amino acid residues of a functional binding site are a subset of the amino acid residues of the structural binding site.

The term “HGF β structural binding site” includes all or a portion of a molecule or molecular complex whose shape, distribution of electrostatic charge and/or distribution of non-polar regions is sufficiently similar to at least a portion of a binding site of HGF β for Met as to be expected to bind Met or related structural analogs of Met. In some embodiments, a structurally equivalent ligand binding site is defined by a root mean square deviation from the structure coordinates of the backbone atoms of the amino acids that make up binding sites in HGF β of at most about 0.70 Å, preferably about 0.5 Å.

In some embodiments, a structural binding site for Met receptor on HGF comprises, consists essentially of, or consists of at least one amino acid residue corresponding to residues 513, 516, 533, 534, 537-539, 578, 619, 647, 656, 668-670, 673, 692-697, 699, 702, 705 or 707, or mixtures thereof. In some embodiments, a functional binding site comprises, consists essentially of, or consists of at least one amino acid residue corresponding to residues 534, 578, 673, 692, 694 to 696, or mixtures thereof.

“Active site” refers to a substrate binding cleft and a catalytic triad typically associated with a polypeptide with enzymatic activity. The substrate binding cleft includes the “S-1 binding site” where a substrate/enzyme interaction arises. The catalytic triad refers to 3 amino acids that are associated with an enzymatic activity of proteolysis. In typical serine proteases, the catalytic triad residues are Asp [c102], Ser[c195], and His[c57]. In a wild-type HGF molecule, the corresponding catalytic triad residues are Asp578, Tyr673, and Gln534. The active site of HGF β also includes amino acids that are a part of the Met binding site.

“Activation site” of HGF refers to a cleavage site that converts a single chain HGF to a two chain form including an alpha and beta chain. The cleavage at this site results in a conformational change in the molecule, including the “activation domain” and formation of a binding site for Met receptor on the HGF β chain. In a wild-type HGF, an activation site is located at, between or adjacent to amino acid residues 494 and 495.

“Activation domain” refers to the region on a HGF β chain that undergoes conformational change upon cleavage of a single chain HGF. Upon of cleavage of scHGF at, between or adjacent to amino acids 494 and 495, the HGF β chain undergoes a conformational change, including the formation of a Met receptor binding site. In some embodiments, the activation domain in a HGF β comprises, consists essentially of, or consists of at least one amino acid residue corresponding to residues of HGF β from about 495 to 498, amino acid residues from about 615 to about 625, or from about 660 to about 670, from about 692 to about 697, from about 642 to about 652, in some instances, amino acid residues from about 550 to about 560, or mixtures thereof.

“Tunnel” refers to a conformation of a polypeptide, or portion thereof, that forms a void. In a HGF β crystal structure, the void is formed by amino acid residues. In some embodiments, the void is formed by at least one amino acid residue in a position that comprises, consists essentially of, or consists of at least one amino acid residue 643, 673, from about 693 to 706, from about 660 to 670, or 691, or mixtures thereof.

“Dimerization domain” refers to a region of a HGF β chain that interacts with another HGF β chain to form a dimer. Upon cleavage of scHGF, the HGF β chain undergoes a conformational change. The HGF-β N-terminal residue 495 forms a salt bridge with residue Asp 672. In some embodiments, the dimerization region of a HGF β comprises, consists essentially of, or consists of at least one amino acid residue corresponding to residues of HGF β from about 495 to 502, the [c140 loop] amino acids including Y619, T620, G621, the [c180] loop amino acids including 662 to 665, or the [c220] loop amino acids including 700, or mixtures thereof.

As used herein, “crystal” refers to one form of solid state of matter in which atoms are arranged in a pattern that repeats periodically in three dimensions, typically forming a lattice.

“Complementary or complement” as used herein, refers to the fit or relationship between two molecules that permits interaction, including for example, space, charge, three-dimensional configuration, and the like.

The term “corresponding” or “corresponds” refers to an amino acid residue or amino acid sequence that is found at the same positions or positions in a sequence when the amino acid position or sequences is aligned with a reference sequence. In some embodiments, the reference sequence is HGF β having a sequence of SEQ ID NO: 1. It will be appreciated that when the amino acid position or sequence is aligned with the reference sequence the numbering of the amino acids may differ from that of the reference sequence or a different numbering system may be employed.

“Heavy atom derivative”, as used herein, refers to a derivative produced by chemically modifying a crystal with a heavy atom such as Hg, Au, or halogen.

“Structural homolog” of HGF β as used herein refers to a protein that contains one or more amino acid substitutions, deletions, additions, or rearrangements with respect to the amino acid sequence of HGF β, but that, when folded into its native conformation, exhibits or is reasonably expected to exhibit at least a portion of the tertiary (three-dimensional) structure of HGF β. Portions of the three dimensional structure include structural features such as the binding site for Met on HGF β, activation domain, activation site, active site, tunnel, dimerization region and combinations thereof. For example, structurally homologous molecules of HGF β include MSP and HGF β variants, preferably variants with one or more conservative amino acid substitutions, preferably only conservative amino acid substitutions. Homolog tertiary structure can be probed, measured, or confirmed by known analytic or diagnostic methods, for example, X-ray, NMR, circular dichroism, a panel of monoclonal antibodies that recognize native HGF β, and like techniques. For example, structurally homologous molecules can contain deletions or additions of one or more contiguous or noncontiguous amino acids, such as a loop or a domain. Structurally homologous molecules also include “modified” HGF β molecules that have been chemically or enzymatically derivatized at one or more constituent amino acids, including side chain modifications, backbone modifications, and N- and C-terminal modifications including acetylation, hydroxylation, methylation, amidation, and the attachment of carbohydrate or lipid moieties, cofactors, and like modifications.

“Ligand”, as used herein, refers to an agent that associates with a binding site on a molecule, for example, Met and/or HGF β binding sites, and may be an antagonist or agonist of Met or the HGF β activity. Ligands include molecules that mimic HGF β binding to Met and in some embodiments, are not capable of activating HGF β/Met signaling pathway.

“Molecular complex”, as used herein, refers to a combination of bound substrate or ligand with polypeptide, such as HGF β bound to Met, or a ligand bound to HGF β.

“Machine-readable data storage medium”, as used herein, refers to a data storage material encoded with machine-readable data, wherein a machine programmed with instructions for using such data is capable of displaying data in the desired format, for example, a graphical three-dimensional representation of molecules or molecular complexes.

“Scalable,” as used herein, refers to the increasing or decreasing of distances between coordinates (configuration of points) by a scalar factor while keeping the angles essentially the same.

“Space group symmetry”, as used herein, refers to the whole symmetry of the crystal that combines the translational symmetry of a crystalline lattice with the point group symmetry. A space group is designated by a capital letter identifying the lattice type (e.g. P, A, F,) followed by the point group symbol in which the rotation and reflection elements are extended to include screw axes and glide planes. Note that the point group symmetry for a given space group can be determined by removing the cell centering symbol of the space group and replacing all screw axes by similar rotation axes and replacing all glide planes with mirror planes. The point group symmetry for a space group describes the true symmetry of its reciprocal lattice.

“Unit cell”, as used herein, refers to the atoms in a crystal that are arranged in a regular repeating pattern, in which the smallest repeating unit is called the unit cell. The entire structure can be reconstructed from knowledge of the unit cell, which is characterized by three lengths (a, b and c) and three angles (α, β and γ). The quantities a and b are the lengths of the sides of the base of the cell and γ is the angle between these two sides. The quantity c is the height of the unit cell. The angles α and β describe the angles between the base and the vertical sides of the unit cell.

“X-ray diffraction pattern” refers to the pattern obtained from X-ray scattering of the periodic assembly of molecules or atoms in a crystal. X-ray crystallography is a technique that exploits the fact that X-rays are diffracted by crystals. X-rays have the proper wavelength (in the Ångström (Å) range, approximately 10⁻⁸ cm) to be scattered by the electron cloud of an atom of comparable size. Based on the diffraction pattern obtained from X-ray scattering of the periodic assembly of molecules or atoms in the crystal, the electron density can be reconstructed. Additional phase information can be extracted either from the diffraction data or from supplementing diffraction experiments to complete the reconstruction. A model is then progressively built into the electron density, refined against the data to produce an accurate molecular structure.

X-ray structure coordinates define a unique configuration of points in space. Those of skill in the art understand that a set of structure coordinates for a protein or a protein/ligand complex, or a portion thereof, define a relative set of points that, in turn, define a configuration in three dimensions. A similar or identical configuration can be defined by an entirely different set of coordinates, provided the distances and angles between coordinates remain essentially the same. In addition, a configuration of points can be defined by increasing or decreasing the distances between coordinates by a scalar factor, while keeping the angles essentially the same.

C. Modes for Carrying Out the Invention

The present disclosure includes a crystalline form of and a crystal structure of hepatocyte growth factor beta chain (HGF β) and methods of using the HGF β crystal structure and structural coordinates to identify homologous proteins and to design or identify agents that can modulate the function of HGF and/or HGF β chain whether alone or as naturally found linked to HGF alpha chain. The present disclosure also includes the three-dimensional configuration of points derived from the structure coordinates of at least a portion of a HGF β molecule or molecular complex, as well as structurally equivalent configurations, as described below. Structurally equivalent configurations can include HGF β variants that have at least one conservative amino acid substitution, preferably all substitutions of a HGF β variant are conservative. The three-dimensional configuration includes points derived from structure coordinates representing the locations of a plurality of the amino acids defining the HGF β binding site, active site, activation domain, tunnel, dimerization region and combinations thereof.

In some embodiments, the three-dimensional configuration includes points derived from structure coordinates representing the locations of the backbone atoms of a plurality of amino acid residues defining the HGF β ligand binding site. Alternatively, the three-dimensional configuration includes points derived from structure coordinates representing the locations of the side chain and the backbone atoms (other than hydrogens) of a plurality of the amino acid residues defining the HGF β ligand binding site, preferably the amino acids listed in Tables 4A and 4B.

The disclosure also includes the three-dimensional configuration of points identifying other structural features of the HGF β domain. Those other structural features include the active site, activation domain, tunnel and/or HGF β dimerization region. A plurality of amino acid residues have been identified as contributing to these structural features of HGF β. In some embodiments, the amino acid residues comprise those listed in Table 4 and/or the figures.

Likewise, the disclosure also includes the three-dimensional configuration of points derived from structure coordinates of molecules or molecular complexes that are structurally homologous to HGF β, as well as structurally equivalent configurations. Structurally homologous molecules or molecular complexes are defined below. Advantageously, structurally homologous molecules can be identified using the structure coordinates of HGF β according to a method of the disclosure.

The configurations of points in space derived from structure coordinates according to the disclosure can be visualized as, for example, a holographic image, a stereodiagram, a model, or a computer-displayed image, and the disclosure thus includes such images, diagrams or models.

The crystal structure and structural coordinates can be used in methods for obtaining structural information of a related molecule, and for identifying and designing agents that modulate HGF β chain activity.

The coordinates of the crystal structure of HGF β have been deposited with the RSCB Databank, Accession No: PDB 1SI5.

1. HGF β Chain Polypeptides, Polynucleotides and Variants Thereof

The present disclosure includes a description of hepatocyte growth factor and/or portions thereof. Hepatocyte growth factor comprises a 69 kDa alpha chain and 34 kDa beta chain. HGF is secreted as a single chain precursor form (scHGF). The 69 kDa alpha chain comprise a N terminal finger domain and four kringle domains (K1-K4). A representative amino acid sequence of human HGF β chain is shown in Table 7 (SEQ ID NO: 1). The sequence of Table 7 has one amino acid change from wild type shown in Table 9; the cysteine at amino acid position 604 is changed to a serine. It would be expected that a wild type HGF β would have an equivalent crystal structure. The amino acids of the alpha and beta chain are numbered based on the amino acid numbering system of scHGF. Numbering in brackets are those amino acid positions of the HGF β that correspond to chymotrypsinogen numbering system.

Native or wild type HGF, HGF α chain or HGF β polypeptides are those polypeptides that have a sequence of a polypeptide obtained from nature. Native or wild type HGF, HGF α or HGF β include naturally occurring variants, secreted and truncated forms. Some domains of the α chain and β chain are known to those of skill in the art. Several isoforms of HGF are known such as isoform 1, isoform 2, isoform 3, isoform 4, and isoform 5. Representative sequences can be found at GenBank Accession Numbers NM_{—}000601, NM_{—}001010931, NM_{—}001010932, NM_{—}001010933, NM_{—}001010934, and NP_{—}000592. A wild type HGF β chain comprises an amino acid sequence of SEQ ID NO:5 as shown in Table 9. A wild type HGF sequence of isoform 1 comprises an amino acid sequence of SEQ ID NO:6 and is shown in Table 10.

The present disclosure also includes a polypeptide comprising, consisting essentially of, or consisting of a portion of HGF β starting at any one of amino acid residues 513 to 534 and ending at any one of amino acid residues 696 to 707 or residues corresponding to these positions. This polypeptide includes amino acid positions that form the binding site for the Met receptor on HGF β and in some embodiments, can bind to the Met receptor. In some embodiments, the polypeptide portion may be fused to a heterologous polypeptide or other compound and, preferably, the fusion protein can bind to the Met receptor.

The present disclosure also includes a polypeptide comprising, consisting essentially of, or consisting of a portion of the HGF β starting at amino acid residue 495 and ending at any one of amino acid residues 696 to 704 or residues corresponding to these positions. This polypeptide includes amino acid residues that form the activation domain and in some embodiments, can bind and/or activate the Met receptor. The activation domain is formed upon cleavage of single chain HGF and a change in conformation of HGF β to provide for binding and/or activation of the Met receptor. In some embodiments, this polypeptide can be fused to a heterologous polypeptide or other compound and the fusion protein preferably can bind and/or activate the Met receptor.

In some embodiments, a polypeptide comprises, consists essentially of, or consists of a portion of the HGF β starting at amino acid residues 532 to 534 and ending at any one of amino acid residues 697 to 707 or residues corresponding to these positions. This polypeptide includes amino acid positions that form an active site and in some embodiments, can bind the Met receptor. The active site includes amino acids that correspond to a catalytic triad typically found in proteases and the substrate binding site. The active site of HGF β includes amino acids Asn578, Gln534, Tyr673, as well as amino acids that are involved in binding the Met receptor. In some embodiments, this polypeptide can be fused to a heterologous polypeptide, or other compound, and the fusion protein can bind to Met receptor.

In some embodiments, a polypeptide comprises, consists essentially of, or consists of a portion of the HGF β starting at any one of amino acid residues 634 to 660 and ending at any one of amino acid residues 696 to 706 or residues corresponding to these positions. This polypeptide includes amino acid residues that form a tunnel in the crystal structure in HGF β. The polypeptide includes some of the amino acids that bind or contact the Met receptor, and in some embodiments, can bind to the Met receptor. The polypeptide portion may be fused to a heterologous polypeptide or compound, and preferably, retains binding to the Met receptor.

In some embodiments, a polypeptide position comprises, consists essentially of, or consists of a portion of HGF β starting at amino acid residue 496 and ending at any one of amino residues 670 to 700 or residues corresponding to those positions. This polypeptide includes amino acids that contact another HGF β molecule to form a dimer, and preferably, can dimerize with another HGF β chain. The polypeptide position may be fused to a heterologous polypeptide or compound, and preferably can dimerize with another HGF β chain.

The present disclosure also includes variants of the HGF β. Variants include those polypeptides that have amino acid substitutions, deletions, and additions. Amino acid substitutions can be made, for example, to replace cysteines and eliminate formation of disulfide bonds. Other variants can be made at the binding site for Met, activation site, active site, activation domain, dimerization region, tunnel or combinations thereof. In some embodiments, variants have alterations at amino acid positions other than those amino acid positions associated with Met receptor binding. In some embodiments, a variant of HGF β has at least 90% sequence identity to a polypeptide comprising an amino acid sequence of SEQ ID NO: 1 or SEQ ID NO:5 and only has conservative amino acid substitutions. Preferably, the conservative amino acid substitutions are at amino acid positions other than those associated with amino acids of the Met receptor binding site such as at least the core amino acids of the Met receptor binding site as shown in Table 4B. In other embodiments, the variants bind to and/or activate the Met receptor. In other embodiments, the variants bind to but do not activate the Met receptor. Some examples of specific embodiments of variants are listed in Table 1.

Fusion Proteins

HGF β chains, structural homologs, or portions thereof, may be fused to a heterologous polypeptide or compound. The heterologous polypeptide is a polypeptide that has a different function than that of the HGF β chain. Examples of a heterologous polypeptide include polypeptides that may act as carriers, may extend half life, may act as epitope tags, or may provide ways to detect or purify the fusion protein. Heterologous polypeptides include KLH, albumin, salvage receptor binding epitopes, immunoglobulin constant regions, and peptide tags. Peptide tags useful for detection or purification include FLAG, gD protein, polyhistidine tags, hemaglutinin influenza virus, T7 tag, S tag, Strep tag, chloramiphenicol acetyl transferase, biotin, glutathione-S transferase, green fluorescent protein and maltose binding protein. Compounds that can be combined with HGF β, or portions thereof, include radioactive labels, protecting groups, and carbohydrate or lipid moieties.

Polynucleotides, Vectors, Host Cells

HGF β chain variants can be prepared by introducing appropriate nucleotide changes into DNA encoding HGF β or by synthesis of the desired polypeptide variants using standard methods.

Amino acid substitutions, include one or more conservative amino acid substitutions. The term “conservative” amino acid substitution as used herein refers to an amino acid substitution which substitutes a functionally equivalent amino acid. Conservative amino acid changes result in silent changes in the amino acid sequence of the resulting polypeptide. For example, one or more amino acids of a similar polarity act as functional equivalents and result in a silent alteration within the amino acid sequence of the peptide. In general, substitutions within a group can be considered conservative with respect to structure and function. However, the skilled artisan will recognize that the role of a particular residue is determined by its context within the three-dimensional structure of the molecule in which it occurs. For example, Cys residues may occur in the oxidized (disulfide) form, which is less polar than the reduced (thiol) form. The long aliphatic portion of the Arg side chain can constitute a feature of its structural or functional role, and this may be best conserved by substitution of a nonpolar, rather than another basic residue. Also, it will be recognized that side chains containing aromatic groups (Trp, Tyr, and Phe) can participate in ionic-aromatic or “cation-pi” interactions. In these cases, substitution of one of these side chains with a member of the acidic or uncharged polar group may be conservative with respect to structure and function. Residues such as Pro, Gly, and Cys (disulfide form) can have direct effects on the main chain conformation, and often may not be substituted without structural distortions.

Amino acid substitutions can be the result of replacing one amino acid with another amino acid having similar structural and/or chemical properties, such as the replacement of a leucine with a serine, i.e., conservative amino acid replacements. Examples of conservative substitutions are shown in Table 11. The variation allowed can be determined by systematically making insertions, deletions or substitutions of amino acids in the sequence and testing the resulting variants for activity exhibited by the native sequence.

TABLE 11

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norieucine | |

Polynucleotide sequences encoding the polypeptides described herein can be obtained using standard recombinant techniques. Desired polynucleotide sequences may be isolated and sequenced from appropriate source cells. Alternatively, polynucleotides can be synthesized using nucleotide synthesizer or PCR techniques. Once obtained, sequences encoding the polypeptides or variant polypeptides are inserted into a recombinant vector capable of replicating and expressing heterologous polynucleotides in a host cell. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication (in particular when the vector is inserted into a prokaryotic cell), a selection marker gene, a promoter, a ribosome binding site (RBS), a signal sequence, the heterologous nucleic acid insert and a transcription termination sequence.

In general, plasmid vectors containing replicon and control sequences, which are derived from a species compatible with the host cell, are used in connection with these hosts. The vector ordinarily carries a replication site, as well as marking sequences, which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

Either constitutive or inducible promoters can be used in the present invention, in accordance with the needs of a particular situation, which can be ascertained by one skilled in the art. A large number of promoters recognized by a variety of potential host cells are well known. The selected promoter can be operably linked to cistron DNA encoding a polypeptide described herein by removing the promoter from the source DNA via restriction enzyme digestion and inserting the isolated promoter sequence into the vector of choice. Both the native promoter sequence and many heterologous promoters may be used to direct amplification and/or expression of the target genes. However, heterologous promoters are preferred, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the polypeptides or variant polypeptides (Siebenlist et al. (1980) Cell 20: 269) using linkers or adaptors to supply any required restriction sites.

In some embodiments, each cistron within a recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

Prokaryotic host cells suitable for expressing polypeptides include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. Preferably, gram-negative cells are used. Preferably the host cell should secrete minimal amounts of proteolytic enzymes, and additional protease inhibitors may desirably be incorporated in the cell culture.

Besides prokaryotic host cells, eukaryotic host cell systems are also well established in the art. Examples of invertebrate cells include insect cells such as Drosophila S2 and Spodoptera Sf9, as well as plants and plant cells. Examples of useful mammalian host cell lines include Chinese hamster ovary (CHO) and COS cells. More specific examples include monkey kidney CV1 line transformed by SV40 (COS-7, ATCC CRL 1651); Chinese hamster ovary cells/-DHFR(CHO, Urlaub and Chasin, Proc. Natl. Acad. Sci. USA, 77:4216 (1980)); mouse sertoli cells (TM4, Mather, Biol. Reprod., 23:243-251 (1980)); and mouse mammary tumor (MMT 060562, ATCC CCL51).

Polypeptide Production

Host cells are transformed or transfected with the above-described expression vectors and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include luria broth (LB) plus necessary nutrient supplements. In preferred embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector, protein expression is induced under conditions suitable for the activation of the promoter. For example, if a PhoA promoter is used for controlling transcription, the transformed host cells may be cultured in a phosphate-limiting medium for induction. A variety of other inducers may be used, according to the vector construct employed, as is known in the art.

Eukaryotic host cells are cultured under conditions suitable for expression of the polypeptides of the invention. The host cells used to produce the polypeptides may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in one or more of Ham et al., 1979, Meth. Enz. 58:44, Barnes et al., 1980, Anal. Biochem. 102: 255, U.S. Pat. No. 4,767,704, U.S. Pat. No. 4,657,866, U.S. Pat. No. 4,927,762, U.S. Pat. No. 4,560,655, or U.S. Pat. No. 5,122,469, WO 90/103430, WO 87/00195, and U.S. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES™), nucleotides (such as adenosine and thymidine), antibiotics (such as GENTAMYCIN™), trace elements (defined as inorganic compounds usually present at final concentrations in the micromolar range), and glucose or an equivalent energy source. Other supplements may also be included at appropriate concentrations that would be known to those skilled in the art. The culture conditions, such as temperature, pH, and the like, are those previously used with the host cell selected for expression, and will be apparent to the ordinarily skilled artisan.

Polypeptides described herein expressed in a host cell may be secreted into and recovered from the periplasm of the host cells. Protein recovery typically involves disrupting the cell, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated there from. Cells may be removed from the culture and the culture supernatant being filtered and concentrated for further purification of the proteins produced. The expressed polypeptides can be further isolated and identified using commonly known methods such as fractionation on immunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel filtration using, for example, Sephadex G-75; hydrophobic affinity resins, ligand affinity using a suitable antigen immobilized on a matrix and Western blot assay.

Polypeptides that are produced may be purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

2. Crystals and Crystal Structure of HGF β Chain

The present disclosure provides crystals of HGF β chain as well as the crystal structure of HGF β chain as determined therefrom. In some embodiments, the crystals can be diffracted to a resolution of 5 Å or better. In some embodiments, the crystal is that of activated HGF β. Activated HGF β refers to the form of HGF β that occurs upon cleavage of scHGF and has a conformational change forming an activation domain and binding site for Met. In some embodiments, HGF β comprises an amino acid sequence of SEQ ID NO:1 or conservative substitutions thereof or portions thereof. In some embodiments, HGF β comprising an amino acid sequence of SEQ ID NO:1 only has conservative amino acid substitutions, preferably at amino acid positions other than those of the binding site for Met.

The crystals are useful to provide the crystal structure and/or to provide a stable form of the molecule for storage. In a specific embodiment, the structure of human HGF β chain comprising SEQ ID NO: 1 was solved by molecular replacement using AMoRe (Navaza, 1994) in space group P3₁21, using parts of the protease domain of coagulation factor VIIa (Dennis et al., 2000) as the search probe. Refinement was performed using X-PLOR98 (MSI, San Diego) and REFMAC (Murshudov et al., 1997). Inspection of electron density maps and model manipulation were performed using XtalView (McRee, 1999).

Each of the constituent amino acids of HGF β is defined by a set of structure coordinates as set forth in Table 5. The term “structure coordinates” refers to Cartesian coordinates derived from mathematical equations related to the patterns obtained on diffraction of a monochromatic beam of X-rays by the atoms (scattering centers) of a HGF β in crystal form. The diffraction data are used to calculate an electron density map of the repeating unit of the crystal. The electron density maps are then used to establish the positions of the individual atoms of the HGF β protein or protein/ligand complex.

Slight variations in structure coordinates can be generated by mathematically manipulating the HGF β or HGF β/ligand structure coordinates. For example, the structure coordinates as set forth in Table 5 could be manipulated by crystallographic permutations of the structure coordinates, fractionalization of the structure coordinates, integer additions or subtractions to sets of the structure coordinates, inversion of the structure coordinates, or any combination of the above. Alternatively, modifications in the crystal structure due to mutations, additions, substitutions, deletions, and combinations thereof, of amino acids, or other changes in any of the components that make up the crystal, could also yield variations in structure coordinates. Such slight variations in the individual coordinates will have little effect on overall shape. If such variations are within an acceptable standard error as compared to the original coordinates, the resulting three-dimensional shape is considered to be structurally equivalent. Structural equivalence is described in more detail below.

It should be noted that slight variations in individual structure coordinates of the HGF β would not be expected to significantly alter the nature of chemical entities such as ligands that could associate with an active site. In this context, the phrase “associating with” refers to a condition of proximity between a ligand, or portions thereof, and a HGF β molecule or portions thereof. The association may be non-covalent, wherein the juxtaposition is energetically favored by hydrogen bonding, van der Waals forces, and/or electrostatic interactions, or it may be covalent.

To better interpret Met receptor binding and activity data from HGF mutants, the HGF β structure at 2.53 Å resolution was solved. Data reduction and refinement statistics and final model metrics appear in Table 3. The crystal of HGF β has a space group symmetry of P3₁21 and comprises a unit cell having dimensions of a=b and c, wherein a and b are about 63.7 angstroms and c is about 135.1 angstroms.

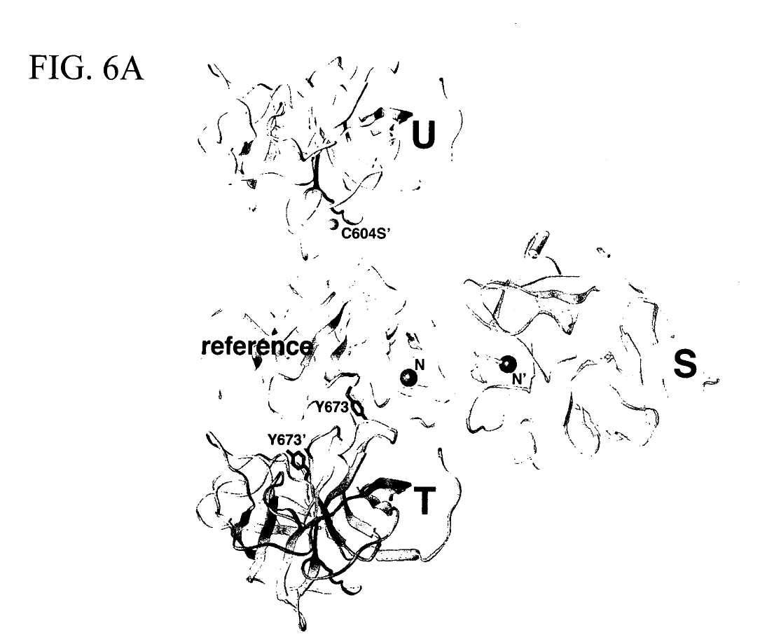

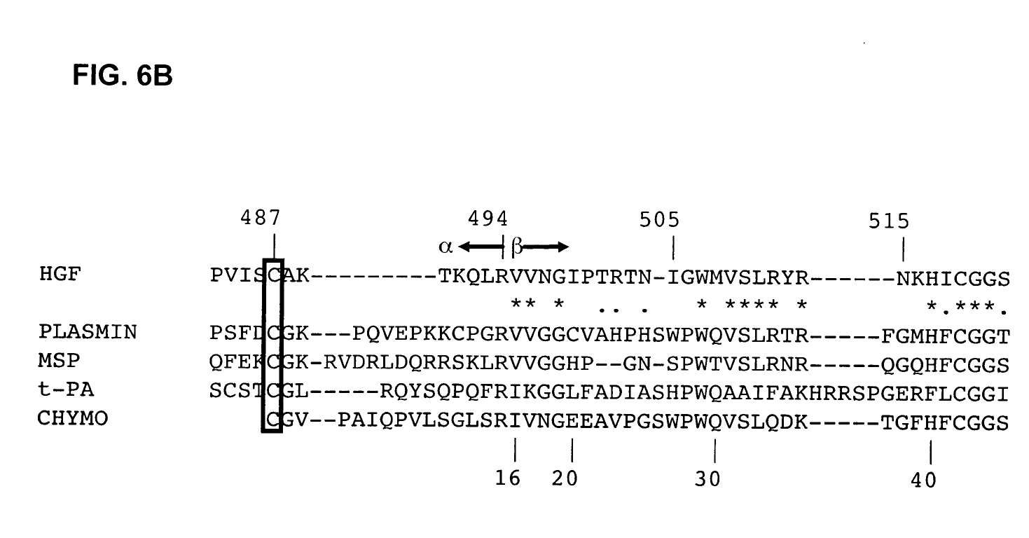

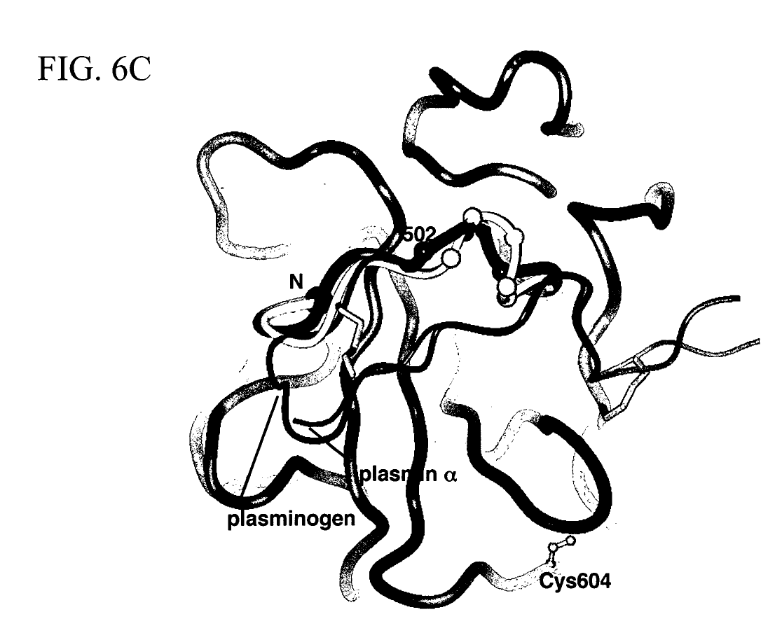

HGF β crystals were formed using three intermolecular contacts for each molecule (FIG. 6A). The smallest contact (about 360 Å² on each side) involves residues in the I550-K562 [c70-c80] loop on one molecule and residues near the putative α-chain connecting Cys604 [c128] (mutated to Ser) site on the other molecule. Two larger intermolecular contacts derive from 2-fold crystallographic symmetry. Residues following the N-terminus (Val496-Arg502 [c17-c23]) plus residues from the [c140]-(617-630) and [c180]-(660-670) loops lose about 640 Å² of solvent accessible area (each side), and residues centered on Gln534 [c57] share a contact area of about 930 Å² (each side).

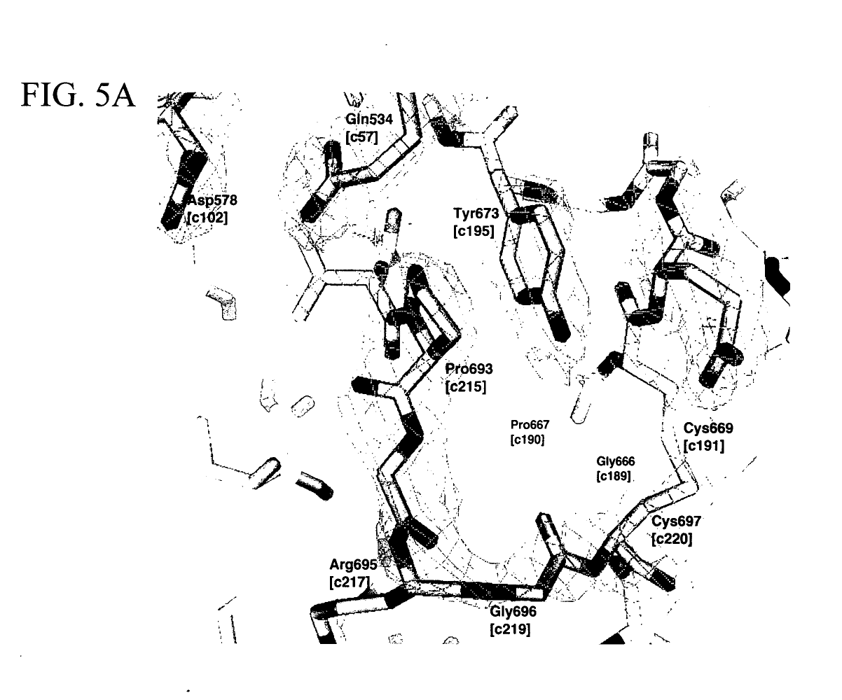

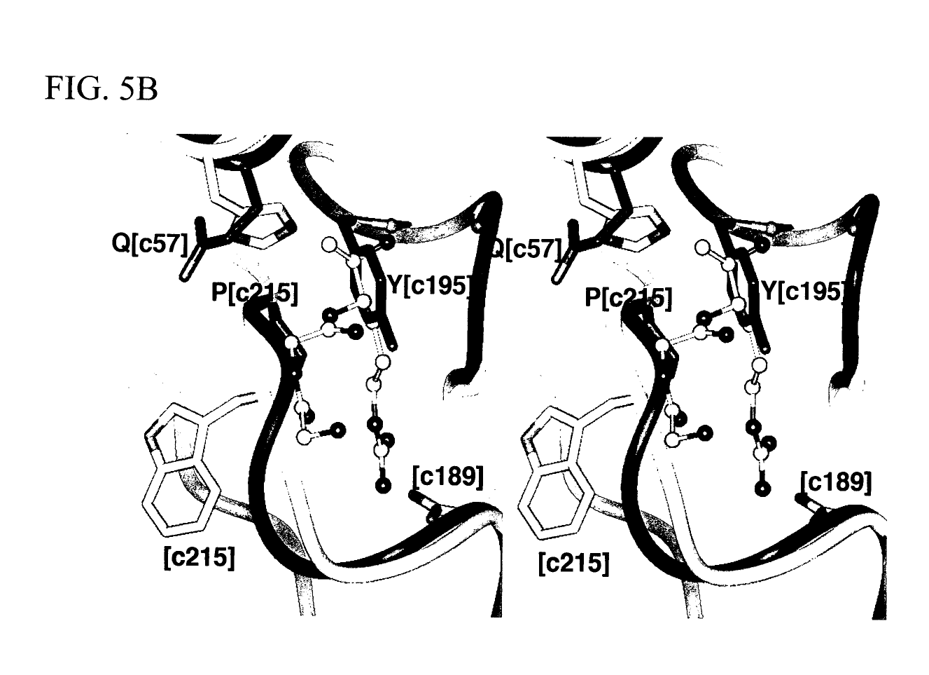

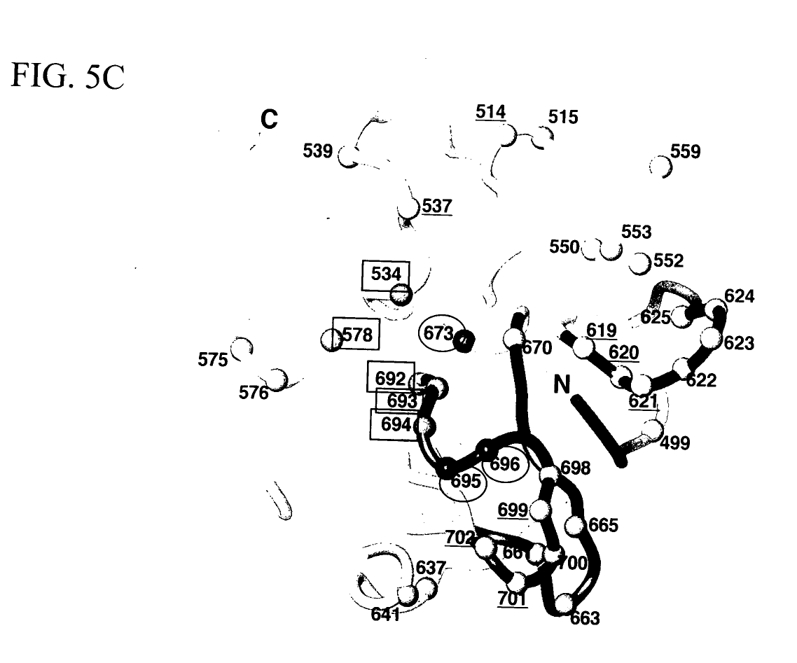

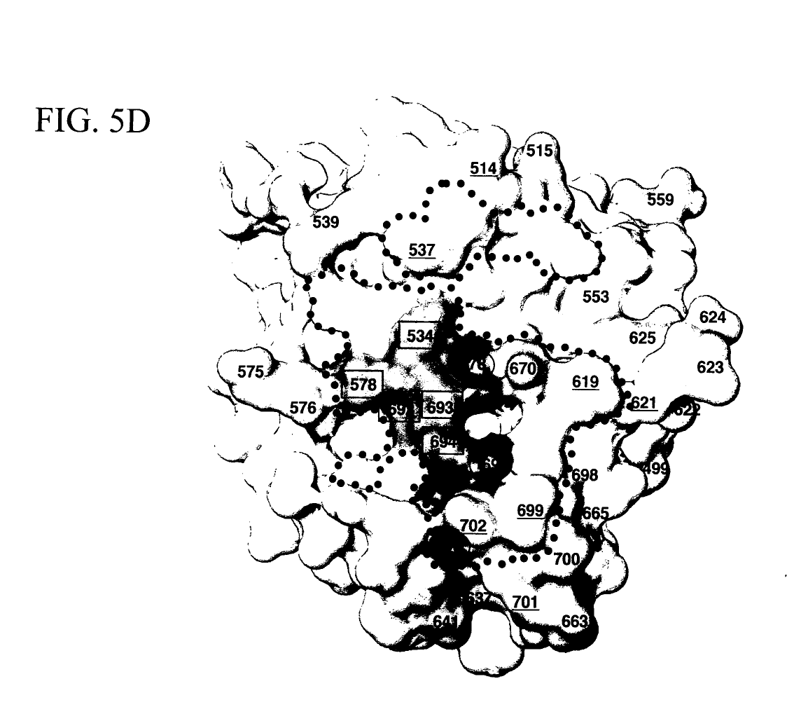

HGF β adopts the fold of chymotrypsin-like serine proteases, comprising two tandem distorted β-barrels. There are two poorly ordered and untraceable segments—His645-Thr651 [c170a-c175] and the C-terminal region beginning with Tyr723 [c245]. The ‘active-site’ region of HGF β clearly differs from those of true enzymes (FIG. 5A). Only Asp578 [c102] of the canonical catalytic triad is present, Ser and His being changed to Tyr673 [c195] and Gln534 [c57], respectively. As a result, the interaction between Ser and His, supported by an Asp-His hydrogen bond, is impossible and Tyr673 [c195] significantly narrows the entrance to the ‘S1 pocket’. In addition to changes in two of the ‘catalytic triad residues’, Pro693 [c215] is distinct from Trp [c215] found in all serine proteases. Indeed, normal substrate binding via main chain hydrogen bonds to segment [c214-c216] would be severely hampered by the main chain conformation and side chains of Val692 [c214] and Pro693 [c215] (FIG. 5B). There are structural differences in the nominal ‘S1 pocket’, where Gly667 [c189] at the bottom of the pocket and Pro668 [c190] are also distinct from residues found in serine proteases. Thus, there is a structural basis to understand why mutations in HGF creating the Asp [c102]-His [c57]-Ser [c195] catalytic triad are insufficient to impart catalytic activity (Lokker et al., 1992).

HGF β residues involved in interactions with Met are shown in FIGS. 5C and 5D as determined according to their relative activities in cell migration assays. When these residues are displayed using the HGF-β crystal structure, they form a compact region centered on the ‘active-site’ region. The electrostatic surface charge distribution in the binding site is diverse, being nonpolar at Tyr673 [c195] and Val692 [c214], polar at Gln534 [c57], negatively charged at Asp578 [c102], and positively charged at Arg695 [c217]. The outer limit of the functional Met binding site extends to distal portions of the [c220]-loop (residues 1699 [c221a] and N701 [c223]), the [c140]-loop (residues Y619, T620, G621 [c143-c145]) and residues R514 [c36] and P537 [c60a] (FIGS. 5C and 5D). These residues form a structure similar to the substrate-processing region of true serine proteases.

The structural binding site identified herein is in excellent agreement with the structural Met binding site revealed in the co-crystal structure of an extracellular fragment including the soluble Met Sema domain bound to HGF β as disclosed in application U.S. Ser. No. 60/568,865, filed May 6, 2004, which application is hereby incorporated by reference. For instance, the co-crystal structure shows that residues on the [c220]-loop, such as R695 [c217] contact the Met receptor.

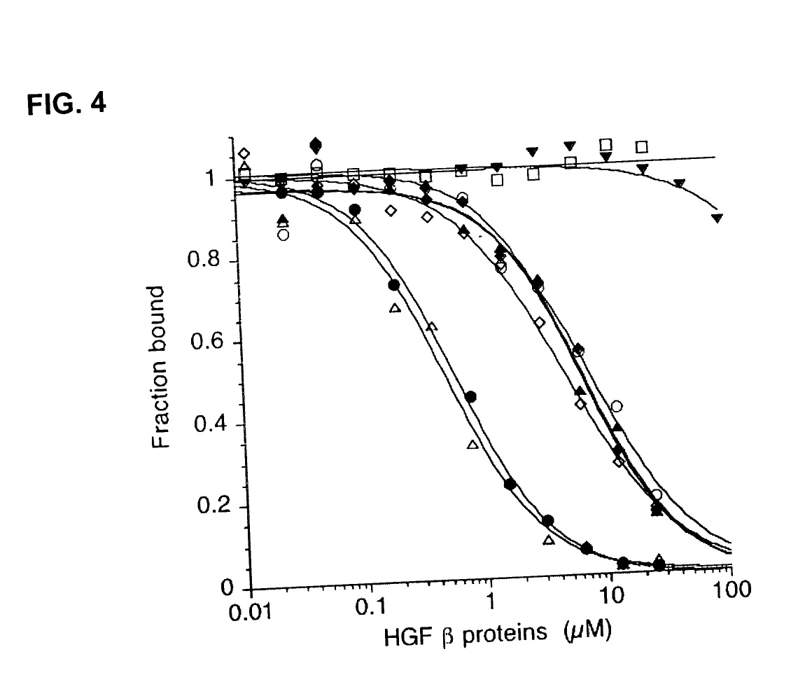

The HGF β chain forms a symmetric dimer in the crystal structure. The amino acid residues that form the dimerization region were identified by making a determination of those residues that lose solvent accessibility when two molecules of HGF β from the crystal structure were analyzed. In some embodiments, the dimerization amino acid residues include at least one amino acid from about 495 to 502, from about 619 to 624, 626, 628, 630, from about 662 to 665, or 700 or mixtures thereof. The HGF β-chain may have functions in receptor activation beyond those involved in direct interactions with Met that would favor a 2:2 complex of HGF:Met. It was found that proHGF β, the single chain ‘unactivated’ form of the HGF β-chain, bound more tightly to Met than several mutants in the ‘activated’ form of HGF β, i.e. Y673A, V692A, and R695A (FIG. 4). All three corresponding full-length HGF mutants show measurable receptor phosphorylation and/or pro-migratory activities, however proHGF does not show such activities, even at concentrations 1,000-fold more than that needed for activity by HGF. This distinction indicates additional functions of the HGF β-chain in receptor activation.

The β-chain of HGF comprises a new interaction site with Met, which is similar to the ‘active-site’ region of serine proteases. HGF is bivalent, having a high affinity Met binding site in the NK1 region of the α-chain and a low affinity binding site in the β chain. Other interactions may occur between two HGF β-chains, two HGF α-chains (Donate et al., 1994), and as found with MSP/Ron between two Met Sema domains. Heparin also plays a role in HGF/Met receptor binding. The identification of a distinct Met binding site on the HGF β-chain can be used to design new classes of HGF or Met modulators, such as antagonists, agonists, and like agents, having therapeutic applications, such as, for treating cancer.

3. Structurally Equivalent Crystal Structures

Various computational analyses can be used to determine whether a molecule or portions of the molecule define structural features that are “structurally equivalent” to all or part of HGF β or its structural features. Such analyses may be carried out in current software applications, such as the Molecular Similarity application of QUANTA (Molecular Simulations Inc., San Diego, Calif.), Version 4.1, and as described in the accompanying User's Guide.

The Molecular Similarity application permits comparisons between different structures, different conformations of the same structure, and different parts of the same structure. A procedure used in Molecular Similarity to compare structures comprises: 1) loading the structures to be compared; 2) defining the atom equivalences in these structures; 3) performing a fitting operation; and 4) analyzing the results.

One structure is identified as the target (i.e., the fixed structure); all remaining structures are working structures (i.e., moving structures). Since atom equivalency within QUANTA is defined by user input, for the purpose of this disclosure equivalent atoms are defined as protein backbone atoms (N, Cα, C, and O) for all conserved residues between the two structures being compared. A conserved residue is defined as a residue that is structurally or functionally equivalent. Only rigid fitting operations are considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in Angstroms, is reported by QUANTA.

Structurally equivalent crystal structures have portions of the two molecules that are substantially identical, within an acceptable margin of error. The margin of error can be calculated by methods known to those of skill in the art. In some embodiments, any molecule or molecular complex or any portion thereof, that has a root mean square deviation of conserved residue backbone atoms (N, Cα, C, O) of less than about 0.70 Å, preferably 0.5 Å. For example, structurally equivalent molecules or molecular complexes are those that are defined by the entire set of structure coordinates listed in Table 5 and/or 6±a root mean square deviation from the conserved backbone atoms of those amino acids of not more than 0.70 Å, preferably 0.5 Å. The term “root mean square deviation” means the square root of the arithmetic mean of the squares of the deviations. It is a way to express the deviation or variation from a trend or object. For purposes of this disclosure, the “root mean square deviation” defines the variation in the backbone of a protein from the backbone of HGF β (as defined by the structure coordinates of HGF β described herein) or a defining structural feature thereof.

4. Structurally Homologous Molecules, Molecular Complexes, and Crystal Structures

Structure coordinates can be used to aid in obtaining structural information about another crystallized molecule or molecular complex. The method of the disclosure allows determination of at least a portion of the three-dimensional structure of molecules or molecular complexes that contain one or more structural features that are similar to structural features of HGF β. In some embodiments, a portion of the three-dimensional structure includes the structural features of a HGF β chain, for example, binding site for Met, activation domain, active site, tunnel and/or dimerization region. These molecules are referred to herein as “structurally homologous” to HGF β. Similar structural features can also include, for example, regions of amino acid identity, conserved active site or binding site motifs, and similarly arranged secondary structural elements (e.g., α helices and β sheets). Preferably, the structural homolog has at least one biological function of HGF β.

Optionally, structural homology is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Two amino acid sequences can be compared using the BLASTP program, version 2.0.9, of the BLAST 2 search algorithm, as described by Tatusova et al. (56), and available at URL www.ncbi.nlm.nih.gov/BLAST/. Preferably, the default values for all BLAST 2 search parameters are used, including matrix=BLOSUM62; open gap penalty=11, extension gap penalty=1, gap x_dropoff=50, expect=10, wordsize=3, and filter on. In the comparison of two amino acid sequences using the BLAST search algorithm, structural similarity is referred to as “identity.”

In some embodiments, a structurally homologous molecule is a protein that has an amino acid sequence sharing at least 80% identity with a native or recombinant amino acid sequence of HGF β. In some embodiments, HGF β has a sequence of SEQ ID NO:1 or SEQ ID NO:5, and the structurally homologous molecule is a variant that has a % sequence identity to SEQ ID NO: 1 or SEQ ID NO:5 of at least 80%, 81%, 82%, 83%, 84%, 85%, 86%, 87%, 88%, 89%, 90%, 91%, 92%, 93%, 94%, 95%, 96%, 97%, 98% or 99% or greater. In some embodiments, HGF β variant or structurally homologous molecule has one or more conservative amino acid substitutions, preferably only conservative amino acid substitutions. In some embodiments, a HGF β variant does not have substitutions in the binding site for Met, including at least the core amino acid residues as shown in Table 4B. In some embodiments, the HGF β variant has about 1-25 conservative amino amino acid substitutions, more preferably about 1-20 conservative amino acids substitutions, more preferably about 1-10 conservative amino acid substitions, more preferably about 1-5 conservative amino acid substitutions, and more preferably about 1-2 conservative amino acid substitutions. Preferably, the variant retains the globular core structure and/or at least one or more domains such as the binding site for Met, activation domain, active site, tunnel and/or dimerization region.

For example, a structurally homologous protein is the wild type HGF β (SEQ ID NO:5), which differs from HGF β of SEQ ID NO:1 due to substitution of a cysteine at position 604 with a serine (99.55% identity to SEQ ID NO:1). The substitution of a serine at this position is unlikely to substantially affect the crystal structure because serine is similar to cysteine in size and functionality. More preferably, a protein that is structurally homologous to HGF β includes at least one contiguous stretch of at least 50 amino acids that shares at least 80% amino acid sequence identity with the corresponding portion of the native or recombinant HGF β and preferably, has only conservative amino acid substitutions that maintain the size and functionality of the substituted amino acid. Methods for generating structural information about the structurally homologous molecule or molecular complex are well known and include, for example, molecular replacement techniques.

Therefore, in another embodiment this disclosure provides a method of utilizing molecular replacement to obtain structural information about a molecule or molecular complex whose structure is unknown or incompletely known, comprising:
(a) generating an X-ray diffraction pattern from a crystallized molecule or molecular complex of unknown structure or incompletely known, for example in embodiments a structural homolog of HGF β; and/or
(b) applying at least a portion of the structural coordinates of HGF β or HGFβ/ligand complex to the X-ray diffraction pattern of the unknown or incompletely known structure to generate a three-dimensional electron density map of the molecule or molecular complex whose structure is unknown or incompletely known.

By using molecular replacement, all or part of the structure coordinates of HGF β or the HGF β/ligand complex as provided by this disclosure can be used to determine the unsolved structure of a crystallized molecule or molecular complex more quickly and efficiently than attempting to determine such information ab initio.

Molecular replacement can provide an accurate estimation of the phases for an unknown structure. Phases are one factor in equations that are used to solve crystal structures, and this factor cannot be determined directly. Obtaining accurate values for the phases, by methods other than molecular replacement, can be a time-consuming process that involves iterative cycles of approximations and refinements and greatly hinders the solution of crystal structures. However, when the crystal structure of a protein containing at least a structurally homologous portion has been solved, molecular replacement using the known structure can provide a useful estimate of the phases for the unknown or incompletely known structure.

Thus, this method involves generating a preliminary model of a molecule or molecular complex whose structure coordinates are unknown or incompletely known, by orienting and positioning the relevant portion of HGF β or a HGF β/ligand complex within the unit cell of the crystal of the unknown or incompletely known molecule or molecular complex. This orientation or positioning is conducted so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure. This map, in turn, can be subjected to established and well-known model building and structure refinement techniques to provide a final, accurate structure of the unknown crystallized molecule or molecular complex (see for example, Lattman, 1985. Methods in Enzymology 115:55-77).

Structural information about a portion of any crystallized molecule or molecular complex that is sufficiently structurally homologous to a portion of HGF β can be resolved by this method. In addition to a molecule that shares one or more structural features with HGF β as described above, a molecule that has similar bioactivity, such as the same catalytic activity, substrate specificity or ligand binding activity as HGF β, may also be sufficiently structurally homologous to HGF β to permit use of the structure coordinates of HGF β to solve its crystal structure or identify structural features that are similar to those identified in HGF β chain described herein. It will be appreciated that amino acid residues in the structurally homologous molecule identified as corresponding to HGF β chain structural feature may have different amino acid numbering.

In one embodiment of the disclosure, the method of molecular replacement is utilized to obtain structural information about a molecule or molecular complex, wherein the molecule or molecular complex includes at least one HGF β fragment or homolog. HGF β is an inhibitor of full length HGF and can be used to identify or design other like inhibitors. In the context of the present disclosure, a “structural homolog” of HGF β includes a protein that comprises one or more amino acid substitutions, deletions, additions, or rearrangements with respect to the amino acid sequence of HGF β, but that, when folded into its native conformation, exhibits or is reasonably expected to exhibit at least a portion of the tertiary (three-dimensional) structure of HGF β as described above. As mentioned above, homolog tertiary structure can be probed, measured, or confirmed by known analytic and/or diagnostic methods, for example, X-ray, NMR, circular dichroism, panel of monoclonal Abs that recognize native HGF beta. For example, structurally homologous molecules can contain deletions or additions of one or more contiguous or noncontiguous amino acids, such as a loop or a domain. Structurally homologous molecules also include “modified” HGF β molecules that have been chemically or enzymatically derivatized at one or more constituent amino acid, including side chain modifications, backbone modifications, and N- and C-terminal modifications including acetylation, hydroxylation, methylation, amidation, and the attachment of carbohydrate or lipid moieties, cofactors, and like modifications.

A heavy atom derivative of HGF β is also included as a HGF β homolog. The term “heavy atom derivative” refers to derivatives of HGF β produced by chemically modifying a crystal of HGF β. In practice, a crystal is soaked in a solution containing heavy metal atom salts, or organometallic compounds, e.g., lead chloride, gold thiomalate, thiomersal or uranyl acetate, which can diffuse through the crystal and bind to the surface of the protein. The location(s) of the bound heavy metal atom(s) can be determined by X-ray diffraction analysis of the soaked crystal. This information, in turn, is used to generate the phase information used to construct three-dimensional structure of the protein (Blundell, et al., 1976, Protein Crystallography, Academic Press, San Diego, Calif.).

The structure coordinates of HGF β provided by this disclosure are particularly useful in solving the structure of HGF β variants. Variants may be prepared, for example, by expression of HGF β cDNA previously altered in its coding sequence by oligonucleotide-directed mutagenesis as described herein. Variants may also be generated by site-specific incorporation of unnatural amino acids into HGF β proteins using known biosynthetic methods (e.g. Noren, et al., 1989, Science 244:182-88). In this method, the codon encoding the amino acid of interest in wild-type HGF β is replaced by a “blank” nonsense codon, TAG, using oligonucleotide-directed mutagenesis. A suppressor tRNA directed against this codon is chemically aminoacylated in vitro with the desired unnatural amino acid. The aminoacylated tRNA is then added to an in vitro translation system to yield a mutant HGF β with the site-specific incorporated unnatural amino acid.

The structure coordinates of HGF β are also particularly useful to solve or model the structure of crystals of HGF β, HGF β variants, or HGF β homologs co-complexed with a variety of ligands. This approach enables the determination of the optimal sites for interaction between ligand entities, including candidate HGF β ligands and HGF β. Potential sites for modification within the various binding sites of the molecule can also be identified. HGF β variants that may bind to the Met receptor but not activate it may also be identified. This information provides an additional tool for determining more efficient binding interactions, for example, increased hydrophobic interactions, between HGF β and a ligand. For example, high-resolution X-ray diffraction data collected from crystals exposed to different types of solvent allows the determination of where each type of solvent molecule resides. Small molecules that bind tightly to those sites can then be designed and synthesized and tested for their HGF β affinity using standard assays.

In another embodiment, homology modeling can be conducted using the structural coordinates of HGF β and a program designed to generate models of structures, such as Protein Explorer, Swiss Model, or RASMOL. The programs can provide a structural model of a homolog or variant of HGF β by providing the structural coordinates such as provided in Table 5 and an alignment of the sequences.

All of the complexes referred to above may be studied using well-known X-ray diffraction techniques and may be refined versus X-ray data extending to between 1.5 and 3.5 Å to an R-factor of about 0.30 or less using computer software, such as X-PLOR (Yale University, distributed by Molecular Simulations, Inc.)(see for example, Blundell, et al. 1976. Protein Crystallography, Academic Press, San Diego, Calif., and Methods in Enzymology, Vol. 114 & 115, H. W. Wyckoff et al., eds., Academic Press (1985)). This information may thus be used to optimize a candidate HGF β modulator or to design new HGF and/or HGF β modulators.

The disclosure also includes the unique three-dimensional configuration defined by a set of points defined by the structure coordinates for a molecule or molecular complex structurally homologous to HGF β as determined using the method of the present disclosure, structurally equivalent configurations, and storage media, such as magnetic media, including such set of structure coordinates.

5. Homology Modeling

Using homology modeling, a computer model of a HGF β homolog can be built or refined without crystallizing the homolog. First, a preliminary model of the HGF β homolog is created by sequence alignment with HGF β, secondary structure prediction, the screening of structural libraries, or any combination of those techniques. Computational software may be used to carry out the sequence alignments and the secondary structure predictions. Programs available for such an analysis include Protein Explorer (eg available at molvissdsc.edu.protexpl.frontdoor.htm), Swiss Model (eg available at swissmodel.expasy.org) and RASMOL. Structural incoherences, e.g., structural fragments around insertions and deletions, can be modeled by screening a structural library for peptides of the desired length and with a suitable conformation. For prediction of the side chain conformation, a side chain rotamer library may be employed. If the HGF β homolog has been crystallized, the final homology model can be used to solve the crystal structure of the homolog by molecular replacement, as described above. Next, the preliminary model is subjected to energy minimization to yield an energy-minimized model. The energy-minimized model may contain regions where stereochemistry restraints are violated, in which case such regions are remodeled to obtain a final homology model. The homology model is positioned according to the results of molecular replacement, and subjected to further refinement including molecular dynamics calculations.

6. Methods for Identification of Modulators of HGF β

Potent and selective ligands that modulate activity (antagonists and agonists) can be identified using the three-dimensional model of HGF β using structural coordinates of a crystal of HGF β, such as all or a portion of the coordinates of Table 5. Using this model, candidate ligands that interact with HGF β are assessed for the desired characteristics (e.g., interaction with HGF β) by fitting against the model, and the result of the interactions is predicted. Alternatively, molecules that can mimic the binding of HGF β for Met and that are altered in the activation of HGF/Met signaling pathway can also be modeled and identified. Agents predicted to be molecules capable of modulating the activity of HGF β can then be further screened or confirmed against known bioassays. For example, the ability of an agent to inhibit the morphogenic or mitogenic effects of HGF can be measured using assays known in the art. Using the modeling information and the assays described, one can identify agents that possess HGF and/or HGF β-modulating properties. Modulators of HGF β of the present disclosure can include compounds or agents having, for example, allosteric regulatory activity.

Ligands which can interact with HGF β can also be identified using commercially available modeling software, such as docking programs, in which all or a portion of the solved crystal structure coordinates of a crystal of HGF β such as those of Table 5 can be computationally represented and screened against a large virtual library of small molecules or virtual fragment molecules for interaction with a site of interest, such as the binding site for Met, activation domain, active site, tunnel and/or dimerization region. Preferred small molecules or fragments identified in this way can be synthesized and contacted with the HGF β. The resulting molecular complex or association can be further analyzed by, for example, NMR or X-ray co-crystallography, to optimize the HGF β-ligand interaction and/or desired biological activity. Fragment-based drug discovery methods are known and computational tools for their use are commercially available, for example “SAR by NMR” (Shukers, S. B., et al., Science, 1996, 274, 1531-1534), “Fragments of Active Structures” (www.stromix.com; Nienaber, V. L., et al., Nat. Biotechnol., 2000, 18, 1105-1108), and “Dynamic Combinatorial X-ray Crystallography” (e.g., permitting self-selection by the protein molecule of self-assembling fragments; Congreve, M. S., et al., Angew. Chem., Int. Ed., 2003, 42, 4479-4482). Still other molecular modeling, docking, and like methods are discussed below and in the Examples.

The present disclosure also includes identification of allosteric modulators of HGF β. “Allosteric regulation” and like terms refers to regulation of a functional site of HGF β by way of large scale conformational changes in the shape of HGF β which can be caused by, for example, the binding of a regulatory molecule elsewhere (i.e., other than at the functional site) in the HGF β molecule. An “allosteric regulator” or signal molecule is any molecule capable of effecting such allosteric regulation or signaling in the HGF β molecule. An allosteric regulator can be either positive (an activator) or negative (an inhibitor) of HGF β activity. Allosteric regulation of HGF β activity can involve cooperativity, which requires cooperative interaction of its multiple protein subunits, or allosteric regulation of HGF β activity can occur without cooperativity in any of the protein subunits.

The methods of the disclosure also include methods of identifying molecules that mimic HGF β binding to a ligand (such as the Met receptor), but do not activate the HGF/Met signaling pathway. HGF β is an inhibitor of full length HGF and can be used to identify or design other like inhibitors. These molecules can be identified using the three-dimensional model of HGF β using the coordinates of Table 5.

In another embodiment, a candidate modulator can be identified using a biological assay such as binding to HGF β, modulating Met phosporylation or modulating HGF induced cell migration. The candidate modulator can then serve as a model to design similar agents and/or to modify the candidate modulator for example, to improve characteristics such as binding to HGF β. Design or modification of candidate modulators can be accomplished using the crystal structure coordinates and available software.

Active Site and Other Structural Features

The disclosure provides information about the structure and shape of the binding site for Met, active site, activation domain, tunnel and dimerization region of HGF β. These structural features can be used in the methods for identification of modulators of HGF and/or HGF β.

The term “structural binding site,” as used herein, refers to a region of a molecule or molecular complex that, as a result of its structure can favorably associate with a ligand. Binding site structure factors can include, for example, the presence and disposition of amino acids residues in the binding region, and the two- or three-dimensional shape or topology of the HGF β molecule in or near the binding region, such as secondary structure (i.e., helices, sheets, or combinations thereof) or tertiary structure (i.e., the three dimensional disposition of molecular chains and features). Thus, a binding site may include or consist of features such as cavities, surfaces, or interfaces between domains. Ligands that may associate with a binding site include, but are not limited to, cofactors, substrates, agonists, and antagonists.

Binding sites are of significant utility in fields such as drug discovery. The association of natural ligands or substrates with the binding sites of their corresponding receptors or enzymes is the basis of many biological mechanisms of action. Similarly, many drugs exert their biological effects through association with the binding sites of receptors and enzymes. Such associations may occur with all or any part of the binding site. An understanding of such associations helps lead to the design of drugs having more favorable associations with their target, and thus improved biological effects. Therefore, this information is valuable in designing potential modulators of HGF and/or HGF β, as discussed in more detail below.

The amino acid constituents of a HGF β binding site for Met as defined herein are positioned in three dimensions. In one aspect, the structure coordinates defining a binding site of HGF β include structure coordinates of all atoms in the constituent amino acids; in another aspect, the structure coordinates of a binding site include structure coordinates of just the backbone atoms of the constituent amino acids.

In some embodiments, the amino acid residues of the structural HGF β binding site for Met comprise, consist essentially of, or consist of at least one or all of amino acid residues at positions 513, 516, 533, 534, 537-539, 578, 619, 647, 656, 668-670, 673,692-697, 699, 702, 705, or 707, or mixtures thereof or residues corresponding to these positions.

In another embodiment, HGF β binding site for Met comprises, consists essentially of, or consist of at least one or more or all of amino acid residues Tyr513, Lys516, Arg533, Gln534, Pro537, Ser538, Arg539, Asp578, Tyr619, Arg 647, Glu656, Pro668, Cys669, Glu670, Tyr673, Val692, Pro693, Gly694, Arg695, Gly696, Cys697, Ile699, Arg702, Ile705, Val707, or mixtures thereof, or conservative substitutions thereof. In other embodiments, the amino acid residues of the binding site comprise, consist essentially of, or consist of at least one or more or all of amino acids at a position 514, 534, 537, 578, 619, 621, 673, 692, 694 to 696, 699, or 701, or mixtures thereof. In another embodiment, the HGF β binding site for Met comprises, consists essentially of, or consists of at least one or more or all amino acid residues comprising Arg519, Gln534, Pro537, Asp578, Tyr619, Gly621, Tyr673, Val692, Gly694, Arg695, Gly696, Ile699, Asn 701, or mixtures thereof, or conservative substitutions thereof.

In another embodiment, the HGF β binding site for Met comprises, consists essentially of, or consists of at least one or all of core amino acid residues in positions 534, 578, 673, 692-694, 695, 696, or mixtures thereof. In a further embodiment, the HGF β binding site for Met comprise, consist essentially of, or consist of at least one or more or or all of core amino acid residues comprising Gln534, Asp578, Tyr673, Val692, Pro693, Gly694, Arg 695, Gly696, or mixtures thereof, or conservative substitutions thereof. In yet another embodiment, the binding site for Met on HGF β comprises, consists essentially of, or consists of at least one or more or all core amino acid or all amino acid residues in positions 673, 692-694, 695, 696, or mixtures thereof. In a further embodiment, the binding site for Met on HGF β comprises, consists essentially of, or consists of at least one or all amino acid residues Tyr673, Val692, Pro693, Gly694, Arg695, Gly696, or mixtures thereof, or conservative substitutions thereof. The numbering of the corresponding amino acid positions that form HGF β structural binding site in a structurally homologous molecule may change depending on the alignment of the structural homologous molecules with HGF β chain.

Alternatively, the structural binding site of HGF β may be defined by those amino acids whose backbone atoms are situated within about 5 Å of one or more constituent atoms of a bound substrate or ligand. In yet another alternative, the binding site for Met on HGF β can be defined by those amino acids whose backbone atoms are situated within a sphere centered on the coordinates representing the alpha carbon atom of a central amino acid residue Gly694, the sphere having a radius of about 5-6 Å, for example about 5.8 Å.

Accordingly, the disclosure provides molecules or molecular complexes including a HGF β structural binding site, as defined by the sets of structure coordinates of Table 5 and/or Table 6. In some embodiments, a structurally equivalent ligand binding site is defined by a root mean square deviation from the structure coordinates of Table 5 of the backbone atoms of the amino acids that make up the binding site in HGF β of at most about 0.70 Å, preferably about 0.5 Å.

Another structural feature of the HGF β chain is an activation domain. The activation domain in the β-chain can be identified by analogy to amino acid residues in serine proteases that undergo conformational change upon cleavage of chymotrypsinogen-like serine protease single chain pro-enzymes to two-chain enzymes. The activation domain includes parts of the Met binding site and other conformation changes in the HGF β chain. In some embodiments, the activation domain comprises, consists essentially of, or consists of one or more or all of amino acid residues in positions from about 495 to 498, 502 to 505, 618 to 627, 637 to 655, 660 to 672, 692 to 704, from 553 to 562, or mixtures thereof or residues corresponding to these positions. In some embodiments, the activation domain of HGF β comprises, consists essentially of, or consists of at least one or more or all amino acid residues Val495, Val496, Asn497, Gly498, Arg502, Thr503, Asn504, Ile505, Val553, His554, Gly555, Arg556, Gly557, Asp558, Glu559, Lys560, Cys561, Lys562, Gly618, Tyr619, Thr620, Gly621, Leu622, Ile623, Asn624, Tyr625, Asp626, Gly627, Met637, Gln638, Asn639, Glu640, Lys641, Cys642, Ser643, Gln644, His645, His646, Arg647, Gly648, Lys649, Val650, Thr651, Leu652, Asn653, Glu654, Ser655, Gly660, Ala661, Glu662, Lys663, Ile664, Gly665, Ser666, Gly667, Pro668, Cys669, Glu670, Gly671, Asp672, Val692, Pro693, Gly694, Arg695, Gly696, Cys697, Ala698, Ile699, Pro700, Asn701, Arg702, Pro703, Gly704, or mixtures thereof or conservative amino acid substitutions thereof.

In other embodiments, the amino acid residues of the activation domain comprise, consist essentially of, or consist of one or more or all amino acid residues from about position 495 to 498, 615 to 625, 660 to 670, 692 to 697, or 550 to 560 or mixtures therof. In some embodiments, the activation domain of HGF β comprises, consists essentially of, or consists of at least one or all amino acid residues Val495, Val496, Asn497, Gly498, Tyr615, Gly616, Trp617, Gly618, Tyr619, Thr620, Gly621, Leu622, Ile623, Asn624, Tyr625, Ile550, His551, Asp552, Val553, His554, Gly555, Arg556, Gly557, Asp558, Glu559, Lys560, Gly660, Ala661, Glu662, Lys663, Ile664, Gly665, Ser666, Gly667, Pro668, Cys669, Glu670, Val692, Pro693, Gly694, Arg695, Gly696, Cys697, or mixtures thereof or conservative amino acids substitutions thereof. In other embodiments, the activation domain of HGF β comprises, consists essentially of or consists of at least one or all core amino acid residue in positions 495-498,618-627, 660-672, 692-704, or mixtures thereof. In some embodiments, the activation domain of HGF β comprises, consists essentially of, or consists of one or more or even all core amino acid residues Val495, Val496, Asn497, Gly498, Gly618, Tyr619, Thr620, Gly621, Leu622, Ile623, Asn624, Tyr625, Asp626, Gly627, Gly660, Ala661, Glu662, Lys663, Ile664, Gly665, Ser666, Pro668, Cys669, Glu670, Gly671, Asp672, Val692, Pro693, Gly694, Arg695, Gly696, Cys697, Ala698, Ile699, Pro700, Asn701, Arg702, Pro703, Gly704, or mixtures thereof or conservative amino acid substitutions thereof.

Another structural feature identified in the HGF β chain crystal structure is an active site. The “active site” of HGF β refers to features analogous to the substrate binding cleft and catalytic amino acid triad capable of substrate cleavage in true serine protease enzymes. In some embodiments, amino acid residues associated with the active-site region of HGF β are summarized in Table 4 and comprise, consist essentially of, or consist of one or more or all amino acid residues corresponding to the catalytic triad, Asp 578, Tyr 673 and Gln534. The active site also includes amino acids that form the Met binding site including one or more or all amino acid residues from about 667 to 673, from about 532-536, from about 690 to 697, from about 637 to 655, or from about 574 to 579, or mixtures thereof. In some embodiments, the active site of HGF β comprises, consists essentially of, or consists of one or more or all amino acid residues Ala532, Arg533, Gln534, Cys535, Phe536, Pro574, Glu575, Gly576, Ser577, Asp578, Leu579, Met 637, Gly 638, Asn 639, Glu640, Lys641, Cys 642, Ser643, Gln644, His645, His646, Arg647, Gly648, Lys659, Val650, Thr651, Leu652, Asn 653, Glu654, Ser655, Gly667, Pro668, Cys669, Glu670, Gly671, Asp672, Tyr673, Val690, Ile691 Val692, Pro693, Gly694, Arg695, Gly696, Cys697, or mixtures thereof or conservative substitutions thereof.

In other embodiments, amino acid residues in the active site comprise, consist essentially of, or consist of some or all core amino acid residues 534, 578, 673, 693, 695, 696, 697, or 699, or mixtures thereof. In some embodiments, the active site of HGF β comprises, consists essentially of, or consists of one or more or all amino acid residues Gln534, Asp578, Tyr673, Pro693, Arg695, Gly696, Cys697, Ile699, or mixtures thereof or conservative substitutions thereof.

Another structural feature identified in the HGF β crystal is a tunnel. “Tunnel” refers to a pore-like void or aperture present in the HGF crystal structure. The amino acid positions forming the tunnel can be identified by determining the solvent accessibility of the amino acid positions in the HGF β crystal structure using standard methods. The “tunnel” feature, has an entrance near amino acid residues Tyr673 and Arg695, and comprises, consists essentially of, or consist of some or all amino acid residues 660 to 670, amino acid residues 693 to 706, amino acid residue 691, or amino acid residue 634, or mixtures thereof or residues corresponding to these positions. In some embodiments, the tunnel is formed by one or more or all amino acid residues comprising Tyr673, Arg695, Leu634, Ile691, Gly660, Ala661, Glu662, Lys663, Ile664, Gly665, Ser666, Gly667, Pro668, Cys669, Glu670, Pro693, Gly695, Gly696, Cys697, Ala698, Ile699, Pro700, Asn701, Arg703, Pro703, Gly704, or mixtures thereof or conservative substitutions thereof.

In other embodiments, the tunnel is formed by at least one or more or all core amino residues in positions comprising 669, 670, 673, 693-697, 662, 663, 701, or mixtures thereof. In some embodiments, the tunnel is formed by at least one or more or all core amino acid residues Cys669, Glu670, Tyr673, Pro693, Gly694, Arg695, Gly696, Cys697, Glu662, Lys663, Asn701, or mixtures thereof or conservative substitutions thereof. The tunnel, especially the tunnel entrance, is a likely interaction site for allosteric regulators of HGF β and/or HGF.

Another structural feature identified in the crystal structure of HGF β includes a dimerization region. In the crystal of HGF β a symmetric dimer is formed. The dimerization region includes amino acid residues that contact another HGF α-chain and are identified as those positions that lose solvent accessibility when two HGF β molecules are analyzed as a dimer. The dimerization region amino acid residues comprise, consist essentially of, or consists of some or all amino acid residues from about 495 to 502, 617-630, 660 to 670, or 700, or mixtures thereof. In some embodiments, the dimerization region of HGF β comprises, consists essentially of, or consists of one or more or all amino acid residues Val495, Val496, Asn497, Gly498, Ile499, Pro500, Thr501, Arg502, Trp617, Gly618, Tyr619, Thr620, Gly621, Leu622, Ile623, Asn624, Tyr625, Asp626, Gly627, Leu628, Leu629, Arg630, Gly660, Ala661, Glu662, Lys663, Ile664, Gly665, Ser666, Gly667, Pro668, Cys669, Glu670, Pro700, or mixtures thereof or conservative substitutions thereof.

In other embodiments, the amino acid positions of the dimerization domain comprise, consist essentially of, or consist of some or all amino acid residues from about 495 to 502, 620 to 624, 626, 628, 630, 662 to 665, or 700, or mixtures thereof. In some embodiments, the dimerization region of HGF β comprises, consists essentially of, or consists of one or more or all amino acid residues Val495, Val496, Asn497, Gly498, Ile499, Pro500, Thr501, Arg502, Trp620, Gly621, Leu622, Ile623, Asn624, Asp626, Gly627, Leu628, Arg630, Gly662, Lys663, Ile664, Gly665, Pro700, or mixtures thereof or conservative substituions thereof.

In some embodiments, the dimerization of HGF β comprises, consists essentially of, or consists of one or more or all core amino acid residues in positions 497, 499, 500, 502, 621-623, 662, 664, or mixtures thereof. In additional embodiments, the dimerization region of HGF β comprises, consists essentially of, or consists of one or more or all core amino acid residues Asn497, Ile499, Pro500, Arg502, Gly621, Leu622, Ile623, Gly662, Ile664 or mixtures thereof or conservative substitutions thereof.

Accordingly, the disclosure provides molecules or molecular complexes including the HGF β activation domain, active site, binding site for Met, tunnel and/or dimerization region as defined by the sets of structural coordinates of a crystal of HGF β, such as provided in Table 5 and/or Table 6. In some embodiments, structurally equivalent sites are defined by a root mean square deviation of at most about 0.70 Å, preferably about 0.50 Å, from the structural coordinates of the backbone of amino acids that makeup the activation domain, active site, binding site for Met, tunnel and/or dimerization region in HGF β. As discussed previously, it is understood that the amino acid numbering of corresponding positions of the structural features defined herein in a structurally homologous molecule may differ than that of the HGF.

Rational Drug Design

Computational techniques can be used to screen, identify, select, design ligands, and combinations thereof, capable of associating with and/or modulating activity of HGF and/or HGF β or structurally homologous molecules. Candidate modulators of HGF and/or HGF β may be identified using functional assays, such as binding to HGF β or inhibiting binding of HGF β to Met, KIRA assay, or cell migration assay as described herein. Novel modulators can then be designed based on the structure of the candidate molecules so identified. Knowledge of the structure coordinates for HGF β permits, for example, the design, the identification of synthetic compounds, and like processes, and the design, the identification of other molecules and like processes, that have a shape complementary to the conformation of the HGF β binding site, activation domain, active site, tunnel and/or dimerization region. In particular, computational techniques can be used to identify or design ligands, such as agonists and/or antagonists, that associate with and/or modulate activity of a HGF β binding site and/or other structural features, such as the active site, activation domain, dimerization region, and/or the tunnel.

Antagonists may bind to or interfere with all or a portion of an active site, activation domain, tunnel, dimerization region or binding site of HGF β, and can be competitive, non-competitive, or uncompetitive inhibitors. Once identified and screened for biological activity, these agonists, antagonists, and combinations thereof, may be used therapeutically or prophylactically, for example, to block HGF and/or HGF β activity and thus prevent the onset and/or further progression of diseases associated with dysregulation of HGF activity. Structure-activity data for analogues of ligands that bind to or interfere with HGF β binding sites, active sites, activation domain, dimerization region and/or tunnel can also be obtained computationally.

Data stored in a machine-readable storage medium that is capable of displaying a graphical three-dimensional representation of the structure of HGF β or a structurally homologous molecule, as identified herein, or portions thereof may thus be advantageously used for drug discovery. The structure coordinates of the ligand are used to generate a three-dimensional image that can be computationally fit to the three-dimensional image of HGF β or a structurally homologous molecule. The three-dimensional molecular structure encoded by the data in the data storage medium can then be computationally evaluated for its ability to associate with ligands. When the molecular structures encoded by the data is displayed in a graphical three-dimensional representation on a computer screen, the protein structure can also be visually inspected for potential association with ligands.

One embodiment of a method of drug design involves evaluating the potential association of a candidate ligand with HGF β or a structurally homologous molecule, particularly with a HGF β binding site. The method of drug design thus includes computationally evaluating the potential of a selected ligand to associate with any of the molecules or molecular complexes set forth above. This method includes the steps of: (a) employing computational means, for example, such as a programmable computer including the appropriate software known in the art or as disclosed herein, to perform a fitting operation between the selected ligand and a ligand binding site or a region nearby the ligand binding site of the molecule or molecular complex; and (b) analyzing the results of the fitting operation to identify and/or quantify the association between the ligand and the ligand binding site.

In another embodiment, the method of drug design involves computer-assisted design of ligands that associate with HGF β, its homologs, or portions thereof. Ligands can be designed in a step-wise fashion, one fragment at a time, or may be designed as a whole or de novo. Ligands can be designed based on the structure of molecules that can modulate at least one biological function of HGF β.

Other embodiments of a method of drug design involves evaluating the potential association of a candidate ligand with other structural features of HGF β or structurally homologous molecule. The method of drug design includes computationally evaluating the potential of the selected ligand to associate with HGF β and/or portion of the HGF β associated with the structural features. The structural features include activation domain, active site, tunnel, and/or dimerization region as described herein. The method comprises: (a) employing a computational means, for example, such as a programmable computer including the appropriate software to perform a fitting operation between the selected ligand and the structural feature of the HGF β; and (b) analyzing the results of the fitting operation to identify and/or quantify the association between the ligand and structural feature of HGF β chain.

Generally, to be a viable drug candidate, the ligand identified or designed according to the method is capable of structurally associating with at least part of a HGF β structural feature, and is able, sterically and energetically, to assume a conformation that allows it to associate with the HGF β structural feature, such as a binding site. Non-covalent molecular interactions important in this association include hydrogen bonding, van der Waals interactions, hydrophobic interactions, and/or electrostatic interactions. In some embodiments, agents may contact at least one, or any successive integer number up to all of the amino acid positions in the HGF β binding site or other structural feature. Conformational considerations include the overall three-dimensional structure and orientation of the ligand in relation to the ligand binding site, and the spacing between various functional groups of a ligand that directly interact with the HGF β binding site or homologs thereof.

Optionally, the potential binding of a ligand to a HGF β structural feature is analyzed using computer modeling techniques prior to the actual synthesis and testing of the ligand. If these computational experiments suggest insufficient interaction and association between it and the HGF β structural feature, testing of the ligand is obviated. However, if computer modeling indicates a sufficiently and/or desirably strong interaction, the molecule may then be synthesized and tested for its ability to bind to or interfere with, for example, a HGF β binding site. Binding assays to determine if a compound actually modulates HGF and/or HGF β activity can also be performed and are well known in the art.

Several methods can be used to screen ligands or fragments for the ability to associate with a HGF β structural feature. This process may begin by visual inspection of, for example, a HGF β structural feature, such as a binding site, on the computer screen based on the HGF β structure coordinates or other coordinates which define a similar shape generated from the machine-readable storage medium. Selected ligands may then be positioned in a variety of orientations, or docked, within the binding site, or other structural feature. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting ligands. Examples include GRID (Hubbard, S. 1999. Nature Struct. Biol. 6:7114); MCSS (Miranker, et al. 1991. Proteins 11:29-34) available from Molecular Simulations, San Diego, Calif.; AUTODOCK (Goodsell, et al. 1990. Proteins 8:195-202) available from Scripps Research Institute, La Jolla, Calif.; and DOCK (Kuntz, et al. 1982. J. Mol. Biol. 161:269-88) available from University of California, San Francisco, Calif.

HGF β binding ligands can be designed to fit a HGF β structural feature, based on the binding of a known modulator. There are many ligand design methods including, without limitation, LUDI (Bohm, 1992. J. Comput. Aided Molec. Design 6:61-78) available from Molecular Simulations Inc., San Diego, Calif.; LEGEND (Nishibata, Y., and Itai, A. 1993. J. Med. Chem. 36:2921-8) available from Molecular Simulations Inc., San Diego, Calif.; LeapFrog, available from Tripos Associates, St. Louis, Mo.; and SPROUT (Gillet, et al. 1993. J. Comput. Aided Mol. Design 7:127-53) available from the University of Leeds, UK.

Once a compound has been designed or selected by the above methods, the efficiency with which that ligand may bind to, modulate and/or interfere with a HGF β binding site or other structural feature may be tested and optimized by computational evaluation. For example, an effective HGF β binding site ligand should preferably demonstrate a relatively small difference in energy between its bound and free states (i.e., a small deformation energy of binding). Thus, an efficient HGF β binding site ligand should preferably be designed with a deformation energy of binding of not greater than about 10 to about 15 kcal/mole, such as about 12 kcal/mole, preferably not greater than about 8 to about 12 kcal/mole, such as about 10 kcal/mole, and more preferably not greater than about 5 to about 10 kcal/mole, such as about 7 kcal/mole. HGF β binding site ligands may interact with the binding site in more than one conformation that is similar in overall binding energy. In those cases, the deformation energy of binding is taken to be the difference between the free energy of the ligand and the average energy of the conformations observed when the ligand binds to the protein.

A ligand designed or selected as binding to, modulating and/or interfering with a HGF β binding site or other structural feature may be further computationally optimized so that in its bound state it would preferably lack repulsive electrostatic interaction with the target molecule and with the surrounding water molecules. Such non-complementary electrostatic interactions include repulsive charge-charge, dipole-dipole, and/or charge-dipole interactions.

Specific computer software is available to evaluate compound deformation energy and electrostatic interactions. Examples of programs designed for such uses include: Gaussian 94, revision C (M. J. Frisch, Gaussian, Inc., Pittsburgh, Pa.); AMBER, version 4.1 (P. A. Kollman, University of California at San Francisco,); QUANTA/CHARMM (Molecular Simulations, Inc., San Diego, Calif.); Insight II/Discover (Molecular Simulations, Inc., San Diego, Calif.); DelPhi (Molecular Simulations, Inc., San Diego, Calif.); and AMSOL (Quantum Chemistry Program Exchange, Indiana University). These programs can be implemented, for instance, using a Silicon Graphics workstation, such as an Indigo2 with IMPACT graphics. Other hardware systems and software packages will be known to those skilled in the art.

Another approach encompassed by this disclosure is the computational screening of small molecule databases for ligands or compounds that can bind in whole, or in part, to a HGF β structural feature, including binding site, active site, activation domain, tunnel, and/or dimerization region. In this screening, the quality of fit of such ligands to the binding site may be judged either by shape complementarity or by estimated interaction energy (Meng, et al., 1992. J. Comp. Chem., 13:505-24).

The disclosure also provides methods of identifying a molecule that mimics HGF β. HGF β is an inhibitor of full length HGF and can be used to identify or design other like inhibitors. One method involves searching a molecular structure database with the structural coordinates of Table 5, and selecting a molecule from the database that mimics the structural coordinates of HGF β. The method may also be conducted with portions of the HGF β structural coordinates that define structural features, such as binding site for Met, activation domain, active site, tunnel and/or dimerization region. The selected molecule can also be analyzed for differences between HGF β and the selected molecule at sites of the structural feature or can be tested for the ability to mimic one of the functional activities of HGF β. HGF β can then be modified to incorporate these differences and tested for functional activity and the modified HGF β can be selected for altered functional activity. In some embodiments, the modified HGF β molecule can bind Met, but not activate Met/HGF β signaling pathway.

Another method involves assessing agents that are antagonists or agonists of HGF β. A method comprises applying at least a portion of the crystallography coordinates of a crystal of HGF β, such as provided in Table 5 to a computer algorithm that generates a three-dimensional model of HGF β suitable for designing molecules that are antagonists or agonists and searching a molecular structure database to identify potential antagonists or agonists. In some embodiments, a portion of the structural coordinates of the crystal such as in Table 5 that define a structural feature, for example, binding site for Met, activation domain, active site, tunnel and/or dimerization region, may be utilized. The method may further comprise synthesizing or obtaining the agonist or antagonist and contacting the agonist or antagonist with HGF β and selecting the antagonist or agonist that modulates the HGF β and/or HGF activity compared to a control without the agonist or antagonists and/or selecting the antagonist or agonist that binds to HGF β. Activities of HGF β include phosphorylation of Met, stimulation of cell proliferation, and stimulation of cell migration.

A compound that is identified or designed as a result of any of these methods can be obtained (or synthesized) and tested for its biological activity, for example, binding to HGF and/or HGF β and/or modulation of HGF and/or HGF β activity. Other modulators of HGF β include, for example, monoclonal antibodies directed against HGF β, peptide(s) that can modulate HGF β function, or small-molecule compounds, such as organic and inorganic molecules, which can be identified with methods of the present disclosure.

7. Machine-Readable Storage Media

Transformation of the structure coordinates for all or a portion of HGF β or the HGF β/ligand complex or one of its ligand binding sites, for structurally homologous molecules (as defined below), or for the structural equivalents of any of these molecules or molecular complexes (as defined above), into three-dimensional graphical representations of the molecule or complex can be conveniently achieved through the use of commercially-available software.

The disclosure thus further provides a machine-readable storage medium including a data storage material encoded with machine-readable data wherein a machine programmed with instructions for using said data displays a graphical three-dimensional representation of any of the molecule or molecular complexes of this disclosure that have been described above. In one embodiment, the machine-readable data storage medium includes a data storage material encoded with machine-readable data wherein a machine programmed with instructions for using the abovementioned data displays a graphical three-dimensional representation of a molecule or molecular complex including all or any parts of a HGF β ligand binding site or a HGF β-like ligand binding site or other structural features, as defined above. In another embodiment, the machine-readable data storage medium includes a data storage material encoded with machine readable data wherein a machine programmed with instructions for using the data displays a graphical three-dimensional representation of a molecule or molecular complex having a root mean square deviation from the atoms of the amino acids of not more than about ±0.05 Å.

In an alternative embodiment, the machine-readable data storage medium can include, for example, a data storage material encoded with a first set of machine readable data which includes the Fourier transform of structure coordinates of HGF β, and wherein a machine programmed with instructions for using the first set of data is combined with a second set of machine readable data including the X-ray diffraction pattern of an unknown or incompletely known molecule or molecular complex to determine at least a portion of the structure coordinates corresponding to the second set of machine readable data.

For example, a system for reading a data storage medium may include a computer including a central processing unit (“CPU”), a working memory which may be, for example, RAM (random access memory) or “core” memory, mass storage memory (such as one or more disk drives or CD-ROM drives), one or more display devices (e.g., cathode-ray tube (“CRT”) displays, light emitting diode (“LED”) displays, liquid crystal displays (“LCDs”), electroluminescent displays, vacuum fluorescent displays, field emission displays (“FEDs”), plasma displays, projection panels, etc.), one or more user input devices (e.g., keyboards, microphones, mice, track balls, touch pads, etc.), one or more input lines, and one or more output lines, all of which are interconnected by a conventional bi-directional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this disclosure may be inputted via the use of a modem or modems connected by a telephone line or dedicated data line. Alternatively or additionally, the input hardware may include CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of a binding site of this disclosure using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this disclosure. Such programs are discussed in reference to the computational methods of drug discovery as described herein. Machine-readable storage devices useful in the present disclosure include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices can include necessary hardware (e.g., drives, controllers, power supplies, etc.) as well as any necessary media (e.g., disks, flash cards, etc.) to enable the storage of data.

8. Therapeutic Use

HGF modulator compounds obtained by methods of the invention are useful in a variety of therapeutic settings. For example, HGF β antagonists designed or identified using the crystal structure of the HGF β can be used to treat disorders or conditions, where inhibition or prevention of HGF and/or HGF β binding or activity is indicated.

Likewise, HGF β agonists designed or identified using the crystal structure of the HGF β can be used to treat disorders or conditions, where induction or stimulation or enhancement of HGF β activity is indicated.

An indication can be, for example, inhibition or stimulation of Met phosphorylation and the concomitant activation of a complex set of intracellular pathways that lead to cell growth, differentiation, and migration in a variety of cell types. The ability of HGF to stimulate mitogenesis, cell motility, and matrix invasion gives it a central role in angiogenensis, tumorogenesis and tissue regeneration. Another indication can be, for example, in inhibition or stimulation of embryonic development, for example, organogenesis. Still another indication can be, for example, in inhibition or stimulation of tissue regeneration. Another indication can be, for example, in inhibition of angiogenesis, mitogenesis and/or vasculogenesis. Expression of HGF has been associated with thyroid cancer, colon cancer, lymphoma, prostate cancer, and multiple myeloma. Yet another indication can be, for example, in inhibition or stimulation of the HGF/Met signaling pathway. Still yet another indication can be, for example, in inhibition of invasive tumor growth and metastasis.

HGF β antagonists are also useful as chemosensitizing agents, useful in combination with other chemotherapeutic drugs or growth inhibitory compounds, in particular, drugs that induce apoptosis. Examples of other chemotherapeutic drugs that can be used in combination with chemosensitizing HGF β inhibitors include topoisomerase I inhibitors (e.g., camptothecin or topotecan), topoisomerase II inhibitors (e.g., daunomycin and etoposide), alkylating agents (e.g., cyclophosphamide, melphalan and BCNU), tubulin-directed agents (e.g., taxol and vinblastine), and biological agents (e.g., antibodies such as anti CD20 antibody, IDEC 8, anti-VEGF antibody, immunotoxins, and cytokines). Other examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmoftur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-1; topoisomerase inhibitor RFS 2000; difluorometlhylornithine (DMFO); retinoids such as retinoic acid; capecitabine; and pharmaceutically acceptable salts, acids or derivatives of any of the above.

Also included in the definition of “chemotherapeutic agent” above are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

9. Other Uses

The HGF β chain, or variants thereof, form crystals in accord with the methods described herein. The crystals also are useful to store and/or deliver HGF β-chain molecules. HGF β may be useful as an inhibitor or antagonist of HGF. Crystals can be prepared and used to store HGF β-chain molecule for later use.

A variety of methods are known to those of skill in the art for formation of crystals. In some embodiments, for crystals prepared for storage, the crystal size and structure does not have to be so uniform or homogenous as for X-ray diffraction. In other embodiments, the crystals effectively diffract x-rays to a resolution of 5 Å or better. Typically, a purified polypeptide is contacted with a precipitant in the presence of a buffer. Precipitants include salts, polymers, or organic molecules. Organic precipitants include isopropanol, ethanol, hexanediol, and 2-methyl-2,4-pentanediol. Polymeric precipitants include polyethylene glycol and polyamines. Salts used include ammonium sulfate, sodium citrate, sodium acetate, ammonium dichloride, sodium chloride and magnesium formate. Many buffers can be utilized and are known to those of skill in the art.

In some cases, crystals can be cross-linked to one another. Such cross-linking may enhance the stability of the crystal. Methods of cross-linking crystals are know to those of skill in the art and have been described, for example, in U.S. Pat. No. 5,849,296.

The crystals can be maintained in crystallization solution, they can be dried, or combined with other carriers and/or other ingredients to form compositions and formulations. In some embodiments, the crystals can be combined with a polymeric carrier for stability and sustained release. In some embodiments, the HGF β has at least one biological activity when resolubilized. Biological activities of HGF β include binding to Met, phosphorylation of Met, stimulation of cell growth, and stimulation of cell migration.

Formulations of crystals of proteins, such as enzymes, receptors, antibodies, and like molecules, or fragments thereof, can include at least one ingredient or excipient. Ingredient or expedients are known to those of skill in the art and include acidifying agents, aerosol propellants, alcohol denaturants, alkalizing agents, anti-caking agents, antifoaming agents, microbial preservatives, anti-antioxidants, buffering agents, lubricants, chelating agents, colors, desiccants, emulsifying agents, filtering aids, flavors and perfumes, humectants, ointments, plasticizers, solvents (e.g. oils or organic), sorbents, carbon dioxide sorbents, stiffening agents, suppository bases, suspending or viscosity increasing agents, sweetening agents, tablet binders, table or capsule diluents, tablet disintegrants, tablet or capsule lubricants, tonicity agent, flavored or sweetened vehicles, oleaginous vehicles, solid carrier vehicles, water repelling agent, and wetting or solubilizing agents.

In some embodiments, the ingredients enhance storage stability. In other embodiments, the ingredient or excipient is preferably selected from the group consisting of albumin, sucrose, trehalose, lactitol, gelatin, and hydroxyproyl-β-cyclodextran.

All publications, patents, and patent documents are incorporated by reference herein, as though individually incorporated by reference. The disclosure has been described with reference to various specific and preferred embodiments and techniques. However, it should be understood that many variations and modifications can be made while remaining within the spirit and scope of the disclosure.

### EXAMPLE 1

### Expression and Purification of HGF β Proteins

HGF β proteins were expressed in insect cells using baculovirus secretion vector pAcGP67 (Pharmingen, San Diego, Calif.). All constructs contained a His₆ tag at the carboxy terminus and were purified to homogeneity (>95% purity) by Ni NTA metal chelate and gel filtration chromatography. For wildtype HGF β (SEQ ID NO:5), a cDNA fragment encoding the HGF β-chain from residues Val495 [c16] to Ser728 [c250] was cloned by PCR such that Val495 [c16] was inserted immediately after the secretion signal sequence. Site-directed mutagenesis was carried out using QuikChange™ (Stratagene, La Jolla, Calif.) with oligonucleotide 5′CCTAATTATGGATCCACAATTCCTG3′ (SEQ ID NO: 2) to make HGF β containing a Cys604 to Ser mutation (HGF β) (SEQ ID NO:1) HGF β mutants of SEQ ID NO:1 include Q534A [c57], D578A [c102], Y673A [c195], V692A [c214] and R695A [c217] were made as above in the HGFβ construct.

proHGF β (SEQ ID NO:7) encodes HGF from residues Asn479 to Ser728 and has a R494E mutation made using the oligonucleotide 5′CAAAACGAAACAATTGGAAGTTGTAAATGGGATTC 3′ (SEQ ID NO: 3). The cysteine was not altered in this construct to allow putative disulfide formation between Cys487 and Cys604.

Numbering for all amino acid sequences is as follows: full length HGF sequence starting with MWV . . . as numbers 1-3 [chymotrypsinogen numbering is shown in the brackets]. It will be readily apparent that the numbering of amino acids in other isoforms of HGF β may be different than that of the HGF β numbering disclosed herein. The disclosure provides sequential numbering based on sequence only. In some embodiments, an isoform may have structural “differences”, for example, if it carries insertion(s) or deletion(s) relative to the HGF β reference sequence. The chymotrypsinogen numbering convention may be useful for comparison.

The amino acid sequence of a HGF β (SEQ ID NO:1) is shown in Table 7. The amino acid sequence of wild type HGF β (SEQ ID NO:5) is shown in Table 9 and a full length HGF comprising an amino acid sequence of SEQ ID NO:6 is shown in Table 10. Other sequences are known to those of skill in the art.

Baculovirus vectors containing the desired inserts were transfected into Spodoptera frugiperda (Sf 9) cells on plates in TNM-FH media via the Baculogold™ Expression System according to manufacturer's instructions (Pharmingen, San Diego, Calif.). After 24 rounds of virus amplification, 10 mL of viral stock was used to infect 1 L of High Five™ cells (Invitrogen, San Diego, Calif.) in suspension at 5×10⁵ cells/mL in TNM-FH media. Cultures were incubated at 27° C. for 72 h before harvesting the culture media by centrifugation at 8,000×g for 15 min. Cell culture media was applied to a 4 mL Ni-NTA agarose column (Qiagen, Valencia, Calif.). After washing with 4 column volumes of 50 mM Tris.HCl, pH 8.0, 500 mM NaCl, 5 mM imidazole, HGF β proteins were eluted with 50 mM Tris.HCl pH 8.0, 500 mM NaCl, 500 mM imidazole. The eluate was pooled and applied to a Superdex™-200 column (Amersham Biosciences, Piscataway, N.J.) equilibrated in 10 mM HEPES pH 7.2, 150 mM NaCl, 5 mM CaCl₂. Protein peaks were collected and concentrated using a Centriprep™ YM-10 (Millipore, Bedford, Mass.). Fractions were analyzed by 12% SDS-PAGE stained with Coomassie blue. All mutations were verified by DNA sequencing and mass spectrometry. Protein concentration was determined by quantitative amino acid analysis. N-terminal sequencing revealed a single correct N-terminus present for proHGF β and HGF β. Purified proteins showed the correct molecular mass on SDS-PAGE; multiple bands observed were likely due to heterogeneous glycosylation, consistent with the mass spectrometry data having molecular masses about 2 kDa higher than predicted from the sequence.

Construction, Expression, and Purification of Full Length Variant HGF Proteins

Recombinant proteins were produced in 1 L cultures of Chinese hamster ovary (CHO) cells by transient transfection (Peek et al., 2002). pRK5.1 vectors used for CHO expression (Lokker 1992). Amino acid changes were introduced by site-directed mutagenesis (Kunkel, 1985) and verified by DNA sequencing. The expression medium (F-12/Dulbecco's modified Eagle's medium) contained 1% (v/v) ultra low IgG fetal bovine serum (FBS) (Gibco, Grand Island, N.Y.). After 8 days the medium was harvested and supplemented with FBS to give a final content of 5-10% (v/v). Additional incubation for 2-3 days at 37° C. resulted in complete single-chain HGF conversion. This step was omitted for expression of proHGF, an uncleavable single chain form, which has amino acid changes at the activation cleavage site (R494E) and at a protease-susceptible site in the α-chain (R424A) (Peek et al., 2002). Mutant proteins were purified from the medium by HiTrap-Sepharose SP cation exchange chromatography (Amersham Biosciences, Piscataway, N.J.) as described (Peek et al., 2002). Examination by SDS-PAGE (4-20% gradient gel) under reducing conditions and staining with Simply Blue Safestain showed that all mutant HGF proteins were >95% pure and were fully converted into α/β-heterodimers except for proHGF, which remained as a single-chain form. Protein concentration for each mutant was determined by quantitative amino acid analysis.

Expression and Purification of MetECD

The mature form of the Met ECD (Glu25 to Gln929) (SEQ ID NO:4) domain containing a C-terminal His₆ tag were expressed in insect cells and purified by Ni-NTA metal chelate and gel filtration chromatography using standard protocols described above. Met-IgG fusion protein was obtained as previously described (Mark et al., 1992). A representative amino acid sequence of wild type extracellular domain of the Met receptor is shown in Table 8. (SEQ ID NO: 4) Other sequences are known to those of skill in the art.

### EXAMPLE 2

### Characterization of HGF and HGF Variants

Materials and Methods

HGF β and Met Binding Affinity by Surface Plasmon Resonance

The binding affinity between HGF β and Met was determined by surface plasmon resonance using a Biacore 3000 instrument (Biacore, Inc., Piscataway, N.J.). The Met ECD domain was immobilized on a CM5 chip using amine coupling at about 2000 resonance units according to the manufacturer's instructions. A series of concentrations of HGF β in 10 mM HEPES pH 7.2, 150 mM NaCl, 5 mM CaCl₂ ranging from 12.5 nM to 100 nM were injected at a flow rate of 20 μL/min for 40 s. Bound HGF β was allowed to dissociate for 10 min. Appropriate background subtraction was carried out. The association (kₒₙ) and dissociation (k_{off}) rate constants were obtained by a global fitting program provided with the instrument; the ratio of k_{off}kₒₙ was used to calculate the dissociation constant (K_{d}).

Binding of HGF β to Met and Competition Binding ELISA

Microtiter plates (Nunc, Roskilde, Denmark) were coated overnight at 4° C. with 2 μg/mL of rabbit anti-human IgG Fc specific antibody (Jackson ImmunoResearch Laboratory, West Grove, Pa.) in 50 mM sodium carbonate buffer, pH 9.6. After blocking with 1% BSA in HBS buffer (50 mM HEPES pH 7.2, 150 mM NaCl, 5 mM CaCl₂ and 0.1% Tween-20), 1 μg/mL Met-IgG fusion protein (Mark et al., 1992) was added and plates were incubated for 1 h with gentle shaking at room temperature. After washing with HBS buffer, HGF β proteins were added for 1 h. Bound HGF β was detected using anti-His-HRP (Qiagen, Valencia, Calif.) followed by addition of TMB/H₂O₂ substrate (KPL, Gaithersburg, Md.). The reaction was stopped with 1M H₃PO₄ and the A₄₅₀ was measured on a Molecular Devices SpectraMax Plus³⁸⁴ microplate reader. The effective concentration to give half-maximal binding (EC₅₀) was determined by a four parameter fit using Kaleidagraph (Synergy Software, Reading, Pa.).

In order to develop a competition ELISA, wildtype HGF β was biotinylated using a 20-fold molar excess of biotin-maleimide (Pierce, Rockford, Ill.) at room temperature for 2 h. Plates were treated as above except biotinylated wildtype HGF β was used and detected using HRP-neutravidin (Pierce, Rockford, Ill.). Competition assays contained a mixture of 250 nM biotinylated wildtype HGF β and various concentrations of unlabeled HGF β variants, HGF or proHGF. After incubation for 1 h at room temperature, the amount of biotinylated wildtype HGF β bound on the plate was measured as described above. IC₅₀ values were determined by fitting the data to a four-parameter equation (Kaleidagraph, Synergy Software, Reading, Pa.).

Binding of HGF Mutants to Met

Biotinylated HGF was prepared using the Sigma immunoprobe biotinylation kit (Sigma, St. Louis, Mo.). Microtiter plates were coated with rabbit anti-human IgG Fc specific antibody as above. Plates were washed in PBS 0.05% (v/v) Tween-20 followed by a 1 h incubation with 0.5% (w/v) of BSA, 0.05% Tween-20 in PBS, pH 7.4 at room temperature. After washing, 1 nM biotinylated HGF and 0.2 nM Met-IgG fusion protein together with various concentrations of HGF mutants were added to the wells and incubated for 2 h. After washing, bound biotinylated HGF was detected by addition of diluted (1:3000) streptavidin horseradish peroxidase conjugate (Zymed, South San Francisco, Calif.) followed by SureBlue TMB peroxidase substrate and stop solution TMB STOP (KPL, Gaithersburg, Md.). The A₄₅₀ was measured and IC₅₀ values were determined as described above. Relative binding affinities are expressed as the IC₅₀(mutant)/IC₅₀(wildtype HGF).

HGF Dependent Phosphorylation of Met

The kinase receptor activation assay (KIRA) was run as follows. Confluent cultures of lung carcinoma A549 cells (CCL-185, ATCC, Manassas, Va.), previously maintained in growth medium (Ham's F12/DMEM 50:50 (Gibco, Grand Island, N.Y.) containing 10% FBS, (Sigma, St. Louis, Mo.), were detached using Accutase (ICN, Aurora, Ohio) and seeded in 96 well plates at a density of 50,000 cells per well. After overnight incubation at 37° C., growth media was removed and cells were serum starved for 30 to 60 min in medium containing 0.1% FBS. Met phosphorylation activity by HGF, HGF mutants or HGF β-chain was determined from addition of serial dilutions from 500 to 0.2 ng/mL in medium containing 0.1% FBS followed by a 10 minute incubation at 37° C., removal of media and cell lysis with 1× cell lysis buffer (Cat. #9803, Cell Signaling Technologies, Beverly, Mass.) supplemented with 1× protease inhibitor cocktail set I (Cat. No. 539131, Calbiochem, San Diego, Calif.). Inhibition of HGF dependent Met phosphorylation activity by HGF β-chain was determined from addition of serial dilutions from 156 to 0.06 nM to assay plates followed by a 15 min incubation at 37° C., addition of HGF at 12.5, 25 or 50 nM, an additional 10 min incubation at 37° C., removal of media and cell lysis as above. Cell lysates were analyzed for phosphorylated Met via an electrochemiluminescence assay using an ORIGEN M-Series instrument (IGEN International, Gaithersburg, Md.). Anti-phosphotyrosine mAb 4G10 (Upstate, Lake Placid, N.Y.) was labeled with ORI-TAG via NHS-ester chemistry according to manufacturer's directions (IGEN). Anti-Met ECD mAb 1928 (Genentech) was biotinylated using biotin-X-NHS (Research Organics, Cleveland, Ohio). The ORI-TAG-labeled 4G10 and biotinylated anti-Met mAb were diluted in assay buffer (PBS, 0.5% Tween-10, 0.5% BSA) and the cocktail was added to the cell lysates. After incubation at room temperature with vigorous shaking for 1.5 to 2 h, addition of streptavidin magnetic beads (Dynabeads, IGEN), and another incubation for 45 min, plates were read on the ORIGEN instrument.

Cell Migration Assay

Breast cancer cells MDA-MB-435 (HTB-129, ATCC, Manassas, Va.) were cultured in recommended serum-supplemented medium. Confluent cells were detached in PBS containing 10 mM EDTA and diluted with serum-free medium to a final concentration of 0.6-0.8×10⁵ cells/mL. 0.2 mL of this suspension (1.2-1.6×10⁵ total cells) was added in triplicate to the upper chambers of 24-well transwell plates (8 μm pore size) (HTS Multiwell™ Insert System, Falcon, Franklin Lakes, N.J.) pre-coated with 10 μg/mL of rat tail collagen Type I (Upstate, Lake Placid, N.Y.). Wildtype HGF or HGF mutants were added to the lower chamber at 100 ng/mL in serum-free medium, unless specified otherwise. After incubation for 13-14 h cells on the apical side of the membrane were removed and those that migrated to the basal side were fixed in 4% paraformaldehyde followed by staining with a 0.5% crystal violet solution. After washing and air-drying, cells were solubilized in 10% acetic acid and the A₅₆₀ was measured on a Molecular Devices microplate-reader. Pro-migratory activities of HGF mutants were expressed as percent of HGF controls after subtracting basal migration in the absence of HGF. Photographs of stained cells were taken with a Spot digital camera (Diagnostics Instruments, Inc., Sterling Heights, Mich.) connected to a Leitz microscope (Leica Mikroskope & Systeme GmbH, Wetzlar, Germany). Pictures were acquired by Adobe Photoshop 4.0.1 (Adobe Systems Inc., San Jose, Calif.).

Results

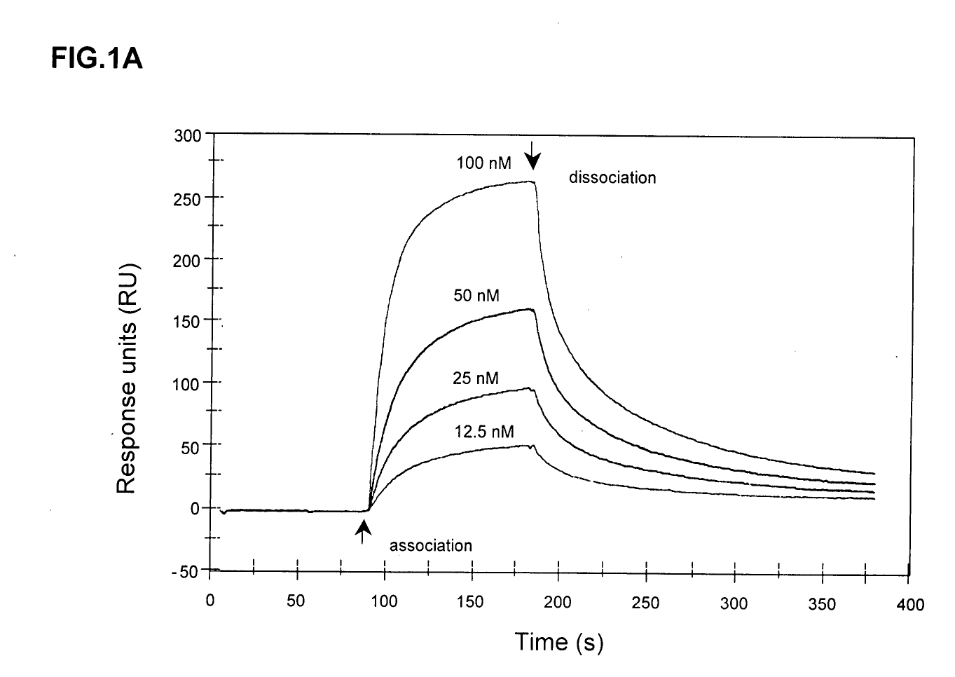

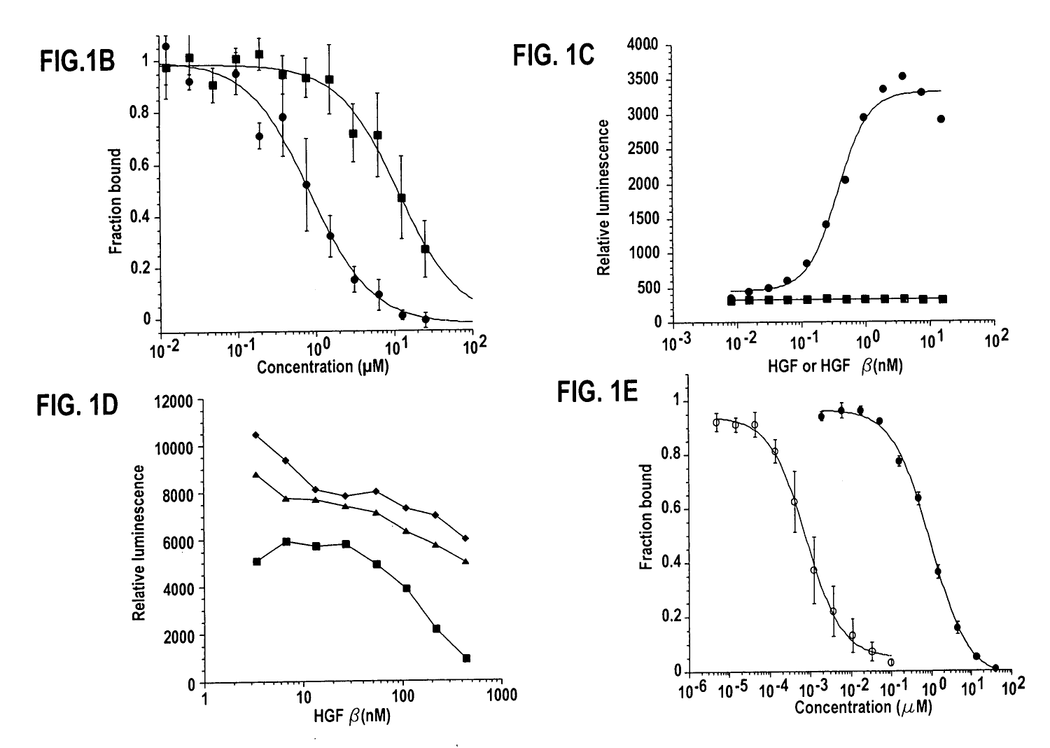

HGF β binding to Met was assessed from the change in resonance units measured by surface plasmon resonance on a CM5 chip derivatized with the extracellular domain of Met (Met ECD). The results show that HGF β binds to Met ECD with a K_{d} of 90 nM calculated from relatively fast association (kₒₙ=1.2×10⁵ M⁻¹s⁻¹) and dissociation rate constants (k_{off}=0.011 s⁻¹) (FIG. 1A). Binding of HGF β to Met was also confirmed by a second independent method using a plate ELISA. Following incubation of biotinylated HGF β with a properly oriented Met-IgG fusion bound to an immobilized anti-Fc antibody and detection with HRP-neutravidin, an EC₅₀ value of 320±140 nM was determined (n=6; data not shown)

Since single-chain HGF binds to Met with comparable affinity to two-chain HGF, but does not induce Met phosphorylation (Lokker et al., 1992; Hartmann et al., 1992). This may be due to the lack of a Met binding site in the uncleaved form of the β-chain. proHGF β, a zymogen-like form of HGF β containing the C-terminal 16 residues from the HGF α-chain and a mutation at the cleavage site (R494E) to ensure that the single-chain form remained intact was expressed and purified. Binding of HGF β and proHGF β to Met was determined with a competition binding ELISA, resulting in IC₅₀ values of 0.86±0.17 and 11.6±1.8 μM, respectively (FIG. 1B). The 13.5-fold reduced binding shows that while a Met binding site on the zymogen-like HGF β does in fact exist, it is not optimal.

Although HGF β binds to Met, it does not induce Met phosphorylation (FIG. 1C). However, HGF β does inhibit HGF dependent phosphorylation of Met in a concentration dependent manner (FIG. 1D), although the inhibition was incomplete at the highest concentration used. Inhibition of Met phosphorylation is consistent with a direct competition with HGF for Met binding. In agreement with this, competition binding assays show that HGF β inhibits full length HGF binding to Met (FIG. 1E), albeit at rather high concentrations (IC₅₀=830±26 nM; n=3). By comparison, full length wildtype HGF had an IC₅₀ value of 0.86±0.47 nM (n=3) in this assay.

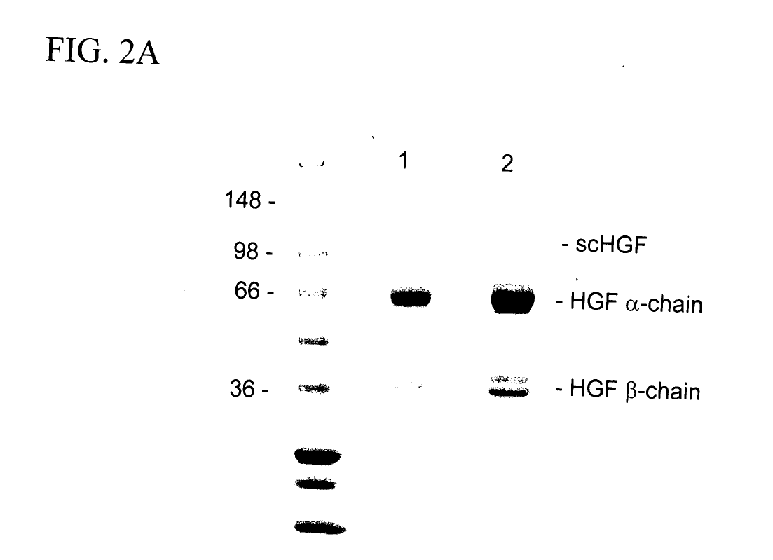

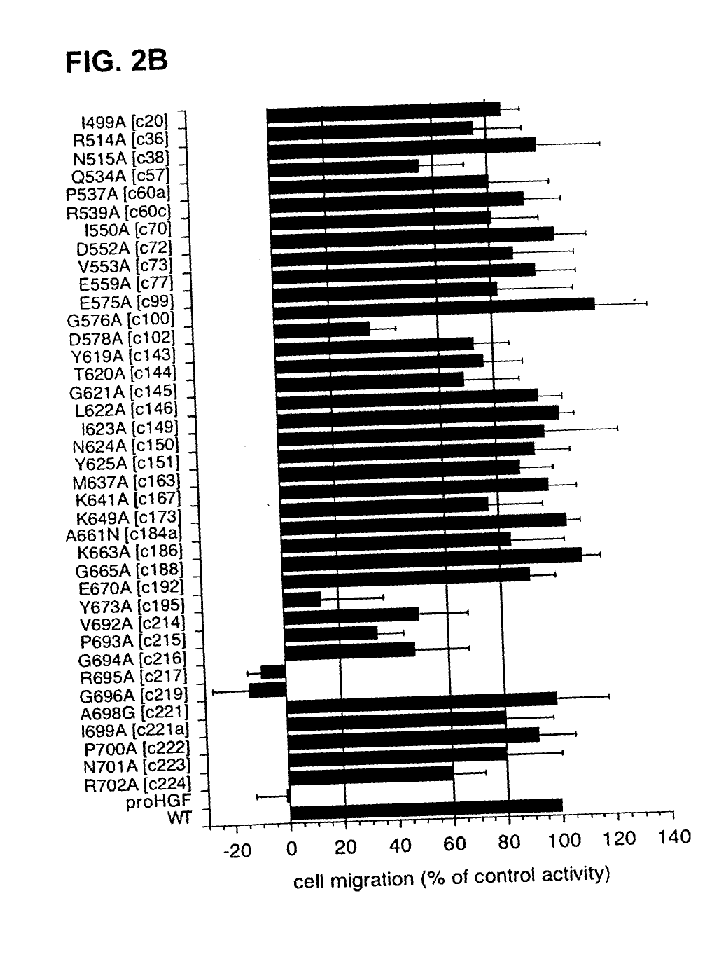

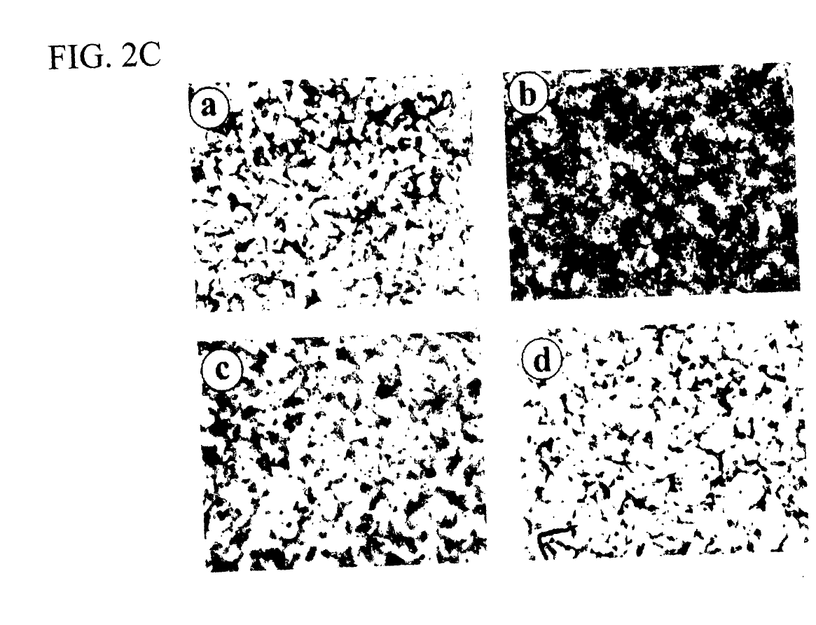

To identify the Met binding site in the β-chain, residues were systematically changed in regions corresponding to the activation-domain and the active-site of serine proteases. Initial expression of HGF mutants in CHO cells yielded a mixture of single- and two-chain HGF forms, exemplified by mutant HGF 1623A (FIG. 2A). Complete conversion of residual uncleaved HGF was accomplished by additional exposure of the harvested culture medium to 5-10% serum for several days (FIG. 2A). The purity of HGF 1623A following purification by cation exchange chromatography is representative of all HGF mutants (FIG. 2A).

The functional consequence of mutating β-chain residues in HGF was assessed by determining the ability of the HGF mutants to stimulate migration of MDA-MB435 cells. The results showed that 3 HGF mutants, R695A [c217], G696A [c219] and Y673A [c195] were severely impaired, having less than 20% of wildtype activity, while 4 mutants Q534A [c57], D578A [c102], V692A [c214] and G694A [c216] had 20%-60% of wildtype activity (FIG. 2B). An additional set of 9 mutants (R514A, P537A, Y619A, T620A, G621A, K694A, I699A and N701A) and R702A had 60-80% of wildtype activity. The remaining 21 mutants had activities >80% that of the wildtype and were considered essentially unchanged from HGF. As expected, proHGF did not stimulate cell migration (FIG. 2B). The complete inability of 1 nM R695A [c217] or G696A [c219] to promote cell migration is illustrated in FIG. 2C, showing that migration in the presence of either mutant is similar to basal migration in the absence of HGF.

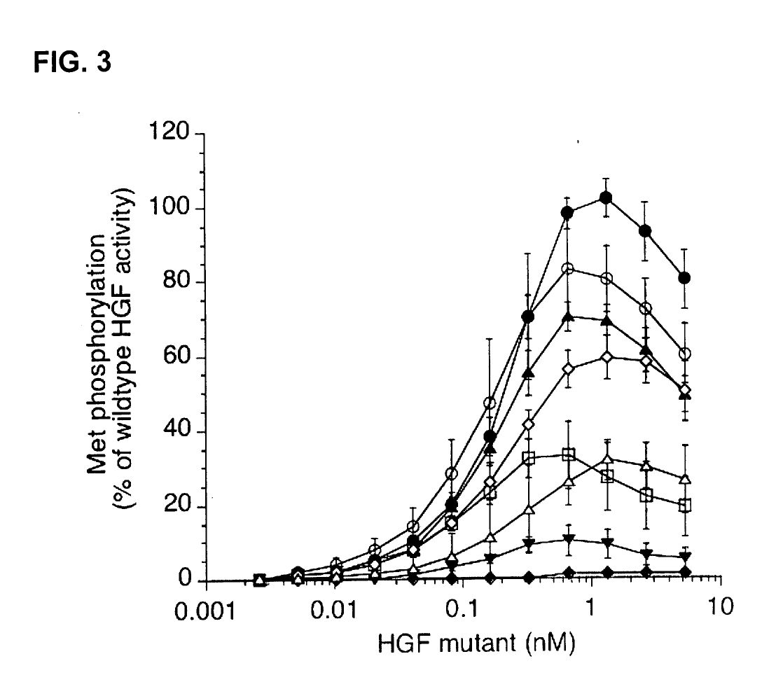

To examine whether reduced activities in cell migration correlated with reduced Met phosphorylation, a subset of HGF mutants was examined in a kinase receptor assay (KIRA). For wildtype HGF and HGF mutants, maximal Met phosphorylation was observed at concentrations between 0.63 and 1.25 nM (FIG. 3). The maximal Met phosphorylation achieved by mutants Y673A [c195], R695A [c217] and G696A [c219] was less than 30% of wildtype, agreeing with their minimal or absent pro-migratory activities. Mutants Q534A [c57], D578A [c102] and V692A [c214] had intermediate activities (30-60%) in cell migration assays; they also had intermediate levels of Met phosphorylation, having 56%-83% that of wildtype HGF. In agreement with its lack of cell migration activity, proHGF had no Met phosphorylation activity (FIG. 3).

The affinity of each mutant for Met-IgG fusion protein was analyzed by HGF competition binding; 34 HGF mutants had essentially the same binding affinity as two-chain HGF (IC₅₀=0.83±0.32 nM; n=30), indicated by their IC₅₀ ratios (IC₅₀mut/IC₅₀WT), which ranged from 0.36 to 2.0 (Table 1). HGF Y673A [c195], K649A, and proHGF showed about a 4-fold weaker binding to Met-IgG compared to HGF (Table 1). The cell migration activities of selected mutants at 10- and 50-fold higher concentrations was examined; no increase in pro-migratory activity was observed (Table 2). Therefore, the impaired function of HGF mutants is not due to reduced binding to Met, since an increase in concentration of up to 50-fold had no compensatory effect.

TABLE 1
Binding of HGF mutants to Met

| | IC₅₀mut/ | | IC₅₀mut/ |
|---|---|---|---|
| HGF mutant | IC₅₀WT ± SD | HGF mutant | IC₅₀WT ± SD |
| I499A [c20] | 0.52 | N624A [c150] | 0.71 ± 0.18 |
| R514A [c36] | 1.41 ± 0.23 | Y625A [c151] | 0.65 ± 0.26 |
| N515A [c38] | 1.16 | M637A [c163] | 1.38 |
| Q534A [c57] | 2.04 ± 0.86 | K641A [c167] | 1.04 |
| P537A [c60a] | 1.67 | A661N [c184a] | 1.04 ± 0.34 |
| R539A [c60c] | 0.94 | K663A [c186] | 0.73 ± 0.20 |
| I550A [c70] | 1.39 | G665A [c188] | 0.36 ± 0.03 |
| D552A [c72] | 1.23 | E670A [c192] | 1.77 |
| V553A [c73] | 0.99 ± 0.26 | Y673A [c195] | 4.41 ± 1.03 |
| E559A [c77] | 1.34 ± 0.07 | V692A [c214] | 1.74 ± 0.16 |
| E575A [c99] | 1.19 ± 0.05 | G694A [c216] | 1.76 ± 0.72 |
| G576A [c100] | 0.78 | R695A [c217] | 1.48 ± 0.52 |
| D578A [c102] | 1.86 ± 0.82 | G696A [c219] | 2.03 ± 1.04 |
| Y619A [c143] | 1.52 ± 0.28 | A698G [c221] | 0.73 ± 0.35 |
| T620A [c144] | 1.89 ± 0.32 | I699A [c221a] | 1.79 ± 0.70 |
| G621A [c145] | 1.08 ± 0.30 | P700A [c222] | 1.30 ± 0.46 |
| L622A [c146] | 1.04 ± 0.22 | N701A [c223] | 1.48 ± 0.59 |
| I623A [c149] | 0.49 ± 0.10 | proHGF | 4.03 ± 1.05 |
| K649 [c173] | 3.66 | R702A [c224] | 2.25 |

TABLE 2
Pro-migratory activities of HGF mutants at different concentrations.

| | Pro-migratory | Pro-migratory | Pro-migratory |
|---|---|---|---|
| | activity | activity | activity |
| | at 1 nM | at 10 nM | at 50 nM |
| Mutant | (% of control) | (% of control) | (% of control) |
| Y673A | 13.9 ± 8.9 | 9.8 ± 8.3 | 9.1 ± 8.6 |
| V692A | 49.5 ± 17.7 | 20.9 ± 6.9 | 29.8 ± 6.3 |
| G694A | 47.6 ± 19.7 | 23.2 ± 11.4 | 21.2 ± 5.9 |
| R695A | -8.9 ± 5.4 | -4.4 ± 11.6 | 5.3 ± 10.2 |
| G696A | -13.6 ± 13.7 | 4.0 ± 19.8 | 2.8 ± 7.1 |

The poor correlation between HGF binding to Met and either HGF dependent cell migration or Met phosphorylation is likely due to the relatively high affinity between Met and the HGF α-chain, which could mask any reduced affinity due to the β-chain. Therefore, selected mutations in HGF β itself were made to eliminate any α-chain effects. HGF β mutants Q534A [c57], D578A [c102], Y673A [c195], V692A [c214] and R695A [c217] were tested in a competition ELISA with biotinylated HGF β binding to Met-IgG (FIG. 4). Mutants were then normalized to HGF β, which had an IC₅₀=0.47±0.34 μM (n=14), to determine their relative affinities (FIG. 4). The relative IC₅₀ values±SD (n≧3) are as follows: HGF β: 1, Q534A: 12.5±3.6, D578A: 16.6±8.2, Y673A: >>100, V692A: >50, R695A: >>100 and proHGF β: 21±10. Mutants R695A, G696A and Y673A had the greatest loss in migration activity (in the 2 chain form) and also had the greatest loss in Met binding as HGF β mutants. A strong correlation for reduced activity of full-length two-chain HGF mutants with reduced binding of the corresponding mutant of HGF β was seen.

HGF acquires biological activity upon proteolytic conversion of the single chain precursor form into two-chain HGF (Naka et al., 1992; Hartmann et al., 1992; Lokker et al., 1992; Naldini et al. 1992). Based on the structural similarity of HGF with chymotrypsin-like serine proteases (Perona and Craik, 1995; Rawlings et al., 2002; Donate et al., 1994) and plasminogen in particular, whether this activation process is associated with structural changes occurring in the HGF β-chain was studied.

Binding studies with purified HGF β-chains revealed that the ‘activated’ form of HGF β (Val495-Ser728) binds to Met with about a 13-fold higher affinity than its precursor form, proHGF β (Asn479-Ser728), consistent with the view that optimization of the Met binding site is contingent upon processing of single-chain HGF. This suggested that the Met binding site includes the HGF region undergoing conformational rearrangements after scHGF cleavage, i.e. the ‘activation domain’. Indeed, functional analysis of HGF variants with amino acid substitutions in the ‘activation domain’ led to the identification of the functional Met binding site. However, HGF mutants with the greatest losses in pro-migratory activities (Q534A, D578A, Y673A, V692A, G694A, R695A, G696A) displayed essentially unchanged binding affinities for Met, except for Y673A (4-fold loss), because HGF affinity is dominated by the HGF α-chain (Lokker et al., 1994; Okigaki et al., 1992). Consistent with this, the reduced activities remained unchanged upon increasing the concentration of HGF mutants by more than 50-fold. Therefore, the reduced activities of HGF mutants were interpreted as resulting from perturbed molecular interactions of HGF β-chain with its specific, low affinity, binding site on Met. In support of this, it was found that the reduced biological activities of selected HGF mutants were well correlated with reduced Met binding of the corresponding HGF β mutants.

### EXAMPLE 3

### Crystallization and Three Dimensional Analysis of HGF

Materials and Methods

HGF β X-Ray Structure

Purified HGF β (SEQ ID NO:1) was concentrated to 10 mg/mL using a Centriprep® YM-10 in 10 mM HEPES pH 7.2, 150 mM NaCl, 5 mM CaCl₂. Hanging drops (1 microliter protein and 1 microliter 30% PEG-1500) over a reservoir containing 500 microliter 30% PEG-1500 (Hampton Research, Laguna Niguel, Calif.) yielded crystalline rods (about 25×25×500 micrometers) during incubation at 19° C. overnight. A crystal fragment was preserved directly from the mother liquor by immersion in liquid nitrogen. Data extending to 2.53 Å resolution were collected on a Quantum 4 CCD detector (ADSC, Poway, Calif.) at ALS beam line 5.0.2 with 1.0 Å wavelength X-rays. Data processing and reduction were performed using HKL (Otwinowski and Minor, 1996) (HKL Research, Charlottesville, Va.) and ccp4 (CCP4, 1994).

The structure was solved by molecular replacement using AMoRe (Navaza, 1994) in space group P3₁21, using parts of the protease domain of coagulation factor VIIa (Dennis et al., 2000) as the search probe. Refinement was performed using X-PLOR98 (MSI, San Diego) and REFMAC (Murshudov et al., 1997). Inspection of electron density maps and model manipulation were performed using XtalView (McRee, 1999) (Syrrx, San Diego, Calif.). The number in parenthesisis the number of atoms assigned zero occupancy.

TABLE 3
Structure Statistics for HGF β.
Data: space group P3₁21 a = 63.7 Å, c = 135.1 Å

| Reso- | | | | | | | |
|---|---|---|---|---|---|---|---|
| lution | | | Com- | | | | |
| (Å) | Nmeas¹ | Nref² | plete³ | I/σ | Rmerge⁴ | Rwork⁵ | Rfree⁶ |
| 5.45- | 5835 | 1219 | 100 | 44 | 0.032 | 0.274 | 0.309 |
| 50.0 | | | | | | | |
| 4.33- | 5882 | 1143 | 100 | 43 | 0.035 | 0.211 | 0.277 |
| 5.45 | | | | | | | |
| 3.78- | 5896 | 1134 | 100 | 36 | 0.043 | 0.216 | 0.260 |
| 4.33 | | | | | | | |
| 3.43- | 5790 | 1107 | 100 | 28 | 0.060 | 0.237 | 0.291 |
| 3.78 | | | | | | | |
| 3.19- | 5724 | 1097 | 100 | 20 | 0.086 | 0.265 | 0.330 |
| 3.43 | | | | | | | |
| 3.00- | 5903 | 1115 | 100 | 13 | 0.126 | 0.295 | 0.352 |
| 3.19 | | | | | | | |
| 2.85- | 5875 | 1117 | 100 | 8.8 | 0.190 | 0.287 | 0.356 |
| 3.00 | | | | | | | |
| 2.73- | 5575 | 1072 | 100 | 5.9 | 0.269 | 0.278 | 0.327 |
| 2.85 | | | | | | | |
| 2.62- | 5005 | 1077 | 98 | 3.6 | 0.367 | 0.294 | 0.253 |
| 2.73 | | | | | | | |
| 2.53- | 3350 | 886 | 83 | 2.7 | 0.368 | 0.323 | 0.385 |
| 2.62 | | | | | | | |
| 2.53- | 54835 | 10967 | 98 | 24 | 0.064 | 0.246 | 0.303 |
| 50.0 | | | | | | | |

Final Model

contents of model

r.m.s deviations

| residues | atoms⁷ | waters | bonds | angles | B-factor |
|---|---|---|---|---|---|
| 227 | 1798(106) | 33 | 0.012 Å | 1.5° | 5 Å² |

Data collection
Resolution 50.0-2.53 Å (outer shell = 2.62-2.53)
Rsym 0.064 (0.368 for the outer shell)
No. observations 54835
unique reflections 10967
completeness 98%(83% in the outer shell)
Refinement
resolution 50-2.53 Å
number reflections 10,967
R, Rfree 0.246, 0.303
¹Nmeas is the total number of observations measured.
²Nref is the number of unique reflections measured at least once.
³Complete is the percentage of possible reflections actually measured at least once.
⁴Rmerge = Σ||I| − |<I>||/Σ|<I>|, where I is the intensity of a single observation and <I> the average intensity for symmetry equivalent observations.
⁵Rwork = Σ|Fo − Fc|/Σ|Fo|, where Fo and Fc are observed and calculated structure factor amplitudes, respectively.
⁶Rfree = Rwork for 531 reflections (5%) sequestered from refinement, selected at random from 99 resolution shells. R for all reflections is 0.249.
number solvent molecules 33
number non-H atoms 1,798

X-Ray Crystallographic Analysis

Each of the constituent amino acids of HGF β is defined by a set of structure coordinates as set forth in Table 5. The term “structure coordinates” refers to Cartesian coordinates derived from mathematical equations related to the patterns obtained on diffraction of a monochromatic beam of X-rays by the atoms (scattering centers) of a HGF β in crystal form. The diffraction data are used to calculate an electron density map of the repeating unit of the crystal. The electron density maps are then used to establish the positions of the individual atoms of the HGF β protein or protein/ligand complex.

Slight variations in structure coordinates can be generated by mathematically manipulating the HGF β or HGF β/ligand structure coordinates. For example, the structure coordinates as set forth in Table 5 could be manipulated by crystallographic permutations of the structure coordinates, fractionalization of the structure coordinates, integer additions or subtractions to sets of the structure coordinates, inversion of the structure coordinates, or any combination of the above. Alternatively, modifications in the crystal structure due to mutations, additions, substitutions, deletions, and combinations thereof, of amino acids, or other changes in any of the components that make up the crystal, could also yield variations in structure coordinates. Such slight variations in the individual coordinates will have little effect on overall shape. If such variations are within an acceptable standard error as compared to the original coordinates, the resulting three-dimensional shape is considered to be structurally equivalent. Structural equivalence is described in more detail below.

It should be noted that slight variations in individual structure coordinates of the HGF β would not be expected to significantly alter the nature of chemical entities such as ligands that could associate with an active site. In this context, the phrase “associating with” refers to a condition of proximity between a ligand, or portions thereof, and a HGF β molecule or portions thereof. The association may be non-covalent, wherein the juxtaposition is energetically favored by hydrogen bonding, van der Waals forces, and/or electrostatic interactions, or it may be covalent.

Modeling of the HGF β Domain

Resolution of the HGF β crystal structure revealed several structural features including the activation-domain, “active-site” region, a binding site for Met, a tunnel, dimerization region and the nature of the catalytic triad.

As shown in the examples, modeling of the crystal structure revealed a novel ligand-binding site for Met on HGF β. In some embodiments, amino acids defining HGF β structural features include those amino acids summarized in Table 4A. In some embodiments, amino acids defining a “core” set of HGF β structural features include those amino acids summarized in Table 4B.

TABLE 4A
Summary of Amino Acids Associated with Structural Features of HGF β

| Structural Feature | Associated Amino Acid Residues |
|---|---|
| activation-domain | 495-498, 502-505, 618-627, 553-562, 660- |
| | 672, 692-704, 637-655 |
| active-site region | 667-673, 532-536, 690-697, 637-655, 574- |
| | 579 |
| binding site | 513, 516, 533, 534, 537-539, 578, 619, 647, |
| | 656, 668-670, 673, 692-697, 699, 702, 705, |
| | 707 |
| tunnel | 673, 693-706, 660-670, 691, 634 |
| dimerization region | 496-502, 620-624, 626, 628, 630, 662-665, |
| | and 700 |

TABLE 4B
Summary of “Core” Amino Acids Associated with Structural
Features of HGF β

| Structural Feature | Associated Amino Acid Residues |
|---|---|
| activation-domain | 495-498, 618-627, 660-672, 692-704 |
| active-site region | 534, 578, 673 |
| binding site | mini: 673, 69-694, 695, 696 |
| | medium: 534, 578, 673, 692-694, 695, 696 |
| tunnel | 669, 670, 673, 693-697, 662, 663, 701 |
| dimerization region | 497, 499, 500, 502, 621-623, 662, 664 |

The atomic coordinates of HGF β are summarized in Table 5. The atomic coordinates of HGF β secondary structural features are summarized in Table 6.

Results

To better interpret Met binding and activity data from HGF mutants, the HGF β structure at 2.53 Å resolution was solved. Data reduction and refinement statistics and final model metrics appear in Table 3.

HGF β crystals were formed using three intermolecular contacts for each molecule (FIG. 6A). The smallest contact (about 360 Å² on each side) involves residues in the 1550-K562 [c70-c80] loop on one molecule and residues near the putative α-chain connecting Cys604 [c128] (mutated to Ser in this construct) site on the other molecule. Two larger intermolecular contacts derive from 2-fold crystallographic symmetry. Residues following the N-terminus (Val496-Arg502 [c17-c23]) plus residues from the [c140]- and [c180]-loops lose about 640 Å² of solvent accessible area (each side), and residues centered on Gln534 [c57] share a contact area of about 930 Å² (each side).

HGF β adopts the fold of chymotrypsin-like serine proteases, comprising two tandem distorted β-barrels. There are two poorly ordered and untraceable segments—His645-Thr651 [c170a-c175] and the C-terminal region beginning with Tyr723 [c245]. The ‘active-site’ region of HGF β clearly differs from those of true enzymes (FIG. 5A). Only Asp578 [c102] of the canonical catalytic triad is present, Ser and His being changed to Tyr673 [c195] and Gln534 [c57], respectively. As a result, the interaction between Ser and His, supported by an Asp-His hydrogen bond, is impossible and Tyr673 [c195] significantly narrows the entrance to the ‘S1 pocket’. In addition to changes in two of the ‘catalytic triad residues’, Pro693 [c215] is distinct from Tip [c215] found in all serine proteases. Indeed, normal substrate binding via main chain hydrogen bonds to segment [c214-c216] would be severely hampered by the main chain conformation and side chains of Val692 [c214] and Pro693 [c215] (FIG. 5B). Furthermore, there are structural differences in the nominal ‘S1 pocket’, where Gly667 [c189] at the bottom of the pocket and Pro668 [c190] are also distinct from residues found in serine proteases. Thus, there is a structural basis to understand why mutations in HGF creating the Asp [c102]-His [c57]-Ser [c195] catalytic triad are still insufficient to impart catalytic activity (Lokker et al., 1992).

HGF β residues that interact with Met are shown in FIGS. 5C and 5D according to their relative activities in cell migration assays. The Met binding site is compact and centered on the ‘active-site’ region. The electrostatic surface charge distribution in the binding site is diverse, being nonpolar at Tyr673 [c195] and Val692 [c214], polar at Gln534 [c57], negatively charged at Asp578 [c102], and positively charged at Arg695 [c217]. The outer limit of the functional Met binding site extends to distal portions of the [c220]-loop (residues I699 [c221a] and N701 [c223]), the [c 140]-loop (residues Y619, T620, G621 [c143-c145]) and residues R514 [c36] and P537 [c60a] (FIGS. 5C and 5D). Together, these residues resemble the substrate-processing region of true serine proteases. This finding agrees with an earlier study, which identified Y673 and V692 as important residues for Met activation (Lokker et al., 1992). The normal activity measured for the HGF variant Q534H in that study may reflect functional compensation of Gln by His, a relatively close isostere.

The functional importance of the [c220]-loop has precedent in the well-described family of chymotrypsin-like serine proteases (Perona and Craik, 1994; Hedstrom, 2002). The extended canonical conformation of substrates and inhibitors includes residues that can form main chain interactions with amino acid residues 692-696 [c214-c218]. This peptide segment has an amino acid which is inappropriate for “substrate” binding (Pro693) and overall the wrong conformation for “substrate” binding. This region is also recognized as an allosteric regulator of thrombin catalytic activity (Di Cera et al., 1995) and as an interaction site with its inhibitor hirudin (Stubbs and Bode, 1993). In addition, residues in Factor VIIa and thrombin that correspond to HGF R695 [c217] are important for enzyme-catalyzed substrate processing (Tsiang et al., 1995; Dickinson et al., 1996). Moreover, the corresponding residue in MSP, R683 [c217], plays a pivotal role in the high affinity interaction of MSP β-chain with its receptor Ron (Danilkovitch et al., 1999). MSP R683 [c217] is part of a cluster of five surface exposed arginine residues proposed to be involved in high affinity binding to Ron (Miller and Leonard, 1998). Although only R695 [c217] and possibly K649 [c173] are conserved in HGF, these residues are all located within the Met binding region of the HGF β-chain.

The binding site identified herein is in excellent agreement with the Met binding site revealed in the co-crystal structure of soluble Met Sema domain bound to HGF β as disclosed in the abovementioned copending application U.S. Ser. No. 60/568,865, filed May 6, 2005. For instance, the co-crystal structure shows that residues on the [c220]-loop, such as R695 [c217], make contacts to the Met receptor.

Our results are in contrast with previous studies demonstrating that HGF β-chain itself neither binds to nor inhibits HGF binding to Met (Hartmann et al., 1992; Matsumoto et al., 1998). In one instance, the HGF β-chain was different from ours, having extra α-chain residues derived from elastase cleavage of HGF, which could adversely affect Met binding. However, it is more likely that, for example, the concentrations used, the sensitivity of the assays, or the extent of pro-HGF processing may have been insufficient to observe binding to this low affinity site (Matsumoto et al., 1998). HGF β-chain has been reported to bind to Met although only in the presence of NK4 fragment from the α-chain (Matsumoto et al., 1998).

In principle, the existence of two Met binding sites in one HGF molecule could support a 2:1 model of a Met:HGF signaling complex, analogous to the proposed 2:1 model of Ron:MSP (Miller and Leonard, 1998). In the related MSP/Ron ligand/receptor system, individual α- and β-chains of MSP, which are devoid of signaling activity, can bind to Ron and compete with full length MSP for receptor binding (Danilkovitch et al., 1999). The same is true in the HGF/Met system. However, biochemical studies have not identified any 2:1 complexes of Met:HGF (Gherardi et al., 2003). In addition, this model of receptor activation requires some as yet unknown molecular mechanism that would prevent one HGF molecule from simultaneously binding to one Met receptor through its α- and β-chains.

The results suggest that the HGF β-chain may have functions in receptor activation beyond those involved in direct interactions with Met that would favor a 2:2 complex of HGF:Met. It was found that proHGF β the single chain ‘unactivated’ form of the HGF β-chain, bound more tightly to Met than several mutants in the ‘activated’ form of HGF β, i.e. Y673A, V692A, and R695A (FIG. 4). Importantly, all three corresponding full-length HGF mutants show measurable receptor phosphorylation and/or pro-migratory activities, however proHGF does not show such activities, even at concentrations 1,000-fold more than that needed for activity by HGF. This significant distinction suggests additional functions of the HGF β-chain in receptor activation.

Although no structure exists for proHGF β, the most dramatic molecular change between activated and unactivated HGF β-chain almost certainly occurs at the activation cleavage site, where the new N-terminus inserts into the protein to form the salt bridge with the side chain of D672 [c194], akin to molecular changes seen with zymogens and proteases. In the crystal structure, HGF β forms a symmetric dimer. Upon inspection of intermolecular contacts seen in the HGF β crystal lattice, one of the dimer interfaces (FIG. 6A) borders the Met binding site and comprises parts of the N-terminal peptide (V496-Arg502 [c17-c23]) and adjacent residues from the [c140]-620-624, 626, 628, 630 and [c180]-loops 662-665. This contact site must be very different in scHGF because it includes the activation cleavage site. If such an HGF β-chain dimer interaction is important for Met signaling, it would explain why scHGF completely lacks biological activity, despite weak Met interaction through its incompletely formed ‘active-site’ region. In this model the HGF β-chain interaction with Met would serve to properly orient the β-chain/β-chain interaction site. While this HGF β-chain/β-chain contact may be a crystallization artifact, the presence of the identical contact in the crystal lattice of the HGF β/Met Sema domain co-crystals as disclosed in the abovementioned copending application U.S. Ser. No. 60/568,865, filed May 6, 2005, also supports this model. A dimeric arrangement of HGF β modules in the HGF/Met signaling complex would favor a 2:2 model in which two individual HGF/Met complexes form a higher order signaling complex consisting of two HGF and two Met molecules (Donate et al., 1994). An interaction between two HGF β-chains would likely be very weak and perhaps only found when bound to the membrane form of Met.

In conclusion, the results presented herein show that the β-chain of HGF contains a new interaction site with Met, which is similar to the ‘active-site’ region of serine proteases. Thus, HGF is bivalent, having a high affinity Met binding site in the NK1 region of the α-chain and a low affinity binding site on the HGF β chain. Other important interactions may occur between two HGF β-chains, two HGF α-chains (Donate et al., 1994), and as found with MSP/Ron (Angeloni et al., JBC, in press), between two Met Sema domains. Furthermore, heparin also plays a key role in HGF/Met receptor binding. The identification of a distinct Met binding site on the HGF β-chain can be used to design new classes of HGF and/or Met modulators, such as antagonists, agonists, inhibitors, and like agents, having therapeutic applications, such as, for treating cancer.

### EXAMPLE 4

### Comparison of HGF to Other Proteins

Comparison of HGF β and Plasmin Structures

Among proteins with reported molecular structures, the amino acid sequence of HGF β is most homologous with that of plasmin/plasminogen, having 37% identity. Superimposition (Cohen, 1997) of the plasmin protease domain 1BUI (Berman et al., 2000; Parry et al., 1998) with HGF β using Cα atoms yields an rmsd of 1.2 Å for 192 atom pairs (out of 227 in our HGF β structure). A structure-based sequence alignment with plasmin shows HGF β has single amino acid deletions immediately before and after the sequence ⁵⁰⁵IGWMVSLRYR⁵¹⁴ (FIG. 6B), another single amino acid deletion following QCF⁵³⁶ (Gln534 is homologous with His [c57]), and a two amino acid insertion between His554 [c74] and Gly557 [c75]. The deletions following Arg514 and Phe536, and the insertion after His554 are in loop regions where length heterogeneity among homologous proteins is common. However, the first deletion, preceding Ile505 [c27], is unusual. It is thought that it appears only in HGF and its closest relative MSP among homologous human protein sequences. In comparison with plasmin, the trace of HGF β in this segment is more direct between Thr503 and Gly506.

The plasmin structure (Parry et al., 1998) includes the C-terminal fragment from the plasmin A-chain, which is connected to the protease domain with two disulfide bonds (FIG. 6B). In HGF, the α-chain to β-chain link homologous to plasmin Cys567/Cys685 is made between Cys487 and Cys604 (Donate et al., 1994); however this may not be the case. The path adopted by plasmin A-chain residues Cys567-Arg580 (FIG. 6C) is similar to the one used by the analogous segments of chymotrypsinogen (Wang et al., 1985) and single-chain t-PA (Renatus et al., 1997). Inspection of the superimposed HGF β and plasmin structures (FIG. 6C) does not suggest a likely path for the HGF α-chain from Cys487, which forms a disulfide link with Cys604 (Donate et al., 1994), to Val495 (FIG. 6B). The reasons are twofold, first, there is a poor structural alignment between HGF Cys604 and plasmin Cys685, and second, there is a smaller number of amino acids in HGF between Cys487 and Val495.

These features lead to the conclusions that plasminogen is a poor structural model for proHGF in the region where the activating cleavage occurs and that is more different from HGF than plasminogen is from plasmin. Based on the MSP pro-sequence, the same conclusions are not applicable to MSP. This result suggests that pro-HGF is unlike single chain MSP or single chain chymotrypsin. This implication, coupled with the result showing that HGF-β (as would be found in 2-chain HGF) is reasonably similar to chymotrypsin, leads to a conclusion that the structural differences between single chain and 2-chain HGF are larger than differences between single chain and 2-chain forms of MSP, or chymotrypsin. This tends to supports the view that HGF-β conversion from single chain to 2-chain form mediates receptor activation.

Comparison of HGF β and Other Proteins

The nonenzymatic ‘catalytic triad’ of HGF is shared by the acute phase plasma protein haptoglobin (Kurosky et al., 1980), the Trypanosome lytic factor binding protein haptoglobin-related protein (Drain et al. 2001) and the blood coagulation cofactor protein Z (Broze et al., 2001). Like HGF, they retain the intact ‘catalytic triad residue’ Asp [c102], but have changes in residues [c57] (Lys or Gln) and [c195] (Ala or Gly). MSP, the other member of the plasminogen-related growth factors, also has a nonenzymatic ‘catalytic triad’ in which residues [c57] and [c102] are each changed to Gln. Except for MSP, which uses the β-chain for a high affinity interaction with its receptor tyrosine kinase Ron, the role of these other nonenzymatic protease-like domains is not well understood. Their function may involve activation dependent formation of a protein binding epitope similar to that found on the β-chains of HGF and MSP.

Although zymogen forms of proteases are generally not catalytically competent, some are still capable of binding and even cleaving substrates. For example, single-chain forms of t-PA and u-PA still have catalytic activity, albeit somewhat reduced from the corresponding activated forms, (Boose et al., 1989; Lijnen et al., 1990). Thus, binding of the zymogen-like β-chain of scHGF to Met, would not be without precedent; our binding data of proHGF β to Met supports this idea.

Another HGF β-chain region with the potential for protein-protein interactions corresponds to exosite I of thrombin (fibrinogen binding exosite). Exosite I is present as zymogen and active forms (Vijayalakshmi et al., 1994) and contains a positively charged patch centered around the [c70-80]-loop (Stubbs and Bode, 1993), which is involved in interactions with substrates, cofactors and inhibitors (Stubbs and Bode, 1993). HGF β also has a positively charged surface in this region, suggesting a potential role in protein interactions. Although two mutational changes introduced in this region (I550-E559 [c70-c77]) did not affect HGF function in cell migration assays, the possibility remains of it interacting with cell surface co-stimulatory factors of Met signaling. The positive charge observed is consistent with heparin interactions. Heparin modulates HGF activity. The positively charged region comprises, consists essentially of, or consists of some or all residues 512, 515-517, 545, 547, 550, 553-565 or mixtures thereof. In some embodiments, the amino acid residues comprise, consist essentially of, or consist of one or more of Arg512, Asn515, Lys516, His517, Glu545, Trp547, Ile550, Val553, His554, Gly555, Arg556, Gly557, Asp558, Glu559, Lys560, Cys561, Lys562, Gln563, Val564, Leu565, or mixtures thereof.

TABLE 5
Atomic Coordinates of HGF β

| | | | Amino Acid | | | | | | | Temp | Atom |
|---|---|---|---|---|---|---|---|---|---|---|---|
| Atom Number | | | Residue | | | X | Y | Z | Occ. | Factor | Type |
| ATOM | 1 | N | VAL | H | 495 | 53.287 | −0.680 | 54.295 | 1.00 | 16.08 | N |
| ATOM | 2 | CA | VAL | H | 495 | 52.270 | −1.420 | 53.478 | 1.00 | 17.77 | C |
| ATOM | 3 | CB | VAL | H | 495 | 50.934 | −0.629 | 53.352 | 1.00 | 18.35 | C |
| ATOM | 4 | CG1 | VAL | H | 495 | 49.829 | −1.533 | 52.716 | 1.00 | 12.84 | C |
| ATOM | 5 | CG2 | VAL | H | 495 | 50.478 | −0.060 | 54.734 | 1.00 | 4.66 | C |
| ATOM | 6 | C | VAL | H | 495 | 52.806 | −1.679 | 52.069 | 1.00 | 21.12 | C |
| ATOM | 7 | O | VAL | H | 495 | 53.345 | −0.750 | 51.408 | 1.00 | 11.04 | O |
| ATOM | 8 | N | VAL | H | 496 | 52.660 | −2.936 | 51.635 | 1.00 | 14.17 | N |
| ATOM | 9 | CA | VAL | H | 496 | 53.141 | −3.382 | 50.335 | 1.00 | 13.48 | C |
| ATOM | 10 | CB | VAL | H | 496 | 54.072 | −4.669 | 50.465 | 1.00 | 16.55 | C |
| ATOM | 11 | CG1 | VAL | H | 496 | 54.397 | −5.297 | 49.123 | 1.00 | 10.12 | C |
| ATOM | 12 | CG2 | VAL | H | 496 | 55.428 | −4.368 | 51.222 | 1.00 | 15.42 | C |
| ATOM | 13 | C | VAL | H | 496 | 51.898 | −3.632 | 49.443 | 1.00 | 20.08 | C |
| ATOM | 14 | O | VAL | H | 496 | 50.940 | −4.247 | 49.879 | 1.00 | 17.47 | O |
| ATOM | 15 | N | ASN | H | 497 | 51.930 | −3.132 | 48.200 | 1.00 | 18.19 | N |
| ATOM | 16 | CA | ASN | H | 497 | 50.814 | −3.152 | 47.268 | 1.00 | 13.48 | C |
| ATOM | 17 | CB | ASN | H | 497 | 50.528 | −4.556 | 46.718 | 1.00 | 16.62 | C |
| ATOM | 18 | CG | ASN | H | 497 | 51.712 | −5.118 | 45.935 | 1.00 | 17.86 | C |
| ATOM | 19 | OD1 | ASN | H | 497 | 52.540 | −4.362 | 45.398 | 1.00 | 15.01 | O |
| ATOM | 20 | ND2 | ASN | H | 497 | 51.821 | −6.442 | 45.892 | 1.00 | 10.88 | N |
| ATOM | 21 | C | ASN | H | 497 | 49.574 | −2.508 | 47.874 | 1.00 | 22.80 | C |
| ATOM | 22 | O | ASN | H | 497 | 48.475 | −3.105 | 47.900 | 1.00 | 19.58 | O |
| ATOM | 23 | N | GLY | H | 498 | 49.783 | −1.289 | 48.384 | 1.00 | 16.54 | N |
| ATOM | 24 | CA | GLY | H | 498 | 48.707 | −0.422 | 48.802 | 1.00 | 17.01 | C |
| ATOM | 25 | C | GLY | H | 498 | 48.892 | 0.887 | 48.061 | 1.00 | 20.79 | C |
| ATOM | 26 | O | GLY | H | 498 | 49.797 | 1.013 | 47.248 | 1.00 | 16.61 | O |
| ATOM | 27 | N | ILE | H | 499 | 48.066 | 1.882 | 48.358 | 1.00 | 23.23 | N |
| ATOM | 28 | CA | ILE | H | 499 | 48.210 | 3.170 | 47.685 | 1.00 | 25.29 | C |
| ATOM | 29 | CB | ILE | H | 499 | 47.102 | 3.341 | 46.614 | 1.00 | 30.64 | C |
| ATOM | 30 | CG1 | ILE | H | 499 | 45.747 | 3.511 | 47.292 | 1.00 | 23.56 | C |
| ATOM | 31 | CD1 | ILE | H | 499 | 44.588 | 3.300 | 46.387 | 1.00 | 33.00 | C |
| ATOM | 32 | CG2 | ILE | H | 499 | 47.159 | 2.196 | 45.590 | 1.00 | 31.49 | C |
| ATOM | 33 | C | ILE | H | 499 | 48.143 | 4.290 | 48.695 | 1.00 | 19.29 | C |
| ATOM | 34 | O | ILE | H | 499 | 47.666 | 4.078 | 49.805 | 1.00 | 17.80 | O |
| ATOM | 35 | N | PRO | H | 500 | 48.626 | 5.473 | 48.318 | 1.00 | 18.24 | N |
| ATOM | 36 | CA | PRO | H | 500 | 48.581 | 6.626 | 49.212 | 1.00 | 18.66 | C |
| ATOM | 37 | CB | PRO | H | 500 | 49.214 | 7.747 | 48.379 | 1.00 | 18.97 | C |
| ATOM | 38 | CG | PRO | H | 500 | 50.037 | 7.028 | 47.330 | 1.00 | 16.28 | C |
| ATOM | 39 | CD | PRO | H | 500 | 49.253 | 5.806 | 47.021 | 1.00 | 17.56 | C |
| ATOM | 40 | C | PRO | H | 500 | 47.148 | 6.966 | 49.528 | 1.00 | 23.47 | C |
| ATOM | 41 | O | PRO | H | 500 | 46.259 | 6.690 | 48.729 | 1.00 | 22.88 | O |
| ATOM | 42 | N | THR | H | 501 | 46.933 | 7.530 | 50.709 | 1.00 | 28.17 | N |
| ATOM | 43 | CA | THR | H | 501 | 45.641 | 8.084 | 51.090 | 1.00 | 25.91 | C |
| ATOM | 44 | CB | THR | H | 501 | 45.534 | 8.205 | 52.611 | 1.00 | 22.50 | C |
| ATOM | 45 | OG1 | THR | H | 501 | 46.698 | 8.875 | 53.116 | 1.00 | 22.17 | O |
| ATOM | 46 | CG2 | THR | H | 501 | 45.547 | 6.825 | 53.279 | 1.00 | 20.00 | C |
| ATOM | 47 | C | THR | H | 501 | 45.525 | 9.466 | 50.475 | 1.00 | 24.36 | C |
| ATOM | 48 | O | THR | H | 501 | 46.528 | 10.180 | 50.348 | 1.00 | 22.08 | O |
| ATOM | 49 | N | ARG | H | 502 | 44.311 | 9.838 | 50.085 | 1.00 | 30.28 | N |
| ATOM | 50 | CA | ARG | H | 502 | 44.083 | 11.165 | 49.515 | 1.00 | 40.27 | C |
| ATOM | 51 | CB | ARG | H | 502 | 42.614 | 11.357 | 49.145 | 1.00 | 47.30 | C |
| ATOM | 52 | CG | ARG | H | 502 | 42.030 | 10.221 | 48.327 | 1.00 | 54.82 | C |
| ATOM | 53 | CD | ARG | H | 502 | 40.596 | 10.462 | 47.920 | 1.00 | 63.65 | C |
| ATOM | 54 | NE | ARG | H | 502 | 39.722 | 9.356 | 48.298 | 1.00 | 69.68 | N |
| ATOM | 55 | CZ | ARG | H | 502 | 39.061 | 9.278 | 49.454 | 1.00 | 74.40 | C |
| ATOM | 56 | NH1 | ARG | H | 502 | 39.165 | 10.241 | 50.370 | 1.00 | 75.23 | N |
| ATOM | 57 | NH2 | ARG | H | 502 | 38.287 | 8.231 | 49.698 | 1.00 | 75.09 | N |
| ATOM | 58 | C | ARG | H | 502 | 44.531 | 12.232 | 50.514 | 1.00 | 41.69 | C |
| ATOM | 59 | O | ARG | H | 502 | 45.322 | 13.123 | 50.191 | 1.00 | 36.97 | O |
| ATOM | 60 | N | THR | H | 503 | 44.036 | 12.102 | 51.738 | 1.00 | 46.47 | N |
| ATOM | 61 | CA | THR | H | 503 | 44.365 | 13.023 | 52.818 | 1.00 | 51.60 | C |
| ATOM | 62 | CB | THR | H | 503 | 43.175 | 13.997 | 53.104 | 1.00 | 50.43 | C |
| ATOM | 63 | OG1 | THR | H | 503 | 43.513 | 14.843 | 54.205 | 1.00 | 54.45 | O |
| ATOM | 64 | CG2 | THR | H | 503 | 41.929 | 13.245 | 53.603 | 1.00 | 49.18 | C |
| ATOM | 65 | C | THR | H | 503 | 44.726 | 12.200 | 54.051 | 1.00 | 50.17 | C |
| ATOM | 66 | O | THR | H | 503 | 44.707 | 10.972 | 54.003 | 1.00 | 47.77 | O |
| ATOM | 67 | N | ASN | H | 504 | 45.045 | 12.875 | 55.150 | 1.00 | 47.39 | N |
| ATOM | 68 | CA | ASN | H | 504 | 45.443 | 12.170 | 56.356 | 1.00 | 49.03 | C |
| ATOM | 69 | CB | ASN | H | 504 | 46.322 | 13.045 | 57.248 | 1.00 | 55.37 | C |
| ATOM | 70 | CG | ASN | H | 504 | 45.872 | 14.483 | 57.271 | 1.00 | 59.13 | C |
| ATOM | 71 | OD1 | ASN | H | 504 | 44.934 | 14.827 | 57.986 | 1.00 | 59.15 | O |
| ATOM | 72 | ND2 | ASN | H | 504 | 46.539 | 15.338 | 56.483 | 1.00 | 58.94 | N |
| ATOM | 73 | C | ASN | H | 504 | 44.248 | 11.613 | 57.107 | 1.00 | 42.83 | C |
| ATOM | 74 | O | ASN | H | 504 | 43.132 | 12.100 | 56.948 | 1.00 | 36.84 | O |
| ATOM | 75 | N | ILE | H | 505 | 44.507 | 10.554 | 57.878 | 1.00 | 38.72 | N |
| ATOM | 76 | CA | ILE | H | 505 | 43.497 | 9.855 | 58.655 | 1.00 | 36.37 | C |
| ATOM | 77 | CB | ILE | H | 505 | 43.656 | 8.340 | 58.473 | 1.00 | 31.76 | C |
| ATOM | 78 | CG1 | ILE | H | 505 | 43.724 | 7.972 | 56.972 | 1.00 | 34.23 | C |
| ATOM | 79 | CD1 | ILE | H | 505 | 42.382 | 7.901 | 56.224 | 1.00 | 33.81 | C |
| ATOM | 80 | CG2 | ILE | H | 505 | 42.551 | 7.615 | 59.147 | 1.00 | 26.97 | C |
| ATOM | 81 | C | ILE | H | 505 | 43.675 | 10.274 | 60.119 | 1.00 | 41.62 | C |
| ATOM | 82 | O | ILE | H | 505 | 44.764 | 10.117 | 60.693 | 1.00 | 40.37 | O |
| ATOM | 83 | N | GLY | H | 506 | 42.603 | 10.822 | 60.700 | 1.00 | 39.15 | N |
| ATOM | 84 | CA | GLY | H | 506 | 42.652 | 11.500 | 61.989 | 1.00 | 34.15 | C |
| ATOM | 85 | C | GLY | H | 506 | 43.035 | 10.643 | 63.175 | 1.00 | 31.48 | C |
| ATOM | 86 | O | GLY | H | 506 | 43.724 | 11.102 | 64.083 | 1.00 | 25.54 | O |
| ATOM | 87 | N | TRP | H | 507 | 42.584 | 9.395 | 63.166 | 1.00 | 30.66 | N |
| ATOM | 88 | CA | TRP | H | 507 | 42.891 | 8.480 | 64.249 | 1.00 | 33.59 | C |
| ATOM | 89 | CB | TRP | H | 507 | 41.783 | 7.452 | 64.399 | 1.00 | 39.93 | C |
| ATOM | 90 | CG | TRP | H | 507 | 41.124 | 7.133 | 63.122 | 1.00 | 46.33 | C |
| ATOM | 91 | CD1 | TRP | H | 507 | 40.110 | 7.829 | 62.523 | 1.00 | 47.75 | C |
| ATOM | 92 | NE1 | TRP | H | 507 | 39.759 | 7.233 | 61.339 | 1.00 | 51.39 | N |
| ATOM | 93 | CE2 | TRP | H | 507 | 40.544 | 6.123 | 61.168 | 1.00 | 52.01 | C |
| ATOM | 94 | CD2 | TRP | H | 507 | 41.420 | 6.046 | 62.272 | 1.00 | 45.26 | C |
| ATOM | 95 | CE3 | TRP | H | 507 | 42.329 | 5.005 | 62.328 | 1.00 | 47.03 | C |
| ATOM | 96 | CZ3 | TRP | H | 507 | 42.345 | 4.097 | 61.314 | 1.00 | 52.99 | C |
| ATOM | 97 | CH2 | TRP | H | 507 | 41.479 | 4.200 | 60.227 | 1.00 | 56.32 | C |
| ATOM | 98 | CZ2 | TRP | H | 507 | 40.567 | 5.206 | 60.136 | 1.00 | 53.30 | C |
| ATOM | 99 | C | TRP | H | 507 | 44.251 | 7.772 | 64.138 | 1.00 | 37.09 | C |
| ATOM | 100 | O | TRP | H | 507 | 44.570 | 6.952 | 64.992 | 1.00 | 38.96 | O |
| ATOM | 101 | N | MET | H | 508 | 45.053 | 8.097 | 63.120 | 1.00 | 33.59 | N |
| ATOM | 102 | CA | MET | H | 508 | 46.334 | 7.418 | 62.910 | 1.00 | 29.91 | C |
| ATOM | 103 | CB | MET | H | 508 | 46.668 | 7.227 | 61.413 | 1.00 | 32.44 | C |
| ATOM | 104 | CG | MET | H | 508 | 45.901 | 6.085 | 60.718 | 1.00 | 31.67 | C |
| ATOM | 105 | SD | MET | H | 508 | 46.274 | 4.404 | 61.309 | 1.00 | 37.99 | S |
| ATOM | 106 | CE | MET | H | 508 | 48.105 | 4.485 | 61.266 | 1.00 | 23.29 | C |
| ATOM | 107 | C | MET | H | 508 | 47.497 | 8.082 | 63.627 | 1.00 | 26.16 | C |
| ATOM | 108 | O | MET | H | 508 | 47.716 | 9.291 | 63.531 | 1.00 | 32.91 | O |
| ATOM | 109 | N | VAL | H | 509 | 48.249 | 7.269 | 64.350 | 1.00 | 25.77 | N |
| ATOM | 110 | CA | VAL | H | 509 | 49.416 | 7.763 | 65.081 | 1.00 | 28.48 | C |
| ATOM | 111 | CB | VAL | H | 509 | 49.305 | 7.433 | 66.582 | 1.00 | 28.28 | C |
| ATOM | 112 | CG1 | VAL | H | 509 | 50.387 | 8.197 | 67.374 | 1.00 | 21.69 | C |
| ATOM | 113 | CG2 | VAL | H | 509 | 47.914 | 7.765 | 67.097 | 1.00 | 21.13 | C |
| ATOM | 114 | C | VAL | H | 509 | 50.695 | 7.131 | 64.559 | 1.00 | 16.82 | C |
| ATOM | 115 | O | VAL | H | 509 | 50.710 | 5.946 | 64.270 | 1.00 | 20.59 | O |
| ATOM | 116 | N | SER | H | 510 | 51.736 | 7.933 | 64.415 | 1.00 | 14.57 | N |
| ATOM | 117 | CA | SER | H | 510 | 53.080 | 7.432 | 64.118 | 1.00 | 20.85 | C |
| ATOM | 118 | CB | SER | H | 510 | 53.807 | 8.394 | 63.164 | 1.00 | 22.11 | C |
| ATOM | 119 | OG | SER | H | 510 | 55.146 | 7.963 | 62.873 | 1.00 | 23.40 | O |
| ATOM | 120 | C | SER | H | 510 | 53.864 | 7.332 | 65.421 | 1.00 | 22.84 | C |
| ATOM | 121 | O | SER | H | 510 | 54.243 | 8.366 | 65.997 | 1.00 | 16.34 | O |
| ATOM | 122 | N | LEU | H | 511 | 54.098 | 6.111 | 65.903 | 1.00 | 30.55 | N |
| ATOM | 123 | CA | LEU | H | 511 | 54.988 | 5.933 | 67.049 | 1.00 | 27.15 | C |
| ATOM | 124 | CB | LEU | H | 511 | 54.785 | 4.569 | 67.704 | 1.00 | 26.53 | C |
| ATOM | 125 | CG | LEU | H | 511 | 55.183 | 4.427 | 69.184 | 1.00 | 30.35 | C |
| ATOM | 126 | CD1 | LEU | H | 511 | 54.796 | 3.077 | 69.684 | 1.00 | 17.74 | C |
| ATOM | 127 | CD2 | LEU | H | 511 | 56.642 | 4.605 | 69.353 | 1.00 | 33.86 | C |
| ATOM | 128 | C | LEU | H | 511 | 56.436 | 6.091 | 66.579 | 1.00 | 30.75 | C |
| ATOM | 129 | O | LEU | H | 511 | 56.898 | 5.301 | 65.746 | 1.00 | 36.07 | O |
| ATOM | 130 | N | ARG | H | 512 | 57.121 | 7.128 | 67.095 | 1.00 | 30.93 | N |
| ATOM | 131 | CA | ARG | H | 512 | 58.541 | 7.389 | 66.839 | 1.00 | 28.29 | C |
| ATOM | 132 | CB | ARG | H | 512 | 58.780 | 8.879 | 66.778 | 1.00 | 32.11 | C |
| ATOM | 133 | CG | ARG | H | 512 | 57.602 | 9.672 | 66.295 | 1.00 | 39.45 | C |
| ATOM | 134 | CD | ARG | H | 512 | 57.373 | 9.541 | 64.824 | 1.00 | 40.26 | C |
| ATOM | 135 | NE | ARG | H | 512 | 58.289 | 10.382 | 64.060 | 1.00 | 41.23 | N |
| ATOM | 136 | CZ | ARG | H | 512 | 58.163 | 10.595 | 62.749 | 1.00 | 40.52 | C |
| ATOM | 137 | NH1 | ARG | H | 512 | 57.157 | 10.015 | 62.074 | 1.00 | 35.67 | N |
| ATOM | 138 | NH2 | ARG | H | 512 | 59.036 | 11.383 | 62.120 | 1.00 | 35.43 | N |
| ATOM | 139 | C | ARG | H | 512 | 59.420 | 6.796 | 67.936 | 1.00 | 32.99 | C |
| ATOM | 140 | O | ARG | H | 512 | 59.065 | 6.824 | 69.128 | 1.00 | 36.72 | O |
| ATOM | 141 | N | TYR | H | 513 | 60.559 | 6.246 | 67.535 | 1.00 | 29.17 | N |
| ATOM | 142 | CA | TYR | H | 513 | 61.518 | 5.715 | 68.486 | 1.00 | 25.68 | C |
| ATOM | 143 | CB | TYR | H | 513 | 61.494 | 4.186 | 68.512 | 1.00 | 31.13 | C |
| ATOM | 144 | CG | TYR | H | 513 | 62.609 | 3.544 | 69.325 | 1.00 | 39.04 | C |
| ATOM | 145 | CD1 | TYR | H | 513 | 62.587 | 3.564 | 70.723 | 1.00 | 41.51 | C |
| ATOM | 146 | CE1 | TYR | H | 513 | 63.609 | 2.972 | 71.472 | 1.00 | 38.40 | C |
| ATOM | 147 | CZ | TYR | H | 513 | 64.657 | 2.358 | 70.820 | 1.00 | 36.73 | C |
| ATOM | 148 | OH | TYR | H | 513 | 65.646 | 1.783 | 71.562 | 1.00 | 31.84 | O |
| ATOM | 149 | CE2 | TYR | H | 513 | 64.706 | 2.320 | 69.434 | 1.00 | 34.80 | C |
| ATOM | 150 | CD2 | TYR | H | 513 | 63.689 | 2.908 | 68.695 | 1.00 | 36.25 | C |
| ATOM | 151 | C | TYR | H | 513 | 62.835 | 6.243 | 68.054 | 1.00 | 24.69 | C |
| ATOM | 152 | O | TYR | H | 513 | 63.260 | 6.024 | 66.926 | 1.00 | 28.09 | O |
| ATOM | 153 | N | ARG | H | 514 | 63.472 | 6.990 | 68.941 | 1.00 | 28.98 | N |
| ATOM | 154 | CA | ARG | H | 514 | 64.774 | 7.586 | 68.647 | 1.00 | 29.82 | C |
| ATOM | 155 | CB | ARG | H | 514 | 65.871 | 6.514 | 68.448 | 1.00 | 27.16 | C |
| ATOM | 156 | CG | ARG | H | 514 | 65.925 | 5.352 | 69.468 | 1.00 | 32.33 | C |
| ATOM | 157 | CD | ARG | H | 514 | 66.745 | 5.609 | 70.751 | 1.00 | 31.62 | C |
| ATOM | 158 | NE | ARG | H | 514 | 68.114 | 6.082 | 70.500 | 1.00 | 24.74 | N |
| ATOM | 159 | CZ | ARG | H | 514 | 68.805 | 6.821 | 71.368 | 1.00 | 27.51 | C |
| ATOM | 160 | NH1 | ARG | H | 514 | 68.264 | 7.167 | 72.531 | 1.00 | 23.67 | N |
| ATOM | 161 | NH2 | ARG | H | 514 | 70.037 | 7.229 | 71.073 | 1.00 | 25.52 | N |
| ATOM | 162 | C | ARG | H | 514 | 64.664 | 8.477 | 67.414 | 1.00 | 30.84 | C |
| ATOM | 163 | O | ARG | H | 514 | 65.433 | 8.343 | 66.461 | 1.00 | 38.17 | O |
| ATOM | 164 | N | ASN | H | 515 | 63.703 | 9.395 | 67.446 | 1.00 | 36.99 | N |
| ATOM | 165 | CA | ASN | H | 515 | 63.475 | 10.362 | 66.363 | 1.00 | 39.79 | C |
| ATOM | 166 | CB | ASN | H | 515 | 64.655 | 11.359 | 66.216 | 1.00 | 50.85 | C |
| ATOM | 167 | CG | ASN | H | 515 | 64.952 | 12.123 | 67.495 | 1.00 | 57.78 | C |
| ATOM | 168 | OD1 | ASN | H | 515 | 64.055 | 12.373 | 68.309 | 1.00 | 58.30 | O |
| ATOM | 169 | ND2 | ASN | H | 515 | 66.218 | 12.501 | 67.678 | 1.00 | 57.54 | N |
| ATOM | 170 | C | ASN | H | 515 | 63.184 | 9.710 | 65.016 | 1.00 | 32.96 | C |
| ATOM | 171 | O | ASN | H | 515 | 63.646 | 10.177 | 63.994 | 1.00 | 35.55 | O |
| ATOM | 172 | N | LYS | H | 516 | 62.428 | 8.621 | 65.000 | 1.00 | 31.11 | N |
| ATOM | 173 | CA | LYS | H | 516 | 62.122 | 7.982 | 63.725 | 1.00 | 25.00 | C |
| ATOM | 174 | CB | LYS | H | 516 | 63.341 | 7.256 | 63.172 | 1.00 | 23.67 | C |
| ATOM | 175 | CG | LYS | H | 516 | 63.037 | 6.428 | 61.942 | 1.00 | 16.86 | C |
| ATOM | 176 | CD | LYS | H | 516 | 64.255 | 6.283 | 61.039 | 1.00 | 15.82 | C |
| ATOM | 177 | CE | LYS | H | 516 | 63.974 | 5.217 | 59.976 | 1.00 | 17.19 | C |
| ATOM | 178 | NZ | LYS | H | 516 | 65.012 | 5.276 | 58.930 | 1.00 | 26.75 | N |
| ATOM | 179 | C | LYS | H | 516 | 60.957 | 7.025 | 63.820 | 1.00 | 23.16 | C |
| ATOM | 180 | O | LYS | H | 516 | 60.840 | 6.275 | 64.781 | 1.00 | 19.11 | O |
| ATOM | 181 | N | HIS | H | 517 | 60.100 | 7.044 | 62.803 | 1.00 | 19.62 | N |
| ATOM | 182 | CA | HIS | H | 517 | 58.953 | 6.172 | 62.783 | 1.00 | 15.94 | C |
| ATOM | 183 | CB | HIS | H | 517 | 58.184 | 6.304 | 61.470 | 1.00 | 16.19 | C |
| ATOM | 184 | CG | HIS | H | 517 | 57.045 | 5.338 | 61.348 | 1.00 | 14.04 | C |
| ATOM | 185 | ND1 | HIS | H | 517 | 55.756 | 5.650 | 61.729 | 1.00 | 21.22 | N |
| ATOM | 186 | CE1 | HIS | H | 517 | 54.971 | 4.603 | 61.534 | 1.00 | 19.21 | C |
| ATOM | 187 | NE2 | HIS | H | 517 | 55.707 | 3.618 | 61.054 | 1.00 | 21.06 | N |
| ATOM | 188 | CD2 | HIS | H | 517 | 57.008 | 4.055 | 60.920 | 1.00 | 17.23 | C |
| ATOM | 189 | C | HIS | H | 517 | 59.392 | 4.724 | 62.957 | 1.00 | 19.19 | C |
| ATOM | 190 | O | HIS | H | 517 | 60.429 | 4.317 | 62.418 | 1.00 | 15.08 | O |
| ATOM | 191 | N | ILE | H | 518 | 58.570 | 3.961 | 63.675 | 1.00 | 14.75 | N |
| ATOM | 192 | CA | ILE | H | 518 | 58.760 | 2.544 | 63.856 | 1.00 | 16.08 | C |
| ATOM | 193 | CB | ILE | H | 518 | 59.533 | 2.264 | 65.203 | 1.00 | 17.96 | C |
| ATOM | 194 | CG1 | ILE | H | 518 | 59.623 | 0.757 | 65.476 | 1.00 | 5.85 | C |
| ATOM | 195 | CD1 | ILE | H | 518 | 60.426 | 0.428 | 66.715 | 1.00 | 15.60 | C |
| ATOM | 196 | CG2 | ILE | H | 518 | 58.827 | 2.930 | 66.379 | 1.00 | 12.03 | C |
| ATOM | 197 | C | ILE | H | 518 | 57.400 | 1.836 | 63.849 | 1.00 | 19.60 | C |
| ATOM | 198 | O | ILE | H | 518 | 57.306 | 0.664 | 63.525 | 1.00 | 22.17 | O |
| ATOM | 199 | N | CYS | H | 519 | 56.339 | 2.538 | 64.222 | 1.00 | 24.67 | N |
| ATOM | 200 | CA | CYS | H | 519 | 55.012 | 1.905 | 64.239 | 1.00 | 24.54 | C |
| ATOM | 201 | CB | CYS | H | 519 | 54.789 | 1.152 | 65.545 | 1.00 | 19.24 | C |
| ATOM | 202 | SG | CYS | H | 519 | 55.738 | −0.349 | 65.699 | 1.00 | 29.96 | S |
| ATOM | 203 | C | CYS | H | 519 | 53.850 | 2.876 | 64.046 | 1.00 | 23.64 | C |
| ATOM | 204 | O | CYS | H | 519 | 54.005 | 4.090 | 64.105 | 1.00 | 18.84 | O |
| ATOM | 205 | N | GLY | H | 520 | 52.672 | 2.312 | 63.839 | 1.00 | 22.25 | N |
| ATOM | 206 | CA | GLY | H | 520 | 51.471 | 3.109 | 63.854 | 1.00 | 26.74 | C |
| ATOM | 207 | C | GLY | H | 520 | 50.655 | 2.754 | 65.076 | 1.00 | 32.79 | C |
| ATOM | 208 | O | GLY | H | 520 | 50.819 | 1.679 | 65.655 | 1.00 | 34.59 | O |
| ATOM | 209 | N | GLY | H | 521 | 49.770 | 3.659 | 65.470 | 1.00 | 37.07 | N |
| ATOM | 210 | CA | GLY | H | 521 | 48.835 | 3.368 | 66.538 | 1.00 | 37.03 | C |
| ATOM | 211 | C | GLY | H | 521 | 47.488 | 4.007 | 66.293 | 1.00 | 35.37 | C |
| ATOM | 212 | O | GLY | H | 521 | 47.341 | 4.856 | 65.400 | 1.00 | 32.92 | O |
| ATOM | 213 | N | SER | H | 522 | 46.507 | 3.600 | 67.092 | 1.00 | 30.61 | N |
| ATOM | 214 | CA | SER | H | 522 | 45.188 | 4.208 | 67.047 | 1.00 | 27.88 | C |
| ATOM | 215 | CB | SER | H | 522 | 44.148 | 3.113 | 66.876 | 1.00 | 28.31 | C |
| ATOM | 216 | OG | SER | H | 522 | 44.478 | 2.337 | 65.743 | 1.00 | 37.94 | O |
| ATOM | 217 | C | SER | H | 522 | 44.868 | 5.059 | 68.280 | 1.00 | 27.21 | C |
| ATOM | 218 | O | SER | H | 522 | 44.749 | 4.524 | 69.386 | 1.00 | 23.08 | O |
| ATOM | 219 | N | LEU | H | 523 | 44.714 | 6.368 | 68.069 | 1.00 | 28.06 | N |
| ATOM | 220 | CA | LEU | H | 523 | 44.208 | 7.303 | 69.083 | 1.00 | 30.15 | C |
| ATOM | 221 | CB | LEU | H | 523 | 44.164 | 8.725 | 68.522 | 1.00 | 27.93 | C |
| ATOM | 222 | CG | LEU | H | 523 | 43.963 | 9.887 | 69.507 | 1.00 | 28.77 | C |
| ATOM | 223 | CD1 | LEU | H | 523 | 45.203 | 10.149 | 70.367 | 1.00 | 25.96 | C |
| ATOM | 224 | CD2 | LEU | H | 523 | 43.644 | 11.149 | 68.759 | 1.00 | 26.23 | C |
| ATOM | 225 | C | LEU | H | 523 | 42.795 | 6.939 | 69.467 | 1.00 | 30.11 | C |
| ATOM | 226 | O | LEU | H | 523 | 41.873 | 7.295 | 68.751 | 1.00 | 30.67 | O |
| ATOM | 227 | N | ILE | H | 524 | 42.628 | 6.230 | 70.580 | 1.00 | 27.14 | N |
| ATOM | 228 | CA | ILE | H | 524 | 41.301 | 5.847 | 71.047 | 1.00 | 26.54 | C |
| ATOM | 229 | CB | ILE | H | 524 | 41.254 | 4.390 | 71.560 | 1.00 | 25.50 | C |
| ATOM | 230 | CG1 | ILE | H | 524 | 42.326 | 4.102 | 72.618 | 1.00 | 25.85 | C |
| ATOM | 231 | CD1 | ILE | H | 524 | 42.228 | 2.677 | 73.138 | 1.00 | 21.19 | C |
| ATOM | 232 | CG2 | ILE | H | 524 | 41.388 | 3.426 | 70.402 | 1.00 | 25.92 | C |
| ATOM | 233 | C | ILE | H | 524 | 40.658 | 6.800 | 72.072 | 1.00 | 33.23 | C |
| ATOM | 234 | O | ILE | H | 524 | 39.464 | 6.697 | 72.318 | 1.00 | 38.31 | O |
| ATOM | 235 | N | LYS | H | 525 | 41.445 | 7.699 | 72.664 | 1.00 | 33.30 | N |
| ATOM | 236 | CA | LYS | H | 525 | 40.955 | 8.768 | 73.530 | 1.00 | 37.28 | C |
| ATOM | 237 | CB | LYS | H | 525 | 40.920 | 8.331 | 74.995 | 1.00 | 43.94 | C |
| ATOM | 238 | CG | LYS | H | 525 | 39.762 | 7.409 | 75.398 | 1.00 | 50.33 | C |
| ATOM | 239 | CD | LYS | H | 525 | 38.402 | 8.122 | 75.348 | 1.00 | 57.30 | C |
| ATOM | 240 | CE | LYS | H | 525 | 37.272 | 7.274 | 75.950 | 1.00 | 59.43 | C |
| ATOM | 241 | NZ | LYS | H | 525 | 37.281 | 7.336 | 77.441 | 1.00 | 56.40 | N |
| ATOM | 242 | C | LYS | H | 525 | 41.888 | 9.961 | 73.378 | 1.00 | 39.92 | C |
| ATOM | 243 | O | LYS | H | 525 | 42.914 | 9.868 | 72.716 | 1.00 | 39.04 | O |
| ATOM | 244 | N | GLU | H | 526 | 41.554 | 11.083 | 73.998 | 1.00 | 44.55 | N |
| ATOM | 245 | CA | GLU | H | 526 | 42.374 | 12.281 | 73.835 | 1.00 | 49.39 | C |
| ATOM | 246 | CB | GLU | H | 526 | 41.735 | 13.492 | 74.525 | 1.00 | 56.64 | C |
| ATOM | 247 | CG | GLU | H | 526 | 40.532 | 14.098 | 73.800 | 1.00 | 63.33 | C |
| ATOM | 248 | CD | GLU | H | 526 | 39.192 | 13.483 | 74.209 | 1.00 | 67.88 | C |
| ATOM | 249 | OE1 | GLU | H | 526 | 39.127 | 12.264 | 74.552 | 1.00 | 66.55 | O |
| ATOM | 250 | OE2 | GLU | H | 526 | 38.189 | 14.238 | 74.183 | 1.00 | 69.82 | O |
| ATOM | 251 | C | GLU | H | 526 | 43.790 | 12.068 | 74.356 | 1.00 | 47.89 | C |
| ATOM | 252 | O | GLU | H | 526 | 44.709 | 12.822 | 74.027 | 1.00 | 45.34 | O |
| ATOM | 253 | N | SER | H | 527 | 43.966 | 11.035 | 75.170 | 1.00 | 47.26 | N |
| ATOM | 254 | CA | SER | H | 527 | 45.273 | 10.800 | 75.774 | 1.00 | 51.88 | C |
| ATOM | 255 | CB | SER | H | 527 | 45.307 | 11.365 | 77.212 | 1.00 | 55.39 | C |
| ATOM | 256 | OG | SER | H | 527 | 45.307 | 12.795 | 77.187 | 1.00 | 56.29 | O |
| ATOM | 257 | C | SER | H | 527 | 45.782 | 9.345 | 75.674 | 1.00 | 44.71 | C |
| ATOM | 258 | O | SER | H | 527 | 46.811 | 9.008 | 76.250 | 1.00 | 41.12 | O |
| ATOM | 259 | N | TRP | H | 528 | 45.077 | 8.516 | 74.909 | 1.00 | 39.87 | N |
| ATOM | 260 | CA | TRP | H | 528 | 45.423 | 7.110 | 74.759 | 1.00 | 42.61 | C |
| ATOM | 261 | CB | TRP | H | 528 | 44.414 | 6.240 | 75.491 | 1.00 | 47.22 | C |
| ATOM | 262 | CG | TRP | H | 528 | 44.511 | 6.398 | 76.952 | 1.00 | 58.02 | C |
| ATOM | 263 | CD1 | TRP | H | 528 | 43.951 | 7.387 | 77.710 | 1.00 | 60.27 | C |
| ATOM | 264 | NE1 | TRP | H | 528 | 44.274 | 7.210 | 79.033 | 1.00 | 62.48 | N |
| ATOM | 265 | CE2 | TRP | H | 528 | 45.052 | 6.089 | 79.156 | 1.00 | 65.73 | C |
| ATOM | 266 | CD2 | TRP | H | 528 | 45.228 | 5.557 | 77.859 | 1.00 | 63.43 | C |
| ATOM | 267 | CE3 | TRP | H | 528 | 46.004 | 4.400 | 77.711 | 1.00 | 63.34 | C |
| ATOM | 268 | CZ3 | TRP | H | 528 | 46.569 | 3.822 | 78.841 | 1.00 | 65.49 | C |
| ATOM | 269 | CH2 | TRP | H | 528 | 46.374 | 4.376 | 80.115 | 1.00 | 65.39 | C |
| ATOM | 270 | CZ2 | TRP | H | 528 | 45.621 | 5.505 | 80.293 | 1.00 | 65.55 | C |
| ATOM | 271 | C | TRP | H | 528 | 45.576 | 6.633 | 73.308 | 1.00 | 43.22 | C |
| ATOM | 272 | O | TRP | H | 528 | 44.771 | 6.976 | 72.420 | 1.00 | 44.13 | O |
| ATOM | 273 | N | VAL | H | 529 | 46.614 | 5.823 | 73.089 | 1.00 | 32.74 | N |
| ATOM | 274 | CA | VAL | H | 529 | 46.897 | 5.232 | 71.786 | 1.00 | 17.54 | C |
| ATOM | 275 | CB | VAL | H | 529 | 48.168 | 5.827 | 71.202 | 1.00 | 22.32 | C |
| ATOM | 276 | CG1 | VAL | H | 529 | 48.385 | 5.327 | 69.789 | 1.00 | 28.68 | C |
| ATOM | 277 | CG2 | VAL | H | 529 | 48.119 | 7.381 | 71.235 | 1.00 | 11.69 | C |
| ATOM | 278 | C | VAL | H | 529 | 47.010 | 3.713 | 71.918 | 1.00 | 21.97 | C |
| ATOM | 279 | O | VAL | H | 529 | 47.837 | 3.214 | 72.681 | 1.00 | 28.97 | O |
| ATOM | 280 | N | LEU | H | 530 | 46.141 | 2.972 | 71.233 | 1.00 | 20.55 | N |
| ATOM | 281 | CA | LEU | H | 530 | 46.218 | 1.515 | 71.233 | 1.00 | 20.82 | C |
| ATOM | 282 | CB | LEU | H | 530 | 44.876 | 0.922 | 70.888 | 1.00 | 16.98 | C |
| ATOM | 283 | CG | LEU | H | 530 | 44.799 | −0.591 | 70.817 | 1.00 | 19.50 | C |
| ATOM | 284 | CD1 | LEU | H | 530 | 45.261 | −1.287 | 72.126 | 1.00 | 17.89 | C |
| ATOM | 285 | CD2 | LEU | H | 530 | 43.391 | −0.986 | 70.451 | 1.00 | 19.11 | C |
| ATOM | 286 | C | LEU | H | 530 | 47.224 | 1.074 | 70.182 | 1.00 | 30.61 | C |
| ATOM | 287 | O | LEU | H | 530 | 47.105 | 1.440 | 69.016 | 1.00 | 40.33 | O |
| ATOM | 288 | N | THR | H | 531 | 48.209 | 0.284 | 70.591 | 1.00 | 34.13 | N |
| ATOM | 289 | CA | THR | H | 531 | 49.310 | −0.097 | 69.707 | 1.00 | 28.21 | C |
| ATOM | 290 | CB | THR | H | 531 | 50.403 | 0.995 | 69.722 | 1.00 | 27.64 | C |
| ATOM | 291 | OG1 | THR | H | 531 | 51.383 | 0.725 | 68.711 | 1.00 | 24.02 | O |
| ATOM | 292 | CG2 | THR | H | 531 | 51.165 | 1.017 | 71.044 | 1.00 | 33.75 | C |
| ATOM | 293 | C | THR | H | 531 | 49.836 | −1.501 | 70.021 | 1.00 | 26.11 | C |
| ATOM | 294 | O | THR | H | 531 | 49.136 | −2.285 | 70.698 | 1.00 | 26.68 | O |
| ATOM | 295 | N | ALA | H | 532 | 51.038 | −1.832 | 69.537 | 1.00 | 22.57 | N |
| ATOM | 296 | CA | ALA | H | 532 | 51.551 | −3.213 | 69.666 | 1.00 | 22.72 | C |
| ATOM | 297 | CB | ALA | H | 532 | 51.693 | −3.855 | 68.292 | 1.00 | 17.58 | C |
| ATOM | 298 | C | ALA | H | 532 | 52.847 | −3.366 | 70.460 | 1.00 | 21.23 | C |
| ATOM | 299 | O | ALA | H | 532 | 53.690 | −2.442 | 70.504 | 1.00 | 17.42 | O |
| ATOM | 300 | N | ARG | H | 533 | 53.014 | −4.539 | 71.065 | 1.00 | 20.81 | N |
| ATOM | 301 | CA | ARG | H | 533 | 54.230 | −4.837 | 71.861 | 1.00 | 28.21 | C |
| ATOM | 302 | CB | ARG | H | 533 | 54.141 | −6.214 | 72.559 | 1.00 | 30.08 | C |
| ATOM | 303 | CG | ARG | H | 533 | 55.229 | −6.479 | 73.649 | 1.00 | 29.56 | C |
| ATOM | 304 | CD | ARG | H | 533 | 55.402 | −5.306 | 74.631 | 1.00 | 36.23 | C |
| ATOM | 305 | NE | ARG | H | 533 | 56.251 | −5.577 | 75.793 | 1.00 | 45.45 | N |
| ATOM | 306 | CZ | ARG | H | 533 | 55.943 | −6.424 | 76.794 | 1.00 | 45.73 | C |
| ATOM | 307 | NH1 | ARG | H | 533 | 54.810 | −7.120 | 76.767 | 1.00 | 41.26 | N |
| ATOM | 308 | NH2 | ARG | H | 533 | 56.781 | −6.582 | 77.818 | 1.00 | 41.60 | N |
| ATOM | 309 | C | ARG | H | 533 | 55.551 | −4.731 | 71.078 | 1.00 | 29.47 | C |
| ATOM | 310 | O | ARG | H | 533 | 56.531 | −4.143 | 71.545 | 1.00 | 30.85 | O |
| ATOM | 311 | N | GLN | H | 534 | 55.572 | −5.306 | 69.885 | 1.00 | 30.95 | N |
| ATOM | 312 | CA | GLN | H | 534 | 56.750 | −5.295 | 69.015 | 1.00 | 22.13 | C |
| ATOM | 313 | CB | GLN | H | 534 | 56.416 | −6.063 | 67.736 | 1.00 | 20.77 | C |
| ATOM | 314 | CG | GLN | H | 534 | 55.985 | −7.562 | 67.970 | 1.00 | 20.28 | C |
| ATOM | 315 | CD | GLN | H | 534 | 54.489 | −7.772 | 68.238 | 1.00 | 24.01 | C |
| ATOM | 316 | OE1 | GLN | H | 534 | 53.782 | −6.832 | 68.600 | 1.00 | 26.79 | O |
| ATOM | 317 | NE2 | GLN | H | 534 | 54.010 | −8.995 | 68.061 | 1.00 | 23.49 | N |
| ATOM | 318 | C | GLN | H | 534 | 57.236 | −3.875 | 68.670 | 1.00 | 20.18 | C |
| ATOM | 319 | O | GLN | H | 534 | 58.239 | −3.703 | 67.983 | 1.00 | 20.33 | O |
| ATOM | 320 | N | CYS | H | 535 | 56.537 | −2.854 | 69.157 | 1.00 | 13.52 | N |
| ATOM | 321 | CA | CYS | H | 535 | 56.918 | −1.462 | 68.865 | 1.00 | 16.80 | C |
| ATOM | 322 | CB | CYS | H | 535 | 55.652 | −0.625 | 68.625 | 1.00 | 18.25 | C |
| ATOM | 323 | SG | CYS | H | 535 | 54.759 | −1.294 | 67.192 | 1.00 | 24.84 | S |
| ATOM | 324 | C | CYS | H | 535 | 57.834 | −0.798 | 69.890 | 1.00 | 15.79 | C |
| ATOM | 325 | O | CYS | H | 535 | 58.106 | 0.403 | 69.812 | 1.00 | 15.25 | O |
| ATOM | 326 | N | PHE | H | 536 | 58.338 | −1.566 | 70.847 | 1.00 | 19.42 | N |
| ATOM | 327 | CA | PHE | H | 536 | 59.115 | −0.944 | 71.925 | 1.00 | 32.51 | C |
| ATOM | 328 | CB | PHE | H | 536 | 58.270 | −0.770 | 73.197 | 1.00 | 31.36 | C |
| ATOM | 329 | CG | PHE | H | 536 | 56.972 | −0.069 | 72.968 | 1.00 | 27.76 | C |
| ATOM | 330 | CD1 | PHE | H | 536 | 55.840 | −0.782 | 72.612 | 1.00 | 27.34 | C |
| ATOM | 331 | CE1 | PHE | H | 536 | 54.628 | −0.120 | 72.387 | 1.00 | 24.96 | C |
| ATOM | 332 | CZ | PHE | H | 536 | 54.549 | 1.251 | 72.548 | 1.00 | 23.42 | C |
| ATOM | 333 | CE2 | PHE | H | 536 | 55.669 | 1.973 | 72.920 | 1.00 | 23.65 | C |
| ATOM | 334 | CD2 | PHE | H | 536 | 56.877 | 1.312 | 73.120 | 1.00 | 28.00 | C |
| ATOM | 335 | C | PHE | H | 536 | 60.342 | −1.771 | 72.223 | 1.00 | 37.99 | C |
| ATOM | 336 | O | PHE | H | 536 | 60.299 | −2.694 | 73.041 | 1.00 | 37.64 | O |
| ATOM | 337 | N | PRO | H | 537 | 61.436 | −1.443 | 71.541 | 1.00 | 43.56 | N |
| ATOM | 338 | CA | PRO | H | 537 | 62.708 | −2.136 | 71.751 | 1.00 | 44.12 | C |
| ATOM | 339 | CB | PRO | H | 537 | 63.649 | −1.427 | 70.776 | 1.00 | 38.43 | C |
| ATOM | 340 | CG | PRO | H | 537 | 62.778 | −0.794 | 69.802 | 1.00 | 38.69 | C |
| ATOM | 341 | CD | PRO | H | 537 | 61.557 | −0.369 | 70.537 | 1.00 | 37.76 | C |
| ATOM | 342 | C | PRO | H | 537 | 63.190 | −1.935 | 73.196 | 1.00 | 46.21 | C |
| ATOM | 343 | O | PRO | H | 537 | 63.724 | −2.890 | 73.786 | 1.00 | 46.96 | O |
| ATOM | 344 | N | SER | H | 538 | 62.996 | −0.719 | 73.732 | 1.00 | 43.73 | N |
| ATOM | 345 | CA | SER | H | 538 | 63.395 | −0.357 | 75.101 | 1.00 | 41.03 | C |
| ATOM | 346 | CB | SER | H | 538 | 64.348 | 0.839 | 75.091 | 1.00 | 37.26 | C |
| ATOM | 347 | OG | SER | H | 538 | 63.655 | 2.076 | 74.957 | 1.00 | 30.97 | O |
| ATOM | 348 | C | SER | H | 538 | 62.197 | −0.028 | 75.978 | 1.00 | 42.14 | C |
| ATOM | 349 | O | SER | H | 538 | 61.143 | 0.368 | 75.474 | 1.00 | 39.71 | O |
| ATOM | 350 | N | ARG | H | 539 | 62.372 | −0.187 | 77.290 | 1.00 | 44.32 | N |
| ATOM | 351 | CA | ARG | H | 539 | 61.361 | 0.214 | 78.278 | 1.00 | 37.31 | C |
| ATOM | 352 | CB | ARG | H | 539 | 61.276 | −0.771 | 79.457 | 1.00 | 32.41 | C |
| ATOM | 353 | CG | ARG | H | 539 | 60.674 | −2.157 | 79.153 | 1.00 | 30.27 | C |
| ATOM | 354 | CD | ARG | H | 539 | 60.174 | −2.937 | 80.388 | 1.00 | 27.81 | C |
| ATOM | 355 | NE | ARG | H | 539 | 59.954 | −4.352 | 80.077 | 1.00 | 30.62 | N |
| ATOM | 356 | CZ | ARG | H | 539 | 59.291 | −5.237 | 80.849 | 1.00 | 32.54 | C |
| ATOM | 357 | NH1 | ARG | H | 539 | 58.769 | −4.879 | 82.010 | 1.00 | 29.55 | N |
| ATOM | 358 | NH2 | ARG | H | 539 | 59.147 | −6.501 | 80.454 | 1.00 | 30.77 | N |
| ATOM | 359 | C | ARG | H | 539 | 61.585 | 1.624 | 78.825 | 1.00 | 41.90 | C |
| ATOM | 360 | O | ARG | H | 539 | 60.985 | 1.976 | 79.832 | 1.00 | 45.84 | O |
| ATOM | 361 | N | ASP | H | 540 | 62.417 | 2.453 | 78.195 | 1.00 | 43.43 | N |
| ATOM | 362 | CA | ASP | H | 540 | 62.486 | 3.826 | 78.710 | 1.00 | 51.63 | C |
| ATOM | 363 | CB | ASP | H | 540 | 63.851 | 4.191 | 79.330 | 1.00 | 61.57 | C |
| ATOM | 364 | CG | ASP | H | 540 | 65.003 | 4.018 | 78.383 | 1.00 | 66.80 | C |
| ATOM | 365 | OD1 | ASP | H | 540 | 65.426 | 2.866 | 78.158 | 1.00 | 71.18 | O |
| ATOM | 366 | OD2 | ASP | H | 540 | 65.569 | 4.986 | 77.839 | 1.00 | 71.55 | O |
| ATOM | 367 | C | ASP | H | 540 | 61.862 | 4.941 | 77.859 | 1.00 | 55.25 | C |
| ATOM | 368 | O | ASP | H | 540 | 62.337 | 5.298 | 76.772 | 1.00 | 57.16 | O |
| ATOM | 369 | N | LEU | H | 541 | 60.785 | 5.485 | 78.424 | 1.00 | 55.36 | N |
| ATOM | 370 | CA | LEU | H | 541 | 59.903 | 6.481 | 77.821 | 1.00 | 47.78 | C |
| ATOM | 371 | CB | LEU | H | 541 | 58.966 | 7.053 | 78.885 | 1.00 | 48.67 | C |
| ATOM | 372 | CG | LEU | H | 541 | 58.133 | 6.031 | 79.671 | 1.00 | 53.13 | C |
| ATOM | 373 | CD1 | LEU | H | 541 | 57.331 | 6.710 | 80.803 | 1.00 | 53.35 | C |
| ATOM | 374 | CD2 | LEU | H | 541 | 57.224 | 5.215 | 78.763 | 1.00 | 51.21 | C |
| ATOM | 375 | C | LEU | H | 541 | 60.567 | 7.619 | 77.073 | 1.00 | 49.66 | C |
| ATOM | 376 | O | LEU | H | 541 | 60.025 | 8.073 | 76.062 | 1.00 | 51.52 | O |
| ATOM | 377 | N | LYS | H | 542 | 61.720 | 8.087 | 77.555 | 1.00 | 48.56 | N |
| ATOM | 378 | CA | LYS | H | 542 | 62.383 | 9.244 | 76.947 | 1.00 | 48.24 | C |
| ATOM | 379 | CB | LYS | H | 542 | 63.665 | 9.655 | 77.693 | 1.00 | 55.49 | C |
| ATOM | 380 | CG | LYS | H | 542 | 63.596 | 9.696 | 79.227 | 1.00 | 64.10 | C |
| ATOM | 381 | CD | LYS | H | 542 | 64.005 | 8.348 | 79.856 | 1.00 | 69.91 | C |
| ATOM | 382 | CE | LYS | H | 542 | 64.119 | 8.438 | 81.388 | 1.00 | 73.19 | C |
| ATOM | 383 | NZ | LYS | H | 542 | 64.577 | 7.152 | 82.017 | 1.00 | 69.84 | N |
| ATOM | 384 | C | LYS | H | 542 | 62.714 | 8.970 | 75.480 | 1.00 | 47.21 | C |
| ATOM | 385 | O | LYS | H | 542 | 63.030 | 9.898 | 74.738 | 1.00 | 48.71 | O |
| ATOM | 386 | N | ASP | H | 543 | 62.654 | 7.697 | 75.079 | 1.00 | 45.29 | N |
| ATOM | 387 | CA | ASP | H | 543 | 62.910 | 7.281 | 73.691 | 1.00 | 45.62 | C |
| ATOM | 388 | CB | ASP | H | 543 | 63.296 | 5.792 | 73.633 | 1.00 | 42.94 | C |
| ATOM | 389 | CG | ASP | H | 543 | 64.693 | 5.493 | 74.174 | 1.00 | 41.01 | C |
| ATOM | 390 | OD1 | ASP | H | 543 | 65.595 | 6.348 | 74.054 | 1.00 | 35.27 | O |
| ATOM | 391 | OD2 | ASP | H | 543 | 64.964 | 4.385 | 74.712 | 1.00 | 37.92 | O |
| ATOM | 392 | C | ASP | H | 543 | 61.714 | 7.475 | 72.728 | 1.00 | 46.66 | C |
| ATOM | 393 | O | ASP | H | 543 | 61.846 | 7.236 | 71.523 | 1.00 | 50.24 | O |
| ATOM | 394 | N | TYR | H | 544 | 60.552 | 7.877 | 73.240 | 1.00 | 44.29 | N |
| ATOM | 395 | CA | TYR | H | 544 | 59.339 | 7.913 | 72.406 | 1.00 | 41.03 | C |
| ATOM | 396 | CB | TYR | H | 544 | 58.316 | 6.882 | 72.850 | 1.00 | 24.93 | C |
| ATOM | 397 | CG | TYR | H | 544 | 58.885 | 5.524 | 73.006 | 1.00 | 28.55 | C |
| ATOM | 398 | CD1 | TYR | H | 544 | 59.509 | 5.145 | 74.198 | 1.00 | 28.35 | C |
| ATOM | 399 | CE1 | TYR | H | 544 | 60.038 | 3.867 | 74.357 | 1.00 | 29.44 | C |
| ATOM | 400 | CZ | TYR | H | 544 | 59.964 | 2.955 | 73.304 | 1.00 | 28.68 | C |
| ATOM | 401 | OH | TYR | H | 544 | 60.479 | 1.693 | 73.449 | 1.00 | 29.68 | O |
| ATOM | 402 | CE2 | TYR | H | 544 | 59.353 | 3.311 | 72.103 | 1.00 | 30.79 | C |
| ATOM | 403 | CD2 | TYR | H | 544 | 58.814 | 4.597 | 71.962 | 1.00 | 30.45 | C |
| ATOM | 404 | C | TYR | H | 544 | 58.626 | 9.246 | 72.266 | 1.00 | 44.02 | C |
| ATOM | 405 | O | TYR | H | 544 | 58.689 | 10.119 | 73.134 | 1.00 | 50.15 | O |
| ATOM | 406 | N | GLU | H | 545 | 57.939 | 9.355 | 71.133 | 1.00 | 44.19 | N |
| ATOM | 407 | CA | GLU | H | 545 | 57.073 | 10.462 | 70.780 | 1.00 | 34.74 | C |
| ATOM | 408 | CB | GLU | H | 545 | 57.811 | 11.448 | 69.892 | 1.00 | 31.51 | C |
| ATOM | 409 | CG | GLU | H | 545 | 58.690 | 12.396 | 70.672 | 1.00 | 32.43 | C |
| ATOM | 410 | CD | GLU | H | 545 | 59.361 | 13.433 | 69.810 | 1.00 | 36.21 | C |
| ATOM | 411 | OE1 | GLU | H | 545 | 59.584 | 13.169 | 68.603 | 1.00 | 39.53 | O |
| ATOM | 412 | OE2 | GLU | H | 545 | 59.683 | 14.519 | 70.346 | 1.00 | 42.14 | O |
| ATOM | 413 | C | GLU | H | 545 | 55.911 | 9.882 | 70.013 | 1.00 | 36.05 | C |
| ATOM | 414 | O | GLU | H | 545 | 55.942 | 8.737 | 69.554 | 1.00 | 36.73 | O |
| ATOM | 415 | N | ALA | H | 546 | 54.868 | 10.680 | 69.878 | 1.00 | 37.58 | N |
| ATOM | 416 | CA | ALA | H | 546 | 53.723 | 10.275 | 69.105 | 1.00 | 32.35 | C |
| ATOM | 417 | CB | ALA | H | 546 | 52.570 | 9.906 | 69.997 | 1.00 | 31.66 | C |
| ATOM | 418 | C | ALA | H | 546 | 53.399 | 11.465 | 68.259 | 1.00 | 35.25 | C |
| ATOM | 419 | O | ALA | H | 546 | 53.169 | 12.567 | 68.767 | 1.00 | 42.07 | O |
| ATOM | 420 | N | TRP | H | 547 | 53.444 | 11.256 | 66.954 | 1.00 | 28.68 | N |
| ATOM | 421 | CA | TRP | H | 547 | 53.040 | 12.290 | 66.031 | 1.00 | 31.90 | C |
| ATOM | 422 | CB | TRP | H | 547 | 53.996 | 12.384 | 64.843 | 1.00 | 34.84 | C |
| ATOM | 423 | CG | TRP | H | 547 | 55.372 | 12.846 | 65.220 | 1.00 | 31.89 | C |
| ATOM | 424 | CD1 | TRP | H | 547 | 55.972 | 12.769 | 66.451 | 1.00 | 33.36 | C |
| ATOM | 425 | NE1 | TRP | H | 547 | 57.240 | 13.298 | 66.395 | 1.00 | 29.79 | N |
| ATOM | 426 | CE2 | TRP | H | 547 | 57.490 | 13.700 | 65.111 | 1.00 | 30.30 | C |
| ATOM | 427 | CD2 | TRP | H | 547 | 56.332 | 13.424 | 64.348 | 1.00 | 28.60 | C |
| ATOM | 428 | CE3 | TRP | H | 547 | 56.338 | 13.745 | 62.990 | 1.00 | 27.99 | C |
| ATOM | 429 | CZ3 | TRP | H | 547 | 57.481 | 14.325 | 62.443 | 1.00 | 26.05 | C |
| ATOM | 430 | CH2 | TRP | H | 547 | 58.599 | 14.584 | 63.225 | 1.00 | 29.05 | C |
| ATOM | 431 | CZ2 | TRP | H | 547 | 58.629 | 14.276 | 64.560 | 1.00 | 27.92 | C |
| ATOM | 432 | C | TRP | H | 547 | 51.626 | 11.991 | 65.602 | 1.00 | 31.19 | C |
| ATOM | 433 | O | TRP | H | 547 | 51.311 | 10.874 | 65.199 | 1.00 | 32.19 | O |
| ATOM | 434 | N | LEU | H | 548 | 50.784 | 13.004 | 65.770 | 1.00 | 32.39 | N |
| ATOM | 435 | CA | LEU | H | 548 | 49.389 | 12.996 | 65.382 | 1.00 | 30.88 | C |
| ATOM | 436 | CB | LEU | H | 548 | 48.489 | 13.337 | 66.580 | 1.00 | 36.42 | C |
| ATOM | 437 | CG | LEU | H | 548 | 48.282 | 12.413 | 67.784 | 1.00 | 39.96 | C |
| ATOM | 438 | CD1 | LEU | H | 548 | 47.351 | 11.285 | 67.402 | 1.00 | 46.01 | C |
| ATOM | 439 | CD2 | LEU | H | 548 | 49.595 | 11.869 | 68.344 | 1.00 | 43.31 | C |
| ATOM | 440 | C | LEU | H | 548 | 49.195 | 14.037 | 64.285 | 1.00 | 31.10 | C |
| ATOM | 441 | O | LEU | H | 548 | 50.037 | 14.918 | 64.087 | 1.00 | 25.15 | O |
| ATOM | 442 | N | GLY | H | 549 | 48.087 | 13.913 | 63.559 | 1.00 | 35.32 | N |
| ATOM | 443 | CA | GLY | H | 549 | 47.733 | 14.858 | 62.517 | 1.00 | 36.35 | C |
| ATOM | 444 | C | GLY | H | 549 | 48.732 | 14.969 | 61.386 | 1.00 | 40.62 | C |
| ATOM | 445 | O | GLY | H | 549 | 48.855 | 16.032 | 60.782 | 1.00 | 38.61 | O |
| ATOM | 446 | N | ILE | H | 550 | 49.453 | 13.887 | 61.098 | 1.00 | 40.68 | N |
| ATOM | 447 | CA | ILE | H | 550 | 50.369 | 13.895 | 59.956 | 1.00 | 37.13 | C |
| ATOM | 448 | CB | ILE | H | 550 | 51.798 | 13.445 | 60.331 | 1.00 | 36.28 | C |
| ATOM | 449 | CG1 | ILE | H | 550 | 51.778 | 12.045 | 60.953 | 1.00 | 34.93 | C |
| ATOM | 450 | CD1 | ILE | H | 550 | 53.127 | 11.420 | 60.989 | 1.00 | 36.34 | C |
| ATOM | 451 | CG2 | ILE | H | 550 | 52.484 | 14.494 | 61.212 | 1.00 | 29.19 | C |
| ATOM | 452 | C | ILE | H | 550 | 49.845 | 13.054 | 58.814 | 1.00 | 31.37 | C |
| ATOM | 453 | O | ILE | H | 550 | 49.042 | 12.132 | 59.029 | 1.00 | 27.71 | O |
| ATOM | 454 | N | HIS | H | 551 | 50.287 | 13.402 | 57.605 | 1.00 | 26.30 | N |
| ATOM | 455 | CA | HIS | H | 551 | 49.993 | 12.625 | 56.402 | 1.00 | 25.42 | C |
| ATOM | 456 | CB | HIS | H | 551 | 49.338 | 13.499 | 55.304 | 1.00 | 23.29 | C |
| ATOM | 457 | CG | HIS | H | 551 | 48.780 | 12.713 | 54.157 | 1.00 | 27.81 | C |
| ATOM | 458 | ND1 | HIS | H | 551 | 48.823 | 13.163 | 52.851 | 1.00 | 28.48 | N |
| ATOM | 459 | CE1 | HIS | H | 551 | 48.292 | 12.245 | 52.056 | 1.00 | 30.45 | C |
| ATOM | 460 | NE2 | HIS | H | 551 | 47.911 | 11.215 | 52.798 | 1.00 | 29.51 | N |
| ATOM | 461 | CD2 | HIS | H | 551 | 48.201 | 11.483 | 54.115 | 1.00 | 28.55 | C |
| ATOM | 462 | C | HIS | H | 551 | 51.308 | 12.012 | 55.940 | 1.00 | 24.99 | C |
| ATOM | 463 | O | HIS | H | 551 | 51.361 | 10.831 | 55.604 | 1.00 | 29.65 | O |
| ATOM | 464 | N | ASP | H | 552 | 52.358 | 12.834 | 55.962 | 1.00 | 23.14 | N |
| ATOM | 465 | CA | ASP | H | 552 | 53.718 | 12.456 | 55.623 | 1.00 | 28.09 | C |
| ATOM | 466 | CB | ASP | H | 552 | 54.485 | 13.696 | 55.191 | 1.00 | 31.20 | C |
| ATOM | 467 | CG | ASP | H | 552 | 55.840 | 13.375 | 54.585 | 1.00 | 35.12 | C |
| ATOM | 468 | OD1 | ASP | H | 552 | 55.885 | 12.716 | 53.518 | 1.00 | 34.76 | O |
| ATOM | 469 | OD2 | ASP | H | 552 | 56.912 | 13.760 | 55.102 | 1.00 | 35.72 | O |
| ATOM | 470 | C | ASP | H | 552 | 54.380 | 11.847 | 56.854 | 1.00 | 35.96 | C |
| ATOM | 471 | O | ASP | H | 552 | 53.926 | 12.086 | 57.980 | 1.00 | 44.26 | O |
| ATOM | 472 | N | VAL | H | 553 | 55.433 | 11.049 | 56.662 | 1.00 | 27.34 | N |
| ATOM | 473 | CA | VAL | H | 553 | 56.015 | 10.364 | 57.796 | 1.00 | 28.05 | C |
| ATOM | 474 | CB | VAL | H | 553 | 56.728 | 9.031 | 57.421 | 1.00 | 26.38 | C |
| ATOM | 475 | CG1 | VAL | H | 553 | 57.989 | 9.276 | 56.609 | 1.00 | 23.88 | C |
| ATOM | 476 | CG2 | VAL | H | 553 | 57.039 | 8.231 | 58.669 | 1.00 | 22.70 | C |
| ATOM | 477 | C | VAL | H | 553 | 56.944 | 11.322 | 58.523 | 1.00 | 32.30 | C |
| ATOM | 478 | O | VAL | H | 553 | 57.119 | 11.217 | 59.738 | 1.00 | 21.01 | O |
| ATOM | 479 | N | HIS | H | 554 | 57.513 | 12.264 | 57.770 | 1.00 | 34.06 | N |
| ATOM | 480 | CA | HIS | H | 554 | 58.388 | 13.269 | 58.337 | 1.00 | 39.27 | C |
| ATOM | 481 | CB | HIS | H | 554 | 59.549 | 13.539 | 57.395 | 1.00 | 42.09 | C |
| ATOM | 482 | CG | HIS | H | 554 | 60.317 | 12.313 | 57.043 | 1.00 | 45.44 | C |
| ATOM | 483 | ND1 | HIS | H | 554 | 60.386 | 11.822 | 55.758 | 1.00 | 45.48 | N |
| ATOM | 484 | CE1 | HIS | H | 554 | 61.113 | 10.720 | 55.750 | 1.00 | 44.57 | C |
| ATOM | 485 | NE2 | HIS | H | 554 | 61.518 | 10.481 | 56.986 | 1.00 | 43.78 | N |
| ATOM | 486 | CD2 | HIS | H | 554 | 61.025 | 11.456 | 57.815 | 1.00 | 44.23 | C |
| ATOM | 487 | C | HIS | H | 554 | 57.613 | 14.546 | 58.589 | 1.00 | 43.73 | C |
| ATOM | 488 | O | HIS | H | 554 | 58.198 | 15.578 | 58.914 | 1.00 | 46.20 | O |
| ATOM | 489 | N | GLY | H | 555 | 56.297 | 14.469 | 58.421 | 1.00 | 44.00 | N |
| ATOM | 490 | CA | GLY | H | 555 | 55.420 | 15.605 | 58.603 | 1.00 | 42.82 | C |
| ATOM | 491 | C | GLY | H | 555 | 55.691 | 16.759 | 57.665 | 1.00 | 41.76 | C |
| ATOM | 492 | O | GLY | H | 555 | 55.358 | 17.892 | 58.001 | 1.00 | 47.20 | O |
| ATOM | 493 | N | ARG | H | 556 | 56.265 | 16.480 | 56.496 | 1.00 | 45.35 | N |
| ATOM | 494 | CA | ARG | H | 556 | 56.712 | 17.528 | 55.555 | 1.00 | 54.64 | C |
| ATOM | 495 | CB | ARG | H | 556 | 57.353 | 16.920 | 54.302 | 1.00 | 54.24 | C |
| ATOM | 496 | CG | ARG | H | 556 | 58.829 | 16.571 | 54.458 | 1.00 | 50.59 | C |
| ATOM | 497 | CD | ARG | H | 556 | 59.385 | 15.677 | 53.343 | 1.00 | 51.30 | C |
| ATOM | 498 | NE | ARG | H | 556 | 58.742 | 14.365 | 53.326 | 1.00 | 52.30 | N |
| ATOM | 499 | CZ | ARG | H | 556 | 59.222 | 13.295 | 52.698 | 1.00 | 55.12 | C |
| ATOM | 500 | NH1 | ARG | H | 556 | 60.366 | 13.369 | 52.026 | 1.00 | 56.20 | N |
| ATOM | 501 | NH2 | ARG | H | 556 | 58.565 | 12.144 | 52.749 | 1.00 | 54.96 | N |
| ATOM | 502 | C | ARG | H | 556 | 55.679 | 18.607 | 55.161 | 1.00 | 60.66 | C |
| ATOM | 503 | O | ARG | H | 556 | 55.922 | 19.810 | 55.348 | 1.00 | 69.53 | O |
| ATOM | 504 | N | GLY | H | 557 | 54.544 | 18.200 | 54.610 | 1.00 | 57.91 | N |
| ATOM | 505 | CA | GLY | H | 557 | 53.506 | 19.174 | 54.317 | 1.00 | 55.87 | C |
| ATOM | 506 | C | GLY | H | 557 | 52.598 | 19.472 | 55.502 | 1.00 | 52.27 | C |
| ATOM | 507 | O | GLY | H | 557 | 51.760 | 20.365 | 55.421 | 1.00 | 52.64 | O |
| ATOM | 508 | N | ASP | H | 558 | 52.753 | 18.713 | 56.589 | 1.00 | 51.96 | N |
| ATOM | 509 | CA | ASP | H | 558 | 51.871 | 18.781 | 57.760 | 1.00 | 54.33 | C |
| ATOM | 510 | CB | ASP | H | 558 | 51.829 | 17.425 | 58.466 | 1.00 | 53.52 | C |
| ATOM | 511 | CG | ASP | H | 558 | 51.273 | 16.315 | 57.580 | 1.00 | 54.36 | C |
| ATOM | 512 | OD1 | ASP | H | 558 | 50.144 | 16.457 | 57.066 | 1.00 | 53.73 | O |
| ATOM | 513 | OD2 | ASP | H | 558 | 51.888 | 15.253 | 57.354 | 1.00 | 52.84 | O |
| ATOM | 514 | C | ASP | H | 558 | 52.314 | 19.868 | 58.745 | 1.00 | 62.19 | C |
| ATOM | 515 | O | ASP | H | 558 | 51.856 | 19.914 | 59.895 | 1.00 | 56.78 | O |
| ATOM | 516 | N | GLU | H | 559 | 53.196 | 20.744 | 58.254 | 1.00 | 72.11 | N |
| ATOM | 517 | CA | GLU | H | 559 | 53.794 | 21.864 | 58.985 | 1.00 | 77.34 | C |
| ATOM | 518 | CB | GLU | H | 559 | 54.292 | 22.918 | 57.987 | 1.00 | 82.66 | C |
| ATOM | 519 | CG | GLU | H | 559 | 55.556 | 22.527 | 57.231 | 1.00 | 87.17 | C |
| ATOM | 520 | CD | GLU | H | 559 | 56.288 | 23.733 | 56.662 | 1.00 | 90.35 | C |
| ATOM | 521 | OE1 | GLU | H | 559 | 55.833 | 24.284 | 55.636 | 1.00 | 91.19 | O |
| ATOM | 522 | OE2 | GLU | H | 559 | 57.323 | 24.134 | 57.238 | 1.00 | 91.39 | O |
| ATOM | 523 | C | GLU | H | 559 | 52.931 | 22.542 | 60.059 | 1.00 | 77.40 | C |
| ATOM | 524 | O | GLU | H | 559 | 53.462 | 23.072 | 61.034 | 1.00 | 78.70 | O |
| ATOM | 525 | N | LYS | H | 560 | 51.614 | 22.533 | 59.881 | 1.00 | 74.15 | N |
| ATOM | 526 | CA | LYS | H | 560 | 50.717 | 23.157 | 60.848 | 1.00 | 72.23 | C |
| ATOM | 527 | CB | LYS | H | 560 | 50.023 | 24.359 | 60.211 | 1.00 | 74.03 | C |
| ATOM | 528 | CG | LYS | H | 560 | 49.207 | 24.027 | 58.971 | 1.00 | 74.12 | C |
| ATOM | 529 | CD | LYS | H | 560 | 47.775 | 23.658 | 59.332 | 1.00 | 73.14 | C |
| ATOM | 530 | CE | LYS | H | 560 | 46.874 | 23.727 | 58.111 | 1.00 | 72.84 | C |
| ATOM | 531 | NZ | LYS | H | 560 | 47.063 | 24.990 | 57.346 | 1.00 | 69.77 | N |
| ATOM | 532 | C | LYS | H | 560 | 49.687 | 22.188 | 61.426 | 1.00 | 70.07 | C |
| ATOM | 533 | O | LYS | H | 560 | 49.135 | 22.424 | 62.503 | 1.00 | 68.16 | O |
| ATOM | 534 | N | CYS | H | 561 | 49.433 | 21.109 | 60.690 | 1.00 | 67.44 | N |
| ATOM | 535 | CA | CYS | H | 561 | 48.509 | 20.058 | 61.105 | 1.00 | 62.60 | C |
| ATOM | 536 | CB | CYS | H | 561 | 48.272 | 19.055 | 59.955 | 1.00 | 62.73 | C |
| ATOM | 537 | SG | CYS | H | 561 | 48.226 | 19.735 | 58.270 | 1.00 | 67.99 | S |
| ATOM | 538 | C | CYS | H | 561 | 48.998 | 19.312 | 62.371 | 1.00 | 57.74 | C |
| ATOM | 539 | O | CYS | H | 561 | 48.190 | 19.035 | 63.266 | 1.00 | 49.65 | O |
| ATOM | 540 | N | LYS | H | 562 | 50.308 | 19.026 | 62.458 | 1.00 | 55.80 | N |
| ATOM | 541 | CA | LYS | H | 562 | 50.824 | 18.026 | 63.419 | 1.00 | 53.92 | C |
| ATOM | 542 | CB | LYS | H | 562 | 52.140 | 17.359 | 62.954 | 1.00 | 54.66 | C |
| ATOM | 543 | CG | LYS | H | 562 | 53.448 | 18.157 | 63.027 | 1.00 | 50.24 | C |
| ATOM | 544 | CD | LYS | H | 562 | 54.553 | 17.298 | 63.691 | 1.00 | 46.35 | C |
| ATOM | 545 | CE | LYS | H | 562 | 55.970 | 17.568 | 63.160 | 1.00 | 46.84 | C |
| ATOM | 546 | NZ | LYS | H | 562 | 56.473 | 18.951 | 63.381 | 1.00 | 49.02 | N |
| ATOM | 547 | C | LYS | H | 562 | 50.895 | 18.363 | 64.907 | 1.00 | 54.21 | C |
| ATOM | 548 | O | LYS | H | 562 | 51.154 | 19.496 | 65.290 | 1.00 | 56.23 | O |
| ATOM | 549 | N | GLN | H | 563 | 50.659 | 17.345 | 65.728 | 1.00 | 53.45 | N |
| ATOM | 550 | CA | GLN | H | 563 | 50.805 | 17.438 | 67.171 | 1.00 | 50.96 | C |
| ATOM | 551 | CB | GLN | H | 563 | 49.472 | 17.179 | 67.858 | 1.00 | 49.42 | C |
| ATOM | 552 | CG | GLN | H | 563 | 48.458 | 18.291 | 67.842 | 1.00 | 46.76 | C |
| ATOM | 553 | CD | GLN | H | 563 | 47.147 | 17.806 | 68.416 | 1.00 | 49.59 | C |
| ATOM | 554 | OE1 | GLN | H | 563 | 47.078 | 17.405 | 69.587 | 1.00 | 50.08 | O |
| ATOM | 555 | NE2 | GLN | H | 563 | 46.102 | 17.813 | 67.593 | 1.00 | 51.94 | N |
| ATOM | 556 | C | GLN | H | 563 | 51.782 | 16.364 | 67.636 | 1.00 | 53.65 | C |
| ATOM | 557 | O | GLN | H | 563 | 51.573 | 15.155 | 67.395 | 1.00 | 54.48 | O |
| ATOM | 558 | N | VAL | H | 564 | 52.833 | 16.809 | 68.317 | 1.00 | 51.30 | N |
| ATOM | 559 | CA | VAL | H | 564 | 53.838 | 15.913 | 68.876 | 1.00 | 47.31 | C |
| ATOM | 560 | CB | VAL | H | 564 | 55.261 | 16.347 | 68.470 | 1.00 | 49.19 | C |
| ATOM | 561 | CG1 | VAL | H | 564 | 55.418 | 17.871 | 68.536 | 1.00 | 54.49 | C |
| ATOM | 562 | CG2 | VAL | H | 564 | 56.295 | 15.657 | 69.325 | 1.00 | 48.28 | C |
| ATOM | 563 | C | VAL | H | 564 | 53.687 | 15.857 | 70.390 | 1.00 | 43.24 | C |
| ATOM | 564 | O | VAL | H | 564 | 53.788 | 16.877 | 71.073 | 1.00 | 37.88 | O |
| ATOM | 565 | N | LEU | H | 565 | 53.422 | 14.656 | 70.896 | 1.00 | 42.90 | N |
| ATOM | 566 | CA | LEU | H | 565 | 53.220 | 14.425 | 72.318 | 1.00 | 41.57 | C |
| ATOM | 567 | CB | LEU | H | 565 | 51.786 | 13.999 | 72.550 | 1.00 | 44.13 | C |
| ATOM | 568 | CG | LEU | H | 565 | 50.737 | 14.965 | 72.005 | 1.00 | 48.40 | C |
| ATOM | 569 | CD1 | LEU | H | 565 | 49.428 | 14.231 | 71.858 | 1.00 | 50.29 | C |
| ATOM | 570 | CD2 | LEU | H | 565 | 50.591 | 16.189 | 72.921 | 1.00 | 48.47 | C |
| ATOM | 571 | C | LEU | H | 565 | 54.155 | 13.366 | 72.917 | 1.00 | 46.58 | C |
| ATOM | 572 | O | LEU | H | 565 | 54.299 | 12.275 | 72.355 | 1.00 | 51.06 | O |
| ATOM | 573 | N | ASN | H | 566 | 54.772 | 13.675 | 74.063 | 1.00 | 41.75 | N |
| ATOM | 574 | CA | ASN | H | 566 | 55.580 | 12.689 | 74.787 | 1.00 | 35.84 | C |
| ATOM | 575 | CB | ASN | H | 566 | 56.389 | 13.339 | 75.914 | 1.00 | 34.75 | C |
| ATOM | 576 | CG | ASN | H | 566 | 57.505 | 14.238 | 75.393 | 1.00 | 37.33 | C |
| ATOM | 577 | OD1 | ASN | H | 566 | 58.125 | 13.955 | 74.362 | 1.00 | 31.88 | O |
| ATOM | 578 | ND2 | ASN | H | 566 | 57.765 | 15.336 | 76.111 | 1.00 | 38.81 | N |
| ATOM | 579 | C | ASN | H | 566 | 54.730 | 11.562 | 75.340 | 1.00 | 34.45 | C |
| ATOM | 580 | O | ASN | H | 566 | 53.511 | 11.677 | 75.391 | 1.00 | 34.12 | O |
| ATOM | 581 | N | VAL | H | 567 | 55.370 | 10.470 | 75.753 | 1.00 | 36.02 | N |
| ATOM | 582 | CA | VAL | H | 567 | 54.636 | 9.352 | 76.303 | 1.00 | 38.03 | C |
| ATOM | 583 | CB | VAL | H | 567 | 54.891 | 8.040 | 75.533 | 1.00 | 37.58 | C |
| ATOM | 584 | CG1 | VAL | H | 567 | 54.274 | 6.841 | 76.276 | 1.00 | 38.48 | C |
| ATOM | 585 | CG2 | VAL | H | 567 | 54.309 | 8.109 | 74.140 | 1.00 | 39.22 | C |
| ATOM | 586 | C | VAL | H | 567 | 54.983 | 9.189 | 77.771 | 1.00 | 47.50 | C |
| ATOM | 587 | O | VAL | H | 567 | 56.135 | 8.878 | 78.125 | 1.00 | 50.20 | O |
| ATOM | 588 | N | SER | H | 568 | 53.963 | 9.383 | 78.610 | 1.00 | 48.68 | N |
| ATOM | 589 | CA | SER | H | 568 | 54.103 | 9.402 | 80.068 | 1.00 | 50.43 | C |
| ATOM | 590 | CB | SER | H | 568 | 53.085 | 10.376 | 80.700 | 1.00 | 48.95 | C |
| ATOM | 591 | OG | SER | H | 568 | 51.754 | 9.874 | 80.617 | 1.00 | 40.75 | O |
| ATOM | 592 | C | SER | H | 568 | 53.943 | 8.035 | 80.704 | 1.00 | 49.01 | C |
| ATOM | 593 | O | SER | H | 568 | 54.401 | 7.806 | 81.823 | 1.00 | 55.99 | O |
| ATOM | 594 | N | GLN | H | 569 | 53.264 | 7.132 | 80.018 | 1.00 | 45.75 | N |
| ATOM | 595 | CA | GLN | H | 569 | 53.038 | 5.807 | 80.574 | 1.00 | 46.57 | C |
| ATOM | 596 | CB | GLN | H | 569 | 51.752 | 5.766 | 81.377 | 1.00 | 55.38 | C |
| ATOM | 597 | CG | GLN | H | 569 | 51.777 | 6.484 | 82.691 | 1.00 | 62.83 | C |
| ATOM | 598 | CD | GLN | H | 569 | 50.498 | 6.239 | 83.445 | 1.00 | 69.49 | C |
| ATOM | 599 | OE1 | GLN | H | 569 | 49.879 | 7.178 | 83.957 | 1.00 | 72.81 | O |
| ATOM | 600 | NE2 | GLN | H | 569 | 50.070 | 4.970 | 83.489 | 1.00 | 70.95 | N |
| ATOM | 601 | C | GLN | H | 569 | 52.933 | 4.757 | 79.511 | 1.00 | 44.15 | C |
| ATOM | 602 | O | GLN | H | 569 | 52.835 | 5.056 | 78.321 | 1.00 | 51.64 | O |
| ATOM | 603 | N | LEU | H | 570 | 52.887 | 3.511 | 79.961 | 1.00 | 43.14 | N |
| ATOM | 604 | CA | LEU | H | 570 | 52.923 | 2.380 | 79.062 | 1.00 | 39.99 | C |
| ATOM | 605 | CB | LEU | H | 570 | 54.378 | 2.106 | 78.673 | 1.00 | 43.90 | C |
| ATOM | 606 | CG | LEU | H | 570 | 54.740 | 1.290 | 77.444 | 1.00 | 47.23 | C |
| ATOM | 607 | CD1 | LEU | H | 570 | 53.907 | 1.680 | 76.234 | 1.00 | 42.87 | C |
| ATOM | 608 | CD2 | LEU | H | 570 | 56.212 | 1.550 | 77.180 | 1.00 | 49.12 | C |
| ATOM | 609 | C | LEU | H | 570 | 52.325 | 1.201 | 79.775 | 1.00 | 33.13 | C |
| ATOM | 610 | O | LEU | H | 570 | 52.915 | 0.652 | 80.674 | 1.00 | 34.44 | O |
| ATOM | 611 | N | VAL | H | 571 | 51.127 | 0.817 | 79.397 | 1.00 | 32.97 | N |
| ATOM | 612 | CA | VAL | H | 571 | 50.487 | −0.271 | 80.102 | 1.00 | 33.81 | C |
| ATOM | 613 | CB | VAL | H | 571 | 49.021 | 0.104 | 80.542 | 1.00 | 31.84 | C |
| ATOM | 614 | CG1 | VAL | H | 571 | 48.320 | −1.076 | 81.190 | 1.00 | 30.69 | C |
| ATOM | 615 | CG2 | VAL | H | 571 | 49.017 | 1.286 | 81.497 | 1.00 | 29.32 | C |
| ATOM | 616 | C | VAL | H | 571 | 50.502 | −1.443 | 79.152 | 1.00 | 34.77 | C |
| ATOM | 617 | O | VAL | H | 571 | 50.210 | −1.263 | 77.977 | 1.00 | 36.19 | O |
| ATOM | 618 | N | TYR | H | 572 | 50.836 | −2.628 | 79.659 | 1.00 | 35.15 | N |
| ATOM | 619 | CA | TYR | H | 572 | 50.917 | −3.822 | 78.830 | 1.00 | 37.36 | C |
| ATOM | 620 | CB | TYR | H | 572 | 52.166 | −4.660 | 79.164 | 1.00 | 38.64 | C |
| ATOM | 621 | CG | TYR | H | 572 | 53.492 | −3.917 | 79.030 | 1.00 | 37.99 | C |
| ATOM | 622 | CD1 | TYR | H | 572 | 53.885 | −3.363 | 77.817 | 1.00 | 38.18 | C |
| ATOM | 623 | CE1 | TYR | H | 572 | 55.087 | −2.690 | 77.690 | 1.00 | 41.05 | C |
| ATOM | 624 | CZ | TYR | H | 572 | 55.923 | −2.564 | 78.790 | 1.00 | 44.19 | C |
| ATOM | 625 | OH | TYR | H | 572 | 57.122 | −1.894 | 78.663 | 1.00 | 49.32 | O |
| ATOM | 626 | CE2 | TYR | H | 572 | 55.563 | −3.105 | 80.006 | 1.00 | 40.80 | C |
| ATOM | 627 | CD2 | TYR | H | 572 | 54.353 | −3.787 | 80.119 | 1.00 | 39.01 | C |
| ATOM | 628 | C | TYR | H | 572 | 49.662 | −4.663 | 78.944 | 1.00 | 37.49 | C |
| ATOM | 629 | O | TYR | H | 572 | 49.004 | −4.649 | 79.972 | 1.00 | 43.43 | O |
| ATOM | 630 | N | GLY | H | 573 | 49.336 | −5.396 | 77.880 | 1.00 | 37.77 | N |
| ATOM | 631 | CA | GLY | H | 573 | 48.107 | −6.166 | 77.808 | 1.00 | 38.66 | C |
| ATOM | 632 | C | GLY | H | 573 | 48.304 | −7.492 | 78.492 | 1.00 | 38.32 | C |
| ATOM | 633 | O | GLY | H | 573 | 49.385 | −7.716 | 78.999 | 1.00 | 41.06 | O |
| ATOM | 634 | N | PRO | H | 574 | 47.278 | −8.346 | 78.536 | 1.00 | 42.08 | N |
| ATOM | 635 | CA | PRO | H | 574 | 47.402 | −9.734 | 79.044 | 1.00 | 47.57 | C |
| ATOM | 636 | CB | PRO | H | 574 | 46.036 | −10.363 | 78.694 | 1.00 | 43.93 | C |
| ATOM | 637 | CG | PRO | H | 574 | 45.085 | −9.195 | 78.692 | 1.00 | 42.66 | C |
| ATOM | 638 | CD | PRO | H | 574 | 45.886 | −8.018 | 78.159 | 1.00 | 42.03 | C |
| ATOM | 639 | C | PRO | H | 574 | 48.567 | −10.542 | 78.426 | 1.00 | 52.64 | C |
| ATOM | 640 | O | PRO | H | 574 | 49.541 | −9.935 | 77.995 | 1.00 | 53.35 | O |
| ATOM | 641 | N | GLU | H | 575 | 48.478 | −11.872 | 78.404 | 1.00 | 56.76 | N |
| ATOM | 642 | CA | GLU | H | 575 | 49.572 | −12.708 | 77.908 | 1.00 | 63.20 | C |
| ATOM | 643 | CB | GLU | H | 575 | 49.361 | −14.183 | 78.280 | 1.00 | 64.71 | C |
| ATOM | 644 | CG | GLU | H | 575 | 49.409 | −14.473 | 79.827 | 0.00 | 74.11 | C |
| ATOM | 645 | CD | GLU | H | 575 | 50.810 | −14.441 | 80.471 | 0.00 | 79.11 | C |
| ATOM | 646 | OE1 | GLU | H | 575 | 51.764 | −13.915 | 79.849 | 0.00 | 82.20 | O |
| ATOM | 647 | OE2 | GLU | H | 575 | 50.948 | −14.951 | 81.614 | 0.00 | 79.34 | O |
| ATOM | 648 | C | GLU | H | 575 | 49.769 | −12.532 | 76.394 | 1.00 | 67.74 | C |
| ATOM | 649 | O | GLU | H | 575 | 50.210 | −11.471 | 75.943 | 1.00 | 70.43 | O |
| ATOM | 650 | N | GLY | H | 576 | 49.438 | −13.561 | 75.611 | 1.00 | 66.87 | N |
| ATOM | 651 | CA | GLY | H | 576 | 49.623 | −13.539 | 74.162 | 1.00 | 58.07 | C |
| ATOM | 652 | C | GLY | H | 576 | 48.720 | −12.550 | 73.450 | 1.00 | 53.00 | C |
| ATOM | 653 | O | GLY | H | 576 | 48.024 | −12.890 | 72.490 | 1.00 | 56.59 | O |
| ATOM | 654 | N | SER | H | 577 | 48.730 | −11.314 | 73.926 | 1.00 | 43.31 | N |
| ATOM | 655 | CA | SER | H | 577 | 47.882 | −10.300 | 73.370 | 1.00 | 38.03 | C |
| ATOM | 656 | CB | SER | H | 577 | 47.240 | −9.506 | 74.500 | 1.00 | 34.66 | C |
| ATOM | 657 | OG | SER | H | 577 | 48.176 | −8.640 | 75.098 | 1.00 | 29.49 | O |
| ATOM | 658 | C | SER | H | 577 | 48.715 | −9.402 | 72.468 | 1.00 | 35.06 | C |
| ATOM | 659 | O | SER | H | 577 | 48.331 | −9.112 | 71.348 | 1.00 | 39.63 | O |
| ATOM | 660 | N | ASP | H | 578 | 49.867 | −8.996 | 72.975 | 1.00 | 29.58 | N |
| ATOM | 661 | CA | ASP | H | 578 | 50.806 | −8.117 | 72.282 | 1.00 | 36.84 | C |
| ATOM | 662 | CB | ASP | H | 578 | 51.178 | −8.646 | 70.888 | 1.00 | 38.03 | C |
| ATOM | 663 | CG | ASP | H | 578 | 52.051 | −9.870 | 70.970 | 1.00 | 41.79 | C |
| ATOM | 664 | OD1 | ASP | H | 578 | 53.120 | −9.793 | 71.600 | 1.00 | 45.74 | O |
| ATOM | 665 | OD2 | ASP | H | 578 | 51.748 | −10.959 | 70.467 | 1.00 | 46.43 | O |
| ATOM | 666 | C | ASP | H | 578 | 50.365 | −6.677 | 72.225 | 1.00 | 33.77 | C |
| ATOM | 667 | O | ASP | H | 578 | 50.950 | −5.871 | 71.497 | 1.00 | 35.82 | O |
| ATOM | 668 | N | LEU | H | 579 | 49.356 | −6.340 | 73.017 | 1.00 | 29.55 | N |
| ATOM | 669 | CA | LEU | H | 579 | 48.865 | −4.965 | 73.008 | 1.00 | 24.41 | C |
| ATOM | 670 | CB | LEU | H | 579 | 47.362 | −4.922 | 73.178 | 1.00 | 23.57 | C |
| ATOM | 671 | CG | LEU | H | 579 | 46.700 | −5.577 | 71.974 | 1.00 | 24.08 | C |
| ATOM | 672 | CD1 | LEU | H | 579 | 45.309 | −6.038 | 72.324 | 1.00 | 27.54 | C |
| ATOM | 673 | CD2 | LEU | H | 579 | 46.696 | −4.534 | 70.845 | 1.00 | 24.63 | C |
| ATOM | 674 | C | LEU | H | 579 | 49.547 | −4.125 | 74.052 | 1.00 | 27.54 | C |
| ATOM | 675 | O | LEU | H | 579 | 50.070 | −4.627 | 75.052 | 1.00 | 33.84 | O |
| ATOM | 676 | N | VAL | H | 580 | 49.597 | −2.839 | 73.783 | 1.00 | 28.01 | N |
| ATOM | 677 | CA | VAL | H | 580 | 50.054 | −1.901 | 74.774 | 1.00 | 31.86 | C |
| ATOM | 678 | CB | VAL | H | 580 | 51.612 | −1.735 | 74.813 | 1.00 | 30.08 | C |
| ATOM | 679 | CG1 | VAL | H | 580 | 52.309 | −2.785 | 73.969 | 1.00 | 30.31 | C |
| ATOM | 680 | CG2 | VAL | H | 580 | 52.030 | −0.348 | 74.392 | 1.00 | 25.68 | C |
| ATOM | 681 | C | VAL | H | 580 | 49.322 | −0.575 | 74.558 | 1.00 | 36.73 | C |
| ATOM | 682 | O | VAL | H | 580 | 49.112 | −0.133 | 73.418 | 1.00 | 38.70 | O |
| ATOM | 683 | N | LEU | H | 581 | 48.907 | 0.034 | 75.663 | 1.00 | 32.92 | N |
| ATOM | 684 | CA | LEU | H | 581 | 48.268 | 1.327 | 75.619 | 1.00 | 25.72 | C |
| ATOM | 685 | CB | LEU | H | 581 | 47.075 | 1.359 | 76.559 | 1.00 | 28.78 | C |
| ATOM | 686 | CG | LEU | H | 581 | 45.795 | 0.752 | 76.011 | 1.00 | 31.40 | C |
| ATOM | 687 | CD1 | LEU | H | 581 | 44.902 | 0.313 | 77.149 | 1.00 | 27.82 | C |
| ATOM | 688 | CD2 | LEU | H | 581 | 45.104 | 1.780 | 75.103 | 1.00 | 30.05 | C |
| ATOM | 689 | C | LEU | H | 581 | 49.272 | 2.341 | 76.043 | 1.00 | 21.89 | C |
| ATOM | 690 | O | LEU | H | 581 | 49.789 | 2.280 | 77.136 | 1.00 | 32.83 | O |
| ATOM | 691 | N | MET | H | 582 | 49.599 | 3.243 | 75.143 | 1.00 | 26.34 | N |
| ATOM | 692 | CA | MET | H | 582 | 50.417 | 4.360 | 75.499 | 1.00 | 32.35 | C |
| ATOM | 693 | CB | MET | H | 582 | 51.184 | 4.894 | 74.289 | 1.00 | 30.11 | C |
| ATOM | 694 | CG | MET | H | 582 | 52.124 | 3.946 | 73.545 | 1.00 | 30.27 | C |
| ATOM | 695 | SD | MET | H | 582 | 53.141 | 5.037 | 72.424 | 1.00 | 34.40 | S |
| ATOM | 696 | CE | MET | H | 582 | 51.857 | 5.896 | 71.517 | 1.00 | 36.23 | C |
| ATOM | 697 | C | MET | H | 582 | 49.474 | 5.445 | 76.041 | 1.00 | 40.30 | C |
| ATOM | 698 | O | MET | H | 582 | 48.451 | 5.792 | 75.423 | 1.00 | 35.77 | O |
| ATOM | 699 | N | LYS | H | 583 | 49.820 | 5.974 | 77.204 | 1.00 | 44.99 | N |
| ATOM | 700 | CA | LYS | H | 583 | 49.136 | 7.137 | 77.724 | 1.00 | 47.30 | C |
| ATOM | 701 | CB | LYS | H | 583 | 48.989 | 7.028 | 79.241 | 1.00 | 52.75 | C |
| ATOM | 702 | CG | LYS | H | 583 | 47.912 | 7.905 | 79.849 | 1.00 | 55.81 | C |
| ATOM | 703 | CD | LYS | H | 583 | 48.413 | 9.303 | 80.092 | 1.00 | 59.13 | C |
| ATOM | 704 | CE | LYS | H | 583 | 47.630 | 9.990 | 81.203 | 1.00 | 62.76 | C |
| ATOM | 705 | NZ | LYS | H | 583 | 47.980 | 9.461 | 82.549 | 1.00 | 63.30 | N |
| ATOM | 706 | C | LYS | H | 583 | 49.995 | 8.319 | 77.322 | 1.00 | 44.26 | C |
| ATOM | 707 | O | LYS | H | 583 | 51.212 | 8.295 | 77.471 | 1.00 | 41.27 | O |
| ATOM | 708 | N | LEU | H | 584 | 49.362 | 9.340 | 76.773 | 1.00 | 45.99 | N |
| ATOM | 709 | CA | LEU | H | 584 | 50.102 | 10.476 | 76.271 | 1.00 | 43.75 | C |
| ATOM | 710 | CB | LEU | H | 584 | 49.338 | 11.142 | 75.125 | 1.00 | 42.25 | C |
| ATOM | 711 | CG | LEU | H | 584 | 49.284 | 10.339 | 73.816 | 1.00 | 36.41 | C |
| ATOM | 712 | CD1 | LEU | H | 584 | 48.288 | 10.953 | 72.869 | 1.00 | 29.02 | C |
| ATOM | 713 | CD2 | LEU | H | 584 | 50.668 | 10.338 | 73.179 | 1.00 | 31.80 | C |
| ATOM | 714 | C | LEU | H | 584 | 50.340 | 11.459 | 77.396 | 1.00 | 47.49 | C |
| ATOM | 715 | O | LEU | H | 584 | 49.524 | 11.572 | 78.325 | 1.00 | 43.19 | O |
| ATOM | 716 | N | ALA | H | 585 | 51.447 | 12.193 | 77.296 | 1.00 | 50.51 | N |
| ATOM | 717 | CA | ALA | H | 585 | 51.749 | 13.114 | 78.368 | 1.00 | 55.22 | C |
| ATOM | 718 | CB | ALA | H | 585 | 53.172 | 13.677 | 78.343 | 1.00 | 51.41 | C |
| ATOM | 719 | C | ALA | H | 585 | 50.646 | 14.138 | 78.595 | 1.00 | 65.14 | C |
| ATOM | 720 | O | ALA | H | 585 | 49.956 | 14.031 | 79.605 | 1.00 | 70.29 | O |
| ATOM | 721 | N | ARG | H | 586 | 50.391 | 15.035 | 77.652 | 1.00 | 67.58 | N |
| ATOM | 722 | CA | ARG | H | 586 | 49.342 | 16.052 | 77.791 | 1.00 | 68.58 | C |
| ATOM | 723 | CB | ARG | H | 586 | 50.050 | 17.399 | 77.654 | 1.00 | 70.59 | C |
| ATOM | 724 | CG | ARG | H | 586 | 49.310 | 18.629 | 77.218 | 1.00 | 75.35 | C |
| ATOM | 725 | CD | ARG | H | 586 | 50.219 | 19.868 | 77.252 | 1.00 | 77.93 | C |
| ATOM | 726 | NE | ARG | H | 586 | 51.415 | 19.756 | 76.408 | 1.00 | 77.62 | N |
| ATOM | 727 | CZ | ARG | H | 586 | 51.447 | 20.053 | 75.111 | 1.00 | 76.98 | C |
| ATOM | 728 | NH1 | ARG | H | 586 | 50.344 | 20.460 | 74.491 | 1.00 | 75.99 | N |
| ATOM | 729 | NH2 | ARG | H | 586 | 52.578 | 19.940 | 74.429 | 1.00 | 78.01 | N |
| ATOM | 730 | C | ARG | H | 586 | 48.295 | 15.754 | 76.698 | 1.00 | 67.21 | C |
| ATOM | 731 | O | ARG | H | 586 | 48.667 | 15.239 | 75.657 | 1.00 | 63.31 | O |
| ATOM | 732 | N | PRO | H | 587 | 47.008 | 16.067 | 76.883 | 1.00 | 68.20 | N |
| ATOM | 733 | CA | PRO | H | 587 | 45.955 | 15.609 | 75.952 | 1.00 | 63.50 | C |
| ATOM | 734 | CB | PRO | H | 587 | 44.704 | 16.320 | 76.475 | 1.00 | 67.62 | C |
| ATOM | 735 | CG | PRO | H | 587 | 45.259 | 17.513 | 77.235 | 1.00 | 69.53 | C |
| ATOM | 736 | CD | PRO | H | 587 | 46.443 | 16.922 | 77.945 | 1.00 | 69.56 | C |
| ATOM | 737 | C | PRO | H | 587 | 46.166 | 16.003 | 74.494 | 1.00 | 57.41 | C |
| ATOM | 738 | O | PRO | H | 587 | 46.760 | 17.045 | 74.200 | 1.00 | 59.55 | O |
| ATOM | 739 | N | ALA | H | 588 | 45.663 | 15.167 | 73.593 | 1.00 | 50.43 | N |
| ATOM | 740 | CA | ALA | H | 588 | 45.657 | 15.475 | 72.173 | 1.00 | 47.60 | C |
| ATOM | 741 | CB | ALA | H | 588 | 45.466 | 14.202 | 71.361 | 1.00 | 49.06 | C |
| ATOM | 742 | C | ALA | H | 588 | 44.532 | 16.457 | 71.885 | 1.00 | 41.99 | C |
| ATOM | 743 | O | ALA | H | 588 | 43.422 | 16.290 | 72.368 | 1.00 | 32.66 | O |
| ATOM | 744 | N | VAL | H | 589 | 44.832 | 17.480 | 71.095 | 1.00 | 45.60 | N |
| ATOM | 745 | CA | VAL | H | 589 | 43.851 | 18.497 | 70.719 | 1.00 | 47.16 | C |
| ATOM | 746 | CB | VAL | H | 589 | 44.560 | 19.852 | 70.402 | 1.00 | 49.14 | C |
| ATOM | 747 | CG1 | VAL | H | 589 | 43.586 | 20.926 | 69.896 | 1.00 | 50.70 | C |
| ATOM | 748 | CG2 | VAL | H | 589 | 45.314 | 20.355 | 71.614 | 1.00 | 48.13 | C |
| ATOM | 749 | C | VAL | H | 589 | 43.093 | 17.982 | 69.495 | 1.00 | 43.62 | C |
| ATOM | 750 | O | VAL | H | 589 | 43.622 | 18.010 | 68.380 | 1.00 | 40.45 | O |
| ATOM | 751 | N | LEU | H | 590 | 41.875 | 17.482 | 69.703 | 1.00 | 42.14 | N |
| ATOM | 752 | CA | LEU | H | 590 | 41.019 | 17.089 | 68.574 | 1.00 | 43.96 | C |
| ATOM | 753 | CB | LEU | H | 590 | 39.705 | 16.434 | 69.030 | 1.00 | 35.82 | C |
| ATOM | 754 | CG | LEU | H | 590 | 39.710 | 15.324 | 70.090 | 1.00 | 34.97 | C |
| ATOM | 755 | CD1 | LEU | H | 590 | 38.386 | 14.578 | 70.100 | 1.00 | 31.79 | C |
| ATOM | 756 | CD2 | LEU | H | 590 | 40.864 | 14.331 | 69.944 | 1.00 | 33.40 | C |
| ATOM | 757 | C | LEU | H | 590 | 40.720 | 18.290 | 67.660 | 1.00 | 51.77 | C |
| ATOM | 758 | O | LEU | H | 590 | 40.425 | 19.407 | 68.127 | 1.00 | 55.01 | O |
| ATOM | 759 | N | ASP | H | 591 | 40.829 | 18.054 | 66.357 | 1.00 | 53.30 | N |
| ATOM | 760 | CA | ASP | H | 591 | 40.494 | 19.063 | 65.359 | 1.00 | 53.50 | C |
| ATOM | 761 | CB | ASP | H | 591 | 41.645 | 20.088 | 65.164 | 1.00 | 55.34 | C |
| ATOM | 762 | CG | ASP | H | 591 | 42.984 | 19.445 | 64.782 | 1.00 | 57.68 | C |
| ATOM | 763 | OD1 | ASP | H | 591 | 43.086 | 18.196 | 64.794 | 1.00 | 63.02 | O |
| ATOM | 764 | OD2 | ASP | H | 591 | 43.995 | 20.117 | 64.459 | 1.00 | 49.55 | O |
| ATOM | 765 | C | ASP | H | 591 | 40.049 | 18.421 | 64.041 | 1.00 | 51.49 | C |
| ATOM | 766 | O | ASP | H | 591 | 39.486 | 17.319 | 64.031 | 1.00 | 45.15 | O |
| ATOM | 767 | N | ASP | H | 592 | 40.288 | 19.136 | 62.945 | 1.00 | 53.28 | N |
| ATOM | 768 | CA | ASP | H | 592 | 40.017 | 18.645 | 61.596 | 1.00 | 58.57 | C |
| ATOM | 769 | CB | ASP | H | 592 | 40.090 | 19.800 | 60.590 | 1.00 | 62.72 | C |
| ATOM | 770 | CG | ASP | H | 592 | 38.714 | 20.341 | 60.203 | 1.00 | 67.55 | C |
| ATOM | 771 | OD1 | ASP | H | 592 | 38.042 | 20.972 | 61.063 | 1.00 | 67.31 | O |
| ATOM | 772 | OD2 | ASP | H | 592 | 38.238 | 20.190 | 59.050 | 1.00 | 67.97 | O |
| ATOM | 773 | C | ASP | H | 592 | 40.980 | 17.548 | 61.156 | 1.00 | 57.57 | C |
| ATOM | 774 | O | ASP | H | 592 | 40.658 | 16.768 | 60.271 | 1.00 | 57.45 | O |
| ATOM | 775 | N | PHE | H | 593 | 42.156 | 17.504 | 61.776 | 1.00 | 55.44 | N |
| ATOM | 776 | CA | PHE | H | 593 | 43.234 | 16.612 | 61.374 | 1.00 | 50.11 | C |
| ATOM | 777 | CB | PHE | H | 593 | 44.508 | 17.423 | 61.176 | 1.00 | 55.30 | C |
| ATOM | 778 | CG | PHE | H | 593 | 44.320 | 18.629 | 60.291 | 1.00 | 65.23 | C |
| ATOM | 779 | CD1 | PHE | H | 593 | 44.321 | 18.503 | 58.900 | 1.00 | 68.65 | C |
| ATOM | 780 | CE1 | PHE | H | 593 | 44.147 | 19.618 | 58.074 | 1.00 | 70.46 | C |
| ATOM | 781 | CZ | PHE | H | 593 | 43.967 | 20.881 | 58.641 | 1.00 | 71.10 | C |
| ATOM | 782 | CE2 | PHE | H | 593 | 43.964 | 21.021 | 60.029 | 1.00 | 70.78 | C |
| ATOM | 783 | CD2 | PHE | H | 593 | 44.138 | 19.893 | 60.846 | 1.00 | 69.28 | C |
| ATOM | 784 | C | PHE | H | 593 | 43.457 | 15.476 | 62.371 | 1.00 | 44.90 | C |
| ATOM | 785 | O | PHE | H | 593 | 43.993 | 14.423 | 62.035 | 1.00 | 46.58 | O |
| ATOM | 786 | N | VAL | H | 594 | 43.043 | 15.687 | 63.609 | 1.00 | 43.09 | N |
| ATOM | 787 | CA | VAL | H | 594 | 43.188 | 14.662 | 64.635 | 1.00 | 33.70 | C |
| ATOM | 788 | CB | VAL | H | 594 | 44.098 | 15.127 | 65.776 | 1.00 | 28.32 | C |
| ATOM | 789 | CG1 | VAL | H | 594 | 44.431 | 13.973 | 66.720 | 1.00 | 19.15 | C |
| ATOM | 790 | CG2 | VAL | H | 594 | 45.371 | 15.790 | 65.213 | 1.00 | 22.94 | C |
| ATOM | 791 | C | VAL | H | 594 | 41.834 | 14.353 | 65.198 | 1.00 | 31.33 | C |
| ATOM | 792 | O | VAL | H | 594 | 41.056 | 15.251 | 65.524 | 1.00 | 36.92 | O |
| ATOM | 793 | N | SER | H | 595 | 41.553 | 13.070 | 65.305 | 1.00 | 32.94 | N |
| ATOM | 794 | CA | SER | H | 595 | 40.281 | 12.593 | 65.798 | 1.00 | 33.67 | C |
| ATOM | 795 | CB | SER | H | 595 | 39.254 | 12.470 | 64.668 | 1.00 | 41.32 | C |
| ATOM | 796 | OG | SER | H | 595 | 39.675 | 11.556 | 63.678 | 1.00 | 46.54 | O |
| ATOM | 797 | C | SER | H | 595 | 40.566 | 11.251 | 66.393 | 1.00 | 32.17 | C |
| ATOM | 798 | O | SER | H | 595 | 41.690 | 10.750 | 66.294 | 1.00 | 40.82 | O |
| ATOM | 799 | N | THR | H | 596 | 39.535 | 10.679 | 67.007 | 1.00 | 29.23 | N |
| ATOM | 800 | CA | THR | H | 596 | 39.625 | 9.383 | 67.648 | 1.00 | 31.54 | C |
| ATOM | 801 | CB | THR | H | 596 | 39.267 | 9.514 | 69.134 | 1.00 | 37.29 | C |
| ATOM | 802 | OG1 | THR | H | 596 | 37.898 | 9.904 | 69.262 | 1.00 | 41.23 | O |
| ATOM | 803 | CG2 | THR | H | 596 | 40.032 | 10.685 | 69.778 | 1.00 | 36.09 | C |
| ATOM | 804 | C | THR | H | 596 | 38.703 | 8.341 | 66.968 | 1.00 | 39.80 | C |
| ATOM | 805 | O | THR | H | 596 | 37.710 | 8.692 | 66.321 | 1.00 | 43.66 | O |
| ATOM | 806 | N | ILE | H | 597 | 39.027 | 7.059 | 67.123 | 1.00 | 36.79 | N |
| ATOM | 807 | CA | ILE | H | 597 | 38.273 | 6.032 | 66.473 | 1.00 | 34.43 | C |
| ATOM | 808 | CB | ILE | H | 597 | 39.181 | 5.174 | 65.567 | 1.00 | 38.71 | C |
| ATOM | 809 | CG1 | ILE | H | 597 | 38.401 | 3.969 | 65.030 | 1.00 | 40.16 | C |
| ATOM | 810 | CD1 | ILE | H | 597 | 39.112 | 3.227 | 63.931 | 1.00 | 48.03 | C |
| ATOM | 811 | CG2 | ILE | H | 597 | 40.445 | 4.718 | 66.313 | 1.00 | 35.96 | C |
| ATOM | 812 | C | ILE | H | 597 | 37.661 | 5.215 | 67.586 | 1.00 | 38.05 | C |
| ATOM | 813 | O | ILE | H | 597 | 38.290 | 5.012 | 68.624 | 1.00 | 39.06 | O |
| ATOM | 814 | N | ASP | H | 598 | 36.430 | 4.756 | 67.368 | 1.00 | 43.76 | N |
| ATOM | 815 | CA | ASP | H | 598 | 35.706 | 3.953 | 68.348 | 1.00 | 44.80 | C |
| ATOM | 816 | CB | ASP | H | 598 | 34.197 | 3.911 | 68.034 | 1.00 | 49.33 | C |
| ATOM | 817 | CG | ASP | H | 598 | 33.553 | 5.277 | 68.167 | 1.00 | 49.92 | C |
| ATOM | 818 | OD1 | ASP | H | 598 | 33.962 | 6.032 | 69.079 | 1.00 | 52.31 | O |
| ATOM | 819 | OD2 | ASP | H | 598 | 32.671 | 5.701 | 67.395 | 1.00 | 51.81 | O |
| ATOM | 820 | C | ASP | H | 598 | 36.261 | 2.565 | 68.411 | 1.00 | 44.61 | C |
| ATOM | 821 | O | ASP | H | 598 | 36.951 | 2.133 | 67.488 | 1.00 | 48.21 | O |
| ATOM | 822 | N | LEU | H | 599 | 35.947 | 1.891 | 69.517 | 1.00 | 45.80 | N |
| ATOM | 823 | CA | LEU | H | 599 | 36.359 | 0.528 | 69.799 | 1.00 | 43.39 | C |
| ATOM | 824 | CB | LEU | H | 599 | 36.944 | 0.461 | 71.202 | 1.00 | 41.84 | C |
| ATOM | 825 | CG | LEU | H | 599 | 38.212 | 1.306 | 71.369 | 1.00 | 43.53 | C |
| ATOM | 826 | CD1 | LEU | H | 599 | 38.356 | 1.854 | 72.780 | 1.00 | 40.25 | C |
| ATOM | 827 | CD2 | LEU | H | 599 | 39.431 | 0.490 | 70.973 | 1.00 | 45.73 | C |
| ATOM | 828 | C | LEU | H | 599 | 35.122 | −0.339 | 69.723 | 1.00 | 47.43 | C |
| ATOM | 829 | O | LEU | H | 599 | 34.030 | 0.138 | 69.998 | 1.00 | 51.46 | O |
| ATOM | 830 | N | PRO | H | 600 | 35.264 | −1.599 | 69.326 | 1.00 | 47.32 | N |
| ATOM | 831 | CA | PRO | H | 600 | 34.109 | −2.494 | 69.248 | 1.00 | 49.67 | C |
| ATOM | 832 | CB | PRO | H | 600 | 34.676 | −3.715 | 68.530 | 1.00 | 44.89 | C |
| ATOM | 833 | CG | PRO | H | 600 | 36.081 | −3.707 | 68.927 | 1.00 | 45.27 | C |
| ATOM | 834 | CD | PRO | H | 600 | 36.498 | −2.267 | 68.885 | 1.00 | 43.97 | C |
| ATOM | 835 | C | PRO | H | 600 | 33.592 | −2.885 | 70.627 | 1.00 | 56.33 | C |
| ATOM | 836 | O | PRO | H | 600 | 34.208 | −2.519 | 71.639 | 1.00 | 61.22 | O |
| ATOM | 837 | N | ASN | H | 601 | 32.475 | −3.620 | 70.654 | 1.00 | 59.20 | N |
| ATOM | 838 | CA | ASN | H | 601 | 31.893 | −4.114 | 71.900 | 1.00 | 59.73 | C |
| ATOM | 839 | CB | ASN | H | 601 | 30.366 | −4.078 | 71.867 | 1.00 | 66.41 | C |
| ATOM | 840 | CG | ASN | H | 601 | 29.814 | −2.678 | 71.655 | 1.00 | 74.47 | C |
| ATOM | 841 | OD1 | ASN | H | 601 | 30.342 | −1.691 | 72.184 | 1.00 | 76.13 | O |
| ATOM | 842 | ND2 | ASN | H | 601 | 28.733 | −2.587 | 70.885 | 1.00 | 76.66 | N |
| ATOM | 843 | C | ASN | H | 601 | 32.353 | −5.513 | 72.211 | 1.00 | 56.45 | C |
| ATOM | 844 | O | ASN | H | 601 | 32.271 | −6.420 | 71.376 | 1.00 | 52.84 | O |
| ATOM | 845 | N | TYR | H | 602 | 32.833 | −5.653 | 73.437 | 1.00 | 54.77 | N |
| ATOM | 846 | CA | TYR | H | 602 | 33.321 | −6.895 | 74.015 | 1.00 | 56.96 | C |
| ATOM | 847 | CB | TYR | H | 602 | 33.133 | −6.824 | 75.532 | 1.00 | 60.58 | C |
| ATOM | 848 | CG | TYR | H | 602 | 33.286 | −5.421 | 76.105 | 1.00 | 65.84 | C |
| ATOM | 849 | CD1 | TYR | H | 602 | 34.318 | −4.579 | 75.686 | 1.00 | 67.82 | C |
| ATOM | 850 | CE1 | TYR | H | 602 | 34.467 | −3.289 | 76.206 | 1.00 | 68.03 | C |
| ATOM | 851 | CZ | TYR | H | 602 | 33.588 | −2.838 | 77.167 | 1.00 | 67.46 | C |
| ATOM | 852 | OH | TYR | H | 602 | 33.735 | −1.583 | 77.685 | 1.00 | 71.22 | O |
| ATOM | 853 | CE2 | TYR | H | 602 | 32.559 | −3.645 | 77.613 | 1.00 | 68.36 | C |
| ATOM | 854 | CD2 | TYR | H | 602 | 32.407 | −4.937 | 77.078 | 1.00 | 68.49 | C |
| ATOM | 855 | C | TYR | H | 602 | 32.663 | −8.155 | 73.457 | 1.00 | 55.86 | C |
| ATOM | 856 | O | TYR | H | 602 | 31.715 | −8.660 | 74.037 | 1.00 | 61.02 | O |
| ATOM | 857 | N | GLY | H | 603 | 33.167 | −8.656 | 72.331 | 1.00 | 51.82 | N |
| ATOM | 858 | CA | GLY | H | 603 | 32.664 | −9.890 | 71.740 | 1.00 | 50.68 | C |
| ATOM | 859 | C | GLY | H | 603 | 32.070 | −9.792 | 70.337 | 1.00 | 52.27 | C |
| ATOM | 860 | O | GLY | H | 603 | 31.554 | −10.787 | 69.818 | 1.00 | 53.79 | O |
| ATOM | 861 | N | SER | H | 604 | 32.130 | −8.607 | 69.725 | 1.00 | 51.77 | N |
| ATOM | 862 | CA | SER | H | 604 | 31.612 | −8.388 | 68.374 | 1.00 | 47.55 | C |
| ATOM | 863 | CB | SER | H | 604 | 31.942 | −6.979 | 67.898 | 1.00 | 50.94 | C |
| ATOM | 864 | OG | SER | H | 604 | 31.664 | −6.015 | 68.897 | 1.00 | 54.63 | O |
| ATOM | 865 | C | SER | H | 604 | 32.217 | −9.388 | 67.410 | 1.00 | 49.90 | C |
| ATOM | 866 | O | SER | H | 604 | 33.439 | −9.501 | 67.331 | 1.00 | 57.08 | O |
| ATOM | 867 | N | THR | H | 605 | 31.370 | −10.122 | 66.693 | 1.00 | 47.22 | N |
| ATOM | 868 | CA | THR | H | 605 | 31.833 | −11.063 | 65.672 | 1.00 | 46.30 | C |
| ATOM | 869 | CB | THR | H | 605 | 31.073 | −12.431 | 65.773 | 1.00 | 43.53 | C |
| ATOM | 870 | OG1 | THR | H | 605 | 29.772 | −12.276 | 65.555 | 0.00 | 42.67 | O |
| ATOM | 871 | CG2 | THR | H | 605 | 31.381 | −13.017 | 67.191 | 0.00 | 42.70 | C |
| ATOM | 872 | C | THR | H | 605 | 31.668 | −10.419 | 64.287 | 1.00 | 50.54 | C |
| ATOM | 873 | O | THR | H | 605 | 30.538 | −10.149 | 63.871 | 1.00 | 52.11 | O |
| ATOM | 874 | N | ILE | H | 606 | 32.787 | −10.145 | 63.599 | 1.00 | 50.02 | N |
| ATOM | 875 | CA | ILE | H | 606 | 32.772 | −9.600 | 62.227 | 1.00 | 46.20 | C |
| ATOM | 876 | CB | ILE | H | 606 | 34.045 | −8.792 | 61.916 | 1.00 | 46.40 | C |
| ATOM | 877 | CG1 | ILE | H | 606 | 34.474 | −8.071 | 63.195 | 0.00 | 39.95 | C |
| ATOM | 878 | CD1 | ILE | H | 606 | 33.383 | −7.216 | 63.840 | 0.00 | 39.56 | C |
| ATOM | 879 | CG2 | ILE | H | 606 | 33.790 | −7.853 | 60.789 | 0.00 | 40.12 | C |
| ATOM | 880 | C | ILE | H | 606 | 32.596 | −10.703 | 61.189 | 1.00 | 48.16 | C |
| ATOM | 881 | O | ILE | H | 606 | 33.369 | −11.669 | 61.169 | 1.00 | 47.74 | O |
| ATOM | 882 | N | PRO | H | 607 | 31.569 | −10.582 | 60.340 | 1.00 | 50.06 | N |
| ATOM | 883 | CA | PRO | H | 607 | 31.257 | −11.641 | 59.371 | 1.00 | 50.19 | C |
| ATOM | 884 | CB | PRO | H | 607 | 29.876 | −11.235 | 58.832 | 1.00 | 46.87 | C |
| ATOM | 885 | CG | PRO | H | 607 | 29.767 | −9.784 | 59.069 | 1.00 | 44.14 | C |
| ATOM | 886 | CD | PRO | H | 607 | 30.622 | −9.454 | 60.248 | 1.00 | 47.68 | C |
| ATOM | 887 | C | PRO | H | 607 | 32.291 | −11.715 | 58.248 | 1.00 | 51.67 | C |
| ATOM | 888 | O | PRO | H | 607 | 32.940 | −10.707 | 57.944 | 1.00 | 52.02 | O |
| ATOM | 889 | N | GLU | H | 608 | 32.433 | −12.899 | 57.657 | 1.00 | 50.27 | N |
| ATOM | 890 | CA | GLU | H | 608 | 33.395 | −13.145 | 56.593 | 1.00 | 54.38 | C |
| ATOM | 891 | CB | GLU | H | 608 | 33.436 | −14.628 | 56.268 | 1.00 | 59.36 | C |
| ATOM | 892 | CG | GLU | H | 608 | 33.664 | −15.514 | 57.480 | 1.00 | 66.38 | C |
| ATOM | 893 | CD | GLU | H | 608 | 34.006 | −16.940 | 57.090 | 1.00 | 70.96 | C |
| ATOM | 894 | OE1 | GLU | H | 608 | 34.676 | −17.130 | 56.050 | 1.00 | 72.25 | O |
| ATOM | 895 | OE2 | GLU | H | 608 | 33.611 | −17.873 | 57.824 | 1.00 | 72.85 | O |
| ATOM | 896 | C | GLU | H | 608 | 33.087 | −12.378 | 55.319 | 1.00 | 55.92 | C |
| ATOM | 897 | O | GLU | H | 608 | 31.935 | −12.051 | 55.045 | 1.00 | 61.18 | O |
| ATOM | 898 | N | LYS | H | 609 | 34.135 | −12.115 | 54.539 | 1.00 | 53.84 | N |
| ATOM | 899 | CA | LYS | H | 609 | 34.048 | −11.401 | 53.265 | 1.00 | 48.72 | C |
| ATOM | 900 | CB | LYS | H | 609 | 33.063 | −12.086 | 52.310 | 1.00 | 50.57 | C |
| ATOM | 901 | CG | LYS | H | 609 | 33.395 | 13.470 | 51.903 | 0.00 | 45.30 | C |
| ATOM | 902 | CD | LYS | H | 609 | 32.343 | −14.061 | 50.975 | 0.00 | 45.23 | C |
| ATOM | 903 | CE | LYS | H | 609 | 32.233 | −13.277 | 49.674 | 0.00 | 44.75 | C |
| ATOM | 904 | NZ | LYS | H | 609 | 33.505 | −13.289 | 48.896 | 0.00 | 44.80 | N |
| ATOM | 905 | C | LYS | H | 609 | 33.743 | −9.912 | 53.446 | 1.00 | 48.03 | C |
| ATOM | 906 | O | LYS | H | 609 | 33.536 | −9.191 | 52.466 | 1.00 | 47.61 | O |
| ATOM | 907 | N | THR | H | 610 | 33.734 | −9.470 | 54.709 | 1.00 | 48.60 | N |
| ATOM | 908 | CA | THR | H | 610 | 33.585 | −8.064 | 55.092 | 1.00 | 46.45 | C |
| ATOM | 909 | CB | THR | H | 610 | 33.347 | −7.943 | 56.633 | 1.00 | 46.23 | C |
| ATOM | 910 | OG1 | THR | H | 610 | 32.059 | −8.485 | 56.985 | 1.00 | 46.03 | O |
| ATOM | 911 | CG2 | THR | H | 610 | 33.274 | −6.448 | 57.081 | 1.00 | 41.23 | C |
| ATOM | 912 | C | THR | H | 610 | 34.832 | −7.267 | 54.704 | 1.00 | 48.74 | C |
| ATOM | 913 | O | THR | H | 610 | 35.958 | −7.720 | 54.914 | 1.00 | 51.81 | O |
| ATOM | 914 | N | SER | H | 611 | 34.620 | −6.076 | 54.153 | 1.00 | 49.05 | N |
| ATOM | 915 | CA | SER | H | 611 | 35.705 | −5.216 | 53.690 | 1.00 | 46.34 | C |
| ATOM | 916 | CB | SER | H | 611 | 35.174 | −4.262 | 52.617 | 1.00 | 50.03 | C |
| ATOM | 917 | OG | SER | H | 611 | 36.155 | −3.310 | 52.263 | 1.00 | 58.60 | O |
| ATOM | 918 | C | SER | H | 611 | 36.381 | −4.428 | 54.822 | 1.00 | 42.45 | C |
| ATOM | 919 | O | SER | H | 611 | 35.722 | −3.780 | 55.639 | 1.00 | 44.95 | O |
| ATOM | 920 | N | CYS | H | 612 | 37.708 | −4.465 | 54.840 | 1.00 | 39.40 | N |
| ATOM | 921 | CA | CYS | H | 612 | 38.496 | −3.853 | 55.909 | 1.00 | 36.53 | C |
| ATOM | 922 | CB | CYS | H | 612 | 38.933 | −4.934 | 56.906 | 1.00 | 35.64 | C |
| ATOM | 923 | SG | CYS | H | 612 | 37.571 | −5.719 | 57.821 | 1.00 | 39.82 | S |
| ATOM | 924 | C | CYS | H | 612 | 39.706 | −3.113 | 55.314 | 1.00 | 29.53 | C |
| ATOM | 925 | O | CYS | H | 612 | 40.117 | −3.426 | 54.207 | 1.00 | 28.76 | O |
| ATOM | 926 | N | SER | H | 613 | 40.249 | −2.129 | 56.038 | 1.00 | 19.51 | N |
| ATOM | 927 | CA | SER | H | 613 | 41.465 | −1.422 | 55.621 | 1.00 | 22.01 | C |
| ATOM | 928 | CB | SER | H | 613 | 41.131 | −0.009 | 55.157 | 1.00 | 23.34 | C |
| ATOM | 929 | OG | SER | H | 613 | 40.031 | −0.032 | 54.261 | 1.00 | 33.47 | O |
| ATOM | 930 | C | SER | H | 613 | 42.613 | −1.386 | 56.683 | 1.00 | 18.88 | C |
| ATOM | 931 | O | SER | H | 613 | 42.365 | −1.250 | 57.875 | 1.00 | 16.28 | O |
| ATOM | 932 | N | VAL | H | 614 | 43.859 | −1.512 | 56.227 | 1.00 | 13.54 | N |
| ATOM | 933 | CA | VAL | H | 614 | 45.024 | −1.278 | 57.083 | 1.00 | 14.59 | C |
| ATOM | 934 | CB | VAL | H | 614 | 46.033 | −2.452 | 57.072 | 1.00 | 15.36 | C |
| ATOM | 935 | CG1 | VAL | H | 614 | 45.375 | −3.706 | 57.663 | 1.00 | 6.21 | C |
| ATOM | 936 | CG2 | VAL | H | 614 | 46.581 | −2.741 | 55.644 | 1.00 | 3.44 | C |
| ATOM | 937 | C | VAL | H | 614 | 45.663 | −0.013 | 56.638 | 1.00 | 14.63 | C |
| ATOM | 938 | O | VAL | H | 614 | 45.582 | 0.350 | 55.460 | 1.00 | 25.10 | O |
| ATOM | 939 | N | TYR | H | 615 | 46.267 | 0.695 | 57.574 | 1.00 | 16.98 | N |
| ATOM | 940 | CA | TYR | H | 615 | 46.974 | 1.927 | 57.217 | 1.00 | 21.34 | C |
| ATOM | 941 | CB | TYR | H | 615 | 46.211 | 3.141 | 57.776 | 1.00 | 21.81 | C |
| ATOM | 942 | CG | TYR | H | 615 | 44.805 | 3.277 | 57.229 | 1.00 | 23.66 | C |
| ATOM | 943 | CD1 | TYR | H | 615 | 43.753 | 2.550 | 57.770 | 1.00 | 28.02 | C |
| ATOM | 944 | CE1 | TYR | H | 615 | 42.451 | 2.672 | 57.266 | 1.00 | 30.65 | C |
| ATOM | 945 | CZ | TYR | H | 615 | 42.201 | 3.527 | 56.207 | 1.00 | 30.45 | C |
| ATOM | 946 | OH | TYR | H | 615 | 40.910 | 3.654 | 55.710 | 1.00 | 32.41 | O |
| ATOM | 947 | CE2 | TYR | H | 615 | 43.237 | 4.260 | 55.659 | 1.00 | 28.55 | C |
| ATOM | 948 | CD2 | TYR | H | 615 | 44.526 | 4.137 | 56.169 | 1.00 | 26.86 | C |
| ATOM | 949 | C | TYR | H | 615 | 48.377 | 1.871 | 57.798 | 1.00 | 19.08 | C |
| ATOM | 950 | O | TYR | H | 615 | 48.564 | 1.311 | 58.858 | 1.00 | 25.04 | O |
| ATOM | 951 | N | GLY | H | 616 | 49.368 | 2.449 | 57.133 | 1.00 | 19.90 | N |
| ATOM | 952 | CA | GLY | H | 616 | 50.674 | 2.522 | 57.748 | 1.00 | 13.09 | C |
| ATOM | 953 | C | GLY | H | 616 | 51.722 | 3.300 | 56.996 | 1.00 | 17.97 | C |
| ATOM | 954 | O | GLY | H | 616 | 51.608 | 3.521 | 55.797 | 1.00 | 20.03 | O |
| ATOM | 955 | N | TRP | H | 617 | 52.759 | 3.716 | 57.713 | 1.00 | 16.66 | N |
| ATOM | 956 | CA | TRP | H | 617 | 53.931 | 4.283 | 57.078 | 1.00 | 18.67 | C |
| ATOM | 957 | CB | TRP | H | 617 | 54.416 | 5.480 | 57.864 | 1.00 | 19.92 | C |
| ATOM | 958 | CG | TRP | H | 617 | 53.493 | 6.631 | 57.822 | 1.00 | 20.64 | C |
| ATOM | 959 | CD1 | TRP | H | 617 | 53.457 | 7.611 | 56.877 | 1.00 | 18.32 | C |
| ATOM | 960 | NE1 | TRP | H | 617 | 52.493 | 8.535 | 57.200 | 1.00 | 24.45 | N |
| ATOM | 961 | CE2 | TRP | H | 617 | 51.887 | 8.171 | 58.376 | 1.00 | 19.42 | C |
| ATOM | 962 | CD2 | TRP | H | 617 | 52.498 | 6.971 | 58.804 | 1.00 | 21.10 | C |
| ATOM | 963 | CE3 | TRP | H | 617 | 52.072 | 6.385 | 60.023 | 1.00 | 21.58 | C |
| ATOM | 964 | CZ3 | TRP | H | 617 | 51.043 | 6.998 | 60.748 | 1.00 | 20.14 | C |
| ATOM | 965 | CH2 | TRP | H | 617 | 50.454 | 8.198 | 60.282 | 1.00 | 27.17 | C |
| ATOM | 966 | CZ2 | TRP | H | 617 | 50.867 | 8.800 | 59.103 | 1.00 | 21.31 | C |
| ATOM | 967 | C | TRP | H | 617 | 55.087 | 3.290 | 56.931 | 1.00 | 16.18 | C |
| ATOM | 968 | O | TRP | H | 617 | 56.221 | 3.697 | 56.582 | 1.00 | 12.58 | O |
| ATOM | 969 | N | GLY | H | 618 | 54.815 | 2.007 | 57.177 | 1.00 | 11.03 | N |
| ATOM | 970 | CA | GLY | H | 618 | 55.876 | 0.990 | 57.136 | 1.00 | 18.03 | C |
| ATOM | 971 | C | GLY | H | 618 | 56.455 | 0.660 | 55.765 | 1.00 | 18.50 | C |
| ATOM | 972 | O | GLY | H | 618 | 56.354 | 1.436 | 54.822 | 1.00 | 21.42 | O |
| ATOM | 973 | N | TYR | H | 619 | 57.075 | −0.506 | 55.667 | 1.00 | 19.87 | N |
| ATOM | 974 | CA | TYR | H | 619 | 57.767 | −0.971 | 54.458 | 1.00 | 17.30 | C |
| ATOM | 975 | CB | TYR | H | 619 | 58.353 | −2.363 | 54.736 | 1.00 | 19.95 | C |
| ATOM | 976 | CG | TYR | H | 619 | 59.157 | −2.927 | 53.597 | 1.00 | 18.72 | C |
| ATOM | 977 | CD1 | TYR | H | 619 | 60.353 | −2.313 | 53.189 | 1.00 | 17.79 | C |
| ATOM | 978 | CE1 | TYR | H | 619 | 61.096 | −2.823 | 52.129 | 1.00 | 22.45 | C |
| ATOM | 979 | CZ | TYR | H | 619 | 60.646 | −3.964 | 51.467 | 1.00 | 25.00 | C |
| ATOM | 980 | OH | TYR | H | 619 | 61.378 | −4.476 | 50.443 | 1.00 | 29.83 | O |
| ATOM | 981 | CE2 | TYR | H | 619 | 59.462 | −4.598 | 51.846 | 1.00 | 23.82 | C |
| ATOM | 982 | CD2 | TYR | H | 619 | 58.724 | −4.068 | 52.921 | 1.00 | 19.04 | C |
| ATOM | 983 | C | TYR | H | 619 | 56.882 | −1.068 | 53.212 | 1.00 | 14.46 | C |
| ATOM | 984 | O | TYR | H | 619 | 55.841 | −1.707 | 53.248 | 1.00 | 9.95 | O |
| ATOM | 985 | N | THR | H | 620 | 57.317 | −0.473 | 52.095 | 1.00 | 13.77 | N |
| ATOM | 986 | CA | THR | H | 620 | 56.508 | −0.501 | 50.861 | 1.00 | 13.02 | C |
| ATOM | 987 | CB | THR | H | 620 | 56.225 | 0.907 | 50.339 | 1.00 | 13.58 | C |
| ATOM | 988 | OG1 | THR | H | 620 | 57.458 | 1.516 | 49.903 | 1.00 | 12.94 | O |
| ATOM | 989 | CG2 | THR | H | 620 | 55.644 | 1.822 | 51.440 | 1.00 | 11.19 | C |
| ATOM | 990 | C | THR | H | 620 | 57.101 | −1.298 | 49.703 | 1.00 | 15.91 | C |
| ATOM | 991 | O | THR | H | 620 | 56.390 | −1.609 | 48.750 | 1.00 | 20.03 | O |
| ATOM | 992 | N | GLY | H | 621 | 58.395 | −1.591 | 49.765 | 1.00 | 15.20 | N |
| ATOM | 993 | CA | GLY | H | 621 | 59.097 | −2.189 | 48.649 | 1.00 | 15.87 | C |
| ATOM | 994 | C | GLY | H | 621 | 59.489 | −1.194 | 47.558 | 1.00 | 21.03 | C |
| ATOM | 995 | O | GLY | H | 621 | 60.107 | −1.590 | 46.566 | 1.00 | 15.57 | O |
| ATOM | 996 | N | LEU | H | 622 | 59.141 | 0.083 | 47.730 | 1.00 | 13.14 | N |
| ATOM | 997 | CA | LEU | H | 622 | 59.491 | 1.112 | 46.753 | 1.00 | 18.24 | C |
| ATOM | 998 | CB | LEU | H | 622 | 58.585 | 2.329 | 46.933 | 1.00 | 17.09 | C |
| ATOM | 999 | CG | LEU | H | 622 | 57.101 | 2.007 | 46.669 | 1.00 | 12.79 | C |
| ATOM | 1000 | CD1 | LEU | H | 622 | 56.186 | 2.972 | 47.422 | 1.00 | 5.60 | C |
| ATOM | 1001 | CD2 | LEU | H | 622 | 56.779 | 1.982 | 45.175 | 1.00 | 9.82 | C |
| ATOM | 1002 | C | LEU | H | 622 | 60.966 | 1.493 | 46.894 | 1.00 | 18.67 | C |
| ATOM | 1003 | O | LEU | H | 622 | 61.521 | 1.358 | 47.970 | 1.00 | 16.14 | O |
| ATOM | 1004 | N | ILE | H | 623 | 61.607 | 1.922 | 45.804 | 1.00 | 27.91 | N |
| ATOM | 1005 | CA | ILE | H | 623 | 62.991 | 2.390 | 45.834 | 1.00 | 23.14 | C |
| ATOM | 1006 | CB | ILE | H | 623 | 63.521 | 2.587 | 44.392 | 1.00 | 27.11 | C |
| ATOM | 1007 | CG1 | ILE | H | 623 | 63.433 | 1.273 | 43.600 | 1.00 | 27.83 | C |
| ATOM | 1008 | CD1 | ILE | H | 623 | 63.961 | 1.375 | 42.178 | 1.00 | 16.05 | C |
| ATOM | 1009 | CG2 | ILE | H | 623 | 64.969 | 3.160 | 44.394 | 1.00 | 24.30 | C |
| ATOM | 1010 | C | ILE | H | 623 | 63.082 | 3.695 | 46.624 | 1.00 | 24.55 | C |
| ATOM | 1011 | O | ILE | H | 623 | 64.019 | 3.896 | 47.407 | 1.00 | 25.98 | O |
| ATOM | 1012 | N | ASN | H | 624 | 62.135 | 4.588 | 46.373 | 1.00 | 27.42 | N |
| ATOM | 1013 | CA | ASN | H | 624 | 62.012 | 5.848 | 47.089 | 1.00 | 32.05 | C |
| ATOM | 1014 | CB | ASN | H | 624 | 62.407 | 6.998 | 46.176 | 1.00 | 37.43 | C |
| ATOM | 1015 | CG | ASN | H | 624 | 63.898 | 7.117 | 46.023 | 1.00 | 48.80 | C |
| ATOM | 1016 | OD1 | ASN | H | 624 | 64.633 | 7.291 | 47.005 | 1.00 | 53.50 | O |
| ATOM | 1017 | ND2 | ASN | H | 624 | 64.371 | 7.019 | 44.787 | 1.00 | 49.52 | N |
| ATOM | 1018 | C | ASN | H | 624 | 60.590 | 6.087 | 47.563 | 1.00 | 25.94 | C |
| ATOM | 1019 | O | ASN | H | 624 | 59.793 | 6.644 | 46.822 | 1.00 | 29.51 | O |
| ATOM | 1020 | N | TYR | H | 625 | 60.297 | 5.682 | 48.796 | 1.00 | 19.24 | N |
| ATOM | 1021 | CA | TYR | H | 625 | 58.990 | 5.876 | 49.415 | 1.00 | 18.02 | C |
| ATOM | 1022 | CB | TYR | H | 625 | 58.951 | 5.088 | 50.720 | 1.00 | 14.58 | C |
| ATOM | 1023 | CG | TYR | H | 625 | 57.751 | 5.285 | 51.631 | 1.00 | 14.00 | C |
| ATOM | 1024 | CD1 | TYR | H | 625 | 56.447 | 5.388 | 51.128 | 1.00 | 14.02 | C |
| ATOM | 1025 | CE1 | TYR | H | 625 | 55.322 | 5.549 | 52.007 | 1.00 | 11.40 | C |
| ATOM | 1026 | CZ | TYR | H | 625 | 55.540 | 5.582 | 53.379 | 1.00 | 11.88 | C |
| ATOM | 1027 | OH | TYR | H | 625 | 54.499 | 5.717 | 54.268 | 1.00 | 8.70 | O |
| ATOM | 1028 | CE2 | TYR | H | 625 | 56.840 | 5.461 | 53.885 | 1.00 | 12.21 | C |
| ATOM | 1029 | CD2 | TYR | H | 625 | 57.923 | 5.306 | 53.024 | 1.00 | 8.28 | C |
| ATOM | 1030 | C | TYR | H | 625 | 58.753 | 7.357 | 49.655 | 1.00 | 18.50 | C |
| ATOM | 1031 | O | TYR | H | 625 | 59.577 | 8.027 | 50.238 | 1.00 | 26.81 | O |
| ATOM | 1032 | N | ASP | H | 626 | 57.641 | 7.869 | 49.156 | 1.00 | 28.60 | N |
| ATOM | 1033 | CA | ASP | H | 626 | 57.348 | 9.305 | 49.176 | 1.00 | 35.36 | C |
| ATOM | 1034 | CB | ASP | H | 626 | 56.105 | 9.582 | 48.346 | 1.00 | 38.17 | C |
| ATOM | 1035 | CG | ASP | H | 626 | 55.109 | 8.463 | 48.452 | 1.00 | 50.64 | C |
| ATOM | 1036 | OD1 | ASP | H | 626 | 55.233 | 7.483 | 47.670 | 1.00 | 56.52 | O |
| ATOM | 1037 | OD2 | ASP | H | 626 | 54.182 | 8.466 | 49.291 | 1.00 | 54.16 | O |
| ATOM | 1038 | C | ASP | H | 626 | 57.117 | 9.831 | 50.585 | 1.00 | 32.74 | C |
| ATOM | 1039 | O | ASP | H | 626 | 57.288 | 11.008 | 50.822 | 1.00 | 34.11 | O |
| ATOM | 1040 | N | GLY | H | 627 | 56.708 | 8.958 | 51.498 | 1.00 | 30.43 | N |
| ATOM | 1041 | CA | GLY | H | 627 | 56.488 | 9.324 | 52.885 | 1.00 | 27.63 | C |
| ATOM | 1042 | C | GLY | H | 627 | 55.036 | 9.295 | 53.293 | 1.00 | 30.10 | C |
| ATOM | 1043 | O | GLY | H | 627 | 54.711 | 9.332 | 54.485 | 1.00 | 32.28 | O |
| ATOM | 1044 | N | LEU | H | 628 | 54.150 | 9.221 | 52.304 | 1.00 | 30.21 | N |
| ATOM | 1045 | CA | LEU | H | 628 | 52.723 | 9.381 | 52.562 | 1.00 | 28.52 | C |
| ATOM | 1046 | CB | LEU | H | 628 | 51.972 | 9.848 | 51.309 | 1.00 | 24.52 | C |
| ATOM | 1047 | CG | LEU | H | 628 | 52.359 | 11.272 | 50.869 | 1.00 | 30.52 | C |
| ATOM | 1048 | CD1 | LEU | H | 628 | 51.624 | 11.644 | 49.588 | 1.00 | 28.39 | C |
| ATOM | 1049 | CD2 | LEU | H | 628 | 52.128 | 12.330 | 51.985 | 1.00 | 28.50 | C |
| ATOM | 1050 | C | LEU | H | 628 | 52.095 | 8.143 | 53.158 | 1.00 | 30.93 | C |
| ATOM | 1051 | O | LEU | H | 628 | 52.496 | 7.008 | 52.831 | 1.00 | 33.16 | O |
| ATOM | 1052 | N | LEU | H | 629 | 51.118 | 8.368 | 54.043 | 1.00 | 24.84 | N |
| ATOM | 1053 | CA | LEU | H | 629 | 50.392 | 7.270 | 54.653 | 1.00 | 23.90 | C |
| ATOM | 1054 | CB | LEU | H | 629 | 49.300 | 7.808 | 55.538 | 1.00 | 21.70 | C |
| ATOM | 1055 | CG | LEU | H | 629 | 48.480 | 6.744 | 56.255 | 1.00 | 24.47 | C |
| ATOM | 1056 | CD1 | LEU | H | 629 | 49.296 | 6.113 | 57.395 | 1.00 | 17.96 | C |
| ATOM | 1057 | CD2 | LEU | H | 629 | 47.209 | 7.402 | 56.794 | 1.00 | 19.37 | C |
| ATOM | 1058 | C | LEU | H | 629 | 49.775 | 6.464 | 53.531 | 1.00 | 29.34 | C |
| ATOM | 1059 | O | LEU | H | 629 | 49.317 | 7.053 | 52.543 | 1.00 | 32.59 | O |
| ATOM | 1060 | N | ARG | H | 630 | 49.782 | 5.137 | 53.662 | 1.00 | 25.57 | N |
| ATOM | 1061 | CA | ARG | H | 630 | 49.239 | 4.248 | 52.623 | 1.00 | 19.08 | C |
| ATOM | 1062 | CB | ARG | H | 630 | 50.329 | 3.430 | 51.920 | 1.00 | 19.45 | C |
| ATOM | 1063 | CG | ARG | H | 630 | 51.258 | 4.248 | 51.061 | 1.00 | 24.94 | C |
| ATOM | 1064 | CD | ARG | H | 630 | 52.450 | 3.507 | 50.539 | 1.00 | 21.79 | C |
| ATOM | 1065 | NE | ARG | H | 630 | 52.199 | 2.908 | 49.234 | 1.00 | 24.99 | N |
| ATOM | 1066 | CZ | ARG | H | 630 | 52.340 | 3.558 | 48.085 | 1.00 | 18.82 | C |
| ATOM | 1067 | NH1 | ARG | H | 630 | 52.722 | 4.825 | 48.112 | 1.00 | 7.54 | N |
| ATOM | 1068 | NH2 | ARG | H | 630 | 52.126 | 2.931 | 46.918 | 1.00 | 11.24 | N |
| ATOM | 1069 | C | ARG | H | 630 | 48.250 | 3.301 | 53.213 | 1.00 | 18.82 | C |
| ATOM | 1070 | O | ARG | H | 630 | 48.358 | 2.951 | 54.409 | 1.00 | 15.92 | O |
| ATOM | 1071 | N | VAL | H | 631 | 47.298 | 2.881 | 52.365 | 1.00 | 15.55 | N |
| ATOM | 1072 | CA | VAL | H | 631 | 46.226 | 1.976 | 52.766 | 1.00 | 14.83 | C |
| ATOM | 1073 | CB | VAL | H | 631 | 44.878 | 2.730 | 52.815 | 1.00 | 13.65 | C |
| ATOM | 1074 | CG1 | VAL | H | 631 | 44.580 | 3.432 | 51.459 | 1.00 | 11.77 | C |
| ATOM | 1075 | CG2 | VAL | H | 631 | 43.743 | 1.796 | 53.213 | 1.00 | 12.67 | C |
| ATOM | 1076 | C | VAL | H | 631 | 46.117 | 0.762 | 51.838 | 1.00 | 16.14 | C |
| ATOM | 1077 | O | VAL | H | 631 | 46.350 | 0.858 | 50.635 | 1.00 | 19.60 | O |
| ATOM | 1078 | N | ALA | H | 632 | 45.766 | −0.386 | 52.397 | 1.00 | 15.42 | N |
| ATOM | 1079 | CA | ALA | H | 632 | 45.517 | −1.555 | 51.576 | 1.00 | 16.78 | C |
| ATOM | 1080 | CB | ALA | H | 632 | 46.628 | −2.550 | 51.720 | 1.00 | 17.47 | C |
| ATOM | 1081 | C | ALA | H | 632 | 44.210 | −2.168 | 52.002 | 1.00 | 22.23 | C |
| ATOM | 1082 | O | ALA | H | 632 | 43.836 | −2.085 | 53.168 | 1.00 | 28.32 | O |
| ATOM | 1083 | N | HIS | H | 633 | 43.512 | −2.787 | 51.064 | 1.00 | 22.64 | N |
| ATOM | 1084 | CA | HIS | H | 633 | 42.196 | −3.307 | 51.371 | 1.00 | 26.76 | C |
| ATOM | 1085 | CB | HIS | H | 633 | 41.185 | −2.862 | 50.317 | 1.00 | 32.27 | C |
| ATOM | 1086 | CG | HIS | H | 633 | 41.133 | −1.384 | 50.174 | 1.00 | 36.50 | C |
| ATOM | 1087 | ND1 | HIS | H | 633 | 40.647 | −0.567 | 51.171 | 1.00 | 34.69 | N |
| ATOM | 1088 | CE1 | HIS | H | 633 | 40.762 | 0.696 | 50.793 | 1.00 | 41.01 | C |
| ATOM | 1089 | NE2 | HIS | H | 633 | 41.325 | 0.726 | 49.596 | 1.00 | 40.71 | N |
| ATOM | 1090 | CD2 | HIS | H | 633 | 41.584 | −0.565 | 49.194 | 1.00 | 41.83 | C |
| ATOM | 1091 | C | HIS | H | 633 | 42.229 | −4.790 | 51.508 | 1.00 | 26.25 | C |
| ATOM | 1092 | O | HIS | H | 633 | 42.874 | −5.471 | 50.720 | 1.00 | 27.20 | O |
| ATOM | 1093 | N | LEU | H | 634 | 41.513 | −5.279 | 52.513 | 1.00 | 24.73 | N |
| ATOM | 1094 | CA | LEU | H | 634 | 41.558 | −6.679 | 52.888 | 1.00 | 30.83 | C |
| ATOM | 1095 | CB | LEU | H | 634 | 42.405 | −6.855 | 54.159 | 1.00 | 28.46 | C |
| ATOM | 1096 | CG | LEU | H | 634 | 43.829 | −6.292 | 54.131 | 1.00 | 27.11 | C |
| ATOM | 1097 | CD1 | LEU | H | 634 | 44.507 | −6.471 | 55.498 | 1.00 | 29.97 | C |
| ATOM | 1098 | CD2 | LEU | H | 634 | 44.611 | −7.008 | 53.050 | 1.00 | 26.61 | C |
| ATOM | 1099 | C | LEU | H | 634 | 40.141 | −7.148 | 53.135 | 1.00 | 34.94 | C |
| ATOM | 1100 | O | LEU | H | 634 | 39.263 | −6.331 | 53.385 | 1.00 | 38.80 | O |
| ATOM | 1101 | N | TYR | H | 635 | 39.923 | −8.457 | 53.088 | 1.00 | 34.36 | N |
| ATOM | 1102 | CA | TYR | H | 635 | 38.625 | −9.016 | 53.434 | 1.00 | 37.58 | C |
| ATOM | 1103 | CB | TYR | H | 635 | 38.021 | −9.773 | 52.248 | 1.00 | 46.30 | C |
| ATOM | 1104 | CG | TYR | H | 635 | 37.501 | −8.900 | 51.124 | 1.00 | 51.69 | C |
| ATOM | 1105 | CD1 | TYR | H | 635 | 37.155 | −7.569 | 51.340 | 1.00 | 57.35 | C |
| ATOM | 1106 | CE1 | TYR | H | 635 | 36.662 | −6.768 | 50.298 | 1.00 | 62.24 | C |
| ATOM | 1107 | CZ | TYR | H | 635 | 36.497 | −7.312 | 49.037 | 1.00 | 61.91 | C |
| ATOM | 1108 | OH | TYR | H | 635 | 36.011 | −6.544 | 48.011 | 1.00 | 62.12 | O |
| ATOM | 1109 | CE2 | TYR | H | 635 | 36.823 | −8.634 | 48.800 | 1.00 | 62.92 | C |
| ATOM | 1110 | CD2 | TYR | H | 635 | 37.321 | −9.422 | 49.847 | 1.00 | 58.38 | C |
| ATOM | 1111 | C | TYR | H | 635 | 38.752 | −9.937 | 54.625 | 1.00 | 33.39 | C |
| ATOM | 1112 | O | TYR | H | 635 | 39.679 | −10.730 | 54.697 | 1.00 | 32.16 | O |
| ATOM | 1113 | N | ILE | H | 636 | 37.829 | −9.831 | 55.575 | 1.00 | 31.27 | N |
| ATOM | 1114 | CA | ILE | H | 636 | 37.832 | −10.755 | 56.709 | 1.00 | 26.78 | C |
| ATOM | 1115 | CB | ILE | H | 636 | 36.723 | −10.385 | 57.694 | 1.00 | 24.13 | C |
| ATOM | 1116 | CG1 | ILE | H | 636 | 36.956 | −8.970 | 58.233 | 1.00 | 23.26 | C |
| ATOM | 1117 | CD1 | ILE | H | 636 | 37.849 | −8.892 | 59.399 | 1.00 | 23.06 | C |
| ATOM | 1118 | CG2 | ILE | H | 636 | 36.596 | −11.437 | 58.804 | 1.00 | 20.24 | C |
| ATOM | 1119 | C | ILE | H | 636 | 37.647 | −12.187 | 56.220 | 1.00 | 30.51 | C |
| ATOM | 1120 | O | ILE | H | 636 | 36.825 | −12.450 | 55.350 | 1.00 | 36.13 | O |
| ATOM | 1121 | N | MET | H | 637 | 38.403 | −13.114 | 56.790 | 1.00 | 36.95 | N |
| ATOM | 1122 | CA | MET | H | 637 | 38.362 | −14.505 | 56.357 | 1.00 | 39.66 | C |
| ATOM | 1123 | CB | MET | H | 637 | 39.627 | −14.838 | 55.561 | 1.00 | 39.09 | C |
| ATOM | 1124 | CG | MET | H | 637 | 39.888 | −13.898 | 54.399 | 1.00 | 41.31 | C |
| ATOM | 1125 | SD | MET | H | 637 | 41.053 | −14.558 | 53.220 | 1.00 | 50.54 | S |
| ATOM | 1126 | CE | MET | H | 637 | 40.752 | −13.457 | 51.852 | 1.00 | 40.06 | C |
| ATOM | 1127 | C | MET | H | 637 | 38.227 | −15.457 | 57.541 | 1.00 | 43.82 | C |
| ATOM | 1128 | O | MET | H | 637 | 38.438 | −15.065 | 58.700 | 1.00 | 40.83 | O |
| ATOM | 1129 | N | GLY | H | 638 | 37.883 | −16.708 | 57.239 | 1.00 | 47.76 | N |
| ATOM | 1130 | CA | GLY | H | 638 | 37.834 | −17.757 | 58.240 | 1.00 | 51.05 | C |
| ATOM | 1131 | C | GLY | H | 638 | 39.181 | −17.947 | 58.912 | 1.00 | 53.71 | C |
| ATOM | 1132 | O | GLY | H | 638 | 40.214 | −18.012 | 58.238 | 1.00 | 50.94 | O |
| ATOM | 1133 | N | ASN | H | 639 | 39.163 | −18.035 | 60.242 | 1.00 | 59.07 | N |
| ATOM | 1134 | CA | ASN | H | 639 | 40.378 | −18.212 | 61.036 | 1.00 | 64.72 | C |
| ATOM | 1135 | CB | ASN | H | 639 | 40.035 | −18.305 | 62.524 | 1.00 | 69.39 | C |
| ATOM | 1136 | CG | ASN | H | 639 | 40.676 | −17.201 | 63.332 | 1.00 | 72.30 | C |
| ATOM | 1137 | OD1 | ASN | H | 639 | 41.727 | −17.396 | 63.943 | 1.00 | 73.85 | O |
| ATOM | 1138 | ND2 | ASN | H | 639 | 40.052 | −16.030 | 63.335 | 1.00 | 73.98 | N |
| ATOM | 1139 | C | ASN | H | 639 | 41.291 | −19.381 | 60.624 | 1.00 | 66.93 | C |
| ATOM | 1140 | O | ASN | H | 639 | 42.502 | −19.356 | 60.903 | 1.00 | 62.59 | O |
| ATOM | 1141 | N | GLU | H | 640 | 40.705 | −20.385 | 59.965 | 1.00 | 66.49 | N |
| ATOM | 1142 | CA | GLU | H | 640 | 41.437 | −21.556 | 59.472 | 1.00 | 67.87 | C |
| ATOM | 1143 | CB | GLU | H | 640 | 40.463 | −22.685 | 59.083 | 1.00 | 71.58 | C |
| ATOM | 1144 | CG | GLU | H | 640 | 39.682 | −22.505 | 57.773 | 1.00 | 73.03 | C |
| ATOM | 1145 | CD | GLU | H | 640 | 38.530 | −21.506 | 57.849 | 1.00 | 74.83 | C |
| ATOM | 1146 | OE1 | GLU | H | 640 | 38.039 | −21.172 | 58.966 | 1.00 | 76.77 | O |
| ATOM | 1147 | OE2 | GLU | H | 640 | 38.109 | −21.052 | 56.765 | 1.00 | 72.95 | O |
| ATOM | 1148 | C | GLU | H | 640 | 42.424 | −21.252 | 58.323 | 1.00 | 67.74 | C |
| ATOM | 1149 | O | GLU | H | 640 | 43.408 | −21.976 | 58.130 | 1.00 | 64.34 | O |
| ATOM | 1150 | N | LYS | H | 641 | 42.164 | −20.184 | 57.572 | 1.00 | 69.22 | N |
| ATOM | 1151 | CA | LYS | H | 641 | 43.042 | −19.803 | 56.471 | 1.00 | 71.81 | C |
| ATOM | 1152 | CB | LYS | H | 641 | 42.344 | −18.844 | 55.519 | 1.00 | 73.04 | C |
| ATOM | 1153 | CG | LYS | H | 641 | 41.152 | −19.435 | 54.788 | 1.00 | 76.61 | C |
| ATOM | 1154 | CD | LYS | H | 641 | 40.694 | −18.483 | 53.674 | 1.00 | 82.00 | C |
| ATOM | 1155 | CE | LYS | H | 641 | 39.230 | −18.692 | 53.280 | 1.00 | 84.51 | C |
| ATOM | 1156 | NZ | LYS | H | 641 | 38.261 | −18.175 | 54.308 | 1.00 | 83.99 | N |
| ATOM | 1157 | C | LYS | H | 641 | 44.358 | −19.203 | 56.966 | 1.00 | 72.88 | C |
| ATOM | 1158 | O | LYS | H | 641 | 45.360 | −19.224 | 56.247 | 1.00 | 75.94 | O |
| ATOM | 1159 | N | CYS | H | 642 | 44.352 | −18.678 | 58.191 | 1.00 | 70.92 | N |
| ATOM | 1160 | CA | CYS | H | 642 | 45.550 | −18.099 | 58.794 | 1.00 | 70.44 | C |
| ATOM | 1161 | CB | CYS | H | 642 | 45.188 | −16.978 | 59.779 | 1.00 | 64.78 | C |
| ATOM | 1162 | SG | CYS | H | 642 | 44.813 | −15.386 | 59.018 | 1.00 | 59.13 | S |
| ATOM | 1163 | C | CYS | H | 642 | 46.450 | −19.112 | 59.498 | 1.00 | 74.97 | C |
| ATOM | 1164 | O | CYS | H | 642 | 47.628 | −18.825 | 59.717 | 1.00 | 76.32 | O |
| ATOM | 1165 | N | SER | H | 643 | 45.889 | −20.270 | 59.861 | 1.00 | 81.04 | N |
| ATOM | 1166 | CA | SER | H | 643 | 46.581 | −21.326 | 60.627 | 1.00 | 87.51 | C |
| ATOM | 1167 | CB | SER | H | 643 | 46.221 | −22.709 | 60.063 | 1.00 | 85.70 | C |
| ATOM | 1168 | OG | SER | H | 643 | 46.862 | −23.714 | 60.850 | 0.00 | 81.26 | O |
| ATOM | 1169 | C | SER | H | 643 | 48.112 | −21.178 | 60.694 | 1.00 | 92.16 | C |
| ATOM | 1170 | O | SER | H | 643 | 48.824 | −21.810 | 59.903 | 1.00 | 94.13 | O |
| ATOM | 1171 | N | GLN | H | 644 | 48.596 | −20.345 | 61.627 | 1.00 | 92.30 | N |
| ATOM | 1172 | CA | GLN | H | 644 | 50.029 | −20.029 | 61.818 | 1.00 | 93.29 | C |
| ATOM | 1173 | CB | GLN | H | 644 | 50.634 | −20.901 | 62.927 | 1.00 | 93.26 | C |
| ATOM | 1174 | CG | GLN | H | 644 | 50.084 | −20.691 | 64.262 | 0.00 | 85.00 | C |
| ATOM | 1175 | CD | GLN | H | 644 | 50.690 | −21.612 | 65.303 | 0.00 | 85.00 | C |
| ATOM | 1176 | OE1 | GLN | H | 644 | 51.516 | −22.469 | 64.984 | 0.00 | 85.00 | O |
| ATOM | 1177 | NE2 | GLN | H | 644 | 50.284 | −21.439 | 66.554 | 0.00 | 85.00 | N |
| ATOM | 1178 | C | GLN | H | 644 | 50.936 | −20.069 | 60.569 | 1.00 | 94.12 | C |
| ATOM | 1179 | O | GLN | H | 644 | 50.522 | −20.446 | 59.469 | 1.00 | 94.02 | O |
| ATOM | 1180 | N | LEU | H | 652 | 44.163 | −19.179 | 72.145 | 1.00 | 94.35 | N |
| ATOM | 1181 | CA | LEU | H | 652 | 45.024 | −18.846 | 71.015 | 1.00 | 94.03 | C |
| ATOM | 1182 | CB | LEU | H | 652 | 45.861 | −20.058 | 70.563 | 1.00 | 92.78 | C |
| ATOM | 1183 | CG | LEU | H | 652 | 46.972 | −20.554 | 71.507 | 1.00 | 94.80 | C |
| ATOM | 1184 | CD1 | LEU | H | 652 | 47.528 | −21.907 | 71.046 | 1.00 | 93.59 | C |
| ATOM | 1185 | CD2 | LEU | H | 652 | 48.113 | −19.522 | 71.707 | 1.00 | 93.35 | C |
| ATOM | 1186 | C | LEU | H | 652 | 44.262 | −18.236 | 69.836 | 1.00 | 94.73 | C |
| ATOM | 1187 | O | LEU | H | 652 | 44.724 | −17.257 | 69.258 | 1.00 | 98.85 | O |
| ATOM | 1188 | N | ASN | H | 653 | 43.105 | −18.799 | 69.482 | 1.00 | 90.46 | N |
| ATOM | 1189 | CA | ASN | H | 653 | 42.399 | −18.374 | 68.267 | 1.00 | 86.64 | C |
| ATOM | 1190 | CB | ASN | H | 653 | 41.781 | −19.571 | 67.524 | 1.00 | 86.38 | C |
| ATOM | 1191 | CG | ASN | H | 653 | 42.831 | −20.436 | 66.819 | 1.00 | 84.52 | C |
| ATOM | 1192 | OD1 | ASN | H | 653 | 43.758 | −19.938 | 66.185 | 1.00 | 83.48 | O |
| ATOM | 1193 | ND2 | ASN | H | 653 | 42.676 | −21.738 | 66.928 | 1.00 | 84.36 | N |
| ATOM | 1194 | C | ASN | H | 653 | 41.376 | −17.252 | 68.441 | 1.00 | 83.20 | C |
| ATOM | 1195 | O | ASN | H | 653 | 41.302 | −16.353 | 67.610 | 1.00 | 82.34 | O |
| ATOM | 1196 | N | GLU | H | 654 | 40.597 | −17.302 | 69.516 | 1.00 | 81.31 | N |
| ATOM | 1197 | CA | GLU | H | 654 | 39.527 | −16.327 | 69.740 | 1.00 | 77.06 | C |
| ATOM | 1198 | CB | GLU | H | 654 | 38.746 | −16.666 | 70.998 | 1.00 | 81.52 | C |
| ATOM | 1199 | CG | GLU | H | 654 | 37.907 | −17.922 | 70.887 | 1.00 | 85.66 | C |
| ATOM | 1200 | CD | GLU | H | 654 | 37.135 | −18.206 | 72.158 | 1.00 | 88.83 | C |
| ATOM | 1201 | OE1 | GLU | H | 654 | 37.342 | −17.483 | 73.158 | 1.00 | 90.45 | O |
| ATOM | 1202 | OE2 | GLU | H | 654 | 36.318 | −19.149 | 72.160 | 1.00 | 89.91 | O |
| ATOM | 1203 | C | GLU | H | 654 | 39.983 | −14.877 | 69.832 | 1.00 | 71.42 | C |
| ATOM | 1204 | O | GLU | H | 654 | 39.238 | −13.983 | 69.463 | 1.00 | 68.60 | O |
| ATOM | 1205 | N | SER | H | 655 | 41.189 | −14.644 | 70.345 | 1.00 | 68.74 | N |
| ATOM | 1206 | CA | SER | H | 655 | 41.749 | −13.295 | 70.399 | 1.00 | 61.24 | C |
| ATOM | 1207 | CB | SER | H | 655 | 42.935 | −13.245 | 71.350 | 1.00 | 64.06 | C |
| ATOM | 1208 | OG | SER | H | 655 | 43.984 | −14.066 | 70.881 | 1.00 | 64.75 | O |
| ATOM | 1209 | C | SER | H | 655 | 42.195 | −12.777 | 69.030 | 1.00 | 53.67 | C |
| ATOM | 1210 | O | SER | H | 655 | 42.536 | −11.606 | 68.898 | 1.00 | 53.48 | O |
| ATOM | 1211 | N | GLU | H | 656 | 42.178 | −13.640 | 68.018 | 1.00 | 46.07 | N |
| ATOM | 1212 | CA | GLU | H | 656 | 42.724 | −13.301 | 66.705 | 1.00 | 44.94 | C |
| ATOM | 1213 | CB | GLU | H | 656 | 43.790 | −14.320 | 66.316 | 1.00 | 43.79 | C |
| ATOM | 1214 | CG | GLU | H | 656 | 44.905 | −14.375 | 67.347 | 1.00 | 46.81 | C |
| ATOM | 1215 | CD | GLU | H | 656 | 45.933 | −15.452 | 67.090 | 1.00 | 49.31 | C |
| ATOM | 1216 | OE1 | GLU | H | 656 | 45.576 | −16.569 | 66.594 | 1.00 | 48.42 | O |
| ATOM | 1217 | OE2 | GLU | H | 656 | 47.106 | −15.156 | 67.419 | 1.00 | 48.97 | O |
| ATOM | 1218 | C | GLU | H | 656 | 41.707 | −13.128 | 65.573 | 1.00 | 41.77 | C |
| ATOM | 1219 | O | GLU | H | 656 | 40.681 | −13.793 | 65.534 | 1.00 | 44.31 | O |
| ATOM | 1220 | N | ILE | H | 657 | 42.024 | −12.225 | 64.656 | 1.00 | 37.87 | N |
| ATOM | 1221 | CA | ILE | H | 657 | 41.254 | −11.993 | 63.437 | 1.00 | 39.45 | C |
| ATOM | 1222 | CB | ILE | H | 657 | 40.859 | −10.483 | 63.334 | 1.00 | 38.21 | C |
| ATOM | 1223 | CG1 | ILE | H | 657 | 39.694 | −10.159 | 64.264 | 1.00 | 37.32 | C |
| ATOM | 1224 | CD1 | ILE | H | 657 | 39.254 | −8.701 | 64.211 | 1.00 | 40.06 | C |
| ATOM | 1225 | CG2 | ILE | H | 657 | 40.501 | −10.082 | 61.896 | 1.00 | 41.59 | C |
| ATOM | 1226 | C | ILE | H | 657 | 42.114 | −12.408 | 62.239 | 1.00 | 39.10 | C |
| ATOM | 1227 | O | ILE | H | 657 | 43.343 | −12.266 | 62.272 | 1.00 | 34.02 | O |
| ATOM | 1228 | N | CYS | H | 658 | 41.462 | −12.929 | 61.197 | 1.00 | 41.45 | N |
| ATOM | 1229 | CA | CYS | H | 658 | 42.131 | −13.334 | 59.959 | 1.00 | 36.78 | C |
| ATOM | 1230 | CB | CYS | H | 658 | 41.890 | −14.821 | 59.683 | 1.00 | 39.75 | C |
| ATOM | 1231 | SG | CYS | H | 658 | 42.895 | −15.526 | 58.349 | 1.00 | 46.71 | S |
| ATOM | 1232 | C | CYS | H | 658 | 41.617 | −12.500 | 58.793 | 1.00 | 36.07 | C |
| ATOM | 1233 | O | CYS | H | 658 | 40.462 | −12.640 | 58.387 | 1.00 | 34.57 | O |
| ATOM | 1234 | N | ALA | H | 659 | 42.464 | −11.622 | 58.257 | 1.00 | 39.56 | N |
| ATOM | 1235 | CA | ALA | H | 659 | 42.082 | −10.838 | 57.077 | 1.00 | 36.61 | C |
| ATOM | 1236 | CB | ALA | H | 659 | 41.588 | −9.455 | 57.484 | 1.00 | 29.68 | C |
| ATOM | 1237 | C | ALA | H | 659 | 43.166 | −10.774 | 55.967 | 1.00 | 36.62 | C |
| ATOM | 1238 | O | ALA | H | 659 | 44.367 | −10.600 | 56.231 | 1.00 | 32.00 | O |
| ATOM | 1239 | N | GLY | H | 660 | 42.708 | −10.944 | 54.729 | 1.00 | 35.21 | N |
| ATOM | 1240 | CA | GLY | H | 660 | 43.570 | −10.973 | 53.568 | 1.00 | 34.14 | C |
| ATOM | 1241 | C | GLY | H | 660 | 42.960 | −10.360 | 52.326 | 1.00 | 33.46 | C |
| ATOM | 1242 | O | GLY | H | 660 | 41.765 | −10.093 | 52.275 | 1.00 | 33.24 | O |
| ATOM | 1243 | N | ALA | H | 661 | 43.797 | −10.110 | 51.324 | 1.00 | 36.57 | N |
| ATOM | 1244 | CA | ALA | H | 661 | 43.334 | −9.632 | 50.033 | 1.00 | 33.42 | C |
| ATOM | 1245 | CB | ALA | H | 661 | 44.387 | −8.794 | 49.416 | 1.00 | 34.79 | C |
| ATOM | 1246 | C | ALA | H | 661 | 43.080 | −10.870 | 49.187 | 1.00 | 40.10 | C |
| ATOM | 1247 | O | ALA | H | 661 | 43.668 | −11.924 | 49.439 | 1.00 | 42.42 | O |
| ATOM | 1248 | N | GLU | H | 662 | 42.210 | −10.760 | 48.188 | 1.00 | 46.75 | N |
| ATOM | 1249 | CA | GLU | H | 662 | 41.922 | −11.906 | 47.329 | 1.00 | 52.01 | C |
| ATOM | 1250 | CB | GLU | H | 662 | 40.530 | −11.788 | 46.690 | 1.00 | 50.42 | C |
| ATOM | 1251 | CG | GLU | H | 662 | 40.188 | −13.051 | 45.849 | 0.00 | 48.09 | C |
| ATOM | 1252 | CD | GLU | H | 662 | 40.181 | −14.351 | 46.632 | 0.00 | 47.77 | C |
| ATOM | 1253 | OE1 | GLU | H | 662 | 39.246 | −14.557 | 47.434 | 0.00 | 47.24 | O |
| ATOM | 1254 | OE2 | GLU | H | 662 | 41.111 | −15.165 | 46.448 | 0.00 | 47.24 | O |
| ATOM | 1255 | C | GLU | H | 662 | 43.014 | −12.090 | 46.269 | 1.00 | 54.03 | C |
| ATOM | 1256 | O | GLU | H | 662 | 43.120 | −11.278 | 45.344 | 1.00 | 56.76 | O |
| ATOM | 1257 | N | LYS | H | 663 | 43.819 | −13.146 | 46.443 | 1.00 | 51.74 | N |
| ATOM | 1258 | CA | LYS | H | 663 | 44.801 | −13.627 | 45.465 | 1.00 | 53.83 | C |
| ATOM | 1259 | CB | LYS | H | 663 | 44.164 | −13.896 | 44.084 | 1.00 | 59.24 | C |
| ATOM | 1260 | CG | LYS | H | 663 | 43.430 | −15.248 | 43.930 | 1.00 | 64.38 | C |
| ATOM | 1261 | CD | LYS | H | 663 | 44.387 | −16.446 | 43.768 | 1.00 | 71.33 | C |
| ATOM | 1262 | CE | LYS | H | 663 | 45.458 | −16.231 | 42.675 | 1.00 | 73.20 | C |
| ATOM | 1263 | NZ | LYS | H | 663 | 46.832 | −16.673 | 43.104 | 1.00 | 73.77 | N |
| ATOM | 1264 | C | LYS | H | 663 | 46.077 | −12.777 | 45.355 | 1.00 | 50.60 | C |
| ATOM | 1265 | O | LYS | H | 663 | 47.190 | −13.307 | 45.503 | 1.00 | 53.15 | O |
| ATOM | 1266 | N | ILE | H | 664 | 45.932 | −11.479 | 45.094 | 1.00 | 41.60 | N |
| ATOM | 1267 | CA | ILE | H | 664 | 47.088 | −10.583 | 45.089 | 1.00 | 35.39 | C |
| ATOM | 1268 | CB | ILE | H | 664 | 46.678 | −9.148 | 44.697 | 1.00 | 38.13 | C |
| ATOM | 1269 | CG1 | ILE | H | 664 | 46.062 | −8.399 | 45.893 | 1.00 | 33.86 | C |
| ATOM | 1270 | CD1 | ILE | H | 664 | 45.377 | −7.078 | 45.547 | 1.00 | 31.28 | C |
| ATOM | 1271 | CG2 | ILE | H | 664 | 45.760 | −9.157 | 43.462 | 1.00 | 33.96 | C |
| ATOM | 1272 | C | ILE | H | 664 | 47.782 | −10.599 | 46.460 | 1.00 | 38.71 | C |
| ATOM | 1273 | O | ILE | H | 664 | 47.151 | −10.837 | 47.487 | 1.00 | 46.53 | O |
| ATOM | 1274 | N | GLY | H | 665 | 49.081 | −10.362 | 46.483 | 1.00 | 38.09 | N |
| ATOM | 1275 | CA | GLY | H | 665 | 49.797 | −10.364 | 47.743 | 1.00 | 37.60 | C |
| ATOM | 1276 | C | GLY | H | 665 | 49.986 | −8.952 | 48.242 | 1.00 | 40.66 | C |
| ATOM | 1277 | O | GLY | H | 665 | 50.849 | −8.243 | 47.744 | 1.00 | 51.73 | O |
| ATOM | 1278 | N | SER | H | 666 | 49.162 | −8.519 | 49.193 | 1.00 | 36.58 | N |
| ATOM | 1279 | CA | SER | H | 666 | 49.316 | −7.186 | 49.786 | 1.00 | 29.38 | C |
| ATOM | 1280 | CB | SER | H | 666 | 48.519 | −6.137 | 49.011 | 1.00 | 29.00 | C |
| ATOM | 1281 | OG | SER | H | 666 | 47.192 | −6.027 | 49.456 | 1.00 | 32.67 | O |
| ATOM | 1282 | C | SER | H | 666 | 48.982 | −7.160 | 51.287 | 1.00 | 29.07 | C |
| ATOM | 1283 | O | SER | H | 666 | 48.316 | −8.066 | 51.804 | 1.00 | 36.19 | O |
| ATOM | 1284 | N | GLY | H | 667 | 49.466 | −6.144 | 51.991 | 1.00 | 18.40 | N |
| ATOM | 1285 | CA | GLY | H | 667 | 49.225 | −6.071 | 53.410 | 1.00 | 22.43 | C |
| ATOM | 1286 | C | GLY | H | 667 | 50.315 | −5.337 | 54.141 | 1.00 | 19.73 | C |
| ATOM | 1287 | O | GLY | H | 667 | 51.231 | −4.801 | 53.530 | 1.00 | 17.67 | O |
| ATOM | 1288 | N | PRO | H | 668 | 50.185 | −5.277 | 55.458 | 1.00 | 21.30 | N |
| ATOM | 1289 | CA | PRO | H | 668 | 51.125 | −4.528 | 56.310 | 1.00 | 24.50 | C |
| ATOM | 1290 | CB | PRO | H | 668 | 50.356 | −4.395 | 57.621 | 1.00 | 22.34 | C |
| ATOM | 1291 | CG | PRO | H | 668 | 49.577 | −5.688 | 57.675 | 1.00 | 24.60 | C |
| ATOM | 1292 | CD | PRO | H | 668 | 49.118 | −5.925 | 56.233 | 1.00 | 17.71 | C |
| ATOM | 1293 | C | PRO | H | 668 | 52.414 | −5.306 | 56.544 | 1.00 | 22.08 | C |
| ATOM | 1294 | O | PRO | H | 668 | 52.426 | −6.545 | 56.400 | 1.00 | 25.81 | O |
| ATOM | 1295 | N | CYS | H | 669 | 53.467 | −4.580 | 56.913 | 1.00 | 22.30 | N |
| ATOM | 1296 | CA | CYS | H | 669 | 54.809 | −5.136 | 57.160 | 1.00 | 25.75 | C |
| ATOM | 1297 | CB | CYS | H | 669 | 55.635 | −5.118 | 55.870 | 1.00 | 34.00 | C |
| ATOM | 1298 | SG | CYS | H | 669 | 54.834 | −5.866 | 54.451 | 1.00 | 43.69 | S |
| ATOM | 1299 | C | CYS | H | 669 | 55.526 | −4.288 | 58.214 | 1.00 | 20.14 | C |
| ATOM | 1300 | O | CYS | H | 669 | 54.910 | −3.408 | 58.824 | 1.00 | 21.93 | O |
| ATOM | 1301 | N | GLU | H | 670 | 56.821 | −4.525 | 58.428 | 1.00 | 17.39 | N |
| ATOM | 1302 | CA | GLU | H | 670 | 57.553 | −3.738 | 59.433 | 1.00 | 26.38 | C |
| ATOM | 1303 | CB | GLU | H | 670 | 59.060 | −4.090 | 59.542 | 1.00 | 28.98 | C |
| ATOM | 1304 | CG | GLU | H | 670 | 59.857 | −4.157 | 58.251 | 1.00 | 33.68 | C |
| ATOM | 1305 | CD | GLU | H | 670 | 59.956 | −5.580 | 57.747 | 1.00 | 41.73 | C |
| ATOM | 1306 | OE1 | GLU | H | 670 | 59.000 | −6.047 | 57.081 | 1.00 | 42.88 | O |
| ATOM | 1307 | OE2 | GLU | H | 670 | 60.987 | −6.233 | 58.023 | 1.00 | 43.60 | O |
| ATOM | 1308 | C | GLU | H | 670 | 57.333 | −2.232 | 59.284 | 1.00 | 24.10 | C |
| ATOM | 1309 | O | GLU | H | 670 | 57.399 | −1.678 | 58.186 | 1.00 | 32.96 | O |
| ATOM | 1310 | N | GLY | H | 671 | 57.045 | −1.579 | 60.395 | 1.00 | 21.11 | N |
| ATOM | 1311 | CA | GLY | H | 671 | 56.714 | −0.170 | 60.374 | 1.00 | 22.47 | C |
| ATOM | 1312 | C | GLY | H | 671 | 55.212 | 0.097 | 60.380 | 1.00 | 23.35 | C |
| ATOM | 1313 | O | GLY | H | 671 | 54.800 | 1.218 | 60.703 | 1.00 | 29.26 | O |
| ATOM | 1314 | N | ASP | H | 672 | 54.407 | −0.899 | 59.991 | 1.00 | 12.94 | N |
| ATOM | 1315 | CA | ASP | H | 672 | 52.945 | −0.832 | 60.105 | 1.00 | 16.13 | C |
| ATOM | 1316 | CB | ASP | H | 672 | 52.287 | −1.486 | 58.893 | 1.00 | 19.33 | C |
| ATOM | 1317 | CG | ASP | H | 672 | 52.702 | −0.835 | 57.591 | 1.00 | 20.75 | C |
| ATOM | 1318 | OD1 | ASP | H | 672 | 52.902 | 0.413 | 57.605 | 1.00 | 13.71 | O |
| ATOM | 1319 | OD2 | ASP | H | 672 | 52.867 | −1.503 | 56.526 | 1.00 | 17.92 | O |
| ATOM | 1320 | C | ASP | H | 672 | 52.368 | −1.467 | 61.374 | 1.00 | 16.81 | C |
| ATOM | 1321 | O | ASP | H | 672 | 51.174 | −1.325 | 61.653 | 1.00 | 20.37 | O |
| ATOM | 1322 | N | TYR | H | 673 | 53.215 | −2.162 | 62.134 | 1.00 | 19.82 | N |
| ATOM | 1323 | CA | TYR | H | 673 | 52.815 | −2.790 | 63.386 | 1.00 | 18.23 | C |
| ATOM | 1324 | CB | TYR | H | 673 | 54.005 | −3.487 | 64.066 | 1.00 | 24.84 | C |
| ATOM | 1325 | CG | TYR | H | 673 | 54.739 | −4.461 | 63.177 | 1.00 | 23.78 | C |
| ATOM | 1326 | CD1 | TYR | H | 673 | 54.038 | −5.259 | 62.293 | 1.00 | 23.61 | C |
| ATOM | 1327 | CE1 | TYR | H | 673 | 54.674 | −6.153 | 61.464 | 1.00 | 26.82 | C |
| ATOM | 1328 | CZ | TYR | H | 673 | 56.045 | −6.279 | 61.519 | 1.00 | 28.79 | C |
| ATOM | 1329 | OH | TYR | H | 673 | 56.633 | −7.193 | 60.691 | 1.00 | 21.44 | O |
| ATOM | 1330 | CE2 | TYR | H | 673 | 56.790 | −5.505 | 62.412 | 1.00 | 30.35 | C |
| ATOM | 1331 | CD2 | TYR | H | 673 | 56.127 | −4.592 | 63.236 | 1.00 | 25.98 | C |
| ATOM | 1332 | C | TYR | H | 673 | 52.231 | −1.737 | 64.308 | 1.00 | 16.25 | C |
| ATOM | 1333 | O | TYR | H | 673 | 52.684 | −0.578 | 64.311 | 1.00 | 16.85 | O |
| ATOM | 1334 | N | GLY | H | 674 | 51.218 | −2.140 | 65.075 | 1.00 | 16.54 | N |
| ATOM | 1335 | CA | GLY | H | 674 | 50.554 | −1.230 | 65.989 | 1.00 | 24.60 | C |
| ATOM | 1336 | C | GLY | H | 674 | 49.375 | −0.485 | 65.385 | 1.00 | 31.41 | C |
| ATOM | 1337 | O | GLY | H | 674 | 48.460 | −0.101 | 66.113 | 1.00 | 32.97 | O |
| ATOM | 1338 | N | GLY | H | 675 | 49.405 | −0.246 | 64.071 | 1.00 | 25.41 | N |
| ATOM | 1339 | CA | GLY | H | 675 | 48.296 | 0.377 | 63.390 | 1.00 | 19.96 | C |
| ATOM | 1340 | C | GLY | H | 675 | 47.054 | −0.498 | 63.394 | 1.00 | 15.60 | C |
| ATOM | 1341 | O | GLY | H | 675 | 47.090 | −1.637 | 63.822 | 1.00 | 14.31 | O |
| ATOM | 1342 | N | PRO | H | 676 | 45.941 | 0.054 | 62.927 | 1.00 | 25.52 | N |
| ATOM | 1343 | CA | PRO | H | 676 | 44.640 | −0.634 | 62.951 | 1.00 | 20.35 | C |
| ATOM | 1344 | CB | PRO | H | 676 | 43.681 | 0.526 | 63.138 | 1.00 | 14.22 | C |
| ATOM | 1345 | CG | PRO | H | 676 | 44.306 | 1.624 | 62.331 | 1.00 | 21.16 | C |
| ATOM | 1346 | CD | PRO | H | 676 | 45.814 | 1.434 | 62.411 | 1.00 | 27.19 | C |
| ATOM | 1347 | C | PRO | H | 676 | 44.203 | −1.393 | 61.688 | 1.00 | 21.53 | C |
| ATOM | 1348 | O | PRO | H | 676 | 44.494 | −1.023 | 60.557 | 1.00 | 20.45 | O |
| ATOM | 1349 | N | LEU | H | 677 | 43.478 | −2.477 | 61.915 | 1.00 | 19.62 | N |
| ATOM | 1350 | CA | LEU | H | 677 | 42.634 | −3.041 | 60.906 | 1.00 | 18.72 | C |
| ATOM | 1351 | CB | LEU | H | 677 | 42.615 | −4.547 | 61.099 | 1.00 | 14.60 | C |
| ATOM | 1352 | CG | LEU | H | 677 | 41.665 | −5.307 | 60.173 | 1.00 | 20.83 | C |
| ATOM | 1353 | CD1 | LEU | H | 677 | 42.147 | −5.258 | 58.706 | 1.00 | 17.91 | C |
| ATOM | 1354 | CD2 | LEU | H | 677 | 41.510 | −6.756 | 60.636 | 1.00 | 24.66 | C |
| ATOM | 1355 | C | LEU | H | 677 | 41.235 | −2.415 | 61.171 | 1.00 | 27.34 | C |
| ATOM | 1356 | O | LEU | H | 677 | 40.602 | −2.715 | 62.201 | 1.00 | 25.05 | O |
| ATOM | 1357 | N | VAL | H | 678 | 40.784 | −1.507 | 60.297 | 1.00 | 26.49 | N |
| ATOM | 1358 | CA | VAL | H | 678 | 39.466 | −0.869 | 60.466 | 1.00 | 34.34 | C |
| ATOM | 1359 | CB | VAL | H | 678 | 39.534 | 0.695 | 60.483 | 1.00 | 36.21 | C |
| ATOM | 1360 | CG1 | VAL | H | 678 | 40.947 | 1.167 | 60.824 | 1.00 | 34.68 | C |
| ATOM | 1361 | CG2 | VAL | H | 678 | 39.094 | 1.303 | 59.190 | 1.00 | 29.73 | C |
| ATOM | 1362 | C | VAL | H | 678 | 38.446 | −1.343 | 59.451 | 1.00 | 38.71 | C |
| ATOM | 1363 | O | VAL | H | 678 | 38.768 | −1.472 | 58.264 | 1.00 | 42.53 | O |
| ATOM | 1364 | N | CYS | H | 679 | 37.229 | −1.611 | 59.932 | 1.00 | 41.37 | N |
| ATOM | 1365 | CA | CYS | H | 679 | 36.087 | −1.992 | 59.083 | 1.00 | 45.99 | C |
| ATOM | 1366 | CB | CYS | H | 679 | 35.776 | −3.485 | 59.234 | 1.00 | 41.23 | C |
| ATOM | 1367 | SG | CYS | H | 679 | 37.218 | −4.552 | 59.471 | 1.00 | 41.87 | S |
| ATOM | 1368 | C | CYS | H | 679 | 34.827 | −1.146 | 59.392 | 1.00 | 55.61 | C |
| ATOM | 1369 | O | CYS | H | 679 | 34.898 | −0.170 | 60.150 | 1.00 | 59.48 | O |
| ATOM | 1370 | N | GLU | H | 680 | 33.686 | −1.512 | 58.798 | 1.00 | 64.76 | N |
| ATOM | 1371 | CA | GLU | H | 680 | 32.393 | −0.854 | 59.071 | 1.00 | 70.30 | C |
| ATOM | 1372 | CB | GLU | H | 680 | 31.740 | −0.336 | 57.773 | 1.00 | 70.21 | C |
| ATOM | 1373 | CG | GLU | H | 680 | 30.353 | 0.178 | 57.846 | 0.00 | 53.07 | C |
| ATOM | 1374 | CD | GLU | H | 680 | 30.238 | 1.328 | 58.826 | 0.00 | 52.36 | C |
| ATOM | 1375 | OE1 | GLU | H | 680 | 30.693 | 2.445 | 58.498 | 0.00 | 51.76 | O |
| ATOM | 1376 | OE2 | GLU | H | 680 | 29.685 | 1.114 | 59.925 | 0.00 | 51.80 | O |
| ATOM | 1377 | C | GLU | H | 680 | 31.448 | −1.802 | 59.815 | 1.00 | 71.19 | C |
| ATOM | 1378 | O | GLU | H | 680 | 30.692 | −2.555 | 59.195 | 1.00 | 70.21 | O |
| ATOM | 1379 | N | GLN | H | 681 | 31.508 | −1.767 | 61.146 | 1.00 | 75.21 | N |
| ATOM | 1380 | CA | GLN | H | 681 | 30.699 | −2.665 | 61.970 | 1.00 | 80.96 | C |
| ATOM | 1381 | CB | GLN | H | 681 | 31.416 | −3.035 | 63.274 | 1.00 | 79.18 | C |
| ATOM | 1382 | CG | GLN | H | 681 | 30.692 | −4.016 | 64.178 | 0.00 | 59.72 | C |
| ATOM | 1383 | CD | GLN | H | 681 | 31.393 | −4.224 | 65.507 | 0.00 | 58.32 | C |
| ATOM | 1384 | OE1 | GLN | H | 681 | 32.589 | −4.513 | 65.554 | 0.00 | 57.46 | O |
| ATOM | 1385 | NE2 | GLN | H | 681 | 30.650 | −4.072 | 66.597 | 0.00 | 57.48 | N |
| ATOM | 1386 | C | GLN | H | 681 | 29.300 | −2.098 | 62.239 | 1.00 | 85.46 | C |
| ATOM | 1387 | O | GLN | H | 681 | 28.360 | −2.381 | 61.483 | 1.00 | 85.05 | O |
| ATOM | 1388 | N | HIS | H | 682 | 29.165 | −1.297 | 63.300 | 1.00 | 88.45 | N |
| ATOM | 1389 | CA | HIS | H | 682 | 27.865 | −0.737 | 63.684 | 1.00 | 88.55 | C |
| ATOM | 1390 | CB | HIS | H | 682 | 27.832 | −0.360 | 65.171 | 1.00 | 85.93 | C |
| ATOM | 1391 | CG | HIS | H | 682 | 27.880 | −1.436 | 66.160 | 0.00 | 64.43 | C |
| ATOM | 1392 | ND1 | HIS | H | 682 | 27.266 | −2.644 | 65.905 | 0.00 | 63.40 | N |
| ATOM | 1393 | CE1 | HIS | H | 682 | 27.432 | −3.448 | 66.940 | 0.00 | 62.44 | C |
| ATOM | 1394 | NE2 | HIS | H | 682 | 28.131 | −2.805 | 67.858 | 0.00 | 62.45 | N |
| ATOM | 1395 | CD2 | HIS | H | 682 | 28.424 | −1.546 | 67.395 | 0.00 | 63.15 | C |
| ATOM | 1396 | C | HIS | H | 682 | 27.479 | 0.438 | 62.780 | 1.00 | 89.30 | C |
| ATOM | 1397 | O | HIS | H | 682 | 27.086 | 0.229 | 61.629 | 1.00 | 91.98 | O |
| ATOM | 1398 | N | LYS | H | 683 | 27.599 | 1.663 | 63.287 | 1.00 | 87.79 | N |
| ATOM | 1399 | CA | LYS | H | 683 | 27.226 | 2.847 | 62.515 | 1.00 | 86.12 | C |
| ATOM | 1400 | CB | LYS | H | 683 | 26.659 | 3.931 | 63.436 | 1.00 | 86.60 | C |
| ATOM | 1401 | CG | LYS | H | 683 | 27.536 | 4.339 | 64.548 | 0.00 | 62.87 | C |
| ATOM | 1402 | CD | LYS | H | 683 | 26.871 | 5.408 | 65.400 | 0.00 | 60.76 | C |
| ATOM | 1403 | CE | LYS | H | 683 | 27.791 | 5.878 | 66.517 | 0.00 | 59.28 | C |
| ATOM | 1404 | NZ | LYS | H | 683 | 28.156 | 4.774 | 67.450 | 0.00 | 58.13 | N |
| ATOM | 1405 | C | LYS | H | 683 | 28.393 | 3.406 | 61.704 | 1.00 | 84.50 | C |
| ATOM | 1406 | O | LYS | H | 683 | 28.196 | 3.936 | 60.607 | 1.00 | 82.74 | O |
| ATOM | 1407 | N | MET | H | 684 | 29.603 | 3.286 | 62.252 | 1.00 | 82.03 | N |
| ATOM | 1408 | CA | MET | H | 684 | 30.790 | 3.882 | 61.637 | 1.00 | 78.67 | C |
| ATOM | 1409 | CB | MET | H | 684 | 31.032 | 5.291 | 62.216 | 1.00 | 80.80 | C |
| ATOM | 1410 | CG | MET | H | 684 | 30.036 | 6.343 | 61.838 | 0.00 | 62.14 | C |
| ATOM | 1411 | SD | MET | H | 684 | 30.433 | 7.963 | 62.521 | 0.00 | 62.00 | S |
| ATOM | 1412 | CE | MET | H | 684 | 31.446 | 8.644 | 61.210 | 0.00 | 61.07 | C |
| ATOM | 1413 | C | MET | H | 684 | 32.067 | 3.009 | 61.717 | 1.00 | 71.78 | C |
| ATOM | 1414 | O | MET | H | 684 | 31.998 | 1.777 | 61.862 | 1.00 | 67.88 | O |
| ATOM | 1415 | N | ARG | H | 685 | 33.217 | 3.678 | 61.601 | 1.00 | 67.22 | N |
| ATOM | 1416 | CA | ARG | H | 685 | 34.535 | 3.046 | 61.587 | 1.00 | 64.23 | C |
| ATOM | 1417 | CB | ARG | H | 685 | 35.551 | 3.949 | 60.875 | 1.00 | 59.96 | C |
| ATOM | 1418 | CG | ARG | H | 685 | 35.121 | 4.488 | 59.520 | 0.00 | 49.91 | C |
| ATOM | 1419 | CD | ARG | H | 685 | 36.128 | 5.468 | 58.936 | 0.00 | 47.85 | C |
| ATOM | 1420 | NE | ARG | H | 685 | 35.769 | 5.887 | 57.583 | 0.00 | 46.03 | N |
| ATOM | 1421 | CZ | ARG | H | 685 | 35.962 | 5.154 | 56.491 | 0.00 | 45.16 | C |
| ATOM | 1422 | NH1 | ARG | H | 685 | 36.515 | 3.951 | 56.577 | 0.00 | 44.35 | N |
| ATOM | 1423 | NH2 | ARG | H | 685 | 35.601 | 5.627 | 55.305 | 0.00 | 44.31 | N |
| ATOM | 1424 | C | ARG | H | 685 | 35.027 | 2.700 | 62.992 | 1.00 | 62.41 | C |
| ATOM | 1425 | O | ARG | H | 685 | 34.920 | 3.501 | 63.926 | 1.00 | 58.60 | O |
| ATOM | 1426 | N | MET | H | 686 | 35.570 | 1.492 | 63.117 | 1.00 | 61.83 | N |
| ATOM | 1427 | CA | MET | H | 686 | 36.101 | 0.974 | 64.376 | 1.00 | 62.13 | C |
| ATOM | 1428 | CB | MET | H | 686 | 35.083 | 0.027 | 65.024 | 1.00 | 66.02 | C |
| ATOM | 1429 | CG | MET | H | 686 | 33.903 | 0.703 | 65.701 | 1.00 | 70.52 | C |
| ATOM | 1430 | SD | MET | H | 686 | 32.839 | −0.508 | 66.526 | 1.00 | 74.45 | S |
| ATOM | 1431 | CE | MET | H | 686 | 31.401 | −0.468 | 65.525 | 1.00 | 78.03 | C |
| ATOM | 1432 | C | MET | H | 686 | 37.427 | 0.227 | 64.177 | 1.00 | 58.89 | C |
| ATOM | 1433 | O | MET | H | 686 | 37.618 | −0.489 | 63.181 | 1.00 | 57.12 | O |
| ATOM | 1434 | N | VAL | H | 687 | 38.340 | 0.405 | 65.129 | 1.00 | 51.48 | N |
| ATOM | 1435 | CA | VAL | H | 687 | 39.564 | −0.388 | 65.182 | 1.00 | 42.04 | C |
| ATOM | 1436 | CB | VAL | H | 687 | 40.651 | 0.323 | 66.057 | 1.00 | 42.24 | C |
| ATOM | 1437 | CG1 | VAL | H | 687 | 40.212 | 0.435 | 67.513 | 1.00 | 43.67 | C |
| ATOM | 1438 | CG2 | VAL | H | 687 | 42.019 | −0.343 | 65.934 | 1.00 | 38.65 | C |
| ATOM | 1439 | C | VAL | H | 687 | 39.237 | −1.834 | 65.626 | 1.00 | 37.72 | C |
| ATOM | 1440 | O | VAL | H | 687 | 39.129 | −2.148 | 66.816 | 1.00 | 36.10 | O |
| ATOM | 1441 | N | LEU | H | 688 | 39.032 | −2.706 | 64.648 | 1.00 | 34.04 | N |
| ATOM | 1442 | CA | LEU | H | 688 | 38.747 | −4.107 | 64.946 | 1.00 | 33.08 | C |
| ATOM | 1443 | CB | LEU | H | 688 | 37.935 | −4.751 | 63.817 | 1.00 | 36.95 | C |
| ATOM | 1444 | CG | LEU | H | 688 | 36.421 | −4.599 | 63.951 | 1.00 | 46.04 | C |
| ATOM | 1445 | CD1 | LEU | H | 688 | 36.009 | −3.117 | 63.956 | 1.00 | 49.47 | C |
| ATOM | 1446 | CD2 | LEU | H | 688 | 35.727 | −5.366 | 62.846 | 1.00 | 44.20 | C |
| ATOM | 1447 | C | LEU | H | 688 | 39.976 | −4.958 | 65.238 | 1.00 | 28.88 | C |
| ATOM | 1448 | O | LEU | H | 688 | 39.870 | −6.019 | 65.862 | 1.00 | 31.94 | O |
| ATOM | 1449 | N | GLY | H | 689 | 41.135 | −4.504 | 64.782 | 1.00 | 27.49 | N |
| ATOM | 1450 | CA | GLY | H | 689 | 42.328 | −5.320 | 64.846 | 1.00 | 25.72 | C |
| ATOM | 1451 | C | GLY | H | 689 | 43.545 | −4.467 | 64.978 | 1.00 | 27.81 | C |
| ATOM | 1452 | O | GLY | H | 689 | 43.551 | −3.302 | 64.561 | 1.00 | 29.00 | O |
| ATOM | 1453 | N | VAL | H | 690 | 44.578 | −5.029 | 65.585 | 1.00 | 30.69 | N |
| ATOM | 1454 | CA | VAL | H | 690 | 45.855 | −4.333 | 65.682 | 1.00 | 24.58 | C |
| ATOM | 1455 | CB | VAL | H | 690 | 46.291 | −4.141 | 67.142 | 1.00 | 26.11 | C |
| ATOM | 1456 | CG1 | VAL | H | 690 | 47.683 | −3.452 | 67.213 | 1.00 | 27.16 | C |
| ATOM | 1457 | CG2 | VAL | H | 690 | 45.249 | −3.311 | 67.912 | 1.00 | 21.31 | C |
| ATOM | 1458 | C | VAL | H | 690 | 46.850 | −5.177 | 64.907 | 1.00 | 23.42 | C |
| ATOM | 1459 | O | VAL | H | 690 | 46.887 | −6.408 | 65.057 | 1.00 | 27.98 | O |
| ATOM | 1460 | N | ILE | H | 691 | 47.617 | −4.520 | 64.044 | 1.00 | 16.52 | N |
| ATOM | 1461 | CA | ILE | H | 691 | 48.549 | −5.203 | 63.154 | 1.00 | 15.42 | C |
| ATOM | 1462 | CB | ILE | H | 691 | 49.009 | −4.240 | 62.015 | 1.00 | 18.38 | C |
| ATOM | 1463 | CG1 | ILE | H | 691 | 47.803 | −3.732 | 61.187 | 1.00 | 19.32 | C |
| ATOM | 1464 | CD1 | ILE | H | 691 | 48.083 | −2.469 | 60.399 | 1.00 | 6.50 | C |
| ATOM | 1465 | CG2 | ILE | H | 691 | 50.049 | −4.921 | 61.111 | 1.00 | 16.69 | C |
| ATOM | 1466 | C | ILE | H | 691 | 49.748 | −5.669 | 63.981 | 1.00 | 19.23 | C |
| ATOM | 1467 | O | ILE | H | 691 | 50.346 | −4.891 | 64.747 | 1.00 | 16.17 | O |
| ATOM | 1468 | N | VAL | H | 692 | 50.087 | −6.938 | 63.811 | 1.00 | 15.25 | N |
| ATOM | 1469 | CA | VAL | H | 692 | 51.227 | −7.550 | 64.480 | 1.00 | 23.77 | C |
| ATOM | 1470 | CB | VAL | H | 692 | 50.763 | −8.478 | 65.632 | 1.00 | 22.78 | C |
| ATOM | 1471 | CG1 | VAL | H | 692 | 50.186 | −7.672 | 66.757 | 1.00 | 21.97 | C |
| ATOM | 1472 | CG2 | VAL | H | 692 | 49.724 | −9.503 | 65.148 | 1.00 | 20.19 | C |
| ATOM | 1473 | C | VAL | H | 692 | 52.004 | −8.385 | 63.449 | 1.00 | 27.22 | C |
| ATOM | 1474 | O | VAL | H | 692 | 51.381 | −8.929 | 62.529 | 1.00 | 26.79 | O |
| ATOM | 1475 | N | PRO | H | 693 | 53.333 | −8.496 | 63.582 | 1.00 | 29.00 | N |
| ATOM | 1476 | CA | PRO | H | 693 | 54.120 | −9.294 | 62.625 | 1.00 | 34.90 | C |
| ATOM | 1477 | CB | PRO | H | 693 | 55.551 | −9.192 | 63.156 | 1.00 | 30.45 | C |
| ATOM | 1478 | CG | PRO | H | 693 | 55.402 | −8.717 | 64.558 | 1.00 | 30.64 | C |
| ATOM | 1479 | CD | PRO | H | 693 | 54.187 | −7.881 | 64.615 | 1.00 | 27.24 | C |
| ATOM | 1480 | C | PRO | H | 693 | 53.683 | −10.733 | 62.665 | 1.00 | 38.95 | C |
| ATOM | 1481 | O | PRO | H | 693 | 53.104 | −11.164 | 63.654 | 1.00 | 42.74 | O |
| ATOM | 1482 | N | GLY | H | 694 | 53.933 | −11.469 | 61.601 | 1.00 | 46.23 | N |
| ATOM | 1483 | CA | GLY | H | 694 | 53.677 | −12.891 | 61.658 | 1.00 | 58.70 | C |
| ATOM | 1484 | C | GLY | H | 694 | 53.868 | −13.575 | 60.331 | 1.00 | 66.53 | C |
| ATOM | 1485 | O | GLY | H | 694 | 54.989 | −13.916 | 59.947 | 1.00 | 70.50 | O |
| ATOM | 1486 | N | ARG | H | 695 | 52.757 | −13.753 | 59.629 | 1.00 | 68.71 | N |
| ATOM | 1487 | CA | ARG | H | 695 | 52.726 | −14.511 | 58.392 | 1.00 | 73.37 | C |
| ATOM | 1488 | CB | ARG | H | 695 | 51.273 | −14.883 | 58.055 | 1.00 | 76.66 | C |
| ATOM | 1489 | CG | ARG | H | 695 | 50.617 | −15.827 | 59.071 | 1.00 | 77.65 | C |
| ATOM | 1490 | CD | ARG | H | 695 | 49.109 | −16.040 | 58.890 | 1.00 | 77.81 | C |
| ATOM | 1491 | NE | ARG | H | 695 | 48.703 | −16.558 | 57.574 | 1.00 | 78.43 | N |
| ATOM | 1492 | CZ | ARG | H | 695 | 48.977 | −17.778 | 57.105 | 1.00 | 80.25 | C |
| ATOM | 1493 | NH1 | ARG | H | 695 | 49.692 | −18.632 | 57.824 | 1.00 | 80.85 | N |
| ATOM | 1494 | NH2 | ARG | H | 695 | 48.542 | −18.149 | 55.908 | 1.00 | 80.06 | N |
| ATOM | 1495 | C | ARG | H | 695 | 53.391 | −13.745 | 57.244 | 1.00 | 73.05 | C |
| ATOM | 1496 | O | ARG | H | 695 | 52.706 | −13.176 | 56.389 | 1.00 | 76.76 | O |
| ATOM | 1497 | N | GLY | H | 696 | 54.725 | −13.730 | 57.239 | 1.00 | 71.20 | N |
| ATOM | 1498 | CA | GLY | H | 696 | 55.496 | −13.051 | 56.204 | 1.00 | 69.22 | C |
| ATOM | 1499 | C | GLY | H | 696 | 55.060 | −11.613 | 55.952 | 1.00 | 63.06 | C |
| ATOM | 1500 | O | GLY | H | 696 | 54.631 | −10.916 | 56.875 | 1.00 | 62.64 | O |
| ATOM | 1501 | N | CYS | H | 697 | 55.155 | −11.177 | 54.697 | 1.00 | 57.77 | N |
| ATOM | 1502 | CA | CYS | H | 697 | 54.820 | −9.798 | 54.317 | 1.00 | 50.35 | C |
| ATOM | 1503 | CB | CYS | H | 697 | 56.000 | −8.853 | 54.598 | 1.00 | 48.09 | C |
| ATOM | 1504 | SG | CYS | H | 697 | 56.068 | −7.287 | 53.691 | 1.00 | 49.44 | S |
| ATOM | 1505 | C | CYS | H | 697 | 54.363 | −9.716 | 52.871 | 1.00 | 44.48 | C |
| ATOM | 1506 | O | CYS | H | 697 | 55.172 | −9.789 | 51.951 | 1.00 | 44.30 | O |
| ATOM | 1507 | N | ALA | H | 698 | 53.047 | −9.586 | 52.699 | 1.00 | 41.22 | N |
| ATOM | 1508 | CA | ALA | H | 698 | 52.406 | −9.432 | 51.399 | 1.00 | 41.78 | C |
| ATOM | 1509 | CB | ALA | H | 698 | 52.817 | −8.120 | 50.748 | 1.00 | 42.30 | C |
| ATOM | 1510 | C | ALA | H | 698 | 52.661 | −10.624 | 50.470 | 1.00 | 45.03 | C |
| ATOM | 1511 | O | ALA | H | 698 | 52.957 | −10.457 | 49.286 | 1.00 | 45.92 | O |
| ATOM | 1512 | N | ILE | H | 699 | 52.539 | −11.822 | 51.039 | 1.00 | 46.84 | N |
| ATOM | 1513 | CA | ILE | H | 699 | 52.602 | −13.083 | 50.306 | 1.00 | 46.35 | C |
| ATOM | 1514 | CB | ILE | H | 699 | 53.087 | −14.260 | 51.225 | 1.00 | 47.68 | C |
| ATOM | 1515 | CG1 | ILE | H | 699 | 54.375 | −13.906 | 51.992 | 1.00 | 48.96 | C |
| ATOM | 1516 | CD1 | ILE | H | 699 | 55.605 | −13.522 | 51.113 | 1.00 | 52.26 | C |
| ATOM | 1517 | CG2 | ILE | H | 699 | 53.242 | −15.577 | 50.425 | 1.00 | 47.46 | C |
| ATOM | 1518 | C | ILE | H | 699 | 51.213 | −13.390 | 49.753 | 1.00 | 45.72 | C |
| ATOM | 1519 | O | ILE | H | 699 | 50.222 | −13.327 | 50.489 | 1.00 | 45.04 | O |
| ATOM | 1520 | N | PRO | H | 700 | 51.143 | −13.690 | 48.456 | 1.00 | 47.52 | N |
| ATOM | 1521 | CA | PRO | H | 700 | 49.871 | −13.995 | 47.786 | 1.00 | 45.58 | C |
| ATOM | 1522 | CB | PRO | H | 700 | 50.306 | −14.300 | 46.350 | 1.00 | 42.89 | C |
| ATOM | 1523 | CG | PRO | H | 700 | 51.624 | −13.559 | 46.196 | 1.00 | 43.15 | C |
| ATOM | 1524 | CD | PRO | H | 700 | 52.287 | −13.712 | 47.520 | 1.00 | 46.78 | C |
| ATOM | 1525 | C | PRO | H | 700 | 49.162 | −15.190 | 48.421 | 1.00 | 51.18 | C |
| ATOM | 1526 | O | PRO | H | 700 | 49.834 | −16.173 | 48.760 | 1.00 | 56.00 | O |
| ATOM | 1527 | N | ASN | H | 701 | 47.841 | −15.087 | 48.598 | 1.00 | 54.68 | N |
| ATOM | 1528 | CA | ASN | H | 701 | 47.021 | −16.115 | 49.255 | 1.00 | 57.17 | C |
| ATOM | 1529 | CB | ASN | H | 701 | 46.799 | −17.318 | 48.338 | 1.00 | 64.99 | C |
| ATOM | 1530 | CG | ASN | H | 701 | 45.865 | −17.008 | 47.197 | 1.00 | 70.83 | C |
| ATOM | 1531 | OD1 | ASN | H | 701 | 46.265 | −17.024 | 46.033 | 1.00 | 73.99 | O |
| ATOM | 1532 | ND2 | ASN | H | 701 | 44.612 | −16.713 | 47.523 | 1.00 | 71.80 | N |
| ATOM | 1533 | C | ASN | H | 701 | 47.580 | −16.579 | 50.589 | 1.00 | 54.20 | C |
| ATOM | 1534 | O | ASN | H | 701 | 47.729 | −17.774 | 50.840 | 1.00 | 56.38 | O |
| ATOM | 1535 | N | ARG | H | 702 | 47.908 | −15.609 | 51.429 | 1.00 | 52.09 | N |
| ATOM | 1536 | CA | ARG | H | 702 | 48.421 | −15.863 | 52.757 | 1.00 | 48.93 | C |
| ATOM | 1537 | CB | ARG | H | 702 | 49.934 | −15.957 | 52.710 | 1.00 | 50.15 | C |
| ATOM | 1538 | CG | ARG | H | 702 | 50.544 | −16.484 | 53.980 | 1.00 | 55.43 | C |
| ATOM | 1539 | CD | ARG | H | 702 | 51.879 | −15.868 | 54.288 | 1.00 | 62.13 | C |
| ATOM | 1540 | NE | ARG | H | 702 | 52.609 | −16.599 | 55.317 | 1.00 | 67.08 | N |
| ATOM | 1541 | CZ | ARG | H | 702 | 53.218 | −17.762 | 55.123 | 1.00 | 70.67 | C |
| ATOM | 1542 | NH1 | ARG | H | 702 | 53.181 | −18.358 | 53.933 | 1.00 | 69.85 | N |
| ATOM | 1543 | NH2 | ARG | H | 702 | 53.860 | −18.337 | 56.130 | 1.00 | 71.66 | N |
| ATOM | 1544 | C | ARG | H | 702 | 47.979 | −14.693 | 53.638 | 1.00 | 46.12 | C |
| ATOM | 1545 | O | ARG | H | 702 | 48.718 | −13.721 | 53.804 | 1.00 | 48.31 | O |
| ATOM | 1546 | N | PRO | H | 703 | 46.759 | −14.779 | 54.167 | 1.00 | 42.08 | N |
| ATOM | 1547 | CA | PRO | H | 703 | 46.144 | −13.680 | 54.922 | 1.00 | 40.90 | C |
| ATOM | 1548 | CB | PRO | H | 703 | 44.741 | −14.201 | 55.185 | 1.00 | 42.18 | C |
| ATOM | 1549 | CG | PRO | H | 703 | 44.902 | −15.675 | 55.184 | 1.00 | 42.59 | C |
| ATOM | 1550 | CD | PRO | H | 703 | 45.863 | −15.941 | 54.074 | 1.00 | 41.02 | C |
| ATOM | 1551 | C | PRO | H | 703 | 46.853 | −13.415 | 56.242 | 1.00 | 37.14 | C |
| ATOM | 1552 | O | PRO | H | 703 | 47.525 | −14.287 | 56.774 | 1.00 | 39.59 | O |
| ATOM | 1553 | N | GLY | H | 704 | 46.704 | −12.206 | 56.756 | 1.00 | 37.67 | N |
| ATOM | 1554 | CA | GLY | H | 704 | 47.362 | −11.828 | 57.993 | 1.00 | 33.96 | C |
| ATOM | 1555 | C | GLY | H | 704 | 46.485 | −12.049 | 59.209 | 1.00 | 34.72 | C |
| ATOM | 1556 | O | GLY | H | 704 | 45.248 | −11.987 | 59.143 | 1.00 | 29.54 | O |
| ATOM | 1557 | N | ILE | H | 705 | 47.161 | −12.332 | 60.314 | 1.00 | 33.89 | N |
| ATOM | 1558 | CA | ILE | H | 705 | 46.562 | −12.494 | 61.609 | 1.00 | 33.20 | C |
| ATOM | 1559 | CB | ILE | H | 705 | 47.285 | −13.660 | 62.326 | 1.00 | 38.30 | C |
| ATOM | 1560 | CG1 | ILE | H | 705 | 46.536 | −14.088 | 63.580 | 1.00 | 39.60 | C |
| ATOM | 1561 | CD1 | ILE | H | 705 | 46.670 | −13.094 | 64.734 | 1.00 | 42.56 | C |
| ATOM | 1562 | CG2 | ILE | H | 705 | 48.762 | −13.304 | 62.641 | 1.00 | 45.01 | C |
| ATOM | 1563 | C | ILE | H | 705 | 46.632 | −11.143 | 62.379 | 1.00 | 36.32 | C |
| ATOM | 1564 | O | ILE | H | 705 | 47.683 | −10.495 | 62.458 | 1.00 | 34.10 | O |
| ATOM | 1565 | N | PHE | H | 706 | 45.502 | −10.696 | 62.921 | 1.00 | 36.15 | N |
| ATOM | 1566 | CA | PHE | H | 706 | 45.461 | −9.404 | 63.613 | 1.00 | 31.79 | C |
| ATOM | 1567 | CB | PHE | H | 706 | 44.524 | −8.437 | 62.891 | 1.00 | 26.43 | C |
| ATOM | 1568 | CG | PHE | H | 706 | 44.970 | −8.082 | 61.515 | 1.00 | 26.51 | C |
| ATOM | 1569 | CD1 | PHE | H | 706 | 44.780 | −8.967 | 60.458 | 1.00 | 29.11 | C |
| ATOM | 1570 | CE1 | PHE | H | 706 | 45.207 | −8.645 | 59.172 | 1.00 | 24.07 | C |
| ATOM | 1571 | CZ | PHE | H | 706 | 45.814 | −7.426 | 58.946 | 1.00 | 23.81 | C |
| ATOM | 1572 | CE2 | PHE | H | 706 | 46.002 | −6.531 | 59.991 | 1.00 | 23.78 | C |
| ATOM | 1573 | CD2 | PHE | H | 706 | 45.583 | −6.863 | 61.266 | 1.00 | 27.92 | C |
| ATOM | 1574 | C | PHE | H | 706 | 44.947 | −9.652 | 65.004 | 1.00 | 32.49 | C |
| ATOM | 1575 | O | PHE | H | 706 | 44.068 | −10.472 | 65.180 | 1.00 | 32.64 | O |
| ATOM | 1576 | N | VAL | H | 707 | 45.470 | −8.959 | 66.006 | 1.00 | 34.31 | N |
| ATOM | 1577 | CA | VAL | H | 707 | 44.922 | −9.173 | 67.343 | 1.00 | 39.49 | C |
| ATOM | 1578 | CB | VAL | H | 707 | 45.840 | −8.617 | 68.460 | 1.00 | 41.78 | C |
| ATOM | 1579 | CG1 | VAL | H | 707 | 46.242 | −7.186 | 68.188 | 1.00 | 41.34 | C |
| ATOM | 1580 | CG2 | VAL | H | 707 | 45.152 | −8.736 | 69.798 | 1.00 | 43.17 | C |
| ATOM | 1581 | C | VAL | H | 707 | 43.480 | −8.626 | 67.409 | 1.00 | 34.10 | C |
| ATOM | 1582 | O | VAL | H | 707 | 43.239 | −7.500 | 67.026 | 1.00 | 31.18 | O |
| ATOM | 1583 | N | ARG | H | 708 | 42.525 | −9.448 | 67.828 | 1.00 | 33.60 | N |
| ATOM | 1584 | CA | ARG | H | 708 | 41.121 | −9.038 | 67.864 | 1.00 | 36.21 | C |
| ATOM | 1585 | CB | ARG | H | 708 | 40.213 | −10.265 | 67.997 | 1.00 | 37.91 | C |
| ATOM | 1586 | CG | ARG | H | 708 | 38.747 | −10.052 | 67.659 | 1.00 | 43.80 | C |
| ATOM | 1587 | CD | ARG | H | 708 | 37.990 | −11.333 | 67.339 | 1.00 | 49.59 | C |
| ATOM | 1588 | NE | ARG | H | 708 | 37.526 | −11.999 | 68.550 | 1.00 | 59.86 | N |
| ATOM | 1589 | CZ | ARG | H | 708 | 36.270 | −12.002 | 68.981 | 1.00 | 62.44 | C |
| ATOM | 1590 | NH1 | ARG | H | 708 | 35.320 | −11.374 | 68.294 | 1.00 | 62.01 | N |
| ATOM | 1591 | NH2 | ARG | H | 708 | 35.963 | −12.642 | 70.104 | 1.00 | 62.98 | N |
| ATOM | 1592 | C | ARG | H | 708 | 40.882 | −8.082 | 69.023 | 1.00 | 37.22 | C |
| ATOM | 1593 | O | ARG | H | 708 | 40.889 | −8.507 | 70.173 | 1.00 | 40.53 | O |
| ATOM | 1594 | N | VAL | H | 709 | 40.684 | −6.799 | 68.721 | 1.00 | 33.05 | N |
| ATOM | 1595 | CA | VAL | H | 709 | 40.445 | −5.792 | 69.752 | 1.00 | 37.68 | C |
| ATOM | 1596 | CB | VAL | H | 709 | 40.288 | −4.406 | 69.159 | 1.00 | 36.57 | C |
| ATOM | 1597 | CG1 | VAL | H | 709 | 40.044 | −3.405 | 70.252 | 1.00 | 35.92 | C |
| ATOM | 1598 | CG2 | VAL | H | 709 | 41.534 | −4.028 | 68.381 | 1.00 | 42.03 | C |
| ATOM | 1599 | C | VAL | H | 709 | 39.226 | −6.102 | 70.630 | 1.00 | 43.29 | C |
| ATOM | 1600 | O | VAL | H | 709 | 39.261 | −5.889 | 71.839 | 1.00 | 43.65 | O |
| ATOM | 1601 | N | ALA | H | 710 | 38.172 | −6.640 | 70.020 | 1.00 | 46.89 | N |
| ATOM | 1602 | CA | ALA | H | 710 | 36.971 | −7.069 | 70.751 | 1.00 | 47.05 | C |
| ATOM | 1603 | CB | ALA | H | 710 | 35.951 | −7.650 | 69.792 | 1.00 | 44.60 | C |
| ATOM | 1604 | C | ALA | H | 710 | 37.224 | −8.045 | 71.916 | 1.00 | 51.44 | C |
| ATOM | 1605 | O | ALA | H | 710 | 36.468 | −8.065 | 72.896 | 1.00 | 53.63 | O |
| ATOM | 1606 | N | TYR | H | 711 | 38.282 | −8.843 | 71.819 | 1.00 | 48.56 | N |
| ATOM | 1607 | CA | TYR | H | 711 | 38.644 | −9.756 | 72.896 | 1.00 | 48.31 | C |
| ATOM | 1608 | CB | TYR | H | 711 | 39.562 | −10.843 | 72.356 | 1.00 | 47.40 | C |
| ATOM | 1609 | CG | TYR | H | 711 | 39.760 | −11.995 | 73.289 | 1.00 | 48.74 | C |
| ATOM | 1610 | CD1 | TYR | H | 711 | 40.784 | −11.977 | 74.238 | 1.00 | 52.27 | C |
| ATOM | 1611 | CE1 | TYR | H | 711 | 40.977 | −13.048 | 75.112 | 1.00 | 55.27 | C |
| ATOM | 1612 | CZ | TYR | H | 711 | 40.138 | −14.157 | 75.024 | 1.00 | 56.78 | C |
| ATOM | 1613 | OH | TYR | H | 711 | 40.321 | −15.214 | 75.878 | 1.00 | 58.06 | O |
| ATOM | 1614 | CE2 | TYR | H | 711 | 39.112 | −14.202 | 74.080 | 1.00 | 55.51 | C |
| ATOM | 1615 | CD2 | TYR | H | 711 | 38.931 | −13.116 | 73.220 | 1.00 | 52.97 | C |
| ATOM | 1616 | C | TYR | H | 711 | 39.272 | −9.056 | 74.123 | 1.00 | 47.09 | C |
| ATOM | 1617 | O | TYR | H | 711 | 39.093 | −9.491 | 75.258 | 1.00 | 48.10 | O |
| ATOM | 1618 | N | TYR | H | 712 | 39.988 | −7.964 | 73.897 | 1.00 | 42.73 | N |
| ATOM | 1619 | CA | TYR | H | 712 | 40.665 | −7.263 | 74.983 | 1.00 | 43.38 | C |
| ATOM | 1620 | CB | TYR | H | 712 | 42.157 | −7.054 | 74.656 | 1.00 | 40.62 | C |
| ATOM | 1621 | CG | TYR | H | 712 | 42.848 | −8.353 | 74.336 | 1.00 | 38.63 | C |
| ATOM | 1622 | CD1 | TYR | H | 712 | 43.102 | −9.300 | 75.337 | 1.00 | 34.70 | C |
| ATOM | 1623 | CE1 | TYR | H | 712 | 43.713 | −10.496 | 75.048 | 1.00 | 35.95 | C |
| ATOM | 1624 | CZ | TYR | H | 712 | 44.079 | −10.769 | 73.739 | 1.00 | 37.72 | C |
| ATOM | 1625 | OH | TYR | H | 712 | 44.672 | −11.965 | 73.424 | 1.00 | 34.73 | O |
| ATOM | 1626 | CE2 | TYR | H | 712 | 43.836 | −9.851 | 72.735 | 1.00 | 40.39 | C |
| ATOM | 1627 | CD2 | TYR | H | 712 | 43.217 | −8.654 | 73.033 | 1.00 | 38.48 | C |
| ATOM | 1628 | C | TYR | H | 712 | 39.966 | −5.950 | 75.250 | 1.00 | 47.17 | C |
| ATOM | 1629 | O | TYR | H | 712 | 40.535 | −5.007 | 75.828 | 1.00 | 49.43 | O |
| ATOM | 1630 | N | ALA | H | 713 | 38.710 | −5.910 | 74.832 | 1.00 | 49.55 | N |
| ATOM | 1631 | CA | ALA | H | 713 | 37.892 | −4.719 | 74.940 | 1.00 | 49.88 | C |
| ATOM | 1632 | CB | ALA | H | 713 | 36.631 | −4.917 | 74.171 | 1.00 | 50.81 | C |
| ATOM | 1633 | C | ALA | H | 713 | 37.617 | −4.318 | 76.401 | 1.00 | 53.10 | C |
| ATOM | 1634 | O | ALA | H | 713 | 37.732 | −3.136 | 76.751 | 1.00 | 51.74 | O |
| ATOM | 1635 | N | LYS | H | 714 | 37.294 | −5.291 | 77.258 | 1.00 | 55.00 | N |
| ATOM | 1636 | CA | LYS | H | 714 | 37.090 | −4.987 | 78.677 | 1.00 | 57.78 | C |
| ATOM | 1637 | CB | LYS | H | 714 | 36.754 | −6.239 | 79.501 | 1.00 | 60.61 | C |
| ATOM | 1638 | CG | LYS | H | 714 | 35.606 | −7.053 | 78.975 | 0.00 | 51.91 | C |
| ATOM | 1639 | CD | LYS | H | 714 | 34.362 | −6.603 | 79.723 | 0.00 | 52.05 | C |
| ATOM | 1640 | CE | LYS | H | 714 | 33.327 | −7.716 | 79.800 | 0.00 | 51.42 | C |
| ATOM | 1641 | NZ | LYS | H | 714 | 33.838 | −8.912 | 80.527 | 0.00 | 51.68 | N |
| ATOM | 1642 | C | LYS | H | 714 | 38.346 | −4.309 | 79.195 | 1.00 | 56.01 | C |
| ATOM | 1643 | O | LYS | H | 714 | 38.290 | −3.160 | 79.641 | 1.00 | 54.83 | O |
| ATOM | 1644 | N | TRP | H | 715 | 39.475 | −5.019 | 79.077 | 1.00 | 55.39 | N |
| ATOM | 1645 | CA | TRP | H | 715 | 40.781 | −4.541 | 79.529 | 1.00 | 50.97 | C |
| ATOM | 1646 | CB | TRP | H | 715 | 41.917 | −5.509 | 79.106 | 1.00 | 54.63 | C |
| ATOM | 1647 | CG | TRP | H | 715 | 43.293 | −4.950 | 79.427 | 1.00 | 58.31 | C |
| ATOM | 1648 | CD1 | TRP | H | 715 | 43.906 | −4.939 | 80.652 | 1.00 | 60.90 | C |
| ATOM | 1649 | NE1 | TRP | H | 715 | 45.125 | −4.307 | 80.570 | 1.00 | 62.78 | N |
| ATOM | 1650 | CE2 | TRP | H | 715 | 45.329 | −3.888 | 79.280 | 1.00 | 61.61 | C |
| ATOM | 1651 | CD2 | TRP | H | 715 | 44.190 | −4.271 | 78.529 | 1.00 | 60.19 | C |
| ATOM | 1652 | CE3 | TRP | H | 715 | 44.150 | −3.946 | 77.162 | 1.00 | 60.11 | C |
| ATOM | 1653 | CZ3 | TRP | H | 715 | 45.236 | −3.261 | 76.597 | 1.00 | 57.21 | C |
| ATOM | 1654 | CH2 | TRP | H | 715 | 46.349 | −2.902 | 77.376 | 1.00 | 58.24 | C |
| ATOM | 1655 | CZ2 | TRP | H | 715 | 46.415 | −3.200 | 78.711 | 1.00 | 58.45 | C |
| ATOM | 1656 | C | TRP | H | 715 | 41.062 | −3.109 | 79.068 | 1.00 | 46.82 | C |
| ATOM | 1657 | O | TRP | H | 715 | 41.478 | −2.275 | 79.862 | 1.00 | 49.79 | O |
| ATOM | 1658 | N | ILE | H | 716 | 40.824 | −2.817 | 77.790 | 1.00 | 47.34 | N |
| ATOM | 1659 | CA | ILE | H | 716 | 41.084 | −1.476 | 77.263 | 1.00 | 44.52 | C |
| ATOM | 1660 | CB | ILE | H | 716 | 40.768 | −1.411 | 75.755 | 1.00 | 43.14 | C |
| ATOM | 1661 | CG1 | ILE | H | 716 | 41.607 | −2.429 | 74.998 | 1.00 | 44.11 | C |
| ATOM | 1662 | CD1 | ILE | H | 716 | 41.431 | −2.359 | 73.505 | 1.00 | 45.57 | C |
| ATOM | 1663 | CG2 | ILE | H | 716 | 41.024 | −0.014 | 75.195 | 1.00 | 37.55 | C |
| ATOM | 1664 | C | ILE | H | 716 | 40.263 | −0.452 | 78.038 | 1.00 | 42.89 | C |
| ATOM | 1665 | O | ILE | H | 716 | 40.740 | 0.632 | 78.355 | 1.00 | 39.70 | O |
| ATOM | 1666 | N | HIS | H | 717 | 39.030 | −0.819 | 78.363 | 1.00 | 50.49 | N |
| ATOM | 1667 | CA | HIS | H | 717 | 38.173 | 0.088 | 79.114 | 1.00 | 61.27 | C |
| ATOM | 1668 | CB | HIS | H | 717 | 36.687 | −0.255 | 78.962 | 1.00 | 67.94 | C |
| ATOM | 1669 | CG | HIS | H | 717 | 36.075 | 0.326 | 77.722 | 1.00 | 74.61 | C |
| ATOM | 1670 | ND1 | HIS | H | 717 | 35.342 | −0.423 | 76.827 | 1.00 | 78.42 | N |
| ATOM | 1671 | CE1 | HIS | H | 717 | 34.943 | 0.343 | 75.828 | 1.00 | 79.96 | C |
| ATOM | 1672 | NE2 | HIS | H | 717 | 35.414 | 1.562 | 76.029 | 1.00 | 80.40 | N |
| ATOM | 1673 | CD2 | HIS | H | 717 | 36.131 | 1.577 | 77.203 | 1.00 | 76.80 | C |
| ATOM | 1674 | C | HIS | H | 717 | 38.593 | 0.242 | 80.563 | 1.00 | 58.38 | C |
| ATOM | 1675 | O | HIS | H | 717 | 38.678 | 1.367 | 81.061 | 1.00 | 61.56 | O |
| ATOM | 1676 | N | LYS | H | 718 | 38.892 | −0.878 | 81.216 | 1.00 | 56.84 | N |
| ATOM | 1677 | CA | LYS | H | 718 | 39.420 | −0.852 | 82.582 | 1.00 | 58.80 | C |
| ATOM | 1678 | CB | LYS | H | 718 | 39.786 | −2.265 | 83.068 | 1.00 | 57.70 | C |
| ATOM | 1679 | CG | LYS | H | 718 | 38.637 | −3.203 | 83.011 | 0.00 | 49.62 | C |
| ATOM | 1680 | CD | LYS | H | 718 | 39.079 | −4.586 | 83.461 | 0.00 | 48.50 | C |
| ATOM | 1681 | CE | LYS | H | 718 | 37.936 | −5.587 | 83.385 | 0.00 | 47.99 | C |
| ATOM | 1682 | NZ | LYS | H | 718 | 36.799 | −5.212 | 84.272 | 0.00 | 47.43 | N |
| ATOM | 1683 | C | LYS | H | 718 | 40.617 | 0.097 | 82.712 | 1.00 | 58.39 | C |
| ATOM | 1684 | O | LYS | H | 718 | 40.727 | 0.824 | 83.707 | 1.00 | 58.62 | O |
| ATOM | 1685 | N | ILE | H | 719 | 41.484 | 0.105 | 81.693 | 1.00 | 57.94 | N |
| ATOM | 1686 | CA | ILE | H | 719 | 42.718 | 0.897 | 81.722 | 1.00 | 59.70 | C |
| ATOM | 1687 | CB | ILE | H | 719 | 43.839 | 0.254 | 80.832 | 1.00 | 56.18 | C |
| ATOM | 1688 | CG1 | ILE | H | 719 | 44.182 | −1.154 | 81.322 | 1.00 | 54.40 | C |
| ATOM | 1689 | CD1 | ILE | H | 719 | 44.789 | −1.209 | 82.744 | 1.00 | 54.78 | C |
| ATOM | 1690 | CG2 | ILE | H | 719 | 45.107 | 1.120 | 80.832 | 1.00 | 49.74 | C |
| ATOM | 1691 | C | ILE | H | 719 | 42.503 | 2.365 | 81.369 | 1.00 | 59.39 | C |
| ATOM | 1692 | O | ILE | H | 719 | 43.133 | 3.244 | 81.960 | 1.00 | 51.54 | O |
| ATOM | 1693 | N | ILE | H | 720 | 41.617 | 2.620 | 80.409 | 1.00 | 68.44 | N |
| ATOM | 1694 | CA | ILE | H | 720 | 41.333 | 3.988 | 79.966 | 1.00 | 78.79 | C |
| ATOM | 1695 | CB | ILE | H | 720 | 40.441 | 3.966 | 78.715 | 1.00 | 80.70 | C |
| ATOM | 1696 | CG1 | ILE | H | 720 | 41.252 | 3.533 | 77.496 | 1.00 | 79.85 | C |
| ATOM | 1697 | CD1 | ILE | H | 720 | 40.410 | 3.324 | 76.258 | 1.00 | 82.53 | C |
| ATOM | 1698 | CG2 | ILE | H | 720 | 39.839 | 5.345 | 78.464 | 1.00 | 83.82 | C |
| ATOM | 1699 | C | ILE | H | 720 | 40.717 | 4.875 | 81.066 | 1.00 | 82.59 | C |
| ATOM | 1700 | O | ILE | H | 720 | 40.815 | 6.109 | 81.015 | 1.00 | 80.57 | O |
| ATOM | 1701 | N | LEU | H | 721 | 40.089 | 4.237 | 82.052 | 1.00 | 86.87 | N |
| ATOM | 1702 | CA | LEU | H | 721 | 39.502 | 4.934 | 83.199 | 1.00 | 91.05 | C |
| ATOM | 1703 | CB | LEU | H | 721 | 37.974 | 4.754 | 83.206 | 1.00 | 93.07 | C |
| ATOM | 1704 | CG | LEU | H | 721 | 37.203 | 5.120 | 81.932 | 1.00 | 93.84 | C |
| ATOM | 1705 | CD1 | LEU | H | 721 | 35.794 | 4.523 | 81.964 | 1.00 | 93.43 | C |
| ATOM | 1706 | CD2 | LEU | H | 721 | 37.177 | 6.640 | 81.712 | 1.00 | 93.05 | C |
| ATOM | 1707 | C | LEU | H | 721 | 40.118 | 4.451 | 84.528 | 1.00 | 91.27 | C |
| ATOM | 1708 | O | LEU | H | 721 | 39.481 | 3.702 | 85.286 | 1.00 | 90.63 | O |
| ATOM | 1709 | N | THR | H | 722 | 41.359 | 4.876 | 84.793 | 1.00 | 89.38 | N |
| ATOM | 1710 | CA | THR | H | 722 | 42.096 | 4.495 | 86.010 | 1.00 | 85.56 | C |
| ATOM | 1711 | CB | THR | H | 722 | 42.375 | 2.960 | 86.037 | 1.00 | 85.39 | C |
| ATOM | 1712 | OG1 | THR | H | 722 | 42.795 | 2.567 | 87.351 | 1.00 | 84.41 | O |
| ATOM | 1713 | CG2 | THR | H | 722 | 43.554 | 2.584 | 85.131 | 1.00 | 85.32 | C |
| ATOM | 1714 | C | THR | H | 722 | 43.395 | 5.293 | 86.246 | 1.00 | 81.09 | C |
| ATOM | 1715 | O | THR | H | 722 | 43.987 | 5.856 | 85.320 | 1.00 | 76.45 | O |
| ATOM | 1716 | N | TYR | H | 723 | 43.748 | 5.418 | 87.509 | 0.00 | 57.31 | N |
| ATOM | 1717 | CA | TYR | H | 723 | 44.929 | 6.176 | 87.920 | 0.00 | 50.35 | C |
| ATOM | 1718 | CB | TYR | H | 723 | 45.043 | 6.189 | 89.447 | 0.00 | 47.26 | C |
| ATOM | 1719 | CG | TYR | H | 723 | 43.937 | 6.957 | 90.137 | 0.00 | 44.35 | C |
| ATOM | 1720 | CD1 | TYR | H | 723 | 43.952 | 8.351 | 90.182 | 0.00 | 43.30 | C |
| ATOM | 1721 | CE1 | TYR | H | 723 | 42.936 | 9.061 | 90.819 | 0.00 | 42.26 | C |
| ATOM | 1722 | CZ | TYR | H | 723 | 41.893 | 8.374 | 91.418 | 0.00 | 42.04 | C |
| ATOM | 1723 | OH | TYR | H | 723 | 40.887 | 9.070 | 92.049 | 0.00 | 41.79 | O |
| ATOM | 1724 | CE2 | TYR | H | 723 | 41.856 | 6.989 | 91.386 | 0.00 | 42.22 | C |
| ATOM | 1725 | CD2 | TYR | H | 723 | 42.875 | 6.289 | 90.748 | 0.00 | 43.33 | C |
| ATOM | 1726 | C | TYR | H | 723 | 46.246 | 5.701 | 87.307 | 0.00 | 47.60 | C |
| ATOM | 1727 | O | TYR | H | 723 | 46.303 | 4.663 | 86.645 | 0.00 | 47.18 | O |
| ATOM | 1728 | N | LYS | H | 724 | 47.300 | 6.479 | 87.543 | 0.00 | 44.39 | N |
| ATOM | 1729 | CA | LYS | H | 724 | 48.635 | 6.186 | 87.030 | 0.00 | 41.21 | C |
| ATOM | 1730 | CB | LYS | H | 724 | 49.600 | 7.321 | 87.399 | 0.00 | 40.56 | C |
| ATOM | 1731 | CG | LYS | H | 724 | 51.035 | 7.112 | 86.929 | 0.00 | 39.41 | C |
| ATOM | 1732 | CD | LYS | H | 724 | 51.921 | 8.300 | 87.280 | 0.00 | 38.63 | C |
| ATOM | 1733 | CE | LYS | H | 724 | 51.485 | 9.565 | 86.550 | 0.00 | 38.13 | C |
| ATOM | 1734 | NZ | LYS | H | 724 | 51.582 | 9.425 | 85.070 | 0.00 | 37.57 | N |
| ATOM | 1735 | C | LYS | H | 724 | 49.182 | 4.852 | 87.533 | 0.00 | 39.92 | C |
| ATOM | 1736 | O | LYS | H | 724 | 49.361 | 3.918 | 86.749 | 0.00 | 39.32 | O |
| ATOM | 1737 | N | VAL | H | 725 | 49.437 | 4.774 | 88.839 | 0.00 | 38.56 | N |
| ATOM | 1738 | CA | VAL | H | 725 | 49.972 | 3.572 | 89.483 | 0.00 | 37.28 | C |
| ATOM | 1739 | CB | VAL | H | 725 | 49.050 | 2.339 | 89.260 | 0.00 | 37.26 | C |
| ATOM | 1740 | CG1 | VAL | H | 725 | 49.634 | 1.105 | 89.937 | 0.00 | 37.30 | C |
| ATOM | 1741 | CG2 | VAL | H | 725 | 47.652 | 2.622 | 89.795 | 0.00 | 37.30 | C |
| ATOM | 1742 | C | VAL | H | 725 | 51.389 | 3.255 | 88.997 | 0.00 | 36.73 | C |
| ATOM | 1743 | O | VAL | H | 725 | 51.619 | 3.076 | 87.800 | 0.00 | 36.31 | O |
| ATOM | 1744 | N | PRO | H | 726 | 52.359 | 3.190 | 89.928 | 0.00 | 36.28 | N |
| ATOM | 1745 | CA | PRO | H | 726 | 53.766 | 2.898 | 89.627 | 0.00 | 35.98 | C |
| ATOM | 1746 | CB | PRO | H | 726 | 54.379 | 2.733 | 91.017 | 0.00 | 35.95 | C |
| ATOM | 1747 | CG | PRO | H | 726 | 53.594 | 3.708 | 91.830 | 0.00 | 35.98 | C |
| ATOM | 1748 | CD | PRO | H | 726 | 52.179 | 3.433 | 91.371 | 0.00 | 36.07 | C |
| ATOM | 1749 | C | PRO | H | 726 | 53.946 | 1.638 | 88.781 | 0.00 | 35.92 | C |
| ATOM | 1750 | O | PRO | H | 726 | 53.771 | 0.518 | 89.265 | 0.00 | 35.78 | O |
| ATOM | 1751 | N | GLN | H | 727 | 54.285 | 1.839 | 87.511 | 0.00 | 35.80 | N |
| ATOM | 1752 | CA | GLN | H | 727 | 54.488 | 0.741 | 86.570 | 0.00 | 35.76 | C |
| ATOM | 1753 | CB | GLN | H | 727 | 54.357 | 1.250 | 85.131 | 0.00 | 35.71 | C |
| ATOM | 1754 | CG | GLN | H | 727 | 53.028 | 1.927 | 84.827 | 0.00 | 35.80 | C |
| ATOM | 1755 | CD | GLN | H | 727 | 52.963 | 2.492 | 83.421 | 0.00 | 35.80 | C |
| ATOM | 1756 | OE1 | GLN | H | 727 | 51.915 | 2.464 | 82.773 | 0.00 | 35.77 | O |
| ATOM | 1757 | NE2 | GLN | H | 727 | 54.090 | 3.004 | 82.938 | 0.00 | 35.77 | N |
| ATOM | 1758 | C | GLN | H | 727 | 55.848 | 0.076 | 86.769 | 0.00 | 35.83 | C |
| ATOM | 1759 | O | GLN | H | 727 | 55.954 | −0.942 | 87.455 | 0.00 | 35.81 | O |
| ATOM | 1760 | N | SER | H | 728 | 56.885 | 0.663 | 86.173 | 0.00 | 35.71 | N |
| ATOM | 1761 | CA | SER | H | 728 | 58.244 | 0.140 | 86.273 | 0.00 | 35.95 | C |
| ATOM | 1762 | CB | SER | H | 728 | 58.415 | −1.072 | 85.351 | 0.00 | 35.96 | C |
| ATOM | 1763 | OG | SER | H | 728 | 59.713 | −1.632 | 85.474 | 0.00 | 36.27 | O |
| ATOM | 1764 | C | SER | H | 728 | 59.262 | 1.217 | 85.909 | 0.00 | 35.73 | C |
| ATOM | 1765 | O | SER | H | 728 | 59.169 | 1.843 | 84.853 | 0.00 | 35.53 | O |
| ATOM | 1766 | O | HOH | E | 1 | 53.926 | −1.766 | 47.459 | 1.00 | 21.71 | O |
| ATOM | 1767 | O | HOH | E | 2 | 45.945 | 0.405 | 66.551 | 1.00 | 17.96 | O |
| ATOM | 1768 | O | HOH | E | 3 | 67.967 | 1.160 | 70.108 | 1.00 | 18.62 | O |
| ATOM | 1769 | O | HOH | E | 4 | 47.256 | 10.386 | 59.382 | 1.00 | 40.82 | O |
| ATOM | 1770 | O | HOH | E | 5 | 60.365 | −4.292 | 45.784 | 1.00 | 26.90 | O |
| ATOM | 1771 | O | HOH | E | 6 | 38.165 | 5.011 | 71.349 | 1.00 | 36.22 | O |
| ATOM | 1772 | O | HOH | E | 7 | 45.163 | −12.931 | 51.739 | 1.00 | 43.37 | O |
| ATOM | 1773 | O | HOH | E | 8 | 47.666 | −11.890 | 51.054 | 1.00 | 46.32 | O |
| ATOM | 1774 | O | HOH | E | 9 | 50.588 | 1.409 | 60.961 | 1.00 | 14.01 | O |
| ATOM | 1775 | O | HOH | E | 10 | 52.733 | 2.919 | 60.297 | 1.00 | 23.84 | O |
| ATOM | 1776 | O | HOH | E | 11 | 60.521 | 11.735 | 64.427 | 1.00 | 30.61 | O |
| ATOM | 1777 | O | HOH | E | 12 | 64.517 | −1.753 | 78.579 | 1.00 | 14.15 | O |
| ATOM | 1778 | O | HOH | E | 13 | 52.422 | 6.657 | 50.079 | 1.00 | 28.67 | O |
| ATOM | 1779 | O | HOH | E | 14 | 37.949 | −7.017 | 67.321 | 1.00 | 38.28 | O |
| ATOM | 1780 | O | HOH | E | 15 | 35.362 | −9.881 | 65.897 | 1.00 | 41.66 | O |
| ATOM | 1781 | O | HOH | E | 16 | 53.334 | 2.088 | 54.023 | 1.00 | 24.05 | O |
| ATOM | 1782 | O | HOH | E | 17 | 59.048 | 1.391 | 51.927 | 1.00 | 15.67 | O |
| ATOM | 1783 | O | HOH | E | 18 | 42.391 | 7.790 | 51.113 | 1.00 | 28.93 | O |
| ATOM | 1784 | O | HOH | E | 19 | 58.321 | 10.330 | 75.738 | 1.00 | 35.52 | O |
| ATOM | 1785 | O | HOH | E | 20 | 52.208 | −0.076 | 49.074 | 1.00 | 19.70 | O |
| ATOM | 1786 | O | HOH | E | 21 | 36.437 | 8.112 | 69.617 | 1.00 | 25.44 | O |
| ATOM | 1787 | O | HOH | E | 22 | 42.332 | 9.761 | 52.871 | 1.00 | 35.23 | O |
| ATOM | 1788 | O | HOH | E | 23 | 61.517 | 8.942 | 69.123 | 1.00 | 30.48 | O |
| ATOM | 1789 | O | HOH | E | 24 | 60.321 | 8.726 | 60.693 | 1.00 | 28.09 | O |
| ATOM | 1790 | O | HOH | E | 25 | 48.749 | −8.114 | 61.252 | 1.00 | 28.20 | O |
| ATOM | 1791 | O | HOH | E | 26 | 61.093 | −0.206 | 49.928 | 1.00 | 17.70 | O |
| ATOM | 1792 | O | HOH | E | 27 | 38.209 | 15.773 | 65.622 | 1.00 | 38.54 | O |
| ATOM | 1793 | O | HOH | E | 28 | 37.214 | 13.461 | 67.090 | 1.00 | 40.61 | O |
| ATOM | 1794 | O | HOH | E | 29 | 62.600 | 4.633 | 50.239 | 1.00 | 36.81 | O |
| ATOM | 1795 | O | HOH | E | 30 | 50.405 | −10.315 | 43.970 | 1.00 | 35.54 | O |
| ATOM | 1796 | O | HOH | E | 31 | 38.433 | −7.655 | 77.519 | 1.00 | 53.37 | O |
| ATOM | 1797 | O | HOH | E | 32 | 42.000 | 10.749 | 77.405 | 1.00 | 45.80 | O |
| ATOM | 1798 | O | HOH | E | 33 | 35.445 | 6.005 | 64.479 | 1.00 | 48.73 | O |

TABLE 6
Atomic Coordinates of HGF β Secondary Structural Features

| | | | | | | Amino | | | | | | | | | | | | |
|---|---|---|---|---|---|---|---|---|---|---|---|---|---|---|---|---|---|---|
| Structural | | Feature | | | | Acid | | Amino Acid | | | | | | | | | | |
| Feature | | Number | | | | Types | | Numbers | | | | | | | | | | |
| HELIX | 1 | | 1 | ARG | H | 533 | CYS | H | 535 | 5 | | | | | | | | |
| HELIX | 2 | | 2 | LEU | H | 541 | ASP | H | 543 | 5 | | | | | | | | |
| HELIX | 3 | | 3 | ASN | H | 639 | LYS | H | 641 | 5 | | | | | | | | |
| HELIX | 4 | | 4 | VAL | H | 709 | ILE | H | 720 | 5 | | | | | | | | |
| SHEET | 1 | A | 7 | GLN | H | 563 | ASN | H | 566 | 0 | | | | | | | | |
| SHEET | 2 | A | 7 | TYR | H | 544 | LEU | H | 548 | −1 | N | LEU | H | 548 | O | GLN | H | 563 |
| SHEET | 3 | A | 7 | MET | H | 508 | TYR | H | 513 | −1 | N | ARG | H | 512 | O | GLU | H | 545 |
| SHEET | 4 | A | 7 | HIS | H | 517 | LYS | H | 525 | −1 | N | GLY | H | 521 | O | VAL | H | 509 |
| SHEET | 5 | A | 7 | TRP | H | 528 | ALA | H | 532 | −1 | N | LEU | H | 530 | O | SER | H | 522 |
| SHEET | 6 | A | 7 | LEU | H | 579 | LEU | H | 584 | −1 | N | MET | H | 582 | O | VAL | H | 529 |
| SHEET | 7 | A | 7 | VAL | H | 567 | TYR | H | 572 | −1 | N | VAL | H | 571 | O | LEU | H | 581 |
| SHEET | 1 | B | 6 | ARG | H | 630 | TYR | H | 635 | 0 | | | | | | | | |
| SHEET | 2 | B | 6 | SER | H | 611 | GLY | H | 616 | −1 | N | GLY | H | 616 | O | ARG | H | 630 |
| SHEET | 3 | B | 6 | PRO | H | 676 | GLU | H | 680 | −1 | N | VAL | H | 678 | O | SER | H | 613 |
| SHEET | 4 | B | 6 | ARG | H | 685 | ILE | H | 691 | −1 | N | GLY | H | 689 | O | LEU | H | 677 |
| SHEET | 5 | B | 6 | GLY | H | 704 | ARG | H | 708 | −1 | N | VAL | H | 707 | O | VAL | H | 690 |
| SHEET | 6 | B | 6 | GLU | H | 656 | ALA | H | 659 | −1 | N | ALA | H | 659 | O | GLY | H | 704 |

TABLE 7
Amino Acid Sequence of HGF β (SEQ ID NO: 1)

| | |
|---|---|
| 495 | |
| VVNGIPTRTNIGWMVSLRYRNKHICGGSLIKESWVLTARQCFPSRD | 540 |
| 541 | |
| LKDYEAWLGIHDVHGRGDEKCKQVLNVSQLVYGPEGSDLV | 580 |
| 581 | |
| LMKLARPAVLDDFVSTIDLPNYGSTIPEKTSCSVYGWGYT | 620 |
| 621 | |
| GLINYDGLLRVAHLYIMGNEKCSQHHRGKVTLNESEICAG | 660 |
| 661 | |
| AEKIGSGPCEGDYGGPLVGEQHKMRMVLGVIVPGRGCAIP | 700 |
| 701 | |
| NRPGIFVRVAYYAKWIHKIILTYKVPQS | 728 |

TABLE 8

Amino Acid Sequence of ECD of Met Receptor
(SEQ ID NO: 4)

| | | |
|---|---|---|
| | ECKEAL AKSEMNVNMK YQLPNFTAET PIQNVILHEH | 60 |
| 61 | HIFLGATNYI YVLNEEDLQK VAEYKTGPVL EHPDCFPCQD | 120 |
| | CSSKANLSGG VWKDNINMAL | |
| 121 | VVDTYYDDQL LSCGSVNRGT CQRHVFPHNH TADIQSEVHC | 180 |
| | IFSPQIEEPS QCPDCVVSAL | |
| 181 | GAKVLSSVKD RFLNFFVGNT LNSSYFPDHP LHSISVRRLK | 240 |
| | ETKDGFMFLT DQSYIDVLPE | |
| 241 | FRDSYPIKYV HAFESNNFIY FLTVQRETLD AQTFHTRIIR | 300 |
| | FCSINSGLHS YMEMPLECLL | |
| 301 | TEKRKKRSTK KEVFNILQAA YVSKPGAQLA RQIGASLNDD | 360 |
| | LLFGVFAQSK PDSAEPMDRS | |
| 361 | AMCAFPIKYV NDFFNKIVNK NNYRCLQHFY GPNHEHCFNR | 420 |
| | TLLRNSSGCE ARRDEYRTEF | |
| 421 | TTALQRVDLF MGQFSEVLLT SISTFIKGDL TIANLGTSEG | 480 |
| | RFMQVVVSRS GPSTPHVNFL | |
| 481 | LDSHPVSPEV JVEHTLNQNG YTLVITGKKI TKIPLNGLGC | 540 |
| | RHFQSCSQCL SAPPFVQCGW | |
| 541 | CHDKCVRSEE CLSGTWTQQI CLPAIYKVFP NSAPLEGGTR | 600 |
| | LTICGWDFGF RRNNKFDLKK | |
| 601 | TRVLLGNESC TLTLSESTMN TLKCTVGPAM NKHFNMSIII | 660 |
| | SNGHGTTQYS TFSYVDPVIT | |
| 661 | SISPKYGPMA GGTLLTLTGN YLNSGNSRHI SIGGKTCTLK | 720 |
| | SVSNSILECY TPAQTISTEF | |
| 721 | AVKLKIDLAN RETSIFSYRE DPIVYEIHPT KSFISGGSTI | 780 |
| | TGVGKNLNSV SVPRMVLNVH | |
| 781 | EAGRNFTVAC QHRSNSEIIC CTTPSLQQLN LQLPLKTKAF | 840 |
| | FMLDGILSKY FDLIYVHNPV | |
| 841 | FKPFEKPVMI SMGNENVLEI KGNDIDPEAV KGEVLKVGNK | 900 |
| | SCENIHLHSE AVLCTVPNDL | |
| 901 | LKLNSELNIE WKQAISSTVL GKVIVQPDQN | |

TABLE 9
Amino Acid Sequence of Native HGF β (SEQ ID NO: 5)

| | |
|---|---|
| 495 | |
| VVNGIPTRTNIGWMVSLRYRNKHICGGSLIKESWVLTARQCFPSRD | 540 |
| 541 | |
| LKDYEAWLGIHDVHGRGDEKCKQVLNVSQLVYGPEGSDLV | 580 |
| 581 | |
| LMKLARPAVLDDFVSTIDLPNYGCTIPEKTSCSVYGWGYT | 620 |
| 621 | |
| GLINYDGLLRVAHLYIMGNEKCSQHHRGKVTLNESEICAG | 660 |
| 661 | |
| AEKIGSGPCEGDYGGPLVCEQHKMRMVLGVIVPGRGCAIP | 700 |
| 701 | |
| NRPGIFVRVAYYAKWIHKIILTYKVPQS | 728 |

TABLE 10
Amino Acid Sequence of Native HGF (SEQ ID NO: 6)

MWVTKLLPALLLQHVLLHLLLLPIAIPYAEGQRKRRNTIHEFKKSAKTTLIKI
DPALKIKTKKVNTADQCANRCTRNKGLPFTCKAFVFDKARKQCLWFPFNS
MSSGVKKEFGHEFDLYENKDYIRNCIIGKGRSYKGTVSITKSGIKCQPWSSMI
PHEHSFLPSSYRGKDLQENYCRNPRGEEGGPWCFTSNPEVRYEVCDIPQCSE
VECMTCNGESYRGLMDHTESGKICQRWDHQTPHRHKFLPERYPDKGFDDN
YCRNPDGQPRPWGYTLDPHTRWEYCAIKTCADNTMNDTDVPLETTECIQG
QGEGYRGTVNTIWNGIPCQRWDSQYPHEHDMTPENFKCKDLRENYCRNPD
GSESPWCFTTDPNIRVGYCSQIPNCDMSHGQDCYRGNGKNYMGNLSQTRSG
LTCSMWDKNMEDLHRHIFWEPDASKLNENYCRNPDDDAHGPWCYTGNPLI
PWDYGPISRCEGDTTPTIVNLDHPVISCAKTKQLRVVNGIPTRTNIGWMVSL
RYRNKHICGGSLIKESWVLTARQCFPSRDLKDYEAWLGIHDVHGRGDEKCK
QVLNVSQLVYGPEGSDLVLMKLARPAVLDDFVSTIDLPNYGCTIPEKTSCSV
YGWGYTGLINYDGLLRVAHLYIMGNEKCSQHHRGKVTLNESEICAGAEKIG
SGPCEGDYGGPLVCEQHKMRMVLGVIVPGRGCAIPNRPGIFVRVAYYAKWI
HKIILTYKVPQS

### LIST OF REFERENCES

Angeloni, D., Danilkovitch-Miagkova, A., Miagkov, A., Leonard, E. J., and Lerman, M. I. (2003). The soluble sema domain of the RON receptor inhibits MSP-induced receptor activation. J. Biol. Chem. (in press).
Berman, H. M., Westbrook, J., Feng, Z., Gilliland, G., Bhat, T. N., Weissig, H., Shindyalov, I. N., and Bourne, P. E. (2000). The protein data bank. Nucl. Acid Res. 28, 235-242.
Birchmeier, C., Birchmeier, W., Gherardi, E., and Vande Woude, G. F. (2003). Met, metastasis, motility and more. Nature Rev. Mol. Cell Biol. 4, 915-925.
Boose, J. A., Kuismanen, E., Gerard, R., Sambrook, J., and Gething, M.-J. (1989). The single-chain form of tissue-type plasminogen activator has catalytic activity: Studies with a mutant enzyme that lacks the cleavage site. Biochemistry 28, 635-643.
Bottaro, D. P., Rubin, J. S., Faletto, D. L., Chan, A. M., Kmiecik, T. E., Vande Woude, G. F., and Aaronson, S. A. (1991). Identification of the hepatocyte growth factor receptor as the c-met proto-oncogene product. Science 251, 802-804.
Broze Jr., G. J. (2001). Protein Z-dependent regulation of coagulation. Thromb. Haemost. 86, 8-13.
CCP4 (1994). The CCP4 suite: Programs for protein crystallography. Acta Crystallogr D50, 760-763.
Cohen, G. H. (1997). Align—a program to superimpose protein coordinates, accounting for insertions and deletions. J. Appl. Crystallog. 30, 1160-1161.
Cooper, C. S., Park, M., Blair, D. G., Tainsky, M. A., Huebner, K., Croce, C. M., and Vande Woude, G. F. (1984). Molecular cloning of a new transforming gene from a chemically transformed human cell line. Nature 311, 29-33.
Danilkovitch, A., Miller, M., and Leonard, E. J. (1999). Interaction of macrophage-stimulating protein with its receptor. J. Biol. Chem. 274, 29937-29943.
Danilkovitch-Miagkova, A., and Zbar, B. (2002). Dysregulation of Met receptor tyrosine kinase activity in invasive tumors. J. Clin. Invest. 109, 863-867.
Dennis, M. S., Eigenbrot, C., Skelton, N. J., Ultsch, M. H., Santell, L., Dwyer, M. A., O'Connell, M. P., and Lazarus, R. A. (2000). Peptide exosite inhibitors of factor VIIa as anticoagulants. Nature 404, 465-470.
Derksen, P. W. B., Keehnen, R. M. J., Evers, L. M., van Oers, M. H. J., Spaargaren, M., and Pals, S. T. (2002). Cell surface proteoglycan syndecan-1 mediates hepatocyte growth factor binding and promotes Met signaling in multiple myeloma. Blood 99, 1405-1410.
Di Cera, E., Guinto, E. R., Vindigni, A., Dang, Q. D., Ayala, Y. M., Wuyi, M., and Tulinsky, A. (1995). The Na⁺ binding site of thrombin. J. Biol. Chem. 270, 22089-22092.
Dickinson, C. D., Kelly, C. R., and Ruf, W. (1996). Identification of surface residues mediating tissue factor binding and catalytic function of the serine protease factor VIIa. Proc. Natl. Acad. Sci. USA 93, 14379-14384.
Donate, L. E., Gherardi, E., Srinivasan, N., Sowdhamini, R., Aparicio, S., and Blundell, T. L. (1994). Molecular evolution and domain structure of plasminogen-related growth factors (HGF/SF and HGF1/MSP). Protein Sci. 3, 2378-2394.
Drain, J., Bishop, J. R., and Hajduk, S. L. (2001). Haptoglobin-related protein mediates Trypanosome lytic factor binding to Trypanosomes. J. Biol. Chem. 276, 30254-30260.
Gherardi, E., Youles, M. E., Miguel, R. N., Blundell, T. L., Iamele, L., Gough, J., Bandyopadhyay, A., Hartmann, G., and Butler, P. J. G. (2003). Functional map and domain structure of MET, the product of the c-met protooncogene and receptor for hepatocyte growth factor/scatter factor. Proc. Natl. Acad. Sci. USA 100, 12039-12044.
Hartmann, G., Naldini, L., Weidner, K. M., Sachs, M., Vigna, E., Comoglio, P. M., and Birchmeier, W. (1992). A functional domain in the heavy chain of scatter factor/hepatocyte growth factor binds the c-Met receptor and induces cell dissociation but not mitogenesis. Proc. Natl. Acad. Sci. USA 89, 11574-11578.
Hartmann, G., Prospero, T., Brinkmann, V., Ozcelik, O., Winter, G., Hepple, J., Batley, S., Bladt, F., Sachs, M., Birchmeier, C., Birchmeier, W., and Gherardi, E. (1998). Engineered mutants of HGF/SF with reduced binding to heparan sulphate proteoglycans, decreased clearance and enhanced activity in vivo. Curr. Biol. 8, 125-134.
Hedstrom, L. (2002). Serine protease mechanism and specificity. Chem. Rev. 102, 4501-4523.
Huber, R., and Bode, W. (1978). Structural basis of the activation and action of trypsin. Acc Chem. Res. 11, 114-122.
Kunkel, T. A. (1985). Rapid and efficient site-specific mutagenesis without phenotypic selection. Proc. Natl. Acad. Sci. USA 82, 488-492.
Kurosky, A., Barnett, D. R., Lee, T. H., Touchstone, B., Hay, R. E., Arnott, M. S., Bowman, B. H., and Fitch, W. M. (1980). Covalent structure of human haptoglobin: A serine protease homolog. Proc. Natl. Acad. Sci. USA 77, 3388-3392.
Lijnen, H. R., Van Hoef, B., Nelles, L., and Collen, D. (1990). Plasminogen activation with single-chain urokinase-type plasminogen activator (scu-PA). Studies with active site mutagenized plasminogen (Ser⁷⁴⁰→Ala) and plasmin-resistant scu-PA (Lys¹⁵⁸→Glu). J. Biol. Chem. 265, 5232-5236.
Lokker, N. A., Mark, M. R., Luis, E. A., Bennett, G. L., Robbins, K. A., Baker, J. B., and Godowski, P. J. (1992). Structure-function analysis of hepatocyte growth factor: identification of variants that lack mitogenic activity yet retain high affinity receptor binding. EMBO J. 11, 2503-2510.
Lokker, N. A., Presta, L. G., and Godowski, P. J. (1994). Mutational analysis and molecular modeling of the N-terminal kringle-containing domain of hepatocyte growth factor identifies amino acid side chains important for interaction with the c-Met receptor. Protein Eng. 7, 895-903.
Ma, P. C., Maulik, G., Christensen, J., and Salgia, R. (2003). c-Met: structure, functions and potential for therapeutic inhibition. Cancer Metastasis Rev. 22, 309-325.
Malkowski, M. G., Martin, P. D., Guzik, J. C., and Edwards, B. F. P. (1997). The co-crystal structure of unliganded bovine α-thrombin and prethrombin-2: Movement of the Tyr-Pro-Pro-Trp segment and active site residues upon ligand binding. Protein Sci. 6, 1438-1448.
Mark, M. R., Lokker, N. A., Zioncheck, T. F., Luis, E. A., and Godowski, P. J. (1992). Expression and characterization of hepatocyte growth factor receptor-IgG fusion proteins. Effects of mutations in the potential proteolytic cleavage site on processing and ligand binding. J. Biol. Chem. 267, 26166-26171.
Matsumoto, K., Kataoka, H., Date, K., and Nakamura, T. (1998). Cooperative interaction between α- and β-chains of hepatocyte growth factor on c-Met receptor confers ligand-induced receptor tyrosine phosphorylation and multiple biological responses. J. Biol. Chem. 273, 22913-22920.
Maulik, G., Shrikhande, A., Kijima, T., Ma, P. C., Morrison, P. T., and Salgia, R. (2002). Role of the hepatocyte growth factor receptor, c-Met, in oncogenesis and potential for therapeutic inhibition. Cytokine Growth Factor Rev. 13, 41-59.
McRee, D. E. (1999). Practical Protein Crystallography, 2nd ed. (San Diego, Academic Press).
Miller, M., and Leonard, E. J. (1998). Mode of receptor binding and activation by plasminogen-related growth factors. FEBS Lett. 429, 1-3.
Murshudov, G. N., Vagin, A. A., and Dodson, E. J. (1997). Refinement of macromolecular structures by the maximum-likelihood method. Acta Crystallogr D53, 240-255.
Naka, D., Ishii, T., Yoshiyama, Y., Miyazawa, K., Hara, H., Hishida, T., and Kitamura, N. (1992). Activation of hepatocyte growth factor by proteolytic conversion of single chain form to a heterodimer. J. Biol. Chem. 267, 20114-20119.
Nakamura, T., Nishizawa, T., Hagiya, M., Seki, T., Shimonishi, M., Sugimura, A., Tashiro, K., and Shimizu, S. (1989). Molecular cloning and expression of human hepatocyte growth factor. Nature 342, 440-443.
Naldini, L., Tamagnone, L., Vigna, E., Sachs, M., Hartmann, G., Birchmeier, W., Daikuhara, Y., Tsubouchi, H., Blasi, F., and Comoglio, P. M. (1992). Extracellular proteolytic cleavage by urokinase is required for activation of hepatocyte growth factor/scatter factor. EMBO J. 11, 4825-4833.
Navaza, J. (1994). AMoRe: an automated package for molecular replacement. Acta Crystallogr A50, 157-163.
Okigaki, M., Komada, M., Uehara, Y., Miyazawa, K., and Kitamura, N. (1992). Functional characterization of human hepatocyte growth factor mutants obtained by deletion of structural domains. Biochemistry 31, 9555-9561.
Otwinowski, Z., and Minor, W. (1996). Processing of X-ray diffraction data collected in oscillation mode. Methods Enzymol. 276, 307-326.
Parry, M. A., Fernandez-Catalan, C., Bergner, A., Huber, R., Hopfner, K. P., Schlott, B., Guhrs, K. H., and Bode, W. (1998). The ternary microplasmin-staphylokinase-microplasmin complex is a proteinase-cofactor-substrate complex in action. Nat. Struct. Biol. 5, 917-923.
Peek, M., Moran, P., Mendoza, N., Wickramasinghe, D., and Kirchhofer, D. (2002). Unusual proteolytic activation of pro-hepatocyte growth factor by plasma kallikrein and coagulation factor XIa. J. Biol. Chem. 277, 47804-47809.
Peisach, E., Wang, J., de los Santos, T., Reich, E., and Ringe, D. (1999). Crystal structure of the proenzyme domain of plasminogen. Biochemistry 38, 11180-11188.
Perona, J. J., and Craik, C. S. (1995). Structural basis of substrate specificity in the serine proteases. Protein Sci. 4, 337-360.
Rawlings, N. D., O'Brien, E., and Barrett, A. J. (2002). MEROPS: the protease database. Nucl. Acid Res. 30, 343-346.
Renatus, M., Engh, R. A., Stubbs, M. T., Huber, R., Fischer, S., Kohnert, U., and Bode, W. (1997). Lysine 156 promotes the anomalous proenzyme activity of tPA: X-ray crystal structure of single-chain human tPA. EMBO J. 16, 4797-4805.
Stubbs, M. T., and Bode, W. (1993). A player of many parts: The spotlight falls on thrombin's structure. Thromb. Res. 69, 1-58.
Trusolino, L., and Comoglio, P. M. (2002). Scatter-factor and semaphorin receptors: Cell signaling for invasive growth. Nature Rev. Cancer 2, 289-300.
Tsiang, M., Jain, A. K., Dunn, K. E., Rojas, M. E., Leung, L. L. K., and Gibbs, C. S. (1995). Functional mapping of the surface residues of human thrombin. J. Biol. Chem. 270, 16854-16863.
Vijayalakshmi, J., Padmanabhan, K. P., Mann, K. G., and Tulinsky, A. (1994). The isomorphic structures of prethrombin2, hirugen-, and PPACK-thrombin: Changes accompanying activation and exosite binding to thrombin. Protein Sci. 3, 2254-2271.
Wang, D., Bode, W., and Huber. R (1985). Bovine chymotrypsinogen A: X-ray crystal structure analysis and refinement of a new crystal form at 1.8 Å resolution. J. Mol. Biol. 185, 595-624.
Wang, M. H., Julian, F. M., Breathnach, R., Godowski, P. J., Takehara, T., Yoshikawa, W., Hagiya, M., and Leonard, E. J. (1997). Macrophage stimulating protein (MSP) binds to its receptor via the MSP β chain. J. Biol. Chem. 272, 16999-17004.

1. A crystal comprising a human hepatocyte growth factor beta chain (HGF β) comprising SEQ ID NO:1 or conservative substitutions thereof or a portion thereof.

2. Crystalline Hepatocyte Growth Factor β.

3. A crystal of HGF β of claim 1 having a space group symmetry of P3₁21 and comprising a unit cell having the dimensions of a, b and c, where a=b and is about 63.7 Å, and c is about 135 Å.

4. A crystal of HGF β of claim 1 having the three dimensional coordinates of Table 5.

5. The crystal of claim 1, wherein the crystal diffracts x-rays for the determination of atomic coordinates to a resolution of 5 Å or better.

6. A composition comprising a crystal of claim 1, and a carrier.

7. A molecule or molecular complex comprising at least a portion of the HGF β binding site of a polypeptide having an amino acid sequence of SEQ ID NO:1 or conservative substitutions thereof, wherein the binding site comprises at least one amino acid residue selected from the group consisting of 513, 516, 533, 534, 537-539, 578, 619, 647, 656, 668-670, 673, 692-697, 699, 702, 705, 707, and mixtures thereof, and the binding site is defined by a set of points having a root mean square deviation of less than about 0.70 Å from points representing the backbone atoms of the amino acids as represented by the structure coordinates listed in Table 5.

8. (canceled)

9. A three-dimensional configuration of points wherein at least a portion of the points are derived from structure coordinates of Table 5 representing locations of the backbone atoms of at least the core amino acids defining the HGF β binding site for Met.

10. The three-dimensional configuration of points of claim 9 displayed as a holographic image, a stereodiagram, a model, or a computer-displayed image, wherein the HGF β domain forms a crystal having the space group symmetry P3₁21.

11. A machine-readable data storage medium comprising a data storage material encoded with machine-readable data, wherein a machine programmed with instructions for using such data displays a graphical three-dimensional representation of at least one molecule or molecular complex comprising at least a portion of a HGP β binding site for Met, the binding site defined by a set of points having a root mean square deviation of less than about 0.05 Å from points representing the atoms of the amino acids as represented by the structure coordinates listed in Table 5.

12. (canceled)

13. A method for obtaining structural information about a molecule or molecular complex comprising applying at least a portion of the HGF β structure coordinates of a crystal of claim 5 to an X-ray diffraction pattern of the molecule or molecular complex's crystal structure to generate a three-dimensional electron density map of at least a portion of the molecule or molecular complex.

14-16. (canceled)

17. A method of assessing agents that are antagonists or agonists or HGF and/or HGF β comprising:
a) applying at least a portion of the crystallography coordinates of a crystal of claim 5 to a computer algorithm that generates a 3 dimensional model of HGF β suitable for designing molecules that are antagonists or agonists; and
b) searching a molecular structure database to identify potential antagonists or agonists of HGF β.

18. The method of claim 17, further comprising:
(a) synthesizing or obtaining the antagonist or agonist;
(b) contacting the antagonist or agonist with HGF β and selecting the antagonist or agonist that modulates the activity of HGF β.

19-20. (canceled)

21. The method of claim 17, wherein the binding site comprises at least one or more or all amino acid residues in a position comprising 513, 516, 533, 534, 537-539, 578, 619, 647, 656, 668-670, 673, 692-697, 699, 702, 705, or 707, or mixtures thereof.

22. The method of claim 21, wherein the amino acids in the HGF β binding site comprise one or more or all of amino acid residues in a position comprising 513, 534, 537, 578, 619, 621, 673, 692 to 697, 699 or 701, or mixtures thereof.

23. A molecule or molecular complex comprising at least a portion of the HGF β active site of a polypeptide having an amino acid of SEQ ID NO:1 or conservative substitutions thereof, wherein the active site comprises at least one amino acid residue selected from the group consisting of 532-536, 574 to 579, 637 to 655, 667 to 673, 690-697, and mixtures thereof, the active site defined by a set of points having a root mean square deviation of less than about 0.70 Å from points representing the backbone atoms of the amino acids as represented by the structure coordinates listed in Table 5.

24-32. (canceled)

33. A molecule or molecular complex comprising at least a portion of the HGF β activation domain of a polypeptide having an amino acid sequence of SEQ ID NO:1 or conservative amino acid substitutions thereof, wherein the activation domain comprises at least one amino acid residue selected from the group consisting of 495 to 498, 502 to 505, 553 to 562, 618 to 627, 637 to 655, 660 to 672, 697 to 704, and mixtures thereof, and the activation domain is defined by a set of points having a root mean square deviation of less than about 0.70 Å from points representing the backbone atoms of the amino acids as represented by the structure coordinates listed in Table 5.

34-39. (canceled)

40. A molecule or molecular complex comprising at least a portion of the HGF β tunnel of a polypeptide having an amino acid sequence of SEQ ID NO:1 or conservative substitutions thereof, wherein the tunnel comprises at least one amino acid residue selected from the group consisting of 634, 673, 660 to 670, 693 to 706, and mixtures thereof, and the tunnel is defined by a set of points having a root mean square deviation of less than about 0.70 Å from points representing the backbone atoms of the amino acids as represented by the structure coordinates listed in Table 5.

41-46. (canceled)

47. A molecule or molecular complex comprising at least a portion of the HGF β dimerization region of a polypeptide having an amino acid sequence of SEQ ID NO:1 or conservative substitutions thereof, wherein the dimerization region comprises at least one amino acid residue selected from the group consisting of 495 to 502, 617 to 630, 660 to 670, 700, and mixtures thereof, and the dimerization region is defined by a set of points having a root mean square deviation of less than about 0.70 Å from points representing the backbone atoms of the amino acids as represented by the structure coordinates listed in Table 5.

48-51. (canceled)


SEQUENCE LISTING (14 sequences)

SEQ ID NO: 1 - protein, 234 aa, Homo sapiens
VVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGSTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 2 - DNA, 25 bp, Artificial Sequence
  Oligonucleotide
cctaattatggatccacaattcctg

SEQ ID NO: 3 - DNA, 35 bp, Artificial Sequence
  Oligonucleotide
caaaacgaaacaattggaagttgtaaatgggattc

SEQ ID NO: 4 - protein, 906 aa, Homo sapiens
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQN

SEQ ID NO: 5 - protein, 234 aa, Homo sapiens
VVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 6 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 7 - protein, 256 aa, Artificial Sequence
  proHGF-B
NLDHPVISCAKTKQLEVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQSHHHHHH

SEQ ID NO: 8 - protein, 728 aa, Artificial Sequence
  Non-cleavable HGF
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHAHIFWEPDASKLNENYCRNPDDDAHGPWCYTGNPLIPWDYCPISRCEGDTTPTIVNLDHPVISCAKTKQLEVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 9 - protein, 40 aa, Artificial Sequence
  HGF partial sequence
PVISCAKTKQLRVVNGIPTRTNIGWMVSLRYRNKHICGGS

SEQ ID NO: 10 - protein, 48 aa, Artificial Sequence
  Plasmin partial sequence
PSFDCGKPQVEPKKCPGRVVGGCVAHPHSWPWQVSLRTRFGMHFCGGT

SEQ ID NO: 11 - protein, 47 aa, Artificial Sequence
  MSP partial sequence
QFEKCGKRVDRLDQRRSKLRVVGGHPGNSPWTVSLRNRQGQHFCGGS

SEQ ID NO: 12 - protein, 51 aa, Artificial Sequence
  t-PA partial sequence
SCSTCGLRQYSQPQFRIKGGLFADIASHPWQAAIFAKHRRSPGERFLCGGI

SEQ ID NO: 13 - protein, 45 aa, Artificial Sequence
  Chymotrypsin partial sequence
CGVPAIQPVLSGLSRIVNGEEAVPGSWPWQVSLQDKTGFHFCGGS

SEQ ID NO: 14 - protein, 6 aa, Artificial Sequence
  Poly-His6 Tag
HHHHHH